(19) United States
(12) Patent Application Publication
(10) Pub. No.: US 2023/0113866 A1
(43) Pub. Date: Apr. 13, 2023

(54) Methods Of Treating Clonal Hematopoiesis Of Indeterminate Potential (CHIP) With Lymphocyte Antigen 75 (LY75), Cluster Of Differentiation 164 (CD164), Or Poly(ADP-Ribose) Polymerase 1 (PARP1) Inhibitors

(71) Applicant: Regeneron Pharmaceuticals, Inc., Tarrytown, NY (US)

(72) Inventors: Eric Jorgenson, Tarrytown, NY (US); Michael Kessler, Tarrytown, NY (US); Amy Damask, Tarrytown, NY (US); Sean O'Keeffe, Tarrytown, NY (US); Ryan White, Tarrytown, NY (US); David Glass, Tarrytown, NY (US)

(21) Appl. No.: 17/931,396

(22) Filed: Sep. 12, 2022

Related U.S. Application Data
(60) Provisional application No. 63/243,564, filed on Sep. 13, 2021.
(60) Provisional application No. 63/271,171, filed on Oct. 24, 2021.

(51) Int. Cl. C12N 15/113; C12Q 1/6883; A61K 31/7105

(52) U.S. Cl. CPC C12N 15/113; C12Q 1/6883; A61K 31/7105; C12Q 2600/156; C12N 2310/14; C12N 2310/20

(57) ABSTRACT

Methods of treating subjects having clonal hematopoiesis of indeterminate potential (CHIP) with lymphocyte antigen 75 (LY75), Cluster of Differentiation 164 (CD164), or Poly(ADP-Ribose) Polymerase 1 (PARP1) inhibitors, and methods of identifying subjects having an increased risk of developing CHIP are provided herein.

### REFERENCE TO SEQUENCE LISTING

This application includes a Sequence Listing filed electronically as an XML file named 381203582SEQ, created on Sep. 11, 2022, with a size of 1,547 kilobytes. The Sequence Listing is incorporated herein by reference.

### FIELD

The present disclosure relates generally to the treatment of subjects having clonal hematopoiesis of indeterminate potential (CHIP) with Lymphocyte Antigen 75 (LY75), Cluster of Differentiation 164 (CD164), or Poly(ADP-Ribose) Polymerase 1 (PARP1) inhibitors, and methods of identifying subjects having an increased risk of developing CHIP.

### BACKGROUND

CHIP is a genetically defined phenotype reflecting age-related changes to hematopoietic stem cells (HSCs). As a person ages, their HSCs accumulate mutations as a result of DNA replication error and DNA damage repair (so called somatic mutations, such as those acquired after birth). Thus, prevalence rises with age and is roughly 10% among persons aged 70 to 80. Patients undergoing molecular genetic investigation for cytopenia (anemia, leukopenia, thrombocytopenia) are the most likely to be given this diagnosis. Some of these mutations confer growth advantages, which result in: increased proliferation of these cells relative to other cells, increase in frequency of these mutations, and accumulation of additional mutations that drive neoplastic changes. A subset of genes are strongly recurrently mutated along with clonal hematopoiesis; these are considered “CHIP genes” and they include: DNA Methyltransferase 3 Alpha (DNMT3A), Tet Methylcytosine Dioxygenase 2 (TET2), ASXL Transcriptional Regulator 1 (ASXL1), Janus kinase 2 (JAK2), and Splicing factor 3B subunit 1 (SF3B1). CpG=>TpG mutations are very common in CHIP. In addition to the identification in blood DNA of specific recurrent mutations, the clinical definition of CHIP requires the absence of dysplasia and leukemia (<20% blasts). CHIP is associated with increased risk of hematologic cancers, such as myeloid or lymphoid neoplasia, and with increased risk of atherosclerotic cardiovascular disease, such as coronary heart disease, myocardial infarction, and severe calcified aortic valve stenosis.

LY75 is an endocytic receptor that captures antigens from the extracellular space and directs them to a specialized antigen-processing compartment for antigen processing, presentation, and cross-presentation. LY75 can cause reduced proliferation of B-lymphocytes. LY75 is expressed on human dendritic cells, monocytes, B cells, T cells, NK cells, and thymic epithelial cells.

CD164 is an adhesive glycoprotein expressed by HSCs and bone marrow stromal cells that acts as a regulator of hematopoiesis. CD164 belongs to the sialomucin family of secreted or membrane-associated mucins that regulate proliferation, adhesion, and migration of HSCs.

PARP1 is a DNA repair protein involved in PAR polymerization, and recruitments an array of repair molecules to support single and double strand repair, and chromatin remodeling in the context of NER. PARP1 modifies various nuclear proteins by poly(ADP-ribosyl)ation of glutamate, aspartate, serine, or tyrosine residues. The modification is dependent on DNA and is involved in the regulation of various important cellular processes such as differentiation, proliferation, and tumor transformation and also in the regulation of the molecular events involved in the recovery of cells from DNA damage.

### SUMMARY

The present disclosure provides methods of preventing or reducing the development of CHIP in a subject, the methods comprising administering an LY75 inhibitor, a CD164 inhibitor, or a PARP1 inhibitor, or any combination thereof, to the subject.

The present disclosure also provides methods of treating a subject with a therapeutic agent that prevents or reduces development of CHIP, wherein the subject has CHIP or is at risk of developing CHIP, the methods comprising the steps of: determining whether the subject has an LY75 variant nucleic acid molecule, a CD164 variant nucleic acid molecule, and/or a PARP1 variant nucleic acid molecule by: obtaining or having obtained a biological sample from the subject; and performing or having performed a sequence analysis on the biological sample to determine if the subject has a genotype comprising the LY75 variant nucleic acid molecule, the CD164 variant nucleic acid molecule, and/or the PARP1 variant nucleic acid molecule; and administering or continuing to administer the therapeutic agent that prevents or reduces development of CHIP in a standard dosage amount to a subject that is LY75 reference, CD164 reference, and/or PARP1 reference, and/or administering an LY75 inhibitor, a CD164 inhibitor, or a PARP1 inhibitor, or any combination thereof, to the subject; administering or continuing to administer the therapeutic agent that prevents or reduces development of CHIP in an amount that is the same as or less than a standard dosage amount to a subject that is heterozygous for the LY75 variant nucleic acid molecule, the CD164 variant nucleic acid molecule, and/or the PARP1 variant nucleic acid molecule, and/or administering an LY75 inhibitor, a CD164 inhibitor, or a PARP1 inhibitor, or any combination thereof, to the subject; or administering or continuing to administer the therapeutic agent that prevents or reduces development of CHIP in an amount that is the same as or less than a standard dosage amount to a subject that is homozygous for the LY75 variant nucleic acid molecule, the CD164 variant nucleic acid molecule, and/or the PARP1 variant nucleic acid molecule; wherein the presence of a genotype having the LY75 variant nucleic acid molecule, the CD164 variant nucleic acid molecule, and/or the PARP1 variant nucleic acid molecule indicates the subject has a decreased risk of developing CHIP.

The present disclosure also provides methods of identifying a subject having an increased risk of developing CHIP, the methods comprising: determining or having determined the presence or absence of an LY75 variant nucleic acid molecule, a CD164 variant nucleic acid molecule, and/or a PARP1 variant nucleic acid molecule in a biological sample obtained from the subject; wherein: when the subject is LY75 reference, CD164 reference, and/or PARP1 reference, then the subject has an increased risk of developing CHIP; and when the subject is heterozygous or homozygous for the LY75 variant nucleic acid molecule, the CD164 variant nucleic acid molecule, and/or the PARP1 variant nucleic acid molecule, then the subject has a decreased risk of developing CHIP.

The present disclosure also provides therapeutic agents that prevent or reduce CHIP for use in the prevention or reduction of CHIP in a subject identified as having: an LY75 variant genomic nucleic acid molecule, a CD164 variant genomic nucleic acid molecule, and/or a PARP1 variant genomic nucleic acid molecule; an LY75 variant mRNA molecule, a CD164 variant mRNA molecule, and/or a PARP1 variant mRNA molecule; or an LY75 variant cDNA molecule, a CD164 variant cDNA molecule, and/or a PARP1 variant cDNA molecule.

The present disclosure also provides an LY75 inhibitor for use in the prevention or reduction of CHIP in a subject that: a) is reference for an LY75 genomic nucleic acid molecule, an LY75 mRNA molecule, or an LY75 cDNA molecule; or b) is heterozygous for: i) an LY75 variant genomic nucleic acid molecule; ii) an LY75 variant mRNA molecule; or iii) an LY75 variant cDNA molecule.

The present disclosure also provides a CD164 inhibitor for use in the prevention or reduction of CHIP in a subject that: a) is reference fora CD164 genomic nucleic acid molecule, a CD164 mRNA molecule, or a CD164 cDNA molecule; or b) is heterozygous for: i) a CD164 variant genomic nucleic acid molecule; ii) a CD164 variant mRNA molecule; or iii) a CD164 variant cDNA molecule.

The present disclosure also provides a PARP1 inhibitor for use in the prevention or reduction of CHIP in a subject that: a) is reference fora PARP1 genomic nucleic acid molecule, a PARP1 mRNA molecule, or a PARP1 cDNA molecule; or b) is heterozygous for: i) a PARP1 variant genomic nucleic acid molecule; ii) a PARP1 variant mRNA molecule; or iii) a PARP1 variant cDNA molecule.

The present disclosure also provides methods of identifying a subject at risk of developing lung cancer, the method comprising: determining or having determined the presence or absence of one or more CHIP somatic mutations in DNA Methyltransferase 3 Alpha (DNMT3A) and/or ASXL Transcriptional Regulator 1 (ASXL1) in a biological sample obtained from the subject; wherein: when the subject has a CHIP somatic mutation in DNMT3A and/or ASXL1, the subject has an increased risk of developing lung cancer; and when the subject does not have a CHIP somatic mutation in DNMT3A and/or ASXL1, the subject does not have an increased risk of developing lung cancer.

### BRIEF DESCRIPTION OF THE DRAWINGS

The accompanying figures, which are incorporated in and constitute a part of this specification, illustrate several features of the present disclosure.

The patent or application file contains at least one drawing executed in color. Copies of this patent or patent application publication with color drawing(s) will be provided by the Office upon request and payment of the necessary fee.

FIG. 1 shows LY75 burden masks analysis of CHIP risk.

FIG. 2 shows the effect of the ly75 locus stratified by CHIP gene mutation carrier.

FIG. 3 shows CHIP minus malignant blood LY75-CD302 masks.

FIG. 4 shows that the PARP 1:226367601:A:C missense variant significantly associated with CHIP.

FIG. 5 shows PARP missense variants significantly associate with CHIP.

FIG. 6 shows that CHIP carriers are at an elevated risk of developing solid tumors.

FIG. 7 shows functional modeling of identified CHIP-G WAS locus; Panels A and B show systematic tracking of HDR and INDEL percentages in PARPi treated cells; cells were treated with PARPi for 8 days and then subjected to Sanger sequencing across each locus to determine population dynamics; (Panel A) RPE-1 hTERT cells harboring CRISPR mediated knock-in of DNMT3A-R882H allele; (Panel B) CRISPR-mediated TET2-deficieny in HEK293T cells.

FIG. 8 shows Forest plots reflecting the protective associations of two LY75 missense variants (r578446341-A, r5147820690-T) with the DNMT3A-CHIPoverall CHIP phenotype in the UKB and GHS cohorts.

### DESCRIPTION

Various terms relating to aspects of the present disclosure are used throughout the specification and claims. Such terms are to be given their ordinary meaning in the art, unless otherwise indicated. Other specifically defined terms are to be construed in a mamer consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or Alternately phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human. In some embodiments, the human is a patient under the care of a physician.

It has been observed in accordance with the present disclosure that an LY75 variant nucleic acid molecule, a CD164 variant nucleic acid molecule, and a PARP1 variant nucleic acid molecule (whether these variations are homozygous or heterozygous in a particular subject) associate with a decreased risk of developing CHIP or CHIP-related disorders. The identification by the present disclosure of the association between additional variants and gene burden masks indicates that one or more of LY75, CD164, and PARP1 themselves (rather than linkage disequilibrium with variants in another gene) are responsible for a protective effect in CHI) and CHIP-related disorders.

Therefore, subjects that are LY75 reference, or heterozygous for an LY75 variant nucleic acid molecule, may be treated with an LY75 inhibitor; subjects that are CD164 reference, or heterozygous for a CD164 variant nucleic acid molecule, may be treated with a CD164 inhibitor; and subjects that are PARP1 reference, or heterozygous for a PARP1 variant nucleic acid molecules, may be treated with a PARP1 inhibitor, such that CHIP is prevented or inhibited and CHIP-related disorders are inhibited or prevented, the symptoms thereof are reduced or prevented, and/or development of symptoms is repressed or prevented. It is also believed that such subjects having CHIP may further be treated with therapeutic agents that treat or inhibit CHIP or CHIP-related disorders.

For purposes of the present disclosure, any particular subject, such as a human, can be categorized as having one of three LY75 genotypes: i) LY75 reference; ii) heterozygous for an LY75 variant nucleic acid molecule; or iii) homozygous for an LY75 variant nucleic acid molecule. A subject is LY75 reference when the subject does not have a copy of an LY75 variant nucleic acid molecule. A subject is heterozygous for an LY75 variant nucleic acid molecule when the subject has a single copy of an LY75 variant nucleic acid molecule.

In any of the embodiments described herein, an LY75 variant nucleic acid molecule is any nucleic acid molecule (such as, a genomic nucleic acid molecule, an mRNA molecule, or a cDNA molecule) that is a missense variant, a splice-site variant, a stop-gain variant, a start-loss variant, a stop-loss variant, a frameshift variant, an in-frame indel variant, or a variant that encodes a truncated LY75 polypeptide. In any of the embodiments described herein, an LY75 variant nucleic acid molecule can also be any nucleic acid molecule (such as, a genomic nucleic acid molecule, an mRNA molecule, or a cDNA molecule) that encodes an LY75 polypeptide having a partial loss-of-function, a complete loss-of-function, a predicted partial loss-of-function, or a predicted complete loss-of-function. A subject who has an LY75 polypeptide having a partial loss-of-function (or predicted partial loss-of-function) is hypomorphic for LY75. A subject is homozygous for an LY75 variant nucleic acid molecule when the subject has two copies (same or different) of an LY75 variant nucleic acid molecule.

For purposes of the present disclosure, any particular subject, such as a human, can be categorized as having one of three CD164 genotypes: i) CD164 reference; ii) heterozygous for a CD164 variant nucleic acid molecule; or iii) homozygous for a CD164 variant nucleic acid molecule. A subject is CD164 reference when the subject does not have a copy of a CD164 variant nucleic acid molecule. A subject is heterozygous for a CD164 variant nucleic acid molecule when the subject has a single copy of a CD164 variant nucleic acid molecule.

In any of the embodiments described herein, a CD164 variant nucleic acid molecule is any nucleic acid molecule (such as, a genomic nucleic acid molecule, an mRNA molecule, or a cDNA molecule) that is a missense variant, a splice-site variant, a stop-gain variant, a start-loss variant, a stop-loss variant, a frameshift variant, an in-frame indel variant, or a variant that encodes a truncated CD164 polypeptide. In any of the embodiments described herein, a CD164 variant nucleic acid molecule can also be any nucleic acid molecule (such as, a genomic nucleic acid molecule, an mRNA molecule, or a cDNA molecule) that encodes a CD164 polypeptide having a partial loss-of-function, a complete loss-of-function, a predicted partial loss-of-function, or a predicted complete loss-of-function. A subject who has a CD164 polypeptide having a partial loss-of-function (or predicted partial loss-of-function) is hypomorphic for CD164. A subject is homozygous for a CD164 variant nucleic acid molecule when the subject has two copies (same or different) of a CD164 variant nucleic acid molecule.

For purposes of the present disclosure, any particular subject, such as a human, can be categorized as having one of three PARP1 genotypes: i) PARP1 reference; ii) heterozygous for a PARP1 variant nucleic acid molecule; or iii) homozygous for a CD164 variant nucleic acid molecule. A subject is PARP1 reference when the subject does not have a copy of a PARP1 variant nucleic acid molecule. A subject is heterozygous for a PARP1 variant nucleic acid molecule when the subject has a single copy of a PARP1 variant nucleic acid molecule.

In any of the embodiments described herein, a PARP1 variant nucleic acid molecule is any nucleic acid molecule (such as, a genomic nucleic acid molecule, an mRNA molecule, or a cDNA molecule) that is a missense variant, a splice-site variant, a stop-gain variant, a start-loss variant, a stop-loss variant, a frameshift variant, an in-frame indel variant, or a variant that encodes a truncated PARP1 polypeptide. In any of the embodiments described herein, a PARP1 variant nucleic acid molecule can also be any nucleic acid molecule (such as, a genomic nucleic acid molecule, an mRNA molecule, or a cDNA molecule) that encodes a PARP1 polypeptide having a partial loss-of-function, a complete loss-of-function, a predicted partial loss-of-function, or a predicted complete loss-of-function. A subject who has a PARP1 polypeptide having a partial loss-of-function (or predicted partial loss-of-function) is hypomorphic for PARP1. A subject is homozygous for a PARP1 variant nucleic acid molecule when the subject has two copies (same or different) of a PARP1 variant nucleic acid molecule.

For subjects that are genotyped or determined to be LY75, CD164, and PARP1 reference, such subjects have an increased risk of developing CHIP and CHIP-related disorders, such as a hematologic cancer, a myeloid neoplasia, a lymphoid neoplasia, an atherosclerotic cardiovascular disease, a coronary heart disease, a myocardial infarction, and/or a severe calcified aortic valve stenosis. For subjects that are genotyped or determined to be either LY75, CD164, and PARP1 reference or heterozygous for one or more LY75, CD164, or PARP1 variant nucleic acid molecules, such subject or subjects can be treated with an LY75 inhibitor, a CD164 inhibitor, or a PARP1 inhibitor, or any combination thereof.

In any of the embodiments described herein, the subject in whom CHIP is prevented or reduced by administering an LY75 inhibitor, a CD164 inhibitor, or a PARP1 inhibitor, or any combination thereof, can be anyone at risk for developing CHIP including, but not limited to, subjects with CHIP-related disorders. In addition, in some embodiments, the subject is at risk of developing CHIP. In some embodiments, administering an LY75 inhibitor, a CD164 inhibitor, or a PARP1 inhibitor, or any combination thereof, may be carried out to prevent development of an additional CHIP or CHIP-related disorders in a subject who has already had CHIP or a CHIP-related disorder.

In any of the embodiments described herein, the LY75 variant nucleic acid molecule can be any nucleic acid molecule (such as, for example, genomic nucleic acid molecule, mRNA molecule, or cDNA molecule) encoding an LY75 variant polypeptide having a partial loss-of-function, a complete loss-of-function, a predicted partial loss-of-function, or a predicted complete loss-of-function. In some embodiments, the LY75 variant nucleic acid molecules encoding an LY75 predicted loss-of-function polypeptide is associated with a reduced in vitro response to LY75 ligands compared with reference LY75. In some embodiments, the LY75 variant nucleic acid molecules encoding an LY75 predicted loss-of-function polypeptide is an LY75 variant that results or is predicted to result in a premature truncation of an LY75 polypeptide compared to the human reference genome sequence.

In any of the embodiments described herein, the CD164 variant nucleic acid molecule can be any nucleic acid molecule (such as, for example, genomic nucleic acid molecule, mRNA molecule, or cDNA molecule) encoding a CD164 variant polypeptide having a partial loss-of-function, a complete loss-of-function, a predicted partial loss-of-function, or a predicted complete loss-of-function. In some embodiments, the CD164 variant nucleic acid molecules encoding a CD164 predicted loss-of-function polypeptide is associated with a reduced in vitro response to CD164 ligands compared with reference CD164. In some embodiments, the CD164 variant nucleic acid molecules encoding a CD164 predicted loss-of-function polypeptide is a CD164 variant that results or is predicted to result in a premature truncation of a CD164 polypeptide compared to the human reference genome sequence.

In any of the embodiments described herein, the PARP1 variant nucleic acid molecule can be any nucleic acid molecule (such as, for example, genomic nucleic acid molecule, mRNA molecule, or cDNA molecule) encoding a PARP1 variant polypeptide having a partial loss-of-function, a complete loss-of-function, a predicted partial loss-of-function, or a predicted complete loss-of-function. In some embodiments, the PARP1 variant nucleic acid molecules encoding a PARP1 predicted loss-of-function polypeptide is associated with a reduced in vitro response to PARP1 ligands compared with reference PARP1. In some embodiments, the PARP1 variant nucleic acid molecules encoding a PARP1 predicted loss-of-function polypeptide is a PARP1 variant that results or is predicted to result in a premature truncation of a PARP1 polypeptide compared to the human reference genome sequence.

In some embodiments, the LY75, CD164, or PARP1 variant nucleic acid molecules are variants that are predicted to be damaging by in vitro prediction algorithms such as Polyphen, SIFT, or similar algorithms.

In some embodiments, the LY75 variant nucleic acid molecules are variants that cause or are predicted to cause a nonsynonymous amino-acid substitution in an LY75 nucleic acid molecule and whose allele frequency is less than 1/100 alleles in the population from which the subject is selected. In some embodiments, the LY75 variant nucleic acid molecule is any rare variant (allele frequency <0.1%; or 1 in 1,000 alleles), or any splice-site, stop-gain, start-loss, stop-loss, frameshift, or in-frame indel, or other frameshift LY75 variant.

In any of the embodiments described herein, the LY75 predicted loss-of-function polypeptide can be any LY75 polypeptide having a partial loss-of-function, a complete loss-of-function, a predicted partial loss-of-function, or a predicted complete loss-of-function.

In any of the embodiments described herein, the LY75 variant nucleic acid molecule can include variations at positions of chromosome 2 using the nucleotide sequence of the LY75 reference genomic nucleic acid molecule (SEQ ID NO:1; ENSG00000054219.11, chr2:159,803,355-159,904,756 or in the GRCh38/hg38 human genome assembly) as a reference sequence. Numerous genetic variants in LY75 exist which cause subsequent changes in the LY75 polypeptide sequence including, but not limited to rs78446341 (GRCh38/hg38 chr2:159,834,145:G:A) and rs147820690 (GRCh38/hg38 chr2:159,878,663:C:T).

In some embodiments, the CD164 variant nucleic acid molecules are variants that cause or are predicted to cause a nonsynonymous amino-acid substitution in CD164 nucleic acid molecules and whose allele frequency is less than 1/100 alleles in the population from which the subject is selected. In some embodiments, the CD164 variant nucleic acid molecules are any rare variant (allele frequency <0.1%; or 1 in 1,000 alleles), or any splice-site, stop-gain, start-loss, stop-loss, frameshift, or in-frame indel, or other frameshift CD164 variant.

In any of the embodiments described herein, the CD164 predicted loss-of-function polypeptide can be any CD164 polypeptide having a partial loss-of-function, a complete loss-of-function, a predicted partial loss-of-function, or a predicted complete loss-of-function.

In any of the embodiments described herein, the CD164 variant nucleic acid molecule can include variations at positions of chromosome 6 using the nucleotide sequence of the CD164 reference genomic nucleic acid molecule (SEQ ID NO:55; ENSG00000135535.17, chr6:109,366,514-109,381,739 in the GRCh38/hg38 human genome assembly) as a reference sequence. Numerous genetic variants in CD164 exist which cause subsequent changes in the CD164 polypeptide sequence including, but not limited to rs3799840 (GRCh38/hg38 chr6:109381443A:T).

In some embodiments, the PARP1 variant nucleic acid molecules are variants that cause or are predicted to cause a nonsynonymous amino-acid substitution in PARP1 nucleic acid molecules and whose allele frequency is less than 1/100 alleles in the population from which the subject is selected. In some embodiments, the PARP1 variant nucleic acid molecules are any rare variant (allele frequency <0.1%; or 1 in 1,000 alleles), or any splice-site, stop-gain, start-loss, stop-loss, frameshift, or in-frame indel, or other frameshift PARP1 variant.

In any of the embodiments described herein, the PARP1 predicted loss-of-function polypeptide can be any PARP1 polypeptide having a partial loss-of-function, a complete loss-of-function, a predicted partial loss-of-function, or a predicted complete loss-of-function.

In any of the embodiments described herein, the PARP1 variant nucleic acid molecule can include variations at positions of chromosome 1 using the nucleotide sequence of the PARP1 reference genomic nucleic acid molecule (SEQ ID NO:113; ENSG00000143799.14, chr1:226,360,691-226,408,093 in the GRCh38/hg38 human genome assembly) as a reference sequence. Numerous genetic variants in PARP1 exist which cause subsequent changes in the PARP1 polypeptide sequence including, but not limited to s1136410 (GRCh38/hg38 chr1:226367601:A:G), rs2793379 (GRCh38/hg38 chr1:226408985:T:A), rs2570367 (GRCh38/hg38 chr1:226414809T:C), rs1433574 (GRCh38/hg38 chr1:226421638:A:C)and rs2039925 (GRCh38/hg38chr1:226422811:C:G).

Any one or more (i.e., any combination) of the LY75, CD164, or PARP1 variant nucleic acid molecules can be used within any of the methods described herein to determine whether a subject has an increased risk of developing CHIP or a CHIP-related disorder. The combinations of particular variants can form a mask used for statistical analysis of the particular correlation of any one or more LY75, CD164, or PARP1 and decreased risk of developing CHIP.

In any of the embodiments described herein, the CHIP or CHIP-related disorder is a hematologic cancer, a myeloid neoplasia, a lymphoid neoplasia, an atherosclerotic cardiovascular disease, a coronary heart disease, a myocardial infarction, and/or a severe calcified aortic valve stenosis. In some embodiments, the CHIP or CHIP-related disorder is a hematologic cancer. In some embodiments, the CHIP or CHIP-related disorder is a myeloid neoplasia. In some embodiments, the the CHIP or CHIP-related disorder is a lymphoid neoplasia. In some embodiments, the CHIP or CHIP-related disorder is an atherosclerotic cardiovascular disease. In some embodiments, the CHIP or CHIP-related disorder is a coronary heart disease. In some embodiments, the CHIP or CHIP-related disorder is a myocardial infarction. In some embodiments, the CHIP or CHIP-related disorder is a severe calcified aortic valve stenosis.

Symptoms of myeloid neoplasia include, but are not limited to, fever, bone pain, lethargy and fatigue, shortness of breath, pale skin, frequent infections, easy bruising, and unusual bleeding, such as frequent nosebleeds and bleeding from the gums.

Symptoms of lymphoid neoplasia include, but are not limited to, painless swelling of lymph nodes in neck, armpits or groin, persistent fatigue, fever, night sweats, shortness of breath, unexplained weight loss, and itchy skin.

Symptoms of coronary heart disease include, but are not limited to, angina, cold sweats, dizziness, light-headedness, nausea or a feeling of indigestion, neck pain, shortness of breath (especially with activity), sleep disturbances, and weakness.

Symptoms of myocardial infarction include, but are not limited to, pressure or tightness in the chest, pain in the chest, back, jaw, and other areas of the upper body that lasts more than a few minutes or that goes away and comes back, shortness of breath, sweating, nausea, vomiting, anxiety, a cough, dizziness, and a fast heart rate.

Symptoms of severe calcified aortic valve stenosis include, but are not limited to, abnormal heart sound (heart murmur) heard through a stethoscope, chest pain (angina) or tightness (with activity), feeling faint or dizzy or fainting (with activity), shortness of breath, (especially with activity), fatigue (especially during times of increased activity), rapid, fluttering heartbeat (palpitations), not eating enough (mainly in children with aortic valve stenosis), and not gaining enough weight (mainly in children with aortic valve stenosis).

It has also been observed in accordance with the present disclosure that CHIP somatic mutations in either DNMT3A or ASXL1 associate with an increased risk of developing lung cancer. Therefore, subjects that have a CHIP somatic mutation in either DNMT3A or ASXL1 may be monitored more frequently for lung cancer pathology (such as by more frequent chest x-rays, or the like), treatment with palliative agents, or smoking cessation procedures, such that development of lung cancer is prevented or delayed.

In any of the embodiments described herein, a DNMT3A or an ASXL1 somatic mutation is any mutation that is a missense mutation, a splice-site mutation, a stop-gain mutation, a start-loss mutation, a stop-loss mutation, a frameshift mutation, an in-frame indel mutation, or a mutation that results in a truncated DNMT3A or ASXL1 polypeptide. In any of the embodiments described herein, a DNMT3A or an ASXL1 somatic mutation can also be any mutation that results in a DNMT3A or an ASXL1 polypeptide having a partial loss-of-function, a complete loss-of-function, a predicted partial loss-of-function, or a predicted complete loss-of-function.

In any of the embodiments described herein, the DNMT3A mutation can include variations at positions of chromosome 2 using the nucleotide sequence of the DNMT3A reference genomic nucleic acid molecule (SEQ ID NO:212; ENSG00000119772.17, chr2:25,227,855-25,342,590 in the GRCh38/hg38 human genome assembly) as a reference sequence.

In any of the embodiments described herein, the ASXL1 mutation can include variations at positions of chromosome 20 using the nucleotide sequence of the ASXL1 reference genomic nucleic acid molecule (SEQ ID NO:267; ENSG00000171456.20, chr20:32,358,330-32,439,260 in the GRCh38/hg38 human genome assembly) as a reference sequence.

The present disclosure provides methods of preventing or reducing the development of CHIP in a subject, the methods comprising administering an LY75 inhibitor, a CD164 inhibitor, or a PARP1 inhibitor, or any combination thereof, to the subject.

The present disclosure also provides methods of treating a subject having a hematologic cancer or at risk of developing a hematologic cancer, the methods comprising administering an LY75 inhibitor, or a CD164 inhibitor, or a PARP1 inhibitor, or any combination thereof, to the subject.

The present disclosure also provides methods of treating a subject having a myeloid neoplasia or at risk of developing a myeloid neoplasia, the methods comprising administering an LY75 inhibitor, or a CD164 inhibitor, or a PARP1 inhibitor, or any combination thereof, to the subject.

The present disclosure also provides methods of treating a subject having a lymphoid neoplasia or at risk of developing a lymphoid neoplasia, the methods comprising administering an LY75 inhibitor, or a CD164 inhibitor, or a PARP1 inhibitor, or any combination thereof, to the subject.

The present disclosure also provides methods of treating a subject having an atherosclerotic cardiovascular disease or at risk of developing an atherosclerotic cardiovascular disease, the methods comprising administering an LY75 inhibitor, or a CD164 inhibitor, or a PARP1 inhibitor, or any combination thereof, to the subject.

The present disclosure also provides methods of treating a subject having a coronary heart disease or at risk of developing a coronary heart disease, the methods comprising administering an LY75 inhibitor, or a CD164 inhibitor, or a PARP1 inhibitor, or any combination thereof, to the subject.

The present disclosure also provides methods of treating a subject who has or has had a myocardial infarction or at risk of developing a myocardial infarction, the methods comprising administering an LY75 inhibitor, or a CD164 inhibitor, or a PARP1 inhibitor, or any combination thereof, to the subject.

The present disclosure also provides methods of treating a subject having a severe calcified aortic valve stenosis or at risk of developing a severe calcified aortic valve stenosis, the methods comprising administering an LY75 inhibitor, or a CD164 inhibitor, or a PARP1 inhibitor, or any combination thereof, to the subject.

In some embodiments, the LY75 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of an LY75 nucleic acid molecule. In some embodiments, the antisense RNA, siRNA, or shRNA hybridizes to a sequence within an LY75 genomic nucleic acid molecule or mRNA molecule and decreases expression of the LY75 polypeptide in a cell in the subject. In some embodiments, the LY75 inhibitor comprises an antisense molecule that hybridizes to an LY75 genomic nucleic acid molecule or mRNA molecule and decreases expression of the LY75 polypeptide in a cell in the subject. In some embodiments, the LY75 inhibitor comprises an siRNA that hybridizes to an LY75 genomic nucleic acid molecule or mRNA molecule and decreases expression of the LY75 polypeptide in a cell in the subject. In some embodiments, the LY75 inhibitor comprises an shRNA that hybridizes to an LY75 genomic nucleic acid molecule or mRNA molecule and decreases expression of the LY75 polypeptide in a cell in the subject.

In some embodiments, the CD164 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of a CD164 nucleic acid molecule. In some embodiments, the antisense RNA, siRNA, or shRNA hybridizes to a sequence within a CD164 genomic nucleic acid molecule or mRNA molecule and decreases expression of the CD164 polypeptide in a cell in the subject. In some embodiments, the CD164 inhibitor comprises an antisense molecule that hybridizes to a CD164 genomic nucleic acid molecule or mRNA molecule and decreases expression of the CD164 polypeptide in a cell in the subject. In some embodiments, the CD164 inhibitor comprises an siRNA that hybridizes to a CD164 genomic nucleic acid molecule or mRNA molecule and decreases expression of the CD164 polypeptide in a cell in the subject. In some embodiments, the CD164 inhibitor comprises an shRNA that hybridizes to a CD164 genomic nucleic acid molecule or mRNA molecule and decreases expression of the CD164 polypeptide in a cell in the subject.

In some embodiments, the PARP1 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of a PARP1 nucleic acid molecule. In some embodiments, the antisense RNA, siRNA, or shRNA hybridizes to a sequence within a PARP1 genomic nucleic acid molecule or mRNA molecule and decreases expression of the PARP1 polypeptide in a cell in the subject. In some embodiments, the PARP1 inhibitor comprises an antisense molecule that hybridizes to a PARP1 genomic nucleic acid molecule or mRNA molecule and decreases expression of the PARP1 polypeptide in a cell in the subject. In some embodiments, the PARP1 inhibitor comprises an siRNA that hybridizes to a PARP1 genomic nucleic acid molecule or mRNA molecule and decreases expression of the PARP1 polypeptide in a cell in the subject. In some embodiments, the PARP1 inhibitor comprises an shRNA that hybridizes to a PARP1 genomic nucleic acid molecule or mRNA molecule and decreases expression of the PARP1 polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

In any of the embodiments described herein, any of the inhibitory nucleic acid molecules can be formulated as a component of a lipid nanoparticle, and can be delivered to a cell by a lipid nanoparticle.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that camot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula: Sense: mN*mN*/i2FN/mN/i2FN/mN/i2FN/mN/i2FN/mN/i2FN/mN/i2FN/mN/i2FN/mN/i2FN/*mN*/32FN/Antisense: /52FN/*/i2FN/*mN/i2FN/mN/i2FN/mN/i2FN/mN/i2FN/mN/i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the LY75 inhibitor, the CD164 inhibitor, or the PARP1 inhibitor inhibitor comprises a nuclease agent that induces one or more nicks or double-strand breaks at a recognition sequence(s) or a DNA-binding protein that binds to a recognition sequence within an LY75, a CD164, or a PARP1 genomic nucleic acid molecule. The recognition sequence can be located within a coding region of the LY75 gene, the CD164 gene, or the PARP1 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the LY75 gene, the CD164 gene, or the PARP1 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 by for each ZFN, about 36 by fora TALE protein or TALEN, and about 20 by for a CRISPR/Cas guide RNA.

In some embodiments, CRISPR/Cas systems can be used to modify LY75, CD164, and/or PARP1 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of LY75, CD164, and/or PARP1 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an LY75, a CD164, or a PARP1 genomic nucleic acid molecule or it can be a nickase that creates a single-strand break in an LY75, a CD164, or a PARP1 genomic nucleic acid molecule. Additional examples of Cas proteins include, but are not limited to, Cas1, Cas1B, Cast, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (Cas6), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of LY75 genomic nucleic acid molecules can be generated by contacting a cell with a Cas protein and one or more gRNAs that hybridize to one or more gRNA recognition sequences within a target genomic locus in the LY75 genomic nucleic acid molecule. For example, an LY75 gRNA recognition sequence can be located within a region of SEQ ID NO:1. The gRNA recognition sequence can include or be proximate to the start codon of an LY75 genomic nucleic acid molecule or the stop codon of an LY75 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

In some embodiments, targeted genetic modifications of CD164 genomic nucleic acid molecules can be generated by contacting a cell with a Cas protein and one or more gRNAs that hybridize to one or more gRNA recognition sequences within a target genomic locus in the CD164 genomic nucleic acid molecule. For example, a CD164 gRNA recognition sequence can be located within a region of SEQ ID NO:55. The gRNA recognition sequence can include or be proximate to the start codon of a CD164 genomic nucleic acid molecule or the stop codon of a CD164 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

In some embodiments, targeted genetic modifications of PARP1 genomic nucleic acid molecules can be generated by contacting a cell with a Cas protein and one or more gRNAs that hybridize to one or more gRNA recognition sequences within a target genomic locus in the PARP1 genomic nucleic acid molecule. For example, a PARP1 gRNA recognition sequence can be located within a region of SEQ ID NO:113. The gRNA recognition sequence can include or be proximate to the start codon of a PARP1 genomic nucleic acid molecule or the stop codon of a PARP1 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an LY75, a CD164, or a PARP1 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an LY75, a CD164, or a PARP1 genomic nucleic acid molecule. An exemplary gRNA is a gRNA effective to direct a Cas enzyme to bind to or cleave an LY75, a CD164, or a PARP1 genomic nucleic acid molecule, wherein the gRNA comprises a DNA-targeting segment that hybridizes to a gRNA recognition sequence within the LY75, CD164, or PARP1 genomic nucleic acid molecule. Exemplary gRNAs comprise a DNA-targeting segment that hybridizes to a gRNA recognition sequence present within an LY75, a CD164, or a PARP1 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the human LY75 reference gene are set forth in Table 1 as SEQ ID NOs:152-171.

TABLE 1
Guide RNA Recognition Sequences Near LY75

| Strand | gRNA Recognition Sequence | SEQ ID NO: |
|---|---|---|
| - | GGTGGATAAGAATTAGCGAG | 152 |
| - | GTGGAAAGTCCAATCATGTG | 153 |
| - | TATGAATATGACCGAAAGTG | 154 |
| - | GAGTATAACTGGGCAACTGT | 155 |
| - | TGTGTAGTATTAGACACTGA | 156 |
| - | TGTGAGCACCACTCTCTGTA | 157 |
| + | GAATAGTAGCAAATAGCACC | 158 |
| - | GAAACCATTAAATAATACAG | 159 |
| + | GGTTGAGTATTAGGGCACAG | 160 |
| - | GTGTTGAGCCAGATTGAACA | 161 |
| - | AATCACCGTTTACTGGGACG | 162 |
| - | ACACCAGACTGGTACAATCC | 163 |
| + | ACAGGGGTCATCAGGCTTAG | 164 |
| - | GCTATACTCTGCTAGAGGCT | 165 |
| + | CAGGTCCCATCAATTAAGAA | 166 |
| - | AATCAGCTATACTCTGCTAG | 167 |
| + | CAGCTGGAGCCACCTATAGT | 168 |
| + | TTCACAGATGAAGGGCAACT | 169 |
| + | GCCAGGATCCTTGTAAATCT | 170 |
| + | CTCACATTTCCACCACAGCA | 171 |

Examples of suitable gRNA recognition sequences located within the human CD164 reference gene are set forth in Table 2 as SEQ ID NOs:172-191.

TABLE 2
Guide RNA Recognition Sequences NearCD164

| Strand | gRNA Recognition Sequence | SEQ ID NO: |
|---|---|---|
| + | CTGGGTCGTGTTCTTGTCCG | 172 |
| - | AACTCCCCAGAAACCTGTGA | 173 |
| + | CACCGGCGCCGAGGTTACGT | 174 |
| - | ACAGTTAGTGATTGTCAAGT | 175 |
| - | GCCCATCTCCAACGTAACCT | 176 |
| - | CAGTTAGTGATTGTCAAGTG | 177 |
| + | ACCAGCGGGAGGGACGTCAC | 178 |
| + | GGTGCCGGAGTGGTGACCAG | 179 |
| - | CTCCAACGTAACCTCGGCGC | 180 |
| - | GTGCCAACAGCCAATTCTAC | 181 |
| - | CCTCCCGCTGGTCACCACTC | 182 |
| + | TCGACCTTCACAGGTTTCTG | 183 |
| - | AACAGTTAGTGATTGTCAAG | 184 |
| + | GCCGAGGTTACGTTGGAGAT | 185 |
| + | GCAGCTGTTTCGACCTTCAC | 186 |
| + | GAGGGACGTCACCGGCGCCG | 187 |
| + | AGTCGTCACGTTCGGGTGCT | 188 |
| + | AACAACGCTAACATTAAAAC | 189 |
| + | GCAGAGCACGCCCAGGCAGG | 190 |
| + | TTTACATTCTATCCAAAAGC | 191 |

Examples of suitable gRNA recognition sequences located within the human PARP1 reference gene are set forth in Table 3 as SEQ ID NOs:192-211.

TABLE 3
Guide RNA Recognition Sequences Near PARP

| Strand | gRNA Recognition Sequence | SEQ ID NO: |
|---|---|---|
| + | TACCGATCACCGTACCCACA | 192 |
| - | AACTCGGGGGGAAGTTGACG | 193 |
| - | CGATGCCTATTACTGCACTG | 194 |
| - | AGCTAGGCATGATTGACCGC | 195 |
| + | TGGCCATAGTCAATCTCCAG | 196 |
| - | AATTATATGAAGAAAAAACC | 197 |
| - | ATCTTGGACCGAGTAGCTGA | 198 |
| - | GAAGGAGTGGGTAACCCCAA | 199 |
| + | GGCCATAGTCAATCTCCAGG | 200 |
| + | CTGGCCATAGTCAATCTCCA | 201 |
| - | GGAACAGATGCCGTCCAAGG | 202 |
| - | GACACAGACACCCAACCGGA | 203 |
| + | GGAAGTCCTCAGACACAACT | 204 |
| - | GGCCATGATTGAGAAACTCG | 205 |
| - | TCCAACAGAAGTACGTGCAA | 206 |
| - | TGGCCCCAAGAGGGAAGTCA | 207 |
| - | CATCCGGCACCCTGACGTTG | 208 |
| - | CCCAGAAACCAGCGCCTCCG | 209 |
| - | AAAGAGAAAAGGCGATGAGG | 210 |
| - | CCTGGAGATTGACTATGGCC | 211 |

The Cas protein and the gRNA form a complex, and the Cas protein cleaves the target LY75, CD164, or PARP1 genomic nucleic acid molecule. The Cas protein can cleave the nucleic acid molecule at a site within or outside of the nucleic acid sequence present in the target LY75, CD164, or PARP1 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the LY75, CD164, or PARP1 genomic nucleic acid molecule to which a DNA-targeting segment of a gRNA will bind.

Such methods can result, for example, in an LY75 genomic nucleic acid molecule in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the LY75 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks. The CD164 and the PARP1 genomic DNAs can be similarly targeted.

In some embodiments, the methods of prevention and/or reduction further comprise detecting the presence or absence of an LY75 variant nucleic acid molecule in a biological sample from the subject. In some embodiments, the methods of prevention and/or reduction further comprise detecting the presence or absence of a CD164 variant nucleic acid molecule in a biological sample from the subject. In some embodiments, the methods of prevention and/or reduction further comprise detecting the presence or absence of a PARP1 variant nucleic acid molecule in a biological sample from the subject.

The present disclosure also provides methods of treating a subject with a therapeutic agent that treats or inhibits CHIP, wherein the subject is at risk of developing CHIP or a CHIP-related disorder. In some embodiments, the methods comprise determining whether the subject has an LY75 variant nucleic acid molecule by obtaining or having obtained a biological sample from the subject, and performing or having performed a sequence analysis on the biological sample to determine if the subject has a genotype comprising an LY75 variant nucleic acid molecule. In some embodiments, the methods further comprise administering or continuing to administer the therapeutic agent that prevents or reduces development of CHIP in a standard dosage amount to a subject that is LY75, and/or administering an LY75 inhibitor, to the subject. In some embodiments, the methods further comprise administering or continuing to administer the therapeutic agent that prevents or reduces development of CHIP in an amount that is the same as or less than a standard dosage amount to a subject that is heterozygous for the LY75 variant nucleic acid molecule, and/or administering an LY75 inhibitor. In some embodiments, the methods further comprise administering or continuing to administer the therapeutic agent that prevents or reduces development of CHIP in an amount that is the same as or less than a standard dosage amount to a subject that is heterozygous for the LY75 variant nucleic acid molecule, and/or administering an LY75 inhibitor to the subject.

The present disclosure also provides methods of treating a subject with a therapeutic agent that treats or inhibits CHIP, wherein the subject is at risk of developing CHIP or a CHIP-related disorder. In some embodiments, the methods comprise determining whether the subject has a CD164 variant nucleic acid molecule by obtaining or having obtained a biological sample from the subject, and performing or having performed a sequence analysis on the biological sample to determine if the subject has a genotype comprising a CD164 variant nucleic acid molecule. In some embodiments, the methods further comprise administering or continuing to administer the therapeutic agent that prevents or reduces development of CHIP in a standard dosage amount to a subject that is CD164, and/or administering a CD164 inhibitor, to the subject. In some embodiments, the methods further comprise administering or continuing to administer the therapeutic agent that prevents or reduces development of CHIP in an amount that is the same as or less than a standard dosage amount to a subject that is heterozygous for the CD164 variant nucleic acid molecule, and/or administering a CD164 inhibitor. In some embodiments, the methods further comprise administering or continuing to administer the therapeutic agent that prevents or reduces development of CHIP in an amount that is the same as or less than a standard dosage amount to a subject that is heterozygous for the CD164 variant nucleic acid molecule, and/or administering a CD164 inhibitor to the subject.

The present disclosure also provides methods of treating a subject with a therapeutic agent that treats or inhibits CHIP, wherein the subject is at risk of developing CHIP or a CHIP-related disorder. In some embodiments, the methods comprise determining whether the subject has a PARP1 variant nucleic acid molecule by obtaining or having obtained a biological sample from the subject, and performing or having performed a sequence analysis on the biological sample to determine if the subject has a genotype comprising a PARP1 variant nucleic acid molecule. In some embodiments, the methods further comprise administering or continuing to administer the therapeutic agent that prevents or reduces development of CHIP in a standard dosage amount to a subject that is PARP1, and/or administering a PARP1 inhibitor, to the subject. In some embodiments, the methods further comprise administering or continuing to administer the therapeutic agent that prevents or reduces development of CHIP in an amount that is the same as or less than a standard dosage amount to a subject that is heterozygous for the PARP1 variant nucleic acid molecule, and/or administering a PARP1 inhibitor. In some embodiments, the methods further comprise administering or continuing to administer the therapeutic agent that prevents or reduces development of CHIP in an amount that is the same as or less than a standard dosage amount to a subject that is heterozygous for the PARP1 variant nucleic acid molecule, and/or administering a PARP1 inhibitor to the subject.

In some embodiments, the methods further comprise administering or continuing to administer the therapeutic agent that prevents or reduces development of CHIP in an amount that is the same as or less than a standard dosage amount to a subject that is homozygous for an LY75 variant nucleic acid molecule. The presence of a genotype having an LY75 variant nucleic acid molecule indicates the subject has a decreased risk of developing CHIP or a CHIP-related disorder. In some embodiments, the subject is LY75 reference. In some embodiments, the subject is heterozygous for an LY75 variant nucleic acid molecule.

In some embodiments, the methods further comprise administering or continuing to administer the therapeutic agent that prevents or reduces development of CHIP in an amount that is the same as or less than a standard dosage amount to a subject that is homozygous for a CD164 variant nucleic acid molecule. The presence of a genotype having a CD164 variant nucleic acid molecule indicates the subject has a decreased risk of developing CHIP or a CHIP-related disorder. In some embodiments, the subject is CD164 reference. In some embodiments, the subject is heterozygous for a CD164 variant nucleic acid molecule.

In some embodiments, the methods further comprise administering or continuing to administer the therapeutic agent that prevents or reduces development of CHIP in an amount that is the same as or less than a standard dosage amount to a subject that is homozygous for a PARP1 variant nucleic acid molecule. The presence of a genotype having a PARP1 variant nucleic acid molecule indicates the subject has a decreased risk of developing CHIP or a CHIP-related disorder. In some embodiments, the subject is PARP1 reference. In some embodiments, the subject is heterozygous for a PARP1 variant nucleic acid molecule.

For subjects that are genotyped or determined to be either LY75 reference or heterozygous for LY75 variant nucleic acid molecule, such subjects can be administered an LY75 inhibitor, as described herein.

For subjects that are genotyped or determined to be CD164 reference or heterozygous for CD164 variant nucleic acid molecule, such subjects can be administered a CD164 inhibitor, as described herein.

For subjects that are genotyped or determined to be PARP1 reference or heterozygous for PARP1 variant nucleic acid molecule, such subjects can be administered a PARP1 inhibitor, as described herein.

For subjects that are genotyped or determined to be either or both LY75 and CD164 reference, or LY75 reference and heterozygous for a CD164 variant nucleic acid molecule, or CD164 reference and heterozygous for an LY75 variant nucleic acid molecule or heterozygous for both an LY75 variant nucleic acid molecule and a CD164 variant nucleic acid molecule, such subjects can be administered an LY75 inhibitor, a CD164 inhibitor, or both, as described herein.

For subjects that are genotyped or determined to be either or both LY75 and PARP1 reference, or LY75 reference and heterozygous for a PARP1 variant nucleic acid molecule, or PARP1 reference and heterozygous for an LY75 variant nucleic acid molecule or heterozygous for both an LY75 variant nucleic acid molecule and a PARP1 variant nucleic acid molecule, such subjects can be administered an LY75 inhibitor, a PARP1 inhibitor, or both, as described herein.

For subjects that are genotyped or determined to be either or both CD164 and PARP1 reference, or CD164 reference and heterozygous for a PARP1 variant nucleic acid molecule, or PARP1 reference and heterozygous for a CD164 variant nucleic acid molecule or heterozygous for both a CD164 variant nucleic acid molecule and a PARP1 variant nucleic acid molecule, such subjects can be administered a CD164 inhibitor, a PARP1 inhibitor, or both, as described herein.

For subjects that are genotyped or determined to be either LY75, CD164, and PARP1 reference; or LY75 reference and heterozygous for both a CD164 variant nucleic acid molecule and a PARP1 variant nucleic acid molecule; or CD164 reference and heterozygous for both an LY75 variant nucleic acid molecule and a PARP1 variant nucleic acid molecule; or PARP1 reference and heterozygous for both an LY75 variant nucleic acid molecule and a CD164 variant nucleic acid molecule; or heterozygous for an LY75 variant nucleic acid molecule, a CD164 variant nucleic acid molecule, and a PARP1 variant nucleic acid molecule; such subjects can be administered an LY75 inhibitor, a CD164 inhibitor, a PARP1 inhibitor or any combination thereof, as described herein.

Detecting the presence or absence of one or more LY75, CD164, or PARP1 variant nucleic acid molecules in a biological sample from a subject and/or determining whether a subject has one or more LY75, CD164, or PARP1 variant nucleic acid molecules can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is LY75 reference, the subject is administered a therapeutic agent that prevents or reduces development of CHIP in a standard dosage amount. In some embodiments, when the subject is heterozygous LY75 variant nucleic acid molecule, the subject is administered a therapeutic agent that prevents or reduces development of CHIP in a dosage amount that is the same as or less than a standard dosage amount.

In some embodiments, when the subject is CD164 reference, the subject is administered a therapeutic agent that prevents or reduces development of CHIP in a standard dosage amount. In some embodiments, when the subject is heterozygous for CD164 variant nucleic acid molecule, the subject is administered a therapeutic agent that prevents or reduces development of CHIP in a dosage amount that is the same as or less than a standard dosage amount.

In some embodiments, when the subject is PARP1 reference, the subject is administered a therapeutic agent that prevents or reduces development of CHIP in a standard dosage amount. In some embodiments, when the subject is heterozygous for PARP1 variant nucleic acid molecule, the subject is administered a therapeutic agent that prevents or reduces development of CHIP in a dosage amount that is the same as or less than a standard dosage amount.

In some embodiments, the prevention and/or reduction methods comprise detecting the presence or absence of an LY75 predicted loss-of-function polypeptide in a biological sample from the subject. In some embodiments, when the subject does not have an LY75 predicted loss-of-function polypeptide, the subject is administered a therapeutic agent that prevents or reduces development of CHIP in a standard dosage amount. In some embodiments, when the subject has an LY75 predicted loss-of-function polypeptide, the subject is administered a therapeutic agent that prevents or reduces CHIP in a dosage amount that is the same as or less than a standard dosage amount.

In some embodiments, the prevention and/or reduction methods comprise detecting the presence or absence of a CD164 predicted loss-of-function polypeptide in a biological sample from the subject. In some embodiments, when the subject does not have a CD164 predicted loss-of-function polypeptide, the subject is administered a therapeutic agent that prevents or reduces development of CHIP in a standard dosage amount. In some embodiments, when the subject has a CD164 predicted loss-of-function polypeptide, the subject is administered a therapeutic agent that prevents or reduces CHIP in a dosage amount that is the same as or less than a standard dosage amount.

In some embodiments, the prevention and/or reduction methods comprise detecting the presence or absence of a PARP1 predicted loss-of-function polypeptide in a biological sample from the subject. In some embodiments, when the subject does not have a PARP1 predicted loss-of-function polypeptide, the subject is administered a therapeutic agent that prevents or reduces development of CHIP in a standard dosage amount. In some embodiments, when the subject has a PARP1 predicted loss-of-function polypeptide, the subject is administered a therapeutic agent that prevents or reduces CHIP in a dosage amount that is the same as or less than a standard dosage amount.

The present disclosure also provides methods of treating a subject with a therapeutic agent that treats or inhibits a hematologic cancer, wherein the subject has CHIP. In some embodiments, the method comprises determining whether the subject has an LY75 variant nucleic acid molecule by obtaining or having obtained a biological sample from the subject, and performing or having performed a sequence analysis on the biological sample to determine if the subject has an LY75 variant nucleic acid molecule. When the subject does not have an LY75 variant nucleic acid molecule, the therapeutic agent that treats or inhibits a hematologic cancer is administered or continued to be administered to the subject in a standard dosage amount, and/or an LY75 inhibitor is administered to the subject. When the subject has an LY75 variant nucleic acid molecule, the therapeutic agent that treats or inhibits hematologic cancer is administered or continued to be administered to the subject in an amount that is the same as or less than a standard dosage amount, and/or an LY75 inhibitor is administered to the subject. The presence of an LY75 variant nucleic acid molecule indicates the subject has a decreased risk of developing hematologic cancer. In some embodiments, the subject has an LY75 variant nucleic acid molecule. In some embodiments, the subject does not have an LY75 variant nucleic acid molecule.

The present disclosure also provides methods of treating a subject with a therapeutic agent that treats or inhibits a hematologic cancer, wherein the subject has CHIP. In some embodiments, the method comprises determining whether the subject has a CD164 variant nucleic acid molecule by obtaining or having obtained a biological sample from the subject, and performing or having performed a sequence analysis on the biological sample to determine if the subject has a CD164 variant nucleic acid molecule. When the subject does not have a CD164 variant nucleic acid molecule, the therapeutic agent that treats or inhibits a hematologic cancer is administered or continued to be administered to the subject in a standard dosage amount, and/or a CD164 inhibitor is administered to the subject. When the subject has a CD164 variant nucleic acid molecule, the therapeutic agent that treats or inhibits hematologic cancer is administered or continued to be administered to the subject in an amount that is the same as or less than a standard dosage amount, and/or a CD164 inhibitor is administered to the subject. The presence of a CD164 variant nucleic acid molecule indicates the subject has a decreased risk of developing hematologic cancer. In some embodiments, the subject has a CD164 variant nucleic acid molecule. In some embodiments, the subject does not have a CD164 variant nucleic acid molecule.

The present disclosure also provides methods of treating a subject with a therapeutic agent that treats or inhibits a hematologic cancer, wherein the subject has CHIP. In some embodiments, the method comprises determining whether the subject has a PARP1 variant nucleic acid molecule by obtaining or having obtained a biological sample from the subject, and performing or having performed a sequence analysis on the biological sample to determine if the subject has a PARP1 variant nucleic acid molecule. When the subject does not have a PARP1 variant nucleic acid molecule, the therapeutic agent that treats or inhibits a hematologic cancer is administered or continued to be administered to the subject in a standard dosage amount, and/or a PARP1 inhibitor is administered to the subject. When the subject has a PARP1 variant nucleic acid molecule, the therapeutic agent that treats or inhibits hematologic cancer is administered or continued to be administered to the subject in an amount that is the same as or less than a standard dosage amount, and/or a PARP1 inhibitor is administered to the subject. The presence of a PARP1 variant nucleic acid molecule indicates the subject has a decreased risk of developing hematologic cancer. In some embodiments, the subject has a PARP1 variant nucleic acid molecule. In some embodiments, the subject does not have a PARP1 variant nucleic acid molecule.

In some embodiments when the subject has both an LY75 variant nucleic acid molecule and a CD164 variant nucleic acid molecule, the therapeutic agent that treats or inhibits hematologic cancer is administered or continued to be administered to the subject in an amount that is the same as or less than a standard dosage amount, and/or an LY75 inhibitor, or a CD164 inhibitor or both is administered to the subject.

In some embodiments when the subject has both an LY75 variant nucleic acid molecule and a PARP1 variant nucleic acid molecule, the therapeutic agent that treats or inhibits hematologic cancer is administered or continued to be administered to the subject in an amount that is the same as or less than a standard dosage amount, and/or an LY75 inhibitor, or a PARP1 inhibitor, or both is administered to the subject.

In some embodiments when the subject has both a CD164 variant nucleic acid molecule and a PARP1 variant nucleic acid molecule, the therapeutic agent that treats or inhibits hematologic cancer is administered or continued to be administered to the subject in an amount that is the same as or less than a standard dosage amount, and/or a CD164 inhibitor, or a PARP1 inhibitor, or both is administered to the subject.

In some embodiments when the subject has an LY75 variant nucleic acid molecule, a CD164 variant nucleic acid molecule, and a PARP1 variant nucleic acid molecule, the therapeutic agent that treats or inhibits hematologic cancer is administered or continued to be administered to the subject in an amount that is the same as or less than a standard dosage amount, and/or an LY75 inhibitor, a CD164 inhibitor, a PARP1 inhibitor, or any combination thereof is administered to the subject.

The present disclosure also provides methods of treating a subject with a therapeutic agent that treats or inhibits an atherosclerotic cardiovascular disease, wherein the subject has CHIP. In some embodiments, the method comprises determining whether the subject has an LY75 variant nucleic acid molecule by obtaining or having obtained a biological sample from the subject, and performing or having performed a sequence analysis on the biological sample to determine if the subject has an LY75 variant nucleic acid molecule. When the subject does not have an LY75 variant nucleic acid molecule, the therapeutic agent that treats or inhibits an atherosclerotic cardiovascular disease is administered or continued to be administered to the subject in a standard dosage amount, and/or an LY75 inhibitor is administered to the subject. When the subject has an LY75 variant nucleic acid molecule, the therapeutic agent that treats or inhibits atherosclerotic cardiovascular disease is administered or continued to be administered to the subject in an amount that is the same as or less than a standard dosage amount, and/or an LY75 inhibitor is administered to the subject. The presence of an LY75 variant nucleic acid molecule indicates the subject has a decreased risk of developing atherosclerotic cardiovascular disease. In some embodiments, the subject has an LY75 variant nucleic acid molecule. In some embodiments, the subject does not have an LY75 variant nucleic acid molecule.

The present disclosure also provides methods of treating a subject with a therapeutic agent that treats or inhibits an atherosclerotic cardiovascular disease, wherein the subject has CHIP. In some embodiments, the method comprises determining whether the subject has a CD164 variant nucleic acid molecule by obtaining or having obtained a biological sample from the subject, and performing or having performed a sequence analysis on the biological sample to determine if the subject has a CD164 variant nucleic acid molecule. When the subject does not have a CD164 variant nucleic acid molecule, the therapeutic agent that treats or inhibits an atherosclerotic cardiovascular disease is administered or continued to be administered to the subject in a standard dosage amount, and/or a CD164 inhibitor is administered to the subject. When the subject has a CD164 variant nucleic acid molecule, the therapeutic agent that treats or inhibits atherosclerotic cardiovascular disease is administered or continued to be administered to the subject in an amount that is the same as or less than a standard dosage amount, and/or a CD164 inhibitor is administered to the subject. The presence of a CD164 variant nucleic acid molecule indicates the subject has a decreased risk of developing atherosclerotic cardiovascular disease. In some embodiments, the subject has a CD164 variant nucleic acid molecule. In some embodiments, the subject does not have a CD164 variant nucleic acid molecule.

The present disclosure also provides methods of treating a subject with a therapeutic agent that treats or inhibits an atherosclerotic cardiovascular disease, wherein the subject has CHIP. In some embodiments, the method comprises determining whether the subject has a PARP1 variant nucleic acid molecule by obtaining or having obtained a biological sample from the subject, and performing or having performed a sequence analysis on the biological sample to determine if the subject has a PARP1 variant nucleic acid molecule. When the subject does not have a PARP1 variant nucleic acid molecule, the therapeutic agent that treats or inhibits an atherosclerotic cardiovascular disease is administered or continued to be administered to the subject in a standard dosage amount, and/or a PARP1 inhibitor is administered to the subject. When the subject has a PARP1 variant nucleic acid molecule, the therapeutic agent that treats or inhibits atherosclerotic cardiovascular disease is administered or continued to be administered to the subject in an amount that is the same as or less than a standard dosage amount, and/or a PARP1 inhibitor is administered to the subject. The presence of a PARP1 variant nucleic acid molecule indicates the subject has a decreased risk of developing atherosclerotic cardiovascular disease. In some embodiments, the subject has a PARP1 variant nucleic acid molecule. In some embodiments, the subject does not have a PARP1 variant nucleic acid molecule.

In some embodiments when the subject has both an LY75 variant nucleic acid molecule and a CD164 variant nucleic acid molecule, the therapeutic agent that treats or inhibits atherosclerotic cardiovascular disease is administered or continued to be administered to the subject in an amount that is the same as or less than a standard dosage amount, and/or an LY75 inhibitor, or a CD164 inhibitor or both is administered to the subject.

In some embodiments when the subject has both an LY75 variant nucleic acid molecule and a PARP1 variant nucleic acid molecule, the therapeutic agent that treats or inhibits atherosclerotic cardiovascular disease is administered or continued to be administered to the subject in an amount that is the same as or less than a standard dosage amount, and/or an LY75 inhibitor, or a PARP1 inhibitor, or both is administered to the subject.

In some embodiments when the subject has both a CD164 variant nucleic acid molecule and a PARP1 variant nucleic acid molecule, the therapeutic agent that treats or inhibits atherosclerotic cardiovascular disease is administered or continued to be administered to the subject in an amount that is the same as or less than a standard dosage amount, and/or a CD164 inhibitor, or a PARP1 inhibitor, or both is administered to the subject.

In some embodiments when the subject has an LY75 variant nucleic acid molecule, a CD164 variant nucleic acid molecule, and a PARP1 variant nucleic acid molecule, the therapeutic agent that treats or inhibits atherosclerotic cardiovascular disease is administered or continued to be administered to the subject in an amount that is the same as or less than a standard dosage amount, and/or an LY75 inhibitor, a CD164 inhibitor, a PARP1 inhibitor, or any combination thereof is administered to the subject.

In some embodiments, the subject is heterozygous for an LY75 variant nucleic acid molecule, and the subject is further administered or continued to be administered the therapeutic agent that prevents or reduces development of CHIP in an amount that is the same as or less than a standard dosage amount, and is administered the LY75 inhibitor.

In some embodiments, the subject is heterozygous for a CD164 variant nucleic acid molecule, and the subject is further administered or continued to be administered the therapeutic agent that prevents or reduces development of CHIP in an amount that is the same as or less than a standard dosage amount, and is administered the CD164 inhibitor.

In some embodiments, the subject is heterozygous for a PARP1 variant nucleic acid molecule, and the subject is further administered or continued to be administered the therapeutic agent that prevents or reduces development of CHIP in an amount that is the same as or less than a standard dosage amount, and is administered the PARP1 inhibitor.

In some embodiments, the subject is heterozygous for both an LY75 variant nucleic acid molecule and a CD164 variant nucleic acid molecule, and the subject is further administered or continued to be administered the therapeutic agent that prevents or reduces development of CHIP in an amount that is the same as or less than a standard dosage amount, and is administered an LY75 inhibitor, a CD164 inhibitor, or both, as described herein.

In some embodiments, the subject is heterozygous for both an LY75 variant nucleic acid molecule and a PARP1 variant nucleic acid molecule, and the subject is further administered or continued to be administered the therapeutic agent that prevents or reduces development of CHIP in an amount that is the same as or less than a standard dosage amount, and is administered an LY75 inhibitor, a PARP1 inhibitor, or both, as described herein.

In some embodiments, the subject is heterozygous for both a CD164 variant nucleic acid molecule and a PARP1 variant nucleic acid molecule, and the subject is further administered or continued to be administered the therapeutic agent that prevents or reduces development of CHIP in an amount that is the same as or less than a standard dosage amount, and is administered a CD164 inhibitor, a PARP1 inhibitor, or both, as described herein.

In some embodiments, the subject is heterozygous for an LY75 variant nucleic acid molecule, a CD164 variant nucleic acid molecule, and a PARP1 variant nucleic acid molecule; and the subject is further administered or continued to be administered the therapeutic agent that prevents or reduces development of CHIP in an amount that is the same as or less than a standard dosage amount, and is administered an LY75 inhibitor, a CD164 inhibitor, a PARP1 inhibitor or any combination thereof, as described herein.

The present disclosure also provides methods of preventing a subject from developing CHIP by administering a therapeutic agent that prevents or reduces development of CHIP. In some embodiments, the method comprises determining whether the subject has an LY75 variant nucleic acid molecule by obtaining or having obtained a biological sample from the subject, and performing or having performed a sequence analysis on the biological sample to determine if the subject has an LY75 variant nucleic acid molecule. When the subject does not have an LY75 variant nucleic acid molecule, the therapeutic agent that prevents or reduces development of CHIP is administered or continued to be administered to the subject in a standard dosage amount, and/or an LY75 inhibitor, is administered to the subject. When the subject has an LY75 variant nucleic acid molecule, the therapeutic agent that prevents or reduces development of CHIP is administered or continued to be administered to the subject in an amount that is the same as or less than a standard dosage amount, and/or an LY75 inhibitor is administered to the subject.

The present disclosure also provides methods of preventing a subject from developing CHIP by administering a therapeutic agent that prevents or reduces development of CHIP. In some embodiments, the method comprises determining whether the subject has a CD164 variant nucleic acid molecule by obtaining or having obtained a biological sample from the subject, and performing or having performed a sequence analysis on the biological sample to determine if the subject has a CD164 variant nucleic acid molecule. When the subject does not have a CD164 variant nucleic acid molecule, the therapeutic agent that prevents or reduces development of CHIP is administered or continued to be administered to the subject in a standard dosage amount, and/or a CD164 inhibitor, is administered to the subject. When the subject has a CD164 variant nucleic acid molecule, the therapeutic agent that prevents or reduces development of CHIP is administered or continued to be administered to the subject in an amount that is the same as or less than a standard dosage amount, and/or a CD164 inhibitor is administered to the subject.

The present disclosure also provides methods of preventing a subject from developing CHIP by administering a therapeutic agent that prevents or reduces development of CHIP. In some embodiments, the method comprises determining whether the subject has a PARP1 variant nucleic acid molecule by obtaining or having obtained a biological sample from the subject, and performing or having performed a sequence analysis on the biological sample to determine if the subject has a PARP1 variant nucleic acid molecule. When the subject does not have a PARP1 variant nucleic acid molecule, the therapeutic agent that prevents or reduces development of CHIP is administered or continued to be administered to the subject in a standard dosage amount, and/or a PARP1 inhibitor, is administered to the subject. When the subject has a PARP1 variant nucleic acid molecule, the therapeutic agent that prevents or reduces development of CHIP is administered or continued to be administered to the subject in an amount that is the same as or less than a standard dosage amount, and/or a PARP1 inhibitor is administered to the subject.

In some embodiments, when the subject has both an LY75 variant nucleic acid molecule and a CD164 variant nucleic acid molecule, the therapeutic agent that treats or inhibits atherosclerotic cardiovascular disease is administered or continued to be administered to the subject in an amount that is the same as or less than a standard dosage amount, and/or an LY75 inhibitor, or a CD164 inhibitor or both is administered to the subject.

In some embodiments, when the subject has both an LY75 variant nucleic acid molecule and a PARP1 variant nucleic acid molecule, the therapeutic agent that treats or inhibits atherosclerotic cardiovascular disease is administered or continued to be administered to the subject in an amount that is the same as or less than a standard dosage amount, and/or an LY75 inhibitor, or a PARP1 inhibitor, or both is administered to the subject.

In some embodiments when the subject has both a CD164 variant nucleic acid molecule and a PARP1 variant nucleic acid molecule, the therapeutic agent that treats or inhibits atherosclerotic cardiovascular disease is administered or continued to be administered to the subject in an amount that is the same as or less than a standard dosage amount, and/or a CD164 inhibitor, or a PARP1 inhibitor, or both is administered to the subject.

In some embodiments when the subject has an LY75 variant nucleic acid molecule, a CD164 variant nucleic acid molecule, and a PARP1 variant nucleic acid molecule, the therapeutic agent that treats or inhibits atherosclerotic cardiovascular disease is administered or continued to be administered to the subject in an amount that is the same as or less than a standard dosage amount, and/or an LY75 inhibitor, a CD164 inhibitor, a PARP1 inhibitor, or any combination thereof is administered to the subject.

The presence of one or more LY75, CD164, or PARP1 variant nucleic acid molecules indicates the subject has a decreased risk of developing CHIP or a CHIP-related disorder. In some embodiments, the subject has one or more LY75, CD164, or PARP1 variant nucleic acid molecules. In some embodiments, the subject does not have one or more LY75, CD164, or PARP1 variant nucleic acid molecules.

Detecting the presence or absence of one or more LY75, CD164, or PARP1 variant nucleic acid molecules in a biological sample from a subject and/or determining whether a subject has one or more LY75, CD164, or PARP1 variant nucleic acid molecules can be carried out by any of the methods described herein. In some embodiments, these methods can be carried out in vitro. In some embodiments, these methods can be carried out in situ.

In some embodiments, these methods can be carried out in vivo. In any of these embodiments, the polypeptide can be present within a cell obtained from the subject.

In any of the embodiments described herein for nucleic acid molecules, similar methods are also included for polypeptides.

In some embodiments, the LY75 inhibitor is a small molecule. In some embodiments, the LY75 inhibitor is an inhibitory nucleic acid molecule.

In some embodiments, the CD164 inhibitor is a small molecule. In some embodiments, the CD164 inhibitor is atorvastatin. In some embodiments, the CD164 inhibitor is an inhibitory nucleic acid molecule.

In some embodiments, the PARP1 inhibitor is a small molecule. In some embodiments, the PARP1 inhibitor is rucaparib, olaparib, veliparib ABT-888, veliparib, INO-1001, MK4827, CEP-9722, BMN-673, iniparib, AG-14361, NMS-P118, BYK204165, 4-hydroxyquinazoline, pamiparib, E7449, A-966492, niraparib, mortaparib, or ME0238. In some embodiments, the PARP1 inhibitor is an inhibitory nucleic acid molecule.

Examples of therapeutic agents that treat or inhibit myeloid neoplasia include, but are not limited to, arsenic trioxide, azacitidine, cerubidine (daunorubicin hydrochloride), cyclophosphamide, cytarabine, daunorubicin hydrochloride, daunorubicin hydrochloride and cytarabine liposome, daurismo (glasdegib maleate), dexamethasone, doxorubicin hydrochloride, enasidenib mesylate, gemtuzumab ozogamicin, gilteritinib fumarate, glasdegib maleate, idamycin pfs (idarubicin hydrochloride), idarubicin hydrochloride, idhifa (enasidenib mesylate), ivosidenib, midostaurin, mitoxantrone hydrochloride, mylotarg (gemtuzumab ozogamicin), onureg (azacitidine), prednisone, rubidomycin (daunorubicin hydrochloride), rydapt (midostaurin), tabloid (thioguanine), thioguanine, tibsovo (ivosidenib), trisenox (arsenic trioxide), venclexta (venetoclax), venetoclax, vincristine sulfate, vyxeos (daunorubicin hydrochloride and cytarabine liposome), and xospata (gilteritinib fumarate).

Examples of therapeutic agents that treat or inhibit lymphoid neoplasia include, but are not limited to, acalabrutinib, alemtuzumab, arzerra (ofatumumab), bendamustine hydrochloride, bendeka (bendamustine hydrochloride), calquence (acalabrutinib), campath (alemtuzumab), chlorambucil, copiktra (duvelisib), cyclophosphamide, dexamethasone, duvelisib, fludarabine phosphate, gazyva (obinutuzumab), ibrutinib, idelalisib, imbruvica (ibrutinib), leukeran (chlorambucil), obinutuzumab, ofatumumab, prednisone, rituxan (rituximab), rituxan hycela (rituximab and hyaluronidase human), rituximab, rituximab and hyaluronidase human, treanda (bendamustine hydrochloride), truxima (rituximab), venclexta (venetoclax), venetoclax, and zydelig (idelalisib).

Examples of therapeutic agents that treat or inhibit coronary heart disease include, but are not limited to, angiotensin converting enzyme (ACE) inhibitors (such as benazepril, captopril, enalapril, fosinopril, lisinopril, moexipril, perindopril, quinapril, ramipril, or trandolapril); beta blockers (such as acebutolol, atenolol, betaxolol, bisoprolol, bisoprolol/hydrochlorothiazide, metoprolol tartrate, metoprolol succinate, nadolol, pindolol, propranolol, solotol, or timolol); calcium chamel blockers (such as amlodipine, diltiazem, felodipine, isradipine, nicardipine, nifedipine, nisoldipine, orverapamil); metformin; and nitrates (such as nitroglycerin).

Examples of therapeutic agents that treat or inhibit myocardial infarction include, but are not limited to, antiplatelet blood thimers (such as aspirin, clopidogrel, prasugrel, ticagrelor, dipyridamole, or integrilin); angiotensin converting enzyme (ACE) inhibitors (such as benazepril, captopril, enalapril, fosinopril, lisinopril, moexipril, perindopril, quinapril, ramipril, or trandolapril); beta blockers (such as acebutolol, atenolol, betaxolol, bisoprolol, bisoprolol/hydrochlorothiazide, metoprolol tartrate, metoprolol succinate, nadolol, pindolol, propranolol, solotol, or timolol); vasodilators (such as hydralazine or minoxidil); or trombolytics (such as streptokinase, reteplase, alteplase, urokinase, or tenecteplase).

Examples of therapeutic agents that treat or inhibit severe calcified aortic valve stenosis include, but are not limited to, angiotensin converting enzyme (ACE) inhibitors (such as benazepril, captopril, enalapril, fosinopril, lisinopril, moexipril, perindopril, quinapril, ramipril, or trandolapril); beta blockers (such as acebutolol, atenolol, betaxolol, bisoprolol, bisoprolol/hydrochlorothiazide, metoprolol tartrate, metoprolol succinate, nadolol, pindolol, propranolol, solotol, or timolol); diuretics (such as chlorothiazide, chlorthalidone, hydrochlorothiazide, indapamide, metolazone, bumetanide, ethacrynic acid, furosemide, torsemide, amiloride, eplerenone, spironolactone, or triamterene); and antiarrhythmic drugs (such as amiodarone, flecainide, ibutilide, lidocaine, procainamide, propafenone, quinidine, or tocainide).

In some embodiments, the dose of the therapeutic agents that prevent or reduce CHIP can be decreased by about 10%, by about 20%, by about 30%, by about 40%, by about 50%, by about 60%, by about 70%, by about 80%, or by about 90% for subjects that are heterozygous for an LY75 variant nucleic acid molecule, heterozygous for a CD164 variant nucleic acid molecule, or heterozygous for a PARP1 variant nucleic acid molecule (i.e., a less than the standard dosage amount) compared to subjects that are LY75, CD164, and PARP1 reference (who may receive a standard dosage amount). In some embodiments, the dose of the therapeutic agents that prevent or reduce CHIP can be decreased by about 10%, by about 20%, by about 30%, by about 40%, or by about 50%. In addition, the subjects that are heterozygous for an LY75 variant nucleic acid molecule, or heterozygous for a CD164 variant nucleic acid molecule, or heterozygous for a PARP1 variant nucleic acid molecule can be administered less frequently compared to subjects that are LY75, CD164, and PARP1 reference.

In some embodiments, the dose of the therapeutic agents that prevent or reduce CHIP can be decreased by about 10%, by about 20%, by about 30%, by about 40%, by about 50%, for subjects that are homozygous for an LY75 variant nucleic acid molecule compared to subjects that are heterozygous for an LY75 variant nucleic acid molecule. In some embodiments, the dose of the therapeutic agents that prevent or reduce CHIP can be decreased by about 10%, by about 20%, by about 30%, by about 40%, or by about 50%. In addition, the dose of therapeutic agents that prevent or reduce CHIP in subjects that are homozygous for an LY75 variant nucleic acid molecule can be administered less frequently compared to subjects that are heterozygous for an LY75 variant nucleic acid molecule.

In some embodiments, the dose of the therapeutic agents that prevent or reduce CHIP can be decreased by about 10%, by about 20%, by about 30%, by about 40%, by about 50%, for subjects that are homozygous for a CD164 variant nucleic acid molecule compared to subjects that are heterozygous for a CD164 variant nucleic acid molecule. In some embodiments, the dose of the therapeutic agents that prevent or reduce CHIP can be decreased by about 10%, by about 20%, by about 30%, by about 40%, or by about 50%. In addition, the dose of therapeutic agents that prevent or reduce CHIP in subjects that are homozygous for a CD164 variant nucleic acid molecule can be administered less frequently compared to subjects that are heterozygous for a CD164 variant nucleic acid molecule.

In some embodiments, the dose of the therapeutic agents that prevent or reduce CHIP can be decreased by about 10%, by about 20%, by about 30%, by about 40%, by about 50%, for subjects that are homozygous for a PARP1 variant nucleic acid molecule compared to subjects that are heterozygous for a PARP1 variant nucleic acid molecule. In some embodiments, the dose of the therapeutic agents that prevent or reduce CHIP can be decreased by about 10%, by about 20%, by about 30%, by about 40%, or by about 50%. In addition, the dose of therapeutic agents that prevent or reduce CHIP in subjects that are homozygous for a PARP1 variant nucleic acid molecule can be administered less frequently compared to subjects that are heterozygous for a PARP1 variant nucleic acid molecule.

Administration of the therapeutic agents that prevent CHIP and/or LY75 inhibitors, or CD164 inhibitors, PARP1 inhibitors, or any combination thereof can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the therapeutic agents that prevent CHIP and/or LY75 inhibitors, or CD164 inhibitors, PARP1 inhibitors, or any combination thereof can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in CHIP, a decrease/reduction in the severity of CHIP (such as, for example, a reduction or inhibition of development of CHIP), a decrease/reduction in symptoms and CHIP-related effects, delaying the onset of symptoms and CHIP-related effects, reducing the severity of symptoms of c CHIP-related effects, reducing the number of symptoms and CHIP-related effects, reducing the latency of symptoms and CHIP-related effects, an amelioration of symptoms and CHIP-related effects, reducing secondary symptoms, reducing secondary infections, preventing relapse to CHIP, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of CHIP development/progression (such as, for example, a complete or partial avoidance/inhibition or a delay), and an increased survival time of the affected host animal, following administration of a therapeutic protocol. Treatment of CHIP encompasses the treatment of a subject already diagnosed as having any form of CHIP at any clinical stage or manifestation, the delay of the onset or evolution or aggravation or deterioration of the symptoms or signs of CHIP, and/or preventing and/or reducing the severity of CHIP.

In any of the embodiments described herein, for subjects that carry a loss-of-function variant for TET2 (such as, for example, due to the presence of an INDEL; a TET2 somatic mutation deficiency), the methods of treatment and prevention can exclude treatment with a PARP1 inhibitor. Such subjects can be otherwise treated as described herein.

The present disclosure also provides methods of identifying a subject having an increased risk of developing CHIP. In some embodiments, the method comprises determining or having determined in a biological sample obtained from the subject the presence or absence of an LY75 variant nucleic acid molecule, a CD164 variant nucleic acid molecule, and/or a PARP1 variant nucleic acid molecule. When the subject lacks an LY75, a CD164, and/or a PARP1 variant nucleic acid molecule (i.e., the subject is genotypically categorized as LY75, CD164, and/or PARP1 reference), then the subject has an increased risk of developing CHIP. When the subject has one or more of an LY75 variant nucleic acid molecule, a CD164 variant nucleic acid molecule, and/or a PARP1 variant nucleic acid molecule (i.e., the subject is heterozygous or homozygous for one or more of an LY75, a CD164, or a PARP1 variant nucleic acid molecule), then the subject has a decreased risk of developing CHIP.

Having a single copy of an LY75 variant nucleic acid molecule is more protective of a subject from developing CHIP than having no copies of an LY75 variant nucleic acid molecule. Having a single copy of a CD164 variant nucleic acid molecule is more protective of a subject from developing CHIP than having no copies of a CD164 variant nucleic acid molecule. Having a single copy of a PARP1 variant nucleic acid molecule is more protective of a subject from developing CHIP than having no copies of a PARP1 variant nucleic acid molecule.

Without intending to be limited to any particular theory or mechanism of action, it is believed that a single copy of an LY75 variant nucleic acid molecule, a CD164, variant nucleic acid molecule, or a PARP1 variant nucleic acid molecule (i.e., heterozygous for one or more of an LY75, a CD164, or a PARP1 variant nucleic acid molecule) is protective of a subject from developing CHIP and CHIP-related disorders, and it is also believed that having two copies of an LY75 variant nucleic acid molecule, a CD164 variant nucleic acid molecule, or a PARP1 variant nucleic acid molecule (i.e., homozygous for one or more orf an LY75, a CD164, or a PARP1 variant nucleic acid molecule) may be more protective of a subject from developing CHIP and CHIP-related disorders, relative to a subject with a single copy of a corresponding LY75, CD164, or PARP1 variant nucleic acid molecule.

Thus, in some embodiments, a single copy of of an LY75 variant nucleic acid molecule, a CD164 variant nucleic acid molecule, or a PARP1 variant nucleic acid molecule may not be completely protective, but instead, may be partially or incompletely protective of a subject from developing CHIP and CHIP-related disorders. While not desiring to be bound by any particular theory, there may be additional factors or molecules involved in the development of CHIP and CHIP-related disorders that are still present in a subject having a single copy of one or more of an LY75, a CD164, or a PARP1 variant nucleic acid molecule, thus resulting in less than complete protection from the development of CHIP and CHIP-related disorders.

Determining whether a subject has one or more of an LY75, a CD164, or a PARP1 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing CHIP, the subject is administered a therapeutic agent that prevents or reduces development of CHIP, and/or an LY75 inhibitor, a CD164 inhibitor, or a PARP1 inhibitor, or any combination thereof, as described herein. For example, when the subject is LY75 reference, and therefore has an increased risk of developing CHIP, the subject is administered an LY75 inhibitor. In addition, when the subject is CD164 reference, and therefore has an increased risk of developing CHIP, the subject is administered a CD164 inhibitor. In addition, when the subject is PARP1 reference, and therefore has an increased risk of developing CHIP, the subject is administered a PARP1 inhibitor. In some embodiments, such a subject is also administered a therapeutic agent that prevents or reduces development of CHIP.

In some embodiments, such a subject is also administered a therapeutic agent that prevents or reduces development of CHIP. In some embodiments, when the subject is homozygous for one or more of an LY75, a CD164, or a PARP1 variant nucleic acid molecule, the subject is administered the therapeutic agent that prevents or reduces development of CHIP in a dosage amount that is the same as or less than a standard dosage amount.

In some embodiments, when the subject is heterozygous for an LY75 variant nucleic acid molecule, the subject is administered the therapeutic agent that prevents or reduces development of CHIP in a dosage amount that is the same as or less than a standard dosage amount, and is also administered an LY75 inhibitor. In some embodiments, when the subject is heterozygous for a CD164 variant nucleic acid molecule, the subject is administered the therapeutic agent that prevents or reduces development of CHIP in a dosage amount that is the same as or less than a standard dosage amount, and is also administered an CD164 inhibitor. In some embodiments, when the subject is heterozygous for a PARP1 variant nucleic acid molecule, the subject is administered the therapeutic agent that prevents or reduces development of CHIP in a dosage amount that is the same as or less than a standard dosage amount, and is also administered an PARP1 inhibitor.

In some embodiments, the subject is heterozygous for both an LY75 variant nucleic acid molecule and a CD164 variant nucleic acid molecule, and the subject is further administered or continued to be administered the therapeutic agent that prevents or reduces development of CHIP in an amount that is the same as or less than a standard dosage amount, and is administered an LY75 inhibitor, a CD164 inhibitor, or both.

In some embodiments, the subject is heterozygous for both an LY75 variant nucleic acid molecule and a PARP1 variant nucleic acid molecule, and the subject is further administered or continued to be administered the therapeutic agent that prevents or reduces development of CHIP in an amount that is the same as or less than a standard dosage amount, and is administered an LY75 inhibitor, a PARP1 inhibitor, or both.

In some embodiments, the subject is heterozygous for both a CD164 variant nucleic acid molecule and a PARP1 variant nucleic acid molecule, and the subject is further administered or continued to be administered the therapeutic agent that prevents or reduces development of CHIP in an amount that is the same as or less than a standard dosage amount, and is administered a CD164 inhibitor, a PARP1 inhibitor, or both.

In some embodiments, the subject is heterozygous for an LY75 variant nucleic acid molecule, a CD164 variant nucleic acid molecule, and a PARP1 variant nucleic acid molecule, and the subject is further administered or continued to be administered the therapeutic agent that prevents or reduces development of CHIP in an amount that is the same as or less than a standard dosage amount, and is administered an LY75 inhibitor, a CD164 inhibitor, a PARP1 inhibitor or any combination thereof.

In some embodiments, the subject is LY75, CD164, and PARP1 reference. In some embodiments, the subject is heterozygous for one or more of an LY75, a CD164, or a PARP1 variant nucleic acid molecule. In some embodiments, the subject is homozygous for one or more of an LY75, a CD164, or a PARP1 variant nucleic acid molecule.

The present disclosure also provides methods of identifying a subject having an increased risk of developing lung cancer. In some embodiments, the method comprises determining or having determined in a biological sample obtained from the subject the presence or absence of a CHIP somatic mutation in DNMT3A and/or ASXL1. When the subject lacks a CHIP somatic mutation in DNMT3A and/or ASXL1, then the subject does not have an increased risk of developing lung cancer. When the subject has a CHIP somatic mutation in DNMT3A and/or ASXL1, then the subject has an increased risk of developing lung cancer. In some embodiments, the subject is a smoker. In some embodiments, the subject is a non-smoker. In some embodiments, the CHIP somatic mutation is in DNMT3A. In some embodiments, the CHIP somatic mutation is in ASXL1. Subjects not having an increased risk of developing lung cancer because they lack a CHIP somatic mutation in DNMT3A and/or ASXL1, may still have an increased risk relative to the average individual for reasons such as smoking, breathing toxic chemicals at work, city pollution, etc.

Determining whether a subject has one or more CHIP somatic mutations in DNMT3A and/or ASXL1 can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule having the CHIP somatic mutation in DNMT3A and/or ASXL1 can be present within a cell obtained from the subject.

In some embodiments, when a subject is identified as having an increased risk of developing lung cancer, the subject can be subjected to enhanced monitoring, a lifestyle change, and/or lowering exposure to a hazardous substance. For example, a subject can be monitored more frequently for lung cancer pathology and/or symptoms. In some embodiments, such subjects can have chest x-rays, or the like, more frequently compared to subjects that do not have such an increased risk of developing lung cancer. In some embodiments, the subject can undergo additional monitoring for lung cancer-associated somatic mutations (such as EGFR mutations, KRAS mutations, etc.) using DNA from sputum and/or blood (i.e., more frequent cell-free DNA testing/monitoring). In some embodiments, enhanced surveillance options may include earlier magnetic resonance imaging (MRI) monitoring. In some embodiments, such subjects who are smokers can initiate smoking cessation procedures. In some embodiments, the lifestyle change can comprise lowering exposure to an environmental risk factor selected from second-hand smoke, radon, and workplace smoke exposure. In some embodiments, the hazardous substance is selected from asbestos, arsenic, nickel, chromium, beryllium, cadmium, silica, diesel exhaust, tar, or soot, or any combination thereof. In some embodiments, such subjects can undergo palliative or preventative treatment with a therapeutic agent. In some embodiments, the subject can be administered a therapeutically effective amount of erlotinib, 5-(p-methoxyphenyl)-1,2-dithiole-3-thione, deguelin, or iloporost, or any combination thereof.

In some embodiments, the subject has a CHIP somatic mutation in DNMT3A and/or ASXL1. In some embodiments, the subject does not have a CHIP somatic mutation in DNMT3A and/or ASXL1.

In any of the embodiments described herein, for subjects that are determined to have an increased risk of developing CHIP, such subjects that also carry a loss-of-function variant for TET2 (such as, for example, due to the presence of an INDEL; a TET2 somatic mutation deficiency) can undergo a treatment or prevention regimen that excludes treatment with a PARP1 inhibitor. Such subjects can be otherwise treated as described herein. Accordingly, subjects having a loss-of-function variant for TET2 and who have been determined to have an increased risk of developing CHIP can be excluded from the population of subjects amenable for treatment with a PARP1 inhibitor.

The biological sample for detection of a CHIP somatic mutation in DNMT3A and/or ASXL1 can be derived from any cell, tissue, or biological fluid from the subject. The biological sample may comprise any clinically relevant tissue, such as lung tissue or lung cells, such as from a biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. The detection of a CHIP somatic mutation in DNMT3A and/or ASXL1 can be carried out by methods similar to detection of any of the variant nucleic acid molecules described herein using the appropriate primers and probes.

The lung cancer can comprise a non-small cell lung cancer, a small cell lung cancer, mesothelioma, lung carcinoid tumor, or a chest wall tumor. In some embodiments, the lung cancer comprises a non-small cell lung cancer. In some embodiments, the lung cancer comprises a small cell lung cancer. In some embodiments, the lung cancer comprises mesothelioma. In some embodiments, the lung cancer comprises a lung carcinoid tumor. In some embodiments, the lung cancer comprises a chest wall tumor.

In some embodiments, any of the methods described herein can further comprise determining the subject's burden of having one or more of an LY75, a CD164, or a PARP1 variant nucleic acid molecule, and/or one or more of an LY75, a CD164, or a PARP1 predicted loss-of-function variant polypeptide associated with a decreased risk of developing CHIP and CHIP-related disorders. The burden is the sum of all variants in the LY75 gene, the CD164 gene, and/or the PARP1 gene, which can be carried out in an association analysis with CHIP. In some embodiments, the subject is homozygous for one or more LY75 variant nucleic acid molecules associated with a decreased risk of developing CHIP. In some embodiments, the subject is heterozygous for one or more LY75 variant nucleic acid molecules associated with a decreased risk of developing CHIP. In some embodiments, the subject is homozygous for one or more CD164 variant nucleic acid molecules associated with a decreased risk of developing CHIP. In some embodiments, the subject is heterozygous for one or more CD164 variant nucleic acid molecules associated with a decreased risk of developing CHIP. In some embodiments, the subject is homozygous for one or more PARP1 variant nucleic acid molecules associated with a decreased risk of developing CHIP. In some embodiments, the subject is heterozygous for one or more PARP1 variant nucleic acid molecules associated with a decreased risk of developing CHIP.

The result of the association analysis suggests that LY75, CD164, and/or PARP1 variant nucleic acid molecules are associated with decreased risk of developing CHIP. When the subject has a lower burden, the subject is at a higher risk of developing CHIP and the subject is administered or continued to be administered the therapeutic agent that prevents or reduces development of CHIP in a standard dosage amount, and/or an LY75 inhibitor, a CD164 inhibitor, a PARP1 inhibitor, or any combination thereof. When the subject has a greater burden, the subject is at a lower risk of developing CHIP and the subject is administered or continued to be administered the therapeutic agent that prevents or reduces development of CHIP in an amount that is the same as or less than the standard dosage amount. The greater the burden, the lower the risk of developing CHIP. Alternately, the gene burden analysis can comprise determining whether CHIP carriers are more likely to have any of the variants aggregated in the burden framework due to their gene effects.

In some embodiments, the subject's burden of having any one or more of an LY75, a CD164, and/or a PARP1 variant nucleic acid molecule, or an LY75 predicted loss-of-function polypeptide, a CD164 predicted loss-of-function polypeptide, and/or a PARP1 predicted loss-of-function polypeptide represents a weighted sum of a plurality of any of the LY75, CD164, or PARP1 variant nucleic acid molecules or predicted loss-of-function polypeptides. In some embodiments, the burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the LY75 gene, the CD164 gene, or the PARP1 gene where the genetic burden is the number of alleles multiplied by the association estimate with CHIP or related outcome for each allele (e.g., a weighted polygenic burden score). This can include any genetic variants, regardless of their genomic amotation, in proximity to any one of the LY75 gene, the CD164 gene, and/or the PARP1 gene (up to 10 Mb around the gene) that show a non-zero association with CHIP-related traits in a genetic association analysis. In some embodiments, when the subject has a burden above a desired threshold score, the subject has a decreased risk of developing CHIP. In some embodiments, when the subject has a burden below a desired threshold score, the subject has an increased risk of developing CHIP.

In some embodiments, the burden may be divided into quintiles, e.g., top quintile, intermediate quintile, and bottom quintile, wherein the top quintile of burden corresponds to the lowest risk group and the bottom quintile of burden corresponds to the highest risk group. In some embodiments, a subject having a greater burden comprises the highest weighted burdens, including, but not limited to the top 10%, top 20%, top 30%, top 40%, or top 50% of burdens from a subject population. In some embodiments, the genetic variants comprise the genetic variants having association with CHIP in the top 10%, top 20%, top 30%, top 40%, or top 50% of p-value range for the association. In some embodiments, each of the identified genetic variants comprise the genetic variants having association with CHIP with p-value of no more than about 10⁻², no more than about 10⁻³, no more than about 10⁻⁴, no more than about 10⁻⁵, no more than about 10⁻⁶, no more than about 10⁻², no more than about 10⁻⁸, no more than about 10⁻⁹, no more than about 10⁻¹⁰, no more than about 10⁻¹¹, no more than about 10⁻¹², no more than about 10⁻¹³, no more than about 10⁻¹⁴, or no more than about or 10⁻¹⁵. In some embodiments, the identified genetic variants comprise the genetic variants having association with CHIP with p-value of less than 5×10⁻⁸. In some embodiments, the identified genetic variants comprise genetic variants having association with CHIP in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having burdens in the bottom decile, quintile, or tertile in a reference population. The threshold of the burden is determined on the basis of the nature of the intended practical application and the risk difference that would be considered meaningful for that practical application.

In some embodiments, when a subject is identified as having an increased risk of developing CHIP, the subject is further administered a therapeutic agent that prevents or reduces CHIP, and/or an LY75 inhibitor, a CD164 inhibitor, a PARP1 inhibitor, or any combination thereof, as described herein. For example, when the subject is LY75 reference, and therefore has an increased risk of developing CHIP, the subject is administered an LY75 inhibitor. In addition, when the subject CD164 reference, and therefore has an increased risk of developing CHIP, the subject is administered a CD164 inhibitor. In addition, when the subject PARP1 reference, and therefore has an increased risk of developing CHIP, the subject is administered a PARP1 inhibitor.

In some embodiments, such a subject is also administered a therapeutic agent that prevents or reduces development of CHIP. In some embodiments, when the subject is heterozygous for an LY75 variant nucleic acid molecule, the subject is administered the therapeutic agent that prevents or reduces development of CHIP in a dosage amount that is the same as or less than a standard dosage amount, and is also administered an LY75 inhibitor. In some embodiments, when the subject is heterozygous for a CD164 variant nucleic acid molecule, the subject is administered the therapeutic agent that prevents or reduces development of CHIP in a dosage amount that is the same as or less than a standard dosage amount, and is also administered a CD164 inhibitor. In some embodiments, when the subject is heterozygous for a PARP1 variant nucleic acid molecule, the subject is administered the therapeutic agent that prevents or reduces development of CHIP in a dosage amount that is the same as or less than a standard dosage amount, and is also administered a PARP1 inhibitor.

In some embodiments, the subject is LY75, CD164, and PARP1 reference. In some embodiments, the subject is heterozygous for one or more of an LY75, a CD164, or a PARP1 variant nucleic acid molecule. Furthermore, when the subject has a lower burden for having one or more of an LY75, a CD164, or a PARP1 variant nucleic acid molecule, and therefore has an increased risk of developing CHIP, the subject is administered a therapeutic agent that prevents or reduces development of CHIP. In some embodiments, when the subject has a lower burden for having one or more of an LY75, a CD164, or a PARP1 variant nucleic acid molecule, the subject is administered the therapeutic agent that prevents or reduces development of CHIP in a dosage amount that is the same as or greater than the standard dosage amount administered to a subject who has a greater burden for having one or more of an LY75, a CD164, or a PARP1 variant nucleic acid molecule.

In some embodiments, any of the methods described herein can further comprise determining the subject's burden of having a CHIP somatic mutation in DNMT3A and/or ASXL1 associated with an increased risk of developing lung cancer. The burden is the sum of all somatic mutations in the DNMT3A gene and/or ASXL1 gene, which can be carried out in an association analysis with lung cancer. In some embodiments, the subject has a CHIP somatic mutation in DNMT3A associated with an increased risk of developing lung cancer. In some embodiments, the subject does not have a CHIP somatic mutation in DNMT3A associated with an increased risk of developing lung cancer. In some embodiments, the subject has a CHIP somatic mutation in ASXL1 associated with an increased risk of developing lung cancer. In some embodiments, the subject does not have a CHIP somatic mutation in ASXL1 associated with an increased risk of developing lung cancer.

The result of the association analysis may indicate that DNMT3A and/or ASXL1 somatic mutations are associated with an increased risk of developing lung cancer. When the subject has a lower burden, the subject does not have an increased risk of developing lung cancer. When the subject has a greater burden, the subject has an increased risk of developing lung cancer, the subject can undergo any of the procedures described herein related to lung cancer. The greater the burden, the greater the risk of developing lung cancer.

Representative DNMT3A somatic mutations include:

| | | | | |
|---|---|---|---|---|
| 2:25234280:TACA | NA | frameshift | c.2727_2737delTG | p.Phe909fs |
| CACACGCA:T | | | CGTGTGTGT | |
| 2:25234286:C:A | NA | missense | c.2732G > T | p.Cys911Phe |
| 2:25234286:C:T | rs906113912 | missense | c.2732G > A | p.Cys911Tyr |
| 2:25234288:CG:C | | frameshift | c.2729delC | p.Ala910fs |
| 2:25234289:G:A | rs1459396018 | missense | c.2729C > T | p.Ala910Val |
| 2:25234289:G:C | NA | missense | c.2729C > G | p.Ala910Gly |
| 2:25234289:G:GC | NA | frameshift | c.2728dupG | p.Ala910fs |
| 2:25234289:GC:G | NA | frameshift | c.2728delG | p.Ala910fs |
| 2:25234290:C:CA | NA | frameshift | c.2727dupT | p.Ala910fs |
| 2:25234290:C:G | NA | missense | c.2728G > C | p.Ala910Pro |
| 2:25234290:C:T | NA | missense | c.2728G > A | p.Ala910Thr |
| 2:25234290:CA:C | | frameshift | c.2727delT | p.Phe909fs |
| 2:25234290:CAA: | | frameshift | c.2726_2727delTT | p.Phe909fs |
| C | | | | |
| 2:25234291:A:C | NA | missense | c.2727T > G | p.Phe909Leu |
| 2:25234291:A:T | NA | missense | c.2727T > A | p.Phe909Leu |
| 2:25234292:A:C | NA | missense | c.2726T > G | p.Phe909Cys |
| 2:25234292:A:G | NA | missense | c.2726T > C | p.Phe909Ser |
| 2:25234293:A:C | NA | missense | c.2725T > G | p.Phe909Val |
| 2:25234293:A:G | NA | missense | c.2725T > C | p.Phe909Leu |
| 2:25234293:A:T | rs1254967511 | missense | c.2725T > A | p.Phe909Ile |
| 2:25234294:A:C | NA | stop_gained | c.2724T > G | p.Tyr908* |
| 2:25234294:A:T | NA | stop_gained | c.2724T > A | p.Tyr908* |
| 2:25234294:AT:A | NA | frameshift | c.2723delA | p.Tyr908fs |
| 2:25234294:ATAC | NA | frameshift | c.2717_2723delAG | p.Lys906fs |
| TCCT:A | | | GAGTA | |
| 2:25234294:ATAC | NA | frameshift | c.2716_2723delAA | p.Lys906fs |
| TCCTT:A | | | GGAGTA | |
| 2:25234295:T:C | rs780666472 | missense | c.2723A > G | p.Tyr908Cys |
| 2:25234295:T:G | NA | missense | c.2723A > C | p.Tyr908Ser |
| 2:25234295:T:TA | NA | frameshift | c.2722dupT | p.Tyr908fs |
| 2:25234295:T:TA | NA | frameshift | c.2719_2722dupG | p.Tyr908fs |
| CTC | | | AGT | |
| 2:25234295:T:TG | NA | frameshift | c.2722_2723insTC | p.Tyr908fs |
| A | | | | |
| 2:25234295:TA:T | NA | frameshift | c.2722delT | p.Tyr908fs |
| 2:25234295:TACT | NA | frameshift | c.2719_2722delGA | p.Glu907fs |
| C:T | | | GT | |
| 2:25234296:A:C | NA | missense | c.2722T > G | p.Tyr908Asp |
| 2:25234296:A:T | | missense | c.2722T > A | p.Tyr908Asn |
| 2:25234296:AC:A | NA | frameshift | c.2721delG | p.Glu907fs |
| 2:25234297:CT:C | NA | frameshift | c.2720delA | p.Glu907fs |
| 2:25234298:T:A | NA | missense | c.2720A > T | p.Glu907Val |
| 2:25234298:T:C | NA | missense | c.2720A > G | p.Glu907Gly |
| 2:25234298:T:TC | NA | frameshift | c.2719dupG | p.Glu907fs |
| 2:25234298:TC:T | NA | frameshift | c.2719delG | p.Glu907fs |
| 2:25234298:TCCT | NA | frameshift | c.2703_2719delCTT | p.Phe902fs |
| TCAGCGGAGCGAAG: | | | CGCTCCGCTGAAGG | |
| T | | | | |
| 2:25234299:C:A | NA | stop_gained | c.2719G > T | p.Glu907* |
| 2:25234300:C:A | NA | missense | c.2718G > T | p.Lys906Asn |
| 2:25234300:C:CT | NA | frameshift | c.2717dupA | p.Glu907fs |
| 2:25234300:C:G | NA | missense | c.2718G > C | p.Lys906Asn |
| 2:25234300:CT:C | NA | frameshift | c.2717delA | p.Lys906fs |
| 2:25234300:CTTC | NA | frameshift | c.2714_2717delTG | p.Leu905fs |
| A:C | | | AA | |
| 2:25234302:T:A | NA | stop_gained | c.2716A > T | p.Lys906* |
| 2:25234302:T:C | rs1240890824 | missense | c.2716A > G | p.Lys906Glu |
| 2:25234302:TCAG | NA | frameshift | c.2697_2715delCC | p.His900fs |
| CGGAGCGAAGAG | | | ACCTCTTCGCTCCG | |
| GTGG:T | | | CTG | |
| 2:25234303:CA:C | NA | frameshift | c.2714delT | p.Leu905fs |
| 2:25234303:CAGC | NA | frameshift | c.2710_2714delCC | p.Pro904fs |
| GG:C | | | GCT | |
| 2:25234303:CAGC | NA | frameshift | c.2705_2714delTC | p.Phe902fs |
| GGAGCGA:C | | | GCTCCGCT | |
| 2:25234303:CAGC | NA | inframe_indel | c.2703_2714delCTT | p.Phe902_Leu905 |
| GGAGCGAAG:C | | | CGCTCCGCT | del |
| 2:25234304:A:C | rs751868166 | missense | c.2714T > G | p.Leu905Arg |
| 2:25234304:A:G | | missense | c.2714T > C | p.Leu905Pro |
| 2:25234304:A:T | rs751868166 | missense | c.2714T > A | p.Leu905Gln |
| 2:25234304:AGC | NA | frameshift | c.2701_2713delCT | p.Leu901fs |
| GGAGCGAAGAG: | | | CTTCGCTCCGC | |
| A | | | | |
| 2:25234305:G:C | NA | missense | c.2713C > G | p.Leu905Val |
| 2:25234305:G:T | NA | missense | c.2713C > A | p.Leu905Met |
| 2:25234306:C:CG | NA | frameshift | c.2711dupC | p.Leu905fs |
| 2:25234306:CG:C | NA | frameshift | c.2711delC | p.Pro904fs |
| 2:25234306:CGG | NA | frameshift | c.2701_2711delCT | p.Leu901fs |
| AGCGAAGAG:C | | | CTTCGCTCC | |
| 2:25234307:G:A | rs149095705 | missense | c.2711C > T | p.Pro904Leu |
| 2:25234307:G:C | rs149095705 | missense | c.2711C > G | p.Pro904Arg |
| 2:25234307:G:GG | NA | frameshift | c.2707_2710dupGC | p.Pro904fs |
| AGC | | | TC | |
| 2:25234307:G:T | rs149095705 | missense | c.2711C > A | p.Pro904Gln |
| 2:25234307:GGA | NA | frameshift | c.2707_2710delGC | p.Ala903fs |
| GC:G | | | TC | |
| 2:25234307:GGA | NA | frameshift | c.2704_2710delTTC | p.Phe902fs |
| GCGAA:G | | | GCTC | |
| 2:25234308:G:A | NA | missense | c.2710C > T | p.Pro904Ser |
| 2:25234308:G:C | NA | missense | c.2710C > G | p.Pro904Ala |
| 2:25234308:GAG | NA | frameshift | c.2705_2709delTC | p.Phe902fs |
| CGA:G | | | GCT | |
| 2:25234309:AGC: | NA | frameshift | c.2707_2708delGC | p.Ala903fs |
| A | | | | |
| 2:25234309:AGC | NA | frameshift | c.2698_2708delCA | p.His900fs |
| GAAGAGGTG:A | | | CCTCTTCGC | |
| 2:25234310:G:A | NA | missense | c.2708C > T | p.Ala903Val |
| 2:25234310:G:T | NA | missense | c.2708C > A | p.Ala903Asp |
| 2:25234310:GC:G | rs1323968409 | frameshift | c.2707delG | p.Ala903fs |
| 2:25234311:C:CG | NA | frameshift | c.2706dupC | p.Ala903fs |
| 2:25234311:C:CG | NA | frameshift | c.2666_2706dupTG | p.Ala903fs |
| AAGAGGTGGCGG | | | GGCCGGTCATGGA | |
| ATGACTGGCACGC | | | GCGTGCCAGTCATC | |
| TCCATGACCGGCC | | | CGCCACCTCTTC | |
| CA | | | | |
| 2:25234311:C:G | NA | missense | c.2707G > C | p.Ala903Pro |
| 2:25234311:C:T | rs749167103 | missense | c.2707G > A | p.Ala903Thr |
| 2:25234311:CG:C | | frameshift | c.2706delC | p.Phe902fs |
| 2:25234312:G:GA | NA | frameshift | c.2705dupT | p.Ala903fs |
| 2:25234312:G:GT | NA | frameshift | c.2705_2706insA | p.Phe902fs |
| 2:25234312:GAA: | NA | frameshift | c.2704_2705delTT | p.Phe902fs |
| G | | | | |
| 2:25234313:A:AA | NA | frameshift | c.2700_2704dupCC | p.Phe902fs |
| GAGG | | | TCT | |
| 2:25234313:A:G | rs587777510 | missense | c.2705T > C | p.Phe902Ser |
| 2:25234313:A:T | NA | missense | c.2705T > A | p.Phe902Tyr |
| 2:25234314:A:C | NA | missense | c.2704T > G | p.Phe902Val |
| 2:25234315:G:GA | NA | frameshift | c.2702dupT | p.Phe902fs |
| 2:25234315:GA:G | NA | frameshift | c.2702delT | p.Leu901fs |
| 2:25234316:A:C | rs774384800 | missense | c.2702T > G | p.Leu901Arg |
| 2:25234316:A:G | rs774384800 | missense | c.2702T > C | p.Leu901Pro |
| 2:25234316:A:T | NA | missense | c.2702T > A | p.Leu901His |
| 2:25234316:AG:A | NA | frameshift | c.2701delC | p.Leu901fs |
| 2:25234317:G:A | NA | missense | c.2701C > T | p.Leu901Phe |
| 2:25234317:G:C | rs1360815999 | missense | c.2701C > G | p.Leu901Val |
| 2:25234318:GT:G | NA | frameshift | c.2699delA | p.His900fs |
| 2:25234319:T:A | NA | missense | c.2699A > T | p.His900Leu |
| 2:25234319:T:G | NA | missense | c.2699A > C | p.His900Pro |
| 2:25234319:TG:T | NA | frameshift | c.2698delC | p.His900fs |
| 2:25234320:G:A | NA | missense | c.2698C > T | p.His900Tyr |
| 2:25234320:G:GG | NA | frameshift | c.2690_2697dupTC | p.His900fs |
| CGGATGA | | | ATCCGC | |
| 2:25234322:C:A | NA | missense | c.2696G > T | p.Arg899Leu |
| 2:25234322:C:G | rs1480791034 | missense | c.2696G > C | p.Arg899Pro |
| 2:25234322:C:T | NA | missense | c.2696G > A | p.Arg899His |
| 2:25234322:CG:C | NA | frameshift | c.2695delC | p.Arg899fs |
| 2:25234323:G:A | rs771922296 | missense | c.2695C > T | p.Arg899Cys |
| 2:25234323:G:C | NA | missense | c.2695C > G | p.Arg899Gly |
| 2:25234323:G:T | NA | missense | c.2695C > A | p.Arg899Ser |
| 2:25234324:G:GA | NA | frameshift | c.2678_2693dupG | p.Ile898fs |
| TGACTGGCACGCT | | | GAGCGTGCCAGTCA | |
| CC | | | T | |
| 2:25234324:GA:G | NA | frameshift | c.2693delT | p.Ile898fs |
| 2:25234324:GAT | NA | frameshift | c.2678_2693delGG | p.Trp893fs |
| GACTGGCACGCTC | | | AGCGTGCCAGTCAT | |
| C:G | | | | |
| 2:25234325:A:AT | NA | frameshift | c.2692dupA | p.Ile898fs |
| 2:25234325:A:C | NA | missense | c.2693T > G | p.Ile898Ser |
| 2:25234325:A:G | NA | missense | c.2693T > C | p.Ile898Thr |
| 2:25234325:A:T | rs960019580 | missense | c.2693T > A | p.Ile898Asn |
| 2:25234326:T:C | NA | missense | c.2692A > G | p.Ile898Val |
| 2:25234327:GA:G | NA | frameshift | c.2690delT | p.Va1897fs |
| 2:25234327:GACT | NA | inframe_indel | c.2685_2690delGC | p.Pro896_Va1897 |
| GGC:G | | | CAGT | del |
| 2:25234328:A:C | NA | missense | c.2690T > G | p.Va1897Gly |
| 2:25234328:A:T | NA | missense | c.2690T > A | p.Va1897Asp |
| 2:25234328:ACTG | NA | frameshift | c.2686_2689delCC | p.Pro896fs |
| G:A | | | AG | |
| 2:25234329:C:CT | NA | frameshift | c.2688dupA | p.Va1897fs |
| 2:25234329:C:CT | NA | frameshift | c.2688_2689insTG | p.Va1897fs |
| GGCACGGTCA | | | ACCGTGCCA | |
| 2:25234329:C:G | NA | missense | c.2689G > C | p.Va1897Leu |
| 2:25234329:CT:C | NA | frameshift | c.2688delA | p.Va1897fs |
| 2:25234330:T:TG | NA | frameshift | c.2672_2687dupG | p.Ile898fs |
| GCACGCTCCATGA | | | GTCATGGAGCGTGC | |
| CC | | | C | |
| 2:25234330:T:TG | NA | frameshift | c.2687_2688insGC | p.Va1897fs |
| GGC | | | CC | |
| 2:25234330:TG:T | NA | frameshift | c.2687delC | p.Pro896fs |
| 2:25234331:G:A | NA | missense | c.2687C > T | p.Pro896Leu |
| 2:25234332:G:GA | NA | frameshift | c.2685_2686insGA | p.Pro896fs |
| ACGCTC | | | GCGTT | |
| 2:25234333:C:CA | NA | frameshift | c.2684dupT | p.Pro896fs |
| 2:25234333:C:CA | NA | frameshift | c.2680_2684dupA | p.Pro896fs |
| CGCT | | | GCGT | |
| 2:25234334:A:T | NA | missense | c.2684T > A | p.Va1895Glu |
| 2:25234335:C:T | rs762126968 | missense | c.2683G > A | p.Va1895Met |
| 2:25234336:G:C | NA | missense | c.2682C > G | p.Ser894Arg |
| 2:25234336:GCTC | NA | frameshift | c.2659_2681delAG | p.Leu888fs |
| CATGACCGGCCCA | | | ACTGCTGGGCCGGT | |
| GCAGTCT:G | | | CATGGAG | |
| 2:25234337:C:CT | NA | frameshift | c.2680dupA | p.Ser894fs |
| 2:25234337:C:T | NA | missense | c.2681G > A | p.Ser894Asn |
| 2:25234338:T:C | NA | missense | c.2680A > G | p.Ser894Gly |
| 2:25234338:T:TC | NA | frameshift | c.2679dupG | p.Ser894fs |
| 2:25234338:T:TCC | NA | frameshift | c.2667_2679dupG | p.Ser894fs |
| ATGACCGGCCC | | | GGCCGGTCATGG | |
| 2:25234339:C:A | NA | missense | c.2679G > T | p.Trp893Cys |
| 2:25234339:C:T | rs1330526945 | stop_gained | c.2679G > A | p.Trp893* |
| 2:25234340:C:G | NA | missense | c.2678G > C | p.Trp893Ser |
| 2:25234340:C:T | rs750515748 | stop_gained | c.2678G > A | p.Trp893* |
| 2:25234341:A:G | NA | missense | c.2677T > C | p.Trp893Arg |
| 2:25234341:A:T | NA | missense | c.2677T > A | p.Trp893Arg |
| 2:25234342:T:TG | NA | frameshift | c.2675dupC | p.Trp893fs |
| 2:25234342:TGAC | NA | frameshift | c.2672_2675delGG | p.Arg891fs |
| C:T | | | TC | |
| 2:25234343:G:A | rs1355266109 | missense | c.2675C > T | p.Ser892Leu |
| 2:25234343:G:C | NA | stop_gained | c.2675C > G | p.Ser892* |
| 2:25234343:G:T | NA | stop_gained | c.2675C > A | p.Ser892* |
| 2:25234343:GA:G | NA | frameshift | c.2674delT | p.Ser892fs |
| 2:25234344:A:AC | NA | frameshift | c.2670_2673dupCC | p.Ser892fs |
| CGG | | | GG | |
| 2:25234344:AC:A | NA | frameshift | c.2673delG | p.Ser892fs |
| 2:25234344:ACCG | NA | frameshift | c.2670_2673delCC | p.Arg891fs |
| G:A | | | GG | |
| 2:25234344:ACCG | NA | frameshift | c.2660_2673delGA | p.Arg887fs |
| GCCCAGCAGTC:A | | | CTGCTGGGCCGG | |
| 2:25234346:C:T | rs1389510486 | missense | c.2672G > A | p.Arg891Gln |
| 2:25234346:CG:C | rs762357545 | frameshift | c.2671delC | p.Arg891fs |
| 2:25234347:G:A | NA | missense | c.2671C > T | p.Arg891Trp |
| 2:25234347:G:C | NA | missense | c.2671C > G | p.Arg891Gly |
| 2:25234347:G:GG | NA | frameshift | c.2667_2670dupG | p.Arg891fs |
| CCC | | | GGC | |
| 2:25234348:G:GC | rs766070325 | frameshift | c.2669dupG | p.Arg891fs |
| 2:25234348:G:GC | NA | frameshift | c.2668_2669dupG | p.Arg891fs |
| C | | | G | |
| 2:25234348:GC:G | NA | frameshift | c.2669delG | p.Gly890fs |
| 2:25234348:GCC: | NA | frameshift | c.2668_2669delGG | p.Gly890fs |
| G | | | | |
| 2:25234349:C:T | rs752160822 | missense | c.2669G > A | p.Gly890Asp |
| 2:25234349:CCCA | NA | frameshift | c.2659_2668delAG | p.Arg887fs |
| GCAGTCT:C | | | ACTGCTGG | |
| 2:25234350:C:G | NA | missense | c.2668G > C | p.Gly890Arg |
| 2:25234350:C:T | NA | missense | c.2668G > A | p.Gly890Ser |
| 2:25234351:CAGC | NA | frameshift | c.2657_2666delAG | p.Gln886fs |
| AGTCTCT:C | | | AGACTGCT | |
| 2:25234352:A:C | NA | missense | c.2666T > G | p.Leu889Arg |
| 2:25234352:A:G | NA | missense | c.2666T > C | p.Leu889Pro |
| 2:25234352:A:T | NA | missense | c.2666T > A | p.Leu889Gln |
| 2:25234354:C:CA | NA | frameshift | c.2662_2663dupCT | p.Leu889fs |
| G | | | | |
| 2:25234354:CA:C | NA | frameshift | c.2663delT | p.Leu888fs |
| 2:25234354:CAG: | NA | frameshift | c.2662_2663delCT | p.Leu888fs |
| C | | | | |
| 2:25234355:A:G | NA | missense | c.2663T > C | p.Leu888Pro |
| 2:25234355:A:T | NA | missense | c.2663T > A | p.Leu888Gln |
| 2:25234355:AG:A | NA | frameshift | c.2662delC | p.Leu888fs |
| 2:25234355:AGTC | NA | frameshift | c.2644_2662delCG | p.Arg882fs |
| TCTGCCTCGCCAA | | | CTTGGCGAGGCAG | |
| GCG:A | | | AGAC | |
| 2:25234356:G:GT | NA | frameshift | c.2660_2661dupG | p.Leu888fs |
| C | | | A | |
| 2:25234356:GT:G | NA | frameshift | c.2661delA | p.Arg887fs |
| 2:25234356:GTC: | NA | frameshift | c.2660_2661delGA | p.Arg887fs |
| G | | | | |
| 2:25234356:GTCT | NA | frameshift | c.2658_2661delGA | p.Gln886fs |
| C:G | | | GA | |
| 2:25234357:T:G | NA | missense | c.2661A > C | p.Arg887Ser |
| 2:25234357:TCTC | NA | inframe_indel | c.2655_2660delGC | p.Gln886_Arg887 |
| TGC:T | | | AGAG | del |
| 2:25234358:C:A | NA | missense | c.2660G > T | p.Arg887Ile |
| 2:25234359:T:A | NA | stop_gained | c.2659A > T | p.Arg887* |
| 2:25234359:T:C | NA | missense | c.2659A > G | p.Arg887Gly |
| 2:25234360:C:CG | NA | frameshift | c.2657_2658insC | p.Gln886fs |
| 2:25234360:CT:C | NA | frameshift | c.2657delA | p.Gln886fs |
| 2:25234361:T:C | NA | missense | c.2657A > G | p.Gln886Arg |
| 2:25234362:G:A | rs752280049 | stop_gained | c.2656C > T | p.Gln886* |
| 2:25234362:G:C | rs752280049 | missense | c.2656C > G | p.Gln886Glu |
| 2:25234362:G:T | NA | missense | c.2656C > A | p.Gln886Lys |
| 2:25234362:GC:G | NA | frameshift | c.2655delG | p.Arg885fs |
| 2:25234364:C:A | NA | missense | c.2654G > T | p.Arg885Met |
| 2:25234364:C:T | NA | missense | c.2654G > A | p.Arg885Lys |
| 2:25234364:CT:C | NA | frameshift | c.2653delA | p.Arg885fs |
| 2:25234365:TC:T | NA | frameshift | c.2652delG | p.Arg885fs |
| 2:25234366:CGCC | NA | frameshift | c.2638_2651delAT | p.Met880fs |
| AAGCGGCTCAT:C | | | GAGCCGCTTGGC | |
| 2:25234367:G:A | rs559023562 | missense | c.2651C > T | p.Ala884Val |
| 2:25234367:G:GC | NA | frameshift | c.2649_2650dupG | p.Ala884fs |
| C | | | G | |
| 2:25234367:GCG | | frameshift | c.2650delG | p.Ala884fs |
| 2:25234367:GCCA | NA | frameshift | c.2641_2650delAG | p.Ser881fs |
| AGCGGCT:G | | | CCGCTTGG | |
| 2:25234369:C:A | NA | missense | c.2649G > T | p.Leu883Phe |
| 2:25234369:CA:C | NA | frameshift | c.2648delT | p.Leu883fs |
| 2:25234370:A:AC | NA | frameshift | c.2647_2648insG | p.Leu883fs |
| 2:25234370:A:T | rs1307673132 | stop_gained | c.2648T > A | p.Leu883* |
| 2:25234372:GCG | NA | frameshift | c.2630_2645delTCT | p.Va1877fs |
| GCTCATGTTGGAG | | | CCAACATGAGCCG | |
| A:G | | | | |
| 2:25234373:C:A | rs147001633 | missense | c.2645G > T | p.Arg882Leu |
| 2:25234373:C:G | rs147001633 | missense | c.2645G > C | p.Arg882Pro |
| 2:25234373:C:T | rs147001633 | missense | c.2645G > A | p.Arg882His |
| 2:25234373:CG:C | NA | frameshift | c.2644delC | p.Arg882fs |
| 2:25234373:CGG | NA | frameshift | c.2634_2644delCA | p.Asn879fs |
| CTCATGTTG:C | | | ACATGAGCC | |
| 2:25234374:G:A | rs377577594 | missense | c.2644C > T | p.Arg882Cys |
| 2:25234374:G:C | rs377577594 | missense | c.2644C > G | p.Arg882Gly |
| 2:25234374:G:T | rs377577594 | missense | c.2644C > A | p.Arg882Ser |
| 2:25234376:C:CT | NA | frameshift | c.2628_2641dupCG | p.Ser881fs |
| CATGTTGGAGAC | | | TCTCCAACATGA | |
| G | | | | |
| 2:25234376:CT:C | NA | frameshift | c.2641delA | p.Ser881fs |
| 2:25234377:T:TC | NA | frameshift | c.2639_2640dupTG | p.Ser881fs |
| A | | | | |
| 2:25234378:C:A | NA | missense | c.2640G > T | p.Met880Ile |
| 2:25234378:C:CA | NA | frameshift | c.2639dupT | p.Met880fs |
| 2:25234378:C:G | NA | missense | c.2640G > C | p.Met880Ile |
| 2:25234378:C:T | NA | missense | c.2640G > A | p.Met880Ile |
| 2:25234378:CA:C | NA | frameshift | c.2639delT | p.Met880fs |
| 2:25234379:AT:A | NA | frameshift | c.2638delA | p.Met880fs |
| 2:25234379:ATGT | NA | frameshift | c.2635_2638delAA | p.Asn879fs |
| T:A | | | CA | |
| 2:25234379:ATGT | NA | frameshift | c.2614_2638delGT | p.Va1872fs |
| TGGAGACGTCAG | | | CCACTATACTGACG | |
| TATAGTGGAC:A | | | TCTCCAACA | |
| 2:25234380:T:A | NA | missense | c.2638A > T | p.Met880Leu |
| 2:25234380:T:C | rs775365749 | missense | c.2638A > G | p.Met880Val |
| 2:25234380:TG:T | NA | frameshift | c.2637delC | p.Asn879fs |
| 2:25234381:GT:G | NA | frameshift | c.2636delA | p.Asn879fs |
| 2:25234382:T:C | NA | missense | c.2636A > G | p.Asn879Ser |
| 2:25234383:T:C | rs1188120438 | missense | c.2635A > G | p.Asn879Asp |
| 2:25234383:T:TG | NA | frameshift | c.2634dupC | p.Asn879fs |
| 2:25234383:T:TG | NA | frameshift | c.2633_2634dupCC | p.Asn879fs |
| G | | | | |
| 2:25234384:G:GG | NA | frameshift | c.2632_2633dupTC | p.Asn879fs |
| A | | | | |
| 2:25234385:GAG | NA | frameshift | c.2626_2632delGA | p.Asp876fs |
| ACGTC:G | | | CGTCT | |
| 2:25234386:A:G | NA | missense | c.2632T > C | p.Ser878Pro |
| 2:25234387:GA:G | NA | frameshift | c.2630delT | p.Va1877fs |
| 2:25234388:AC:A | NA | frameshift | c.2629delG | p.Va1877fs |
| 2:25234389:C:A | NA | missense | c.2629G > T | p.Va1877Phe |
| 2:25234389:C:CG | NA | frameshift | c.2625_2628dupTG | p.Va1877fs |
| TCA | | | AC | |
| 2:25234389:C:T | NA | missense | c.2629G > A | p.Va1877Ile |
| 2:25234389:CG:C | NA | frameshift | c.2628delC | p.Asp876fs |
| 2:25234389:CGTC | NA | frameshift | c.2625_2628delTG | p.Asp876fs |
| A:C | | | AC | |
| 2:25234390:G:GT | rs774080362 | frameshift | c.2627dupA | p.Asp876fs |
| 2:25234390:G:T | NA | missense | c.2628C > A | p.Asp876Glu |
| 2:25234390:GT:G | NA | frameshift | c.2627delA | p.Asp876fs |
| 2:25234391:T:A | NA | missense | c.2627A > T | p.Asp876Val |
| 2:25234391:T:C | NA | missense | c.2627A > G | p.Asp876Gly |
| 2:25234391:T:G | NA | missense | c.2627A > C | p.Asp876Ala |
| 2:25234391:T:TC | NA | frameshift | c.2626dupG | p.Asp876fs |
| 2:25234391:TC:T | NA | frameshift | c.2626delG | p.Asp876fs |
| 2:25234391:TCA: | NA | frameshift | c.2625_2626delTG | p.Asp876fs |
| T | | | | |
| 2:25234392:C:G | NA | missense | c.2626G > C | p.Asp876His |
| 2:25234392:C:T | rs992291948 | missense | c.2626G > A | p.Asp876Asn |
| 2:25234392:CA:C | NA | frameshift | c.2625delT | p.Asp876fs |
| 2:25234392:CAGT | NA | frameshift | c.2621_2625delAT | p.Tyr874fs |
| AT:C | | | ACT | |
| 2:25234394:G:A | NA | missense | c.2624C > T | p.Thr875Ile |
| 2:25234394:G:C | NA | missense | c.2624C > G | p.Thr875Ser |
| 2:25234394:G:GT | NA | frameshift | c.2622_2623dupTA | p.Thr875fs |
| A | | | | |
| 2:25234394:GTA: | NA | frameshift | c.2622_2623delTA | p.Thr875fs |
| G | | | | |
| 2:25234395:T:C | NA | missense | c.2623A > G | p.Thr875Ala |
| 2:25234396:A:AT | NA | frameshift | c.2621dupA | p.Tyr874fs |
| 2:25234396:A:C | NA | stop_gained | c.2622T > G | p.Tyr874* |
| 2:25234396:A:T | rs770134132 | stop_gained | c.2622T > A | p.Tyr874* |
| 2:25234397:T:A | NA | missense | c.2621A > T | p.Tyr874Phe |
| 2:25234397:T:C | NA | missense | c.2621A > G | p.Tyr874Cys |
| 2:25234397:TA:T | NA | frameshift | c.2620delT | p.Tyr874fs |
| 2:25234398:A:T | NA | missense | c.2620T > A | p.Tyr874Asn |
| 2:25234399:G:C | NA | missense | c.2619C > G | p.His873Gln |
| 2:25234400:T:C | rs1280811819 | missense | c.2618A > G | p.His873Arg |
| 2:25234400:T:TG | NA | frameshift | c.2617dupC | p.His873fs |
| 2:25234400:TG:T | NA | frameshift | c.2617delC | p.His873fs |
| 2:25234400:TGG | NA | frameshift | c.2611_2617delCC | p.Pro871fs |
| ACTGG:T | | | AGTCC | |
| 2:25234401:G:A | rs773610723 | missense | c.2617C > T | p.His873Tyr |
| 2:25234401:G:C | NA | missense | c.2617C > G | p.His873Asp |
| 2:25234403:AC:A | NA | frameshift | c.2614delG | p.Va1872fs |
| 2:25234403:ACTG | NA | frameshift | c.2598_2614delGG | p.Arg866fs |
| GGAAACCAAATAC | | | TATTTGGTTTCCCA | |
| C:A | | | G | |
| 2:25234404:C:T | NA | missense | c.2614G > A | p.Va1872Ile |
| 2:25234404:CT:C | NA | frameshift | c.2613delA | p.Va1872fs |
| 2:25234405:T:TG | NA | frameshift | c.2611_2612dupCC | p.Va1872fs |
| G | | | | |
| 2:25234405:TG:T | rs760010938 | frameshift | c.2612delC | p.Pro871fs |
| 2:25234405:TGG: | NA | frameshift | c.2611_2612delCC | p.Pro871fs |
| T | | | | |
| 2:25234406:G:A | rs1419990763 | missense | c.2612C > T | p.Pro871Leu |
| 2:25234407:G:C | NA | missense | c.2611C > G | p.Pro871Ala |
| 2:25234408:GA:G | NA | frameshift | c.2609delT | p.Phe870fs |
| 2:25234408:GAA: | NA | frameshift | c.2608_2609delTT | p.Phe870fs |
| G | | | | |
| 2:25234409:A:C | NA | missense | c.2609T > G | p.Phe870Cys |
| 2:25234411:AC:A | NA | frameshift | c.2606delG | p.Gly869fs |
| 2:25234412:C:A | NA | missense | c.2606G > T | p.Gly869Val |
| 2:25234412:C:G | NA | missense | c.2606G > C | p.Gly869Ala |
| 2:25234412:C:T | NA | missense | c.2606G > A | p.Gly869Asp |
| 2:25234413:C:CA | NA | frameshift | c.2601_2604dupAT | p.Gly869fs |
| AAT | | | TT | |
| 2:25234413:C:G | NA | missense | c.2605G > C | p.Gly869Arg |
| 2:25234413:C:T | | missense | c.2605G > A | p.Gly869Ser |
| 2:25234413:CA:C | rs753089697 | frameshift | c.2604delT | p.Phe868fs |
| 2:25234414:A:C | rs1239050589 | missense | c.2604T > G | p.Phe868Leu |
| 2:25234414:A:T | NA | missense | c.2604T > A | p.Phe868Leu |
| 2:25234415:A:C | NA | missense | c.2603T > G | p.Phe868Cys |
| 2:25234415:A:G | NA | missense | c.2603T > C | p.Phe868Ser |
| 2:25234415:A:T | NA | missense | c.2603T > A | p.Phe868Tyr |
| 2:25234416:A:C | NA | missense | c.2602T > G | p.Phe868Val |
| 2:25234416:A:G | NA | missense | c.2602T > C | p.Phe868Leu |
| 2:25234417:T:TA | NA | frameshift | c.2600dupT | p.Phe868fs |
| 2:25234417:TACC | NA | splice_acceptor | c.2598- | p.Va1867del |
| CTGGGGGAGAAA | | | 26_2600delTGCCCT | |
| AGGCAGAGAGGG | | | CTCTGCCTTTTCTCC | |
| CA:T | | | CCCAGGGT | |
| 2:25234418:AC:A | rs761283545 | frameshift | c.2599delG | p.Va1867fs |
| 2:25234418:ACCC | NA | frameshift | c.2598- | p.Arg866fs |
| TGGGG:A | | | 6_2599delCCCCAG | |
| | | | GG | |
| 2:25234420:CCTG: | NA | splice_acceptor | c.2598-3_2598- | |
| C | | | 1delCAG | |
| 2:25234420:CCTG | NA | splice_acceptor | c.2598-6_2598- | |
| GGG:C | | | 1delCCCCAG | |
| 2:25234421:C:A | rs766506181 | splice_acceptor | c.2598-1G > T | |
| 2:25234421:C:G | rs766506181 | splice_acceptor | c.2598-1G > C | |
| 2:25234421:C:T | rs766506181 | splice_acceptor | c.2598-1G > A | |
| 2:25234421:CT:C | NA | splice_acceptor | c.2598-2delA | |
| 2:25234422:T:A | NA | splice_acceptor | c.2598-2A > T | |
| 2:25234422:T:C | rs760092360 | splice_acceptor | c.2598-2A > G | |
| 2:25234422:T:G | NA | splice_acceptor | c.2598-2A > C | |
| 2:25235702:GGT | NA | splice_donor | c.2586_2597+4delT | p.Glu863_Arg866 |
| ACCTTTCCATTTCA: | | | GAAATGGAAAGGT | del |
| G | | | AC | |
| 2:25235705:A:C | NA | splice_donor | c.2597+2T > G | |
| 2:25235705:A:G | NA | splice_donor | c.2597+2T > C | |
| 2:25235705:AC:A | NA | splice_donor | c.2597+1delG | |
| 2:25235706:C:T | rs764855628 | splice_donor | c.2597+1G > A | |
| 2:25235707:C:CT | NA | frameshift | c.2596dupA | p.Arg866fs |
| 2:25235707:C:CTT | NA | frameshift | c.2574_2596dupCT | p.Arg866fs |
| TCCATTTCAGTGC | | | TATGGTGCACTGAA | |
| ACCATAAG | | | ATGGAAA | |
| 2:25235707:C:T | NA | missense | c.2597G > A | p.Arg866Lys |
| 2:25235707:CT:C | NA | frameshift | c.2596delA | p.Arg866fs |
| 2:25235710:T:A | rs1187041748 | missense | c.2594A > T | p.Glu865Val |
| 2:25235710:T:C | NA | missense | c.2594A > G | p.Glu865Gly |
| 2:25235710:T:G | NA | missense | c.2594A > C | p.Glu865Ala |
| 2:25235711:C:CC | NA | frameshift | c.2588_2592dupAA | p.Glu865fs |
| ATTT | | | ATG | |
| 2:25235712:CA:C | NA | frameshift | c.2591delT | p.Met864fs |
| 2:25235713:A:G | NA | missense | c.2591T > C | p.Met864Thr |
| 2:25235713:AT:A | NA | frameshift | c.2590delA | p.Met864fs |
| 2:25235713:ATTT | NA | frameshift | c.2566_2590delGA | p.Glu856fs |
| CAGTGCACCATAA | | | GGACATCTTATGGT | |
| GATGTCCTC:A | | | GCACTGAAA | |
| 2:25235716:T:A | NA | missense | c.2588A > T | p.Glu863Val |
| 2:25235717:C:A | NA | stop_gained | c.2587G > T | p.Glu863* |
| 2:25235717:C:T | NA | missense | c.2587G > A | p.Glu863Lys |
| 2:25235718:A:AG | NA | frameshift | c.2584_2585dupAC | p.Glu863fs |
| T | | | | |
| 2:25235718:AGT: | NA | frameshift | c.2584_2585delAC | p.Thr862fs |
| A | | | | |
| 2:25235718:AGT | NA | inframe_indel | c.2577_2585delAT | p.Leu859_Thr862 |
| GCACCAT:A | | | GGTGCAC | delinsPhe |
| 2:25235719:G:A | NA | missense | c.2585C > T | p.Thr862Ile |
| 2:25235719:G:T | NA | missense | c.2585C > A | p.Thr862Asn |
| 2:25235719:GTGC | NA | frameshift | c.2578_2584delTG | p.Trp860fs |
| ACCA:G | | | GTGCA | |
| 2:25235720:TG:T | NA | frameshift | c.2583delC | p.Cys861fs |
| 2:25235721:G:T | NA | stop_gained | c.2583C > A | p.Cys861* |
| 2:25235722:C:T | NA | missense | c.2582G > A | p.Cys861Tyr |
| 2:25235722:CACC | NA | frameshift | c.2578_2581delTG | p.Trp860fs |
| A:C | | | GT | |
| 2:25235723:A:AC | NA | frameshift | c.2580dupG | p.Cys861fs |
| 2:25235723:A:G | NA | missense | c.2581T > C | p.Cys861Arg |
| 2:25235723:ACCA | NA | frameshift | c.2556_2580delGA | p.Met852fs |
| TAAGATGTCCTCT | | | ATGAGAAAGAGGA | |
| TTCTCATTC:A | | | CATCTTATGG | |
| 2:25235724:C:G | NA | missense | c.2580G > C | p.Trp860Cys |
| 2:25235724:C:T | rs376830288 | stop_gained | c.2580G > A | p.Trp860* |
| 2:25235725:C:G | NA | missense | c.2579G > C | p.Trp860Ser |
| 2:25235725:C:T | rs1321116253 | stop_gained | c.2579G > A | p.Trp860* |
| 2:25235725:CAT: | NA | frameshift | c.2577_2578delAT | p.Trp860fs |
| C | | | | |
| 2:25235725:CATA | NA | frameshift | c.2575_2578delTT | p.Leu859fs |
| A:C | | | AT | |
| 2:25235726:A:AT | rs764502627 | frameshift | c.2577dupA | p.Trp860fs |
| 2:25235726:A:C | NA | missense | c.2578T > G | p.Trp860Gly |
| 2:25235726:A:G | rs373014701 | missense | c.2578T > C | p.Trp860Arg |
| 2:25235726:A:T | NA | missense | c.2578T > A | p.Trp860Arg |
| 2:25235726:ATAA | NA | frameshift | c.2571_2577delCA | p.Ile858fs |
| GATG:A | | | TCTTA | |
| 2:25235726:ATAA | NA | frameshift | c.2565_2577delAG | p.Lys855fs |
| GATGTCCTCT:A | | | AGGACATCTTA | |
| 2:25235726:ATAA | NA | frameshift | c.2562_2577delGA | p.Glu854fs |
| GATGTCCTGTTTC: | | | AAGAGGACATCTTA | |
| A | | | | |
| 2:25235727:T:TC | NA | frameshift | c.2576_2577insG | p.Trp860fs |
| 2:25235727:TA:T | NA | frameshift | c.2576delT | p.Leu859fs |
| 2:25235728:A:C | rs1175622747 | stop_gained | c.2576T > G | p.Leu859* |
| 2:25235728:A:T | NA | stop_gained | c.2576T > A | p.Leu859* |
| 2:25235732:TG:T | NA | frameshift | c.2571delC | p.Asp857fs |
| 2:25235734:T:TCC | NA | frameshift | c.2568_2569dupG | p.Asp857fs |
| | | | G | |
| 2:25235734:TC:T | rs1254668110 | frameshift | c.2569delG | p.Asp857fs |
| 2:25235735:C:T | NA | missense | c.2569G > A | p.Asp857Asn |
| 2:25235735:CCT: | rs754004583 | frameshift | c.2567_2568delAG | p.Glu856fs |
| C | | | | |
| 2:25235736:CTCT | NA | frameshift | c.2564_2567delAA | p.Lys855fs |
| T:C | | | GA | |
| 2:25235737:TCTT | NA | frameshift | c.2553_2566delCA | p.Phe851fs |
| TCTCATTCATG:T | | | TGAATGAGAAAG | |
| 2:25235738:C:A | NA | stop_gained | c.2566G > T | p.Glu856* |
| 2:25235738:C:CT | NA | frameshift | c.2565dupA | p.Glu856fs |
| 2:25235738:CT:C | NA | frameshift | c.2565delA | p.Glu856fs |
| 2:25235738:CTT:C | NA | frameshift | c.2564_2565delAA | p.Lys855fs |
| 2:25235740:T:A | NA | missense | c.2564A > T | p.Lys855Ile |
| 2:25235740:TTCT | NA | frameshift | c.2560_2563delGA | p.Glu854fs |
| C:T | | | GA | |
| 2:25235741:T:A | NA | stop_gained | c.2563A > T | p.Lys855* |
| 2:25235742:CTCA | NA | frameshift | c.2557_2561delAA | p.Asn853fs |
| TT:C | | | TGA | |
| 2:25235743:TC:T | NA | frameshift | c.2560delG | p.Glu854fs |
| 2:25235744:CATT | NA | frameshift | c.2553_2559delCA | p.Phe851fs |
| CATG:C | | | TGAAT | |
| 2:25235745:AT:A | NA | frameshift | c.2558delA | p.Asn853fs |
| 2:25235748:C:CA | NA | frameshift | c.2555dupT | p.Met852fs |
| 2:25235749:A:T | NA | missense | c.2555T > A | p.Met852Lys |
| 2:25235749:ATG: | NA | frameshift | c.2553_2554delCA | p.Met852fs |
| A | | | | |
| 2:25235749:ATGA | NA | frameshift | c.2538_2554delGC | p.Gln846fs |
| AGACAGGAAAAT | | | ATTTTCCTGTCTTCA | |
| GC:A | | | | |
| 2:25235750:T:C | rs746967478 | missense | c.2554A > G | p.Met852Val |
| 2:25235750:T:G | NA | missense | c.2554A > C | p.Met852Leu |
| 2:25235750:T:TG | NA | frameshift | c.2553dupC | p.Met852fs |
| 2:25235750:TG:T | rs757223824 | frameshift | c.2553delC | p.Phe851fs |
| 2:25235751:G:GA | NA | frameshift | c.2552dupT | p.Met852fs |
| 2:25235751:GA:G | NA | frameshift | c.2552delT | p.Phe851fs |
| 2:25235751:GAA | NA | frameshift | c.2549_2552delTCT | p.Va1850fs |
| GA:G | | | T | |
| 2:25235751:GAA | NA | frameshift | c.2548_2552delGT | p.Va1850fs |
| GAC:G | | | CTT | |
| 2:25235752:AAG: | rs779108270 | frameshift | c.2550_2551delCT | p.Phe851fs |
| A | | | | |
| 2:25235754:G:GA | NA | frameshift | c.2549dupT | p.Phe851fs |
| 2:25235754:GAC: | NA | frameshift | c.2548_2549delGT | p.Va1850fs |
| G | | | | |
| 2:25235755:AC:A | rs1175091304 | frameshift | c.2548delG | p.Va1850fs |
| 2:25235756:C:G | NA | missense | c.2548G > C | p.Va1850Leu |
| 2:25235756:C:T | NA | missense | c.2548G > A | p.Va1850Ile |
| 2:25235756:CA:C | NA | frameshift | c.2547delT | p.Va1850fs |
| 2:25235756:CAG: | NA | frameshift | c.2546_2547delCT | p.Pro849fs |
| C | | | | |
| 2:25235757:AG:A | rs750600685 | frameshift | c.2546delC | p.Pro849fs |
| 2:25235758:G:A | NA | missense | c.2546C > T | p.Pro849Leu |
| 2:25235758:G:C | NA | missense | c.2546C > G | p.Pro849Arg |
| 2:25235758:G:GG | NA | frameshift | c.2544_2545dupTC | p.Pro849fs |
| A | | | | |
| 2:25235758:G:T | NA | missense | c.2546C > A | p.Pro849His |
| 2:25235759:G:A | NA | missense | c.2545C > T | p.Pro849Ser |
| 2:25235759:G:C | NA | missense | c.2545C > G | p.Pro849Ala |
| 2:25235759:G:GA | NA | frameshift | c.2544dupT | p.Pro849fs |
| 2:25235759:G:T | NA | missense | c.2545C > A | p.Pro849Thr |
| 2:25235759:GA:G | NA | frameshift | c.2544delT | p.Pro849fs |
| 2:25235762:A:C | NA | missense | c.2542T > G | p.Phe848Val |
| 2:25235762:A:T | NA | missense | c.2542T > A | p.Phe848Ile |
| 2:25235763:AT:A | NA | frameshift | c.2540delA | p.His847fs |
| 2:25235764:TG:T | NA | frameshift | c.2539delC | p.His847fs |
| 2:25235764:TGC: | NA | frameshift | c.2538_2539delGC | p.His847fs |
| T | | | | |
| 2:25235764:TGCT | NA | frameshift | c.2535_2539delCC | p.Asp845fs |
| GG:T | | | AGC | |
| 2:25235765:G:T | NA | missense | c.2539C > A | p.His847Asn |
| 2:25235765:GC:G | NA | frameshift | c.2538delG | p.Gln846fs |
| 2:25235766:CT:C | NA | frameshift | c.2537delA | p.Gln846fs |
| 2:25235766:CTG: | NA | frameshift | c.2536_2537delCA | p.Gln846fs |
| C | | | | |
| 2:25235767:TG:T | NA | frameshift | c.2536delC | p.Gln846fs |
| 2:25235768:G:A | rs781139634 | stop_gained | c.2536C > T | p.Gln846* |
| 2:25235769:G:C | NA | missense | c.2535C > G | p.Asp845Glu |
| 2:25235770:TC:T | NA | frameshift | c.2533delG | p.Asp845fs |
| 2:25235771:CTT:C | NA | frameshift | c.2531_2532delAA | p.Lys844fs |
| 2:25235771:CTTT | NA | frameshift | c.2520_2532delAA | p.Ile840fs |
| GCCCTGCTTT:C | | | AGCAGGGCAAA | |
| 2:25235774:T:C | NA | missense | c.2530A > G | p.Lys844Glu |
| 2:25235774:TG:T | NA | frameshift | c.2529delC | p.Asp845fs |
| 2:25235775:GC:G | NA | frameshift | c.2528delG | p.Gly843fs |
| 2:25235776:C:CC | NA | frameshift | c.2524_2527dupCA | p.Gly843fs |
| CTG | | | GG | |
| 2:25235776:C:T | NA | missense | c.2528G > A | p.Gly843Asp |
| 2:25235777:C:G | NA | missense | c.2527G > C | p.Gly843Arg |
| 2:25235778:C:G | NA | missense | c.2526G > C | p.Gln842His |
| 2:25235778:CT:C | NA | frameshift | c.2525delA | p.Gln842fs |
| 2:25235779:T:C | rs771174392 | missense | c.2525A > G | p.Gln842Arg |
| 2:25235779:TG:T | NA | frameshift | c.2524delC | p.Gln842fs |
| 2:25235779:TGCT | NA | frameshift | c.2517_2524delCA | p.Ile840fs |
| TTATG:T | | | TAAAGC | |
| 2:25235780:G:A | rs1015295548 | stop_gained | c.2524C > T | p.Gln842* |
| 2:25235780:G:C | NA | missense | c.2524C > G | p.Gln842Glu |
| 2:25235780:GC:G | NA | frameshift | c.2523delG | p.Lys841fs |
| 2:25235781:C:CTT | NA | frameshift | c.2512_2522dupAA | p.Gln842fs |
| TATGGAGTT | | | CTCCATAAA | |
| 2:25235781:CT:C | NA | frameshift | c.2522delA | p.Lys841fs |
| 2:25235781:CTT:C | NA | frameshift | c.2521_2522delAA | p.Lys841fs |
| 2:25235783:T:C | rs1404111595 | missense | c.2521A > G | p.Lys841Glu |
| 2:25235783:T:G | NA | missense | c.2521A > C | p.Lys841Gln |
| 2:25235783:TTA:T | NA | frameshift | c.2519_2520delTA | p.Ile840fs |
| 2:25235784:TA:T | NA | frameshift | c.2519delT | p.Ile840fs |
| 2:25235786:T:C | NA | missense | c.2518A > G | p.Ile840Val |
| 2:25235786:T:TG | NA | frameshift | c.2517dupC | p.Ile840fs |
| 2:25235786:T:TG | NA | frameshift | c.2516_2517dupCC | p.Ile840fs |
| G | | | | |
| 2:25235786:TG:T | NA | frameshift | c.2517delC | p.Ile840fs |
| 2:25235787:G:GA | NA | frameshift | c.2516_2517insT | p.Ile840fs |
| 2:25235788:G:A | NA | missense | c.2516C > T | p.Ser839Phe |
| 2:25235788:GA:G | NA | frameshift | c.2515delT | p.Ser839fs |
| 2:25235789:A:C | NA | missense | c.2515T > G | p.Ser839Ala |
| 2:25235789:AG:A | NA | frameshift | c.2514delC | p.Ser839fs |
| 2:25235790:G:GT | rs781008582 | frameshift | c.2513dupA | p.Asn838fs |
| 2:25235790:GT:G | NA | frameshift | c.2513delA | p.Asn838fs |
| 2:25235792:T:C | rs961377711 | missense | c.2512A > G | p.Asn838Asp |
| 2:25235794:G:C | rs1374267987 | stop_gained | c.2510C > G | p.Ser837* |
| 2:25235794:G:GA | NA | frameshift | c.2509_2510insTG | p.Ser837fs |
| TCA | | | AT | |
| 2:25235794:G:T | NA | stop_gained | c.2510C > A | p.Ser837* |
| 2:25235795:A:AC | NA | frameshift | c.2508dupG | p.Ser837fs |
| 2:25235797:C:G | NA | missense | c.2507G > C | p.Arg836Thr |
| 2:25235800:G:A | rs1489843853 | missense | c.2504C > T | p.Thr835Met |
| 2:25235800:GT:G | rs1293842202 | frameshift | c.2503delA | p.Thr835fs |
| 2:25235801:T:A | NA | missense | c.2503A > T | p.Thr835Ser |
| 2:25235803:G:A | NA | missense | c.2501C > T | p.Thr834Ile |
| 2:25235803:G:GT | NA | frameshift | c.2500dupA | p.Thr834fs |
| 2:25235804:T:C | NA | missense | c.2500A > G | p.Thr834Ala |
| 2:25235804:T:TA | NA | frameshift | c.2481_2499dupCA | p.Thr834fs |
| ATGGTCCTCACTT | | | GCAAAGTGAGGAC | |
| TGCTG | | | CATT | |
| 2:25235804:TA:T | NA | frameshift | c.2499delT | p.Thr834fs |
| 2:25235806:A:T | NA | missense | c.2498T > A | p.Ile833Asn |
| 2:25235807:T:C | rs745931882 | missense | c.2497A > G | p.Ile833Val |
| 2:25235809:GT:G | NA | frameshift | c.2494delA | p.Thr832fs |
| 2:25235812:C:T | NA | missense | c.2492G > A | p.Arg831Lys |
| 2:25235820:GCTG | NA | frameshift | c.2479- | p.Phe827fs |
| AACTAGATGAAGA: | | | 11_2483delTCTTCA | |
| G | | | TCTAGTTCAG | |
| 2:25235821:CTGA | NA | frameshift | c.2479- | p.Phe827fs |
| ACT:C | | | 2_2482delAGTTCA | |
| 2:25235822:T:TG | NA | stop_gained | c.2481_2482insCTC | p.Phe827_Ser828i |
| AACTAGAG | | | TAGTTC | nsLeuTerPhe |
| 2:25235822:TG:T | rs769831202 | frameshift | c.2481delC | p.Phe827fs |
| 2:25235823:G:GT | NA | frameshift | c.2480_2481insA | p.Phe827fs |
| 2:25235823:G:T | NA | missense | c.2481C > A | p.Phe827Leu |
| 2:25235823:GA:G | NA | frameshift | c.2480delT | p.Phe827fs |
| 2:25235823:GAA: | NA | frameshift | c.2479_2480delTT | p.Phe827fs |
| G | | | | |
| 2:25235825:A:C | NA | missense | c.2479T > G | p.Phe827Val |
| 2:25235826:C:A | NA | splice_acceptor | c.2479-1G > T | |
| 2:25235826:C:G | NA | splice_acceptor | c.2479-1G > C | |
| 2:25235826:C:T | rs775933506 | splice_acceptor | c.2479-1G > A | |
| 2:25235826:CT:C | NA | splice_acceptor | c.2479-2delA | |
| 2:25235827:T:C | rs761171028 | splice_acceptor | c.2479-2A > G | |
| 2:25235827:T:G | NA | splice_acceptor | c.2479-2A > C | |
| 2:25236934:A:AC | NA | splice_donor | c.2478+1dupG | |
| 2:25236934:A:C | rs1327946633 | splice_donor | c.2478+2T > G | |
| 2:25236934:A:G | NA | splice_donor | c.2478+2T > C | |
| 2:25236934:A:T | NA | splice_donor | c.2478+2T > A | |
| 2:25236934:AC:A | NA | splice_donor | c.2478+1delG | |
| 2:25236935:C:A | NA | splice_donor | c.2478+1G > T | |
| 2:25236935:C:G | NA | splice_donor | c.2478+1G > C | |
| 2:25236935:C:T | rs762213449 | splice_donor | c.2478+1G > A | |
| 2:25236935:CCT: | NA | frameshift | c.2477_2478delAG | p.Lys826fs |
| C | | | | |
| 2:25236936:C:G | NA | missense | c.2478G > C | p.Lys826Asn |
| 2:25236936:CT:C | NA | frameshift | c.2477delA | p.Lys826fs |
| 2:25236936:CTTG: | NA | inframe_indel | c.2475_2477delCA | p.Lys826del |
| C | | | A | |
| 2:25236937:T:A | NA | missense | c.2477A > T | p.Lys826Met |
| 2:25236937:T:C | rs770079872 | missense | c.2477A > G | p.Lys826Arg |
| 2:25236937:T:G | rs770079872 | missense | c.2477A > C | p.Lys826Thr |
| 2:25236938:T:C | NA | missense | c.2476A > G | p.Lys826Glu |
| 2:25236939:GGCT | NA | frameshift | c.2468_2474delGG | p.Arg823fs |
| ATCC:G | | | ATAGC | |
| 2:25236940:G:T | NA | missense | c.2474C > A | p.Ala825Asp |
| 2:25236941:C:T | NA | missense | c.2473G > A | p.Ala825Thr |
| 2:25236942:TA:T | NA | frameshift | c.2471delT | p.Ile824fs |
| 2:25236943:A:G | NA | missense | c.2471T > C | p.Ile824Thr |
| 2:25236943:AT:A | NA | frameshift | c.2470delA | p.Ile824fs |
| 2:25236944:T:TC | rs1200243711 | frameshift | c.2469dupG | p.Ile824fs |
| 2:25236944:TC:T | NA | frameshift | c.2469delG | p.Ile824fs |
| 2:25236946:C:T | NA | missense | c.2468G > A | p.Arg823Lys |
| 2:25236946:CT:C | NA | frameshift | c.2467delA | p.Arg823fs |
| 2:25236947:T:C | NA | missense | c.2467A > G | p.Arg823Gly |
| 2:25236948:G:GC | NA | frameshift | c.2465dupG | p.Arg823fs |
| 2:25236948:GC:G | NA | frameshift | c.2465delG | p.Gly822fs |
| 2:25236950:CA:C | NA | frameshift | c.2463delT | p.His821fs |
| 2:25236951:A:C | NA | missense | c.2463T > G | p.His821Gln |
| 2:25236951:A:T | NA | missense | c.2463T > A | p.His821Gln |
| 2:25236951:AT:A | NA | frameshift | c.2462delA | p.His821fs |
| 2:25236952:T:A | NA | missense | c.2462A > T | p.His821Leu |
| 2:25236952:T:C | NA | missense | c.2462A > G | p.His821Arg |
| 2:25236952:T:G | rs772882388 | missense | c.2462A > C | p.His821Pro |
| 2:25236952:TG:T | NA | frameshift | c.2461delC | p.His821fs |
| 2:25236955:T:A | NA | missense | c.2459A > T | p.Glu820Val |
| 2:25236955:T:C | NA | missense | c.2459A > G | p.Glu820Gly |
| 2:25236955:TC:T | NA | frameshift | c.2458delG | p.Glu820fs |
| 2:25236955:TCCA | NA | frameshift | c.2455_2458delCT | p.Leu819fs |
| G:T | | | GG | |
| 2:25236956:C:A | rs1264543822 | stop_gained | c.2458G > T | p.Glu820* |
| 2:25236956:C:G | NA | missense | c.2458G > C | p.Glu820Gln |
| 2:25236956:CCA: | NA | frameshift | c.2456_2457delTG | p.Leu819fs |
| C | | | | |
| 2:25236956:CCAG | NA | frameshift | c.2454_2457delTCT | p.Cys818fs |
| A:C | | | G | |
| 2:25236957:CA:C | NA | frameshift | c.2456delT | p.Leu819fs |
| 2:25236958:A:T | NA | missense | c.2456T > A | p.Leu819Gln |
| 2:25236958:AG:A | NA | frameshift | c.2455delC | p.Leu819fs |
| 2:25236959:G:C | NA | missense | c.2455C > G | p.Leu819Val |
| 2:25236959:G:T | NA | missense | c.2455C > A | p.Leu819Met |
| 2:25236960:A:AC | NA | frameshift | c.2450_2453dupA | p.Cys818fs |
| ACT | | | GTG | |
| 2:25236960:A:AC | NA | frameshift | c.2453_2454insGG | p.Cys818fs |
| C | | | | |
| 2:25236960:A:T | NA | stop_gained | c.2454T > A | p.Cys818* |
| 2:25236960:AC:A | NA | frameshift | c.2453delG | p.Cys818fs |
| 2:25236961:C:A | NA | missense | c.2453G > T | p.Cys818Phe |
| 2:25236961:C:T | NA | missense | c.2453G > A | p.Cys818Tyr |
| 2:25236961:CA:C | NA | frameshift | c.2452delT | p.Cys818fs |
| 2:25236962:A:C | NA | missense | c.2452T > G | p.Cys818Gly |
| 2:25236962:A:G | NA | missense | c.2452T > C | p.Cys818Arg |
| 2:25236962:A:T | NA | missense | c.2452T > A | p.Cys818Ser |
| 2:25236963:C:CT | NA | frameshift | c.2450dupA | p.Cys818fs |
| 2:25236963:CT:C | NA | frameshift | c.2450delA | p.Glu817fs |
| 2:25236964:T:C | NA | missense | c.2450A > G | p.Glu817Gly |
| 2:25236964:TC:T | NA | frameshift | c.2449delG | p.Glu817fs |
| 2:25236965:C:A | rs373873045 | stop_gained | c.2449G > T | p.Glu817* |
| 2:25236966:C:CT | NA | frameshift | c.2444_2447dupTG | p.Gln816fs |
| GCA | | | CA | |
| 2:25236966:CT:C | NA | frameshift | c.2447delA | p.Gln816fs |
| 2:25236967:T:C | NA | missense | c.2447A > G | p.Gln816Arg |
| 2:25236967:TG:T | NA | frameshift | c.2446delC | p.Gln816fs |
| 2:25236968:G:A | rs759187608 | stop_gained | c.2446C > T | p.Gln816* |
| 2:25236969:C:CA | NA | frameshift | c.2444dupT | p.Gln816fs |
| 2:25236969:CA:C | NA | frameshift | c.2444delT | p.Leu815fs |
| 2:25236970:A:G | NA | missense | c.2444T > C | p.Leu815Pro |
| 2:25236970:A:T | NA | missense | c.2444T > A | p.Leu815Gln |
| 2:25236970:AG:A | NA | frameshift | c.2443delC | p.Leu815fs |
| 2:25236971:G:C | NA | missense | c.2443C > G | p.Leu815Val |
| 2:25236971:G:T | NA | missense | c.2443C > A | p.Leu815Met |
| 2:25236972:CT:C | NA | frameshift | c.2441delA | p.Glu814fs |
| 2:25236972:CTCC | NA | frameshift | c.2435_2441delAG | p.Lys812fs |
| AGCT:C | | | CTGGA | |
| 2:25236973:T:C | NA | missense | c.2441A > G | p.Glu814Gly |
| 2:25236973:T:TC | NA | frameshift | c.2440dupG | p.Glu814fs |
| 2:25236974:C:A | rs1174921909 | stop_gained | c.2440G > T | p.Glu814* |
| 2:25236975:CA:C | NA | frameshift | c.2438delT | p.Leu813fs |
| 2:25236977:G:GC | NA | frameshift | c.2430_2436dupTG | p.Leu813fs |
| TTATCA | | | ATAAG | |
| 2:25236977:GC:G | NA | frameshift | c.2436delG | p.Lys812fs |
| 2:25236977:GCTT | NA | frameshift | c.2432_2436delAT | p.Asp811fs |
| AT:G | | | AAG | |
| 2:25236978:CT:C | NA | frameshift | c.2435delA | p.Lys812fs |
| 2:25236979:T:C | NA | missense | c.2435A > G | p.Lys812Arg |
| 2:25236979:T:TTA | NA | frameshift | c.2433_2434dupTA | p.Lys812fs |
| 2:25236980:T:A | NA | stop_gained | c.2434A > T | p.Lys812* |
| 2:25236980:T:TA | rs1287022880 | frameshift | c.2433dupT | p.Lys812fs |
| 2:25236980:TATC | NA | frameshift | c.2420_2433delCC | p.Ser807fs |
| ATTCACAGTGG:T | | | ACTGTGAATGAT | |
| 2:25236981:A:AT | NA | frameshift | c.2429_2432dupAT | p.Asp811fs |
| CAT | | | GA | |
| 2:25236981:A:T | NA | missense | c.2433T > A | p.Asp811Glu |
| 2:25236981:ATC: | NA | frameshift | c.2431_2432delGA | p.Asp811fs |
| A | | | | |
| 2:25236981:ATCA | NA | frameshift | c.2428_2432delAA | p.Asn810fs |
| TT:A | | | TGA | |
| 2:25236983:CA:C | NA | frameshift | c.2430delT | p.Asn810fs |
| 2:25236984:AT:A | NA | frameshift | c.2429delA | p.Asn810fs |
| 2:25236986:TCA: | NA | frameshift | c.2426_2427delTG | p.Va1809fs |
| T | | | | |
| 2:25236989:CA:C | NA | frameshift | c.2424delT | p.Va1809fs |
| 2:25236990:AG:A | NA | frameshift | c.2423delC | p.Thr808fs |
| 2:25236991:GTG | NA | frameshift | c.2419_2422delTC | p.Ser807fs |
| GA:G | | | CA | |
| 2:25236991:GTG | NA | frameshift | c.2409- | p.Arg803fs |
| GATGCCAACGGCC: | | | 1_2422delGGCCGT | |
| G | | | TGGCATCCA | |
| 2:25236994:G:GA | NA | frameshift | c.2416_2419dupGC | p.Ser807fs |
| TGC | | | AT | |
| 2:25236994:G:T | NA | missense | c.2420C > A | p.Ser807Tyr |
| 2:25236997:GC:G | NA | frameshift | c.2416delG | p.Ala806fs |
| 2:25236998:C:T | NA | missense | c.2416G > A | p.Ala806Thr |
| 2:25236999:C:CA | NA | frameshift | c.2414dupT | p.Leu805fs |
| 2:25236999:C:CT | NA | frameshift | c.2414_2415insA | p.Ala806fs |
| 2:25236999:CA:C | NA | frameshift | c.2414delT | p.Leu805fs |
| 2:25237000:A:AA | NA | frameshift | c.2413_2414insATT | p.Leu805fs |
| AAT | | | T | |
| 2:25237000:A:T | NA | stop_gained | c.2414T > A | p.Leu805* |
| 2:25237002:CG:C | NA | frameshift | c.2411delC | p.Pro804fs |
| 2:25237002:CGG | NA | splice_accepto | c.2409- | p.Pro804del |
| CCTAGGAGGCAG | | r | 15_2411delTCTTCT | |
| AAGA:C | | | GCCTCCTAGGCC | |
| 2:25237003:G:A | rs750597155 | missense | c.2411C > T | p.Pro804Leu |
| 2:25237003:G:T | NA | missense | c.2411C > A | p.Pro804Gln |
| 2:25237003:GGC: | NA | frameshift | c.2409_2410delGC | p.Arg803fs |
| G | | | | |
| 2:25237004:G:A | NA | missense | c.2410C > T | p.Pro804Ser |
| 2:25237004:GC:G | rs35824014 | frameshift | c.2409delG | p.Arg803fs |
| 2:25237005:C:A | rs1304201075 | missense | c.2409G > T | p.Arg803Ser |
| 2:25237005:C:G | NA | missense | c.2409G > C | p.Arg803Ser |
| 2:25237006:C:A | rs866917013 | splice_acceptor | c.2409-1G > T | |
| 2:25237006:C:G | rs866917013 | splice_acceptor | c.2409-1G > C | |
| 2:25237006:C:T | rs866917013 | splice_acceptor | c.2409-1G > A | |
| 2:25237007:T:A | NA | splice_acceptor | c.2409-2A > T | |
| 2:25237007:T:C | NA | splice_acceptor | c.2409-2A > G | |
| 2:25237007:T:G | NA | splice_acceptor | c.2409-2A > C | |
| 2:25239128:A:C | NA | splice_donor | c.2408+2T > G | |
| 2:25239128:A:G | NA | splice_donor | c.2408+2T > C | |
| 2:25239128:A:T | NA | splice_donor | c.2408+2T > A | |
| 2:25239129:C:A | NA | splice_donor | c.2408+1G > T | |
| 2:25239129:C:G | NA | splice_donor | c.2408+1G > C | |
| 2:25239129:C:T | rs1457044151 | splice_donor | c.2408+1G > A | |
| 2:25239130:C:A | NA | missense | c.2408G > T | p.Arg803Met |
| 2:25239130:C:G | NA | missense | c.2408G > C | p.Arg803Thr |
| 2:25239130:C:T | rs764146514 | missense | c.2408G > A | p.Arg803Lys |
| 2:25239131:T:A | NA | missense | c.2407A > T | p.Arg803Trp |
| 2:25239131:T:C | NA | missense | c.2407A > G | p.Arg803Gly |
| 2:25239131:TG:T | NA | frameshift | c.2406delC | p.Asn802fs |
| 2:25239131:TGTT | NA | frameshift | c.2393_2406delTTC | p.Leu798fs |
| CATACCGGGAA:T | | | CCGGTATGAAC | |
| 2:25239132:G:GA | NA | frameshift | c.2405_2406insT | p.Arg803fs |
| 2:25239132:GT:G | NA | frameshift | c.2405delA | p.Asn802fs |
| 2:25239135:C:A | NA | missense | c.2403G > T | p.Met801Ile |
| 2:25239135:C:T | NA | missense | c.2403G > A | p.Met801Ile |
| 2:25239136:A:C | rs901395842 | missense | c.2402T > G | p.Met801Arg |
| 2:25239136:A:G | NA | missense | c.2402T > C | p.Met801Thr |
| 2:25239136:A:T | NA | missense | c.2402T > A | p.Met801Lys |
| 2:25239136:AT:A | NA | frameshift | c.2401delA | p.Met801fs |
| 2:25239136:ATAC | NA | frameshift | c.2391_2401delCC | p.Leu798fs |
| CGGGAAGG:A | | | TTCCCGGTA | |
| 2:25239137:T:A | NA | missense | c.2401A > T | p.Met801Leu |
| 2:25239137:T:C | rs753567076 | missense | c.2401A > G | p.Met801Val |
| 2:25239138:AC:A | NA | frameshift | c.2399delG | p.Gly800fs |
| 2:25239139:C:A | NA | missense | c.2399G > T | p.Gly800Val |
| 2:25239139:C:T | NA | missense | c.2399G > A | p.Gly800Asp |
| 2:25239140:C:CG | NA | frameshift | c.2397dupC | p.Gly800fs |
| 2:25239140:C:T | rs757083492 | missense | c.2398G > A | p.Gly800Ser |
| 2:25239140:CG:C | rs998597777 | frameshift | c.2397delC | p.Gly800fs |
| 2:25239142:G:A | rs1004794515 | missense | c.2396C > T | p.Pro799Leu |
| 2:25239142:G:C | rs1004794515 | missense | c.2396C > G | p.Pro799Arg |
| 2:25239143:G:A | rs766564570 | missense | c.2395C > T | p.Pro799Ser |
| 2:25239143:G:C | rs766564570 | missense | c.2395C > G | p.Pro799Ala |
| 2:25239143:G:T | rs766564570 | missense | c.2395C > A | p.Pro799Thr |
| 2:25239144:AAG | NA | frameshift | c.2390_2393delAC | p.Asn797fs |
| GT:A | | | CT | |
| 2:25239144:AAG | NA | frameshift | c.2384_2393delGG | p.Trp795fs |
| GTTACCCC:A | | | GGTAACCT | |
| 2:25239144:AAG | NA | inframe_indel | c.2382_2393delCT | p.Trp795_Leu798 |
| GTTACCCCAG:A | | | GGGGTAACCT | del |
| 2:25239145:A:C | NA | missense | c.2393T > G | p.Leu798Arg |
| 2:25239145:A:G | rs1399762821 | missense | c.2393T > C | p.Leu798Pro |
| 2:25239145:A:T | NA | missense | c.2393T > A | p.Leu798His |
| 2:25239145:AG:A | NA | frameshift | c.2392delC | p.Leu798fs |
| 2:25239145:AGG | NA | frameshift | c.2385_2392delGG | p.Trp795fs |
| TTACCC:A | | | GTAACC | |
| 2:25239146:G:A | NA | missense | c.2392C > T | p.Leu798Phe |
| 2:25239146:G:C | NA | missense | c.2392C > G | p.Leu798Val |
| 2:25239146:G:GG | NA | frameshift | c.2391_2392insTTA | p.Gly800fs |
| TAA | | | C | |
| 2:25239146:G:GG | NA | frameshift | c.2391_2392insGTT | p.Leu798fs |
| TAAC | | | AC | |
| 2:25239147:G:C | NA | missense | c.2391C > G | p.Asn797Lys |
| 2:25239147:G:GC | NA | frameshift | c.2390_2391insG | p.Asn797fs |
| 2:25239147:G:T | | missense | c.2391C > A | p.Asn797Lys |
| 2:25239147:GT:G | rs748836168 | frameshift | c.2390delA | p.Asn797fs |
| 2:25239148:T:A | NA | missense | c.2390A > T | p.Asn797Ile |
| 2:25239148:T:C | rs767588813 | missense | c.2390A > G | p.Asn797Ser |
| 2:25239148:T:G | NA | missense | c.2390A > C | p.Asn797Thr |
| 2:25239148:TTAC | NA | frameshift | c.2383_2389delTG | p.Trp795fs |
| CCCA:T | | | GGGTA | |
| 2:25239149:T:A | NA | missense | c.2389A > T | p.Asn797Tyr |
| 2:25239149:T:C | rs755013083 | missense | c.2389A > G | p.Asn797Asp |
| 2:25239149:T:G | rs755013083 | missense | c.2389A > C | p.Asn797His |
| 2:25239149:TACC | NA | frameshift | c.2381_2388delTCT | p.Phe794fs |
| CCAGA:T | | | GGGGT | |
| 2:25239150:A:AC | NA | frameshift | c.2386_2387dupG | p.Asn797fs |
| C | | | G | |
| 2:25239150:AC:A | rs1025684646 | frameshift | c.2387delG | p.Gly796fs |
| 2:25239150:ACCC | NA | inframe_indel | c.2382_2387delCT | p.Trp795_Gly796 |
| CAG:A | | | GGGG | del |
| 2:25239151:C:A | rs781138077 | missense | c.2387G > T | p.Gly796Val |
| 2:25239151:C:G | NA | missense | c.2387G > C | p.Gly796Ala |
| 2:25239151:C:T | rs781138077 | missense | c.2387G > A | p.Gly796Asp |
| 2:25239152:C:A | NA | missense | c.2386G > T | p.Gly796Cys |
| 2:25239152:C:G | NA | missense | c.2386G > C | p.Gly796Arg |
| 2:25239153:C:A | NA | missense | c.2385G > T | p.Trp795Cys |
| 2:25239153:C:G | NA | missense | c.2385G > C | p.Trp795Cys |
| 2:25239153:C:T | rs1395575712 | stop_gained | c.2385G > A | p.Trp795* |
| 2:25239154:C:A | rs756566100 | missense | c.2384G > T | p.Trp795Leu |
| 2:25239154:C:G | NA | missense | c.2384G > C | p.Trp795Ser |
| 2:25239154:C:T | rs756566100 | stop_gained | c.2384G > A | p.Trp795* |
| 2:25239154:CA:C | NA | frameshift | c.2383delT | p.Trp795fs |
| 2:25239154:CAG: | NA | frameshift | c.2382_2383delCT | p.Phe794fs |
| C | | | | |
| 2:25239154:CAG | NA | inframe_indel | c.2381_2383delTCT | p.Phe794del |
| A:C | | | | |
| 2:25239155:A:G | rs778414705 | missense | c.2383T > C | p.Trp795Arg |
| 2:25239155:A:T | | missense | c.2383T > A | p.Trp795Arg |
| 2:25239156:G:C | NA | missense | c.2382C > G | p.Phe794Leu |
| 2:25239157:A:C | NA | missense | c.2381T > G | p.Phe794Cys |
| 2:25239157:A:G | rs1419173604 | missense | c.2381T > C | p.Phe794Ser |
| 2:25239158:A:C | | missense | c.2380T > G | p.Phe794Val |
| 2:25239158:A:G | rs771474959 | missense | c.2380T > C | p.Phe794Leu |
| 2:25239158:A:T | NA | missense | c.2380T > A | p.Phe794Ile |
| 2:25239159:G:C | rs1418024882 | stop_gained | c.2379C > G | p.Tyr793* |
| 2:25239159:G:T | NA | stop_gained | c.2379C > A | p.Tyr793* |
| 2:25239160:T:G | NA | missense | c.2378A > C | p.Tyr793Ser |
| 2:25239161:A:C | rs1247759759 | missense | c.2377T > G | p.Tyr793Asp |
| 2:25239161:A:G | NA | missense | c.2377T > C | p.Tyr793His |
| 2:25239163:C:G | NA | missense | c.2375G > C | p.Arg792Pro |
| 2:25239163:C:T | rs774100557 | missense | c.2375G > A | p.Arg792His |
| 2:25239163:CG:C | NA | frameshift | c.2374delC | p.Arg792fs |
| 2:25239164:G:A | NA | missense | c.2374C > T | p.Arg792Cys |
| 2:25239164:G:C | NA | missense | c.2374C > G | p.Arg792Gly |
| 2:25239164:G:GG | NA | frameshift | c.2366_2373dupAC | p.Arg792fs |
| GCCCTGT | | | AGGGCC | |
| 2:25239164:GGG | NA | frameshift | c.2364_2373delAC | p.His789fs |
| CCCTGTGT:G | | | ACAGGGCC | |
| 2:25239166:G:A | NA | missense | c.2372C > T | p.Ala791Val |
| 2:25239166:G:T | NA | missense | c.2372C > A | p.Ala791Asp |
| 2:25239166:GCG | NA | frameshift | c.2371delG | p.Ala791fs |
| 2:25239167:C:T | rs1005623505 | missense | c.2371G > A | p.Ala791Thr |
| 2:25239167:CCCT | NA | frameshift | c.2366_2370delAC | p.His789fs |
| GT:C | | | AGG | |
| 2:25239168:C:G | NA | missense | c.2370G > C | p.Arg790Ser |
| 2:25239169:CT:C | NA | frameshift | c.2368delA | p.Arg790fs |
| 2:25239169:CTG: | NA | frameshift | c.2367_2368delCA | p.His789fs |
| C | | | | |
| 2:25239170:T:A | NA | missense | c.2368A > T | p.Arg790Trp |
| 2:25239171:G:T | NA | missense | c.2367C > A | p.His789Gln |
| 2:25239172:T:A | NA | missense | c.2366A > T | p.His789Leu |
| 2:25239173:GT:G | NA | frameshift | c.2364delA | p.His789fs |
| 2:25239175:G:A | rs1277979168 | missense | c.2363C > T | p.Ala788Val |
| 2:25239175:GCA | NA | frameshift | c.2358_2362delAG | p.Ala787fs |
| GCT:G | | | CTG | |
| 2:25239176:C:T | NA | missense | c.2362G > A | p.Ala788Thr |
| 2:25239176:CA:C | NA | frameshift | c.2361delT | p.Ala788fs |
| 2:25239177:AGC: | NA | frameshift | c.2359_2360delGC | p.Ala787fs |
| A | | | | |
| 2:25239178:G:GC | NA | frameshift | c.2359dupG | p.Ala787fs |
| 2:25239178:GCT: | NA | frameshift | c.2358_2359delAG | p.Ala787fs |
| G | | | | |
| 2:25239179:C:A | rs375845138 | missense | c.2359G > T | p.Ala787Ser |
| 2:25239179:C:CT | NA | frameshift | c.2358dupA | p.Ala787fs |
| 2:25239179:C:T | NA | missense | c.2359G > A | p.Ala787Thr |
| 2:25239181:G:A | NA | missense | c.2357C > T | p.Ser786Leu |
| 2:25239181:G:C | rs962973415 | stop_gained | c.2357C > G | p.Ser786* |
| 2:25239181:G:T | NA | stop_gained | c.2357C > A | p.Ser786* |
| 2:25239181:GAC: | NA | frameshift | c.2355_2356delGT | p.Ala787fs |
| G | | | | |
| 2:25239185:C:T | NA | missense | c.2353G > A | p.Va1785Met |
| 2:25239186:TTC:T | NA | frameshift | c.2350_2351delGA | p.Glu784fs |
| 2:25239187:T:TC | NA | frameshift | c.2350_2351insTTT | p.Glu784fs |
| AAA | | | G | |
| 2:25239187:T:TCT | NA | frameshift | c.2323_2350dupTC | p.Glu784fs |
| TTGGCATCAATCA | | | CAACCCTGTGATGA | |
| TCACAGGGTTGG | | | TTGATGCCAAAG | |
| A | | | | |
| 2:25239187:TC:T | NA | frameshift | c.2350delG | p.Glu784fs |
| 2:25239188:C:A | NA | stop_gained | c.2350G > T | p.Glu784* |
| 2:25239188:C:CT | NA | frameshift | c.2349dupA | p.Glu784fs |
| 2:25239188:CT:C | rs1255237375 | frameshift | c.2349delA | p.Glu784fs |
| 2:25239191:T:TG | NA | frameshift | c.2346dupC | p.Lys783fs |
| 2:25239191:TG:T | NA | frameshift | c.2346delC | p.Glu784fs |
| 2:25239193:G:A | NA | missense | c.2345C > T | p.Ala782Val |
| 2:25239194:C:CA | NA | frameshift | c.2340_2343dupTG | p.Ala782fs |
| TCA | | | AT | |
| 2:25239194:C:T | NA | missense | c.2344G > A | p.Ala782Thr |
| 2:25239194:CATC | NA | frameshift | c.2340_2343delTG | p.Ile780fs |
| A:C | | | AT | |
| 2:25239195:AT:A | NA | frameshift | c.2342delA | p.Asp781fs |
| 2:25239197:CA:C | NA | frameshift | c.2340delT | p.Ile780fs |
| 2:25239198:A:C | NA | missense | c.2340T > G | p.Ile780Met |
| 2:25239198:AATC | NA | inframe_indel | c.2334_2339delGA | p.Met779_Ile780 |
| ATC:A | | | TGAT | del |
| 2:25239198:AATC | NA | frameshift | c.2330_2339delCT | p.Pro777fs |
| ATCACAG:A | | | GTGATGAT | |
| 2:25239199:A:C | rs370751539 | missense | c.2339T > G | p.Ile780Ser |
| 2:25239199:A:G | rs370751539 | missense | c.2339T > C | p.Ile780Thr |
| 2:25239199:A:T | rs370751539 | missense | c.2339T > A | p.Ile780Asn |
| 2:25239201:C:CA | NA | frameshift | c.2333_2336dupTG | p.Met779fs |
| TCA | | | AT | |
| 2:25239201:CA:C | NA | frameshift | c.2336delT | p.Met779fs |
| 2:25239202:A:G | NA | missense | c.2336T > C | p.Met779Thr |
| 2:25239202:AT:A | NA | frameshift | c.2335delA | p.Met779fs |
| 2:25239203:TCAC | NA | splice_accepto | c.2323- | p.Ser775_Met779 |
| AGGGTTGGACTA | | r | 11_2334delCTGTTT | del |
| CAAAACAG:T | | | TGTAGTCCAACCCT | |
| | | | GTG | |
| 2:25239204:C:CA | NA | frameshift | c.2333dupT | p.Met779fs |
| 2:25239204:CA:C | NA | frameshift | c.2333delT | p.Va1778fs |
| 2:25239204:CACA | NA | frameshift | c.2323- | p.Ser775fs |
| GGGTTGGACTAC | | | 6_2333delTTGTAG | |
| AA:C | | | TCCAACCCTGT | |
| 2:25239204:CACA | NA | frameshift | c.2323- | p.Ser775fs |
| GGGTTGGACTAC | | | 7_2333delTTTGTA | |
| AAA:C | | | GTCCAACCCTGT | |
| 2:25239205:A:C | rs979932565 | missense | c.2333T > G | p.Va1778Gly |
| 2:25239205:A:G | NA | missense | c.2333T > C | p.Va1778Ala |
| 2:25239205:A:T | NA | missense | c.2333T > A | p.Va1778Glu |
| 2:25239205:AC:A | NA | frameshift | c.2332delG | p.Va1778fs |
| 2:25239206:C:A | NA | missense | c.2332G > T | p.Va1778Leu |
| 2:25239206:C:T | NA | missense | c.2332G > A | p.Va1778Met |
| 2:25239207:AG:A | rs775989176 | frameshift | c.2330delC | p.Pro777fs |
| 2:25239208:G:A | rs752626029 | missense | c.2330C > T | p.Pro777Leu |
| 2:25239208:G:C | rs752626029 | missense | c.2330C > G | p.Pro777Arg |
| 2:25239208:G:T | rs752626029 | missense | c.2330C > A | p.Pro777His |
| 2:25239209:G:A | rs1172777420 | missense | c.2329C > T | p.Pro777Ser |
| 2:25239210:GT:G | NA | frameshift | c.2327delA | p.Asn776fs |
| 2:25239212:T:TG | NA | frameshift | c.2325dupC | p.Asn776fs |
| 2:25239212:TG:T | NA | frameshift | c.2325delC | p.Asn776fs |
| 2:25239214:G:A | NA | missense | c.2324C > T | p.Ser775Phe |
| 2:25239214:GA:G | NA | frameshift | c.2323delT | p.Ser775fs |
| 2:25239215:A:G | rs74708853 | missense | c.2323T > C | p.Ser775Pro |
| 2:25239215:A:T | NA | missense | c.2323T > A | p.Ser775Thr |
| 2:25239216:C:G | NA | splice_acceptor | c.2323-1G > C | |
| 2:25239216:C:T | rs1324347218 | splice_acceptor | c.2323-1G > A | |
| 2:25239217:T:A | rs765045799 | splice_acceptor | c.2323-2A > T | |
| 2:25239217:T:C | rs765045799 | splice_acceptor | c.2323-2A > G | |
| 2:25239217:T:G | NA | splice_acceptor | c.2323-2A > C | |
| 2:25240299:TACC | NA | frameshift | c.2319_2322+2del | p.Glu774fs |
| TCG:T | | | CGAGGT | |
| 2:25240300:A:AC | NA | splice_donor | c.2321_2322+1dup | |
| CT | | | AGG | |
| 2:25240300:A:C | NA | splice_donor | c.2322+2T > G | |
| 2:25240300:A:G | NA | splice_donor | c.2322+2T > C | |
| 2:25240300:AC:A | NA | splice_donor | c.2322+1delG | |
| 2:25240300:ACCT | NA | splice_donor | c.2305_23221+1del | p.Ile769_Glu774 |
| CGAGAAATCGCG | | | ATCTCGCGATTTCT | del |
| AGAT:A | | | CGAGG | |
| 2:25240300:ACCT | NA | splice_donor | c.2302_2322+1del | p.Asp768_Glu774 |
| CGAGAAATCGCG | | | GACATCTCGCGATT | del |
| AGATGTC:A | | | TCTCGAGG | |
| 2:25240301:C:A | rs903011938 | splice_donor | c.2322+1G > T | |
| 2:25240301:C:CC | NA | frameshift | c.2313_2322dupAT | p.Ser775fs |
| TCGAGAAAT | | | TTCTCGAG | |
| 2:25240301:C:G | NA | splice_donor | c.2322+1G > C | |
| 2:25240301:C:T | rs903011938 | splice_donor | c.2322+1G > A | |
| 2:25240301:CCT: | NA | frameshift | c.2321_2322delAG | p.Glu774fs |
| C | | | | |
| 2:25240301:CCTC | NA | frameshift | c.2318_2322delTC | p.Glu774fs |
| GA:C | | | GAG | |
| 2:25240302:C:A | rs753017271 | missense | c.2322G > T | p.Glu774Asp |
| 2:25240302:C:CT | NA | frameshift | c.2314_2321dupTT | p.Glu774fs |
| CGAGAAA | | | TCTCGA | |
| 2:25240302:C:G | rs753017271 | missense | c.2322G > C | p.Glu774Asp |
| 2:25240302:CTCG | NA | frameshift | c.2318_2321delTC | p.Leu773fs |
| A:C | | | GA | |
| 2:25240303:T:A | NA | missense | c.2321A > T | p.Glu774Val |
| 2:25240303:T:C | | missense | c.2321A > G | p.Glu774Gly |
| 2:25240303:T:G | NA | missense | c.2321A > C | p.Glu774Ala |
| 2:25240303:T:TC | NA | frameshift | c.2320dupG | p.Glu774fs |
| 2:25240303:T:TC | rs761980712 | frameshift | c.2311_2320dupCG | p.Glu774fs |
| GAGAAATCG | | | ATTTCTCG | |
| 2:25240303:TC:T | NA | frameshift | c.2320delG | p.Glu774fs |
| 2:25240304:C:A | NA | stop_gained | c.2320G > T | p.Glu774* |
| 2:25240304:C:CG | NA | frameshift | c.2319dupC | p.Glu774fs |
| 2:25240304:C:T | rs760624806 | missense | c.2320G > A | p.Glu774Lys |
| 2:25240304:CGA | NA | frameshift | c.2315_2319delTTC | p.Phe772fs |
| GAA:C | | | TC | |
| 2:25240305:G:GA | NA | frameshift | c.2290_2318dupA | p.Glu774fs |
| GAAATCGCGAGA | | | GTGACAAGAGGGA | |
| TGTCCCTCTTGTC | | | CATCTCGCGATTTC | |
| ACT | | | T | |
| 2:25240305:GAG | NA | inframe_indel | c.2316_2318delTCT | p.Leu773del |
| A:G | | | | |
| 2:25240306:A:C | rs764303486 | missense | c.2318T > G | p.Leu773Arg |
| 2:25240306:A:G | NA | missense | c.2318T > C | p.Leu773Pro |
| 2:25240306:A:T | NA | missense | c.2318T > A | p.Leu773His |
| 2:25240306:AG:A | NA | frameshift | c.2317delC | p.Leu773fs |
| 2:25240307:G:A | NA | missense | c.2317C > T | p.Leu773Phe |
| 2:25240307:G:C | NA | missense | c.2317C > G | p.Leu773Val |
| 2:25240307:G:GA | NA | frameshift | c.2315_2316dupTT | p.Leu773fs |
| A | | | | |
| 2:25240307:G:T | rs754336556 | missense | c.2317C > A | p.Leu773Ile |
| 2:25240307:GA:G | NA | frameshift | c.2316delT | p.Leu773fs |
| 2:25240307:GAA: | NA | frameshift | c.2315_2316delTT | p.Phe772fs |
| G | | | | |
| 2:25240308:A:AA | NA | inframe_indel | c.2283_2315dupG | p.Arg771_Phe772 |
| ATCGCGAGATGTC | | | GGCGTTAGTGACAA | insLeuGlyValSerA |
| CCTCTTGTCACTA | | | GAGGGACATCTCGC | spLysArgAspIleSer |
| ACGCCC | | | GATT | Arg |
| 2:25240308:A:C | NA | missense | c.2316T > G | p.Phe772Leu |
| 2:25240308:A:T | NA | missense | c.2316T > A | p.Phe772Leu |
| 2:25240308:AAAT | NA | frameshift | c.2312_2315delGA | p.Arg771fs |
| C:A | | | TT | |
| 2:25240309:A:AA | NA | frameshift | c.2272_2314dupGT | p.Phe772fs |
| TCGCGAGATGTCC | | | GGTGGCCATGGGC | |
| CTCTTGTCACTAA | | | GTTAGTGACAAGA | |
| CGCCCATGGCCAC | | | GGGACATCTCGCGA | |
| CAC | | | T | |
| 2:25240309:A:AT | NA | frameshift | c.2314_2315insGA | p.Phe772fs |
| C | | | | |
| 2:25240309:A:C | NA | missense | c.2315T > G | p.Phe772Cys |
| 2:25240309:A:G | NA | missense | c.2315T > C | p.Phe772Ser |
| 2:25240309:AATC | NA | frameshift | c.2307_2314delCT | p.Phe772fs |
| GCGAG:A | | | CGCGAT | |
| 2:25240310:A:AT | NA | frameshift | c.2295_2313dupCA | p.Phe772fs |
| CGCGAGATGTCCC | | | AGAGGGACATCTC | |
| TCTTG | | | GCGA | |
| 2:25240310:A:G | NA | missense | c.2314T > C | p.Phe772Leu |
| 2:25240310:A:T | NA | missense | c.2314T > A | p.Phe772Ile |
| 2:25240310:AT:A | NA | frameshift | c.2313delA | p.Leu773fs |
| 2:25240311:T:TC | NA | frameshift | c.2288_2312dupTT | p.Phe772fs |
| GCGAGATGTCCCT | | | AGTGACAAGAGGG | |
| CTTGTCACTAA | | | ACATCTCGCG | |
| 2:25240311:TC:T | NA | frameshift | c.2312delG | p.Arg771fs |
| 2:25240311:TCGC | NA | frameshift | c.2309_2312delCG | p.Ser770fs |
| G:T | | | CG | |
| 2:25240312:C:A | rs757823678 | missense | c.2312G > T | p.Arg771Leu |
| 2:25240312:C:CG | NA | frameshift | c.2311_2312insAA | p.Arg771fs |
| ATTT | | | ATC | |
| 2:25240312:C:G | NA | missense | c.2312G > C | p.Arg771Pro |
| 2:25240312:C:T | rs757823678 | missense | c.2312G > A | p.Arg771Gln |
| 2:25240312:CG:C | NA | frameshift | c.2311delC | p.Arg771fs |
| 2:25240312:CGC | NA | frameshift | c.2289_2311delTA | p.Ser764fs |
| GAGATGTCCCTCT | | | GTGACAAGAGGGA | |
| TGTCACTA:C | | | CATCTCGC | |
| 2:25240313:G:A | rs779626155 | stop_gained | c.2311C > T | p.Arg771* |
| 2:25240313:G:C | NA | missense | c.2311C > G | p.Arg771Gly |
| 2:25240313:G:GC | NA | frameshift | c.2310dupG | p.Arg771fs |
| 2:25240313:GCG | NA | frameshift | c.2292_2310delTG | p.Lys766fs |
| AGATGTCCCTCTT | | | ACAAGAGGGACAT | |
| GTCA:G | | | CTCG | |
| 2:25240314:C:CG | NA | inframe_indel | c.2307_2309dupCT | p.Ser770dup |
| AG | | | C | |
| 2:25240314:CGA: | NA | frameshift | c.2308_2309delTC | p.Ser770fs |
| C | | | | |
| 2:25240315:G:A | rs758845779 | missense | c.2309C > T | p.Ser770Leu |
| 2:25240315:G:C | rs758845779 | missense | c.2309C > G | p.Ser770Trp |
| 2:25240315:G:GA | NA | frameshift | c.2308dupT | p.Ser770fs |
| 2:25240315:G:T | rs758845779 | stop_gained | c.2309C > A | p.Ser770* |
| 2:25240315:GA:G | NA | frameshift | c.2308delT | p.Ser770fs |
| 2:25240316:A:C | NA | missense | c.2308T > G | p.Ser770Ala |
| 2:25240316:A:G | rs1275276226 | missense | c.2308T > C | p.Ser770Pro |
| 2:25240316:AG:A | NA | frameshift | c.2307delC | p.Ser770fs |
| 2:25240317:G:C | NA | missense | c.2307C > G | p.Ile769Met |
| 2:25240317:G:GT | NA | frameshift | c.2306_2307insA | p.Ser770fs |
| 2:25240317:GA:G | NA | frameshift | c.2306delT | p.Ile769fs |
| 2:25240318:A:C | rs746704362 | missense | c.2306T > G | p.Ile769Ser |
| 2:25240318:A:G | rs746704362 | missense | c.2306T > C | p.Ile769Thr |
| 2:25240318:A:T | rs746704362 | missense | c.2306T > A | p.Ile769Asn |
| 2:25240318:AT:A | NA | frameshift | c.2305delA | p.Ile769fs |
| 2:25240319:T:C | NA | missense | c.2305A > G | p.Ile769Val |
| 2:25240319:T:G | NA | missense | c.2305A > C | p.Ile769Leu |
| 2:25240320:G:C | NA | missense | c.2304C > G | p.Asp768Glu |
| 2:25240320:G:T | NA | missense | c.2304C > A | p.Asp768Glu |
| 2:25240320:GT:G | NA | frameshift | c.2303delA | p.Asp768fs |
| 2:25240321:T:A | NA | missense | c.2303A > T | p.Asp768Val |
| 2:25240321:T:C | rs1181120114 | missense | c.2303A > G | p.Asp768Gly |
| 2:25240321:TC:T | NA | frameshift | c.2302delG | p.Asp768fs |
| 2:25240322:C:A | rs767983115 | missense | c.2302G > T | p.Asp768Tyr |
| 2:25240322:C:G | NA | missense | c.2302G > C | p.Asp768His |
| 2:25240322:C:T | NA | missense | c.2302G > A | p.Asp768Asn |
| 2:25240323:C:CC | NA | frameshift | c.2299_2300dupA | p.Asp768fs |
| T | | | G | |
| 2:25240324:CT:C | NA | frameshift | c.2299delA | p.Arg767fs |
| 2:25240324:CTCT | NA | frameshift | c.2295_2299delCA | p.Asp765fs |
| TG:C | | | AGA | |
| 2:25240325:T:C | NA | missense | c.2299A > G | p.Arg767Gly |
| 2:25240325:TC:T | NA | frameshift | c.2298delG | p.Arg767fs |
| 2:25240326:C:A | NA | missense | c.2298G > T | p.Lys766Asn |
| 2:25240326:C:G | NA | missense | c.2298G > C | p.Lys766Asn |
| 2:25240326:CT:C | NA | frameshift | c.2297delA | p.Lys766fs |
| 2:25240326:CTT:C | rs750475955 | frameshift | c.2296_2297delAA | p.Lys766fs |
| 2:25240327:TTG: | NA | frameshift | c.2295_2296delCA | p.Asp765fs |
| T | | | | |
| 2:25240328:T:A | NA | stop_gained | c.2296A > T | p.Lys766* |
| 2:25240328:T:C | NA | missense | c.2296A > G | p.Lys766Glu |
| 2:25240328:T:TA | NA | frameshift | c.2295_2296insT | p.Lys766fs |
| 2:25240328:TG:T | NA | frameshift | c.2295delC | p.Asp765fs |
| 2:25240329:G:GT | NA | frameshift | c.2294dupA | p.Asp765fs |
| 2:25240330:T:C | NA | missense | c.2294A > G | p.Asp765Gly |
| 2:25240331:C:A | NA | missense | c.2293G > T | p.Asp765Tyr |
| 2:25240332:AC:A | NA | frameshift | c.2291delG | p.Ser764fs |
| 2:25240333:C:CT | NA | frameshift | c.2290dupA | p.Ser764fs |
| 2:25240333:CT:C | NA | frameshift | c.2290delA | p.Ser764fs |
| 2:25240334:T:TA | NA | frameshift | c.2289dupT | p.Ser764fs |
| 2:25240334:TA:T | NA | frameshift | c.2289delT | p.Ser764fs |
| 2:25240336:A:T | rs1178258952 | missense | c.2288T > A | p.Va1763Asp |
| 2:25240336:AC:A | NA | frameshift | c.2287delG | p.Va1763fs |
| 2:25240337:C:CA | NA | frameshift | c.2286_2287insT | p.Va1763fs |
| 2:25240337:C:CG | NA | frameshift | c.2286_2287insTA | p.Va1763fs |
| TTA | | | AC | |
| 2:25240337:C:T | rs1369746569 | missense | c.2287G > A | p.Va1763Ile |
| 2:25240338:GC:G | NA | frameshift | c.2285delG | p.Gly762fs |
| 2:25240338:GCCC | NA | frameshift | c.2276_2285delTG | p.Va1759fs |
| ATGGCCA:G | | | GCCATGGG | |
| 2:25240342:A:C | NA | missense | c.2282T > G | p.Met761Arg |
| 2:25240342:A:G | rs963262801 | missense | c.2282T > C | p.Met761Thr |
| 2:25240342:ATG | NA | inframe_indel | c.2270_2281delAT | p.Asn757_Ala760 |
| GCCACCACAT:A | | | GTGGTGGCCA | del |
| 2:25240343:T:C | rs780695582 | missense | c.2281A > G | p.Met761Val |
| 2:25240343:T:G | NA | missense | c.2281A > C | p.Met761Leu |
| 2:25240343:T:TG | NA | frameshift | c.2280dupC | p.Met761fs |
| 2:25240343:TG:T | NA | frameshift | c.2280delC | p.Met761fs |
| 2:25240345:G:A | NA | missense | c.2279C > T | p.Ala760Val |
| 2:25240345:G:C | NA | missense | c.2279C > G | p.Ala760Gly |
| 2:25240345:G:GC | NA | frameshift | c.2278dupG | p.Ala760fs |
| 2:25240345:GCG | NA | frameshift | c.2278delG | p.Ala760fs |
| 2:25240345:GCCA: | NA | inframe_indel | c.2276_2278delTG | p.Va1759del |
| G | | | G | |
| 2:25240346:CCAC | NA | frameshift | c.2267_2277delAG | p.Glu756fs |
| CACATTCT:C | | | AATGTGGTG | |
| 2:25240347:CA:C | NA | frameshift | c.2276delT | p.Va1759fs |
| 2:25240348:AC:A | NA | frameshift | c.2275delG | p.Va1759fs |
| 2:25240349:C:G | NA | missense | c.2275G > C | p.Va1759Leu |
| 2:25240349:C:T | NA | missense | c.2275G > A | p.Va1759Met |
| 2:25240349:CCA: | NA | frameshift | c.2273_2274delTG | p.Va1758fs |
| C | | | | |
| 2:25240352:C:G | NA | missense | c.2272G > C | p.Va1758Leu |
| 2:25240352:C:T | NA | missense | c.2272G > A | p.Va1758Met |
| 2:25240352:CA:C | NA | frameshift | c.2271delT | p.Asn757fs |
| 2:25240352:CATT | NA | frameshift | c.2256_2271delCT | p.Phe752fs |
| CTCAAAGAGCCAG: | | | GGCTCTTTGAGAAT | |
| C | | | | |
| 2:25240353:A:C | NA | missense | c.2271T > G | p.Asn757Lys |
| 2:25240353:A:T | NA | missense | c.2271T > A | p.Asn757Lys |
| 2:25240353:AT:A | NA | frameshift | c.2270delA | p.Asn757fs |
| 2:25240353:ATTC | NA | frameshift | c.2248_2270delCC | p.Pro750fs |
| TCAAAGAGCCAG | | | CTTGTTCTGGCTCTT | |
| AAGAAGGG:A | | | TGAGAA | |
| 2:25240354:T:A | rs747716263 | missense | c.2270A > T | p.Asn757Ile |
| 2:25240354:T:C | NA | missense | c.2270A > G | p.Asn757Ser |
| 2:25240354:T:TTC | NA | frameshift | c.2268_2269dupG | p.Asn757fs |
| | | | A | |
| 2:25240355:T:A | NA | missense | c.2269A > T | p.Asn757Tyr |
| 2:25240356:C:A | rs769718039 | missense | c.2268G > T | p.Glu756Asp |
| 2:25240356:C:G | NA | missense | c.2268G > C | p.Glu756Asp |
| 2:25240356:CTCA | NA | frameshift | c.2251_2267delTTC | p.Phe751fs |
| AAGAGCCAGAAG | | | TTCTGGCTCTTTGA | |
| AA:C | | | | |
| 2:25240357:T:C | NA | missense | c.2267A > G | p.Glu756Gly |
| 2:25240357:T:TC | NA | frameshift | c.2265_2266dupTG | p.Glu756fs |
| A | | | | |
| 2:25240358:C:A | NA | stop_gained | c.2266G > T | p.Glu756* |
| 2:25240358:C:CA | rs1386755076 | frameshift | c.2265dupT | p.Glu756fs |
| 2:25240358:C:T | rs1418213272 | missense | c.2266G > A | p.Glu756Lys |
| 2:25240358:CA:C | rs752551332 | frameshift | c.2265delT | p.Phe755fs |
| 2:25240358:CAA: | rs755800729 | frameshift | c.2264_2265delTT | p.Phe755fs |
| C | | | | |
| 2:25240358:CAAA | NA | frameshift | c.2261_2265delTCT | p.Leu754fs |
| GA:C | | | TT | |
| 2:25240359:A:AT | NA | frameshift | c.2264_2265insA | p.Phe755fs |
| 2:25240359:A:C | NA | missense | c.2265T > G | p.Phe755Leu |
| 2:25240359:A:T | NA | missense | c.2265T > A | p.Phe755Leu |
| 2:25240360:A:C | NA | missense | c.2264T > G | p.Phe755Cys |
| 2:25240360:A:G | rs536841393 | missense | c.2264T > C | p.Phe755Ser |
| 2:25240360:AAG: | NA | frameshift | c.2262_2263delCT | p.Phe755fs |
| A | | | | |
| 2:25240360:AAG | NA | inframe_indel | c.2258_2263delGG | p.Trp753_Leu754 |
| AGCC:A | | | CTCT | del |
| 2:25240360:AAG | NA | frameshift | c.2250_2263delCTT | p.Phe751fs |
| AGCCAGAAGAAG: | | | CTTCTGGCTCT | |
| A | | | | |
| 2:25240361:A:C | NA | missense | c.2263T > G | p.Phe755Val |
| 2:25240361:A:G | NA | missense | c.2263T > C | p.Phe755Leu |
| 2:25240361:A:T | rs369230209 | missense | c.2263T > A | p.Phe755Ile |
| 2:25240361:AGA | NA | frameshift | c.2246_2262delGC | p.Arg749fs |
| GCCAGAAGAAGG | | | CCCTTCTTCTGGCTC | |
| GGC:A | | | | |
| 2:25240362:GA:G | NA | frameshift | c.2261delT | p.Leu754fs |
| 2:25240362:GAG | NA | frameshift | c.2258_2261delGG | p.Trp753fs |
| CC:G | | | CT | |
| 2:25240363:A:C | rs762636955 | missense | c.2261T > G | p.Leu754Arg |
| 2:25240363:A:G | NA | missense | c.2261T > C | p.Leu754Pro |
| 2:25240363:A:T | NA | missense | c.2261T > A | p.Leu754His |
| 2:25240364:G:C | NA | missense | c.2260C > G | p.Leu754Val |
| 2:25240364:GCCA | NA | frameshift | c.2243_2259delAT | p.Asp748fs |
| GAAGAAGGGGCG | | | CGCCCCTTCTTCTG | |
| AT:G | | | G | |
| 2:25240365:C:A | NA | missense | c.2259G > T | p.Trp753Cys |
| 2:25240365:C:CTT | NA | frameshift | c.2258_2259insAA | p.Trp753fs |
| 2:25240365:C:G | rs770785915 | missense | c.2259G > C | p.Trp753Cys |
| 2:25240365:C:T | NA | stop_gained | c.2259G > A | p.Trp753* |
| 2:25240365:CCAG: | NA | inframe_indel | c.2256_2258delCT | p.Phe752_Trp753 |
| C | | | G | delinsLeu |
| 2:25240366:C:A | rs775537912 | missense | c.2258G > T | p.Trp753Leu |
| 2:25240366:C:CA | NA | frameshift | c.2256_2257dupCT | p.Trp753fs |
| G | | | | |
| 2:25240366:C:G | | missense | c.2258G > C | p.Trp753Ser |
| 2:25240366:C:T | | stop_gained | c.2258G > A | p.Trp753* |
| 2:25240366:CAG: | NA | frameshift | c.2256_2257delCT | p.Phe752fs |
| C | | | | |
| 2:25240366:CAG | rs749132507 | inframe_indel | c.2255_2257delTCT | p.Phe752del |
| A:C | | | | |
| 2:25240366:CAG | NA | frameshift | c.2245_2257delCG | p.Arg749fs |
| AAGAAGGGGCG: | | | CCCCTTCTTCT | |
| C | | | | |
| 2:25240367:A:C | NA | missense | c.2257T > G | p.Trp753Gly |
| 2:25240367:A:G | NA | missense | c.2257T > C | p.Trp753Arg |
| 2:25240367:A:T | rs1189107128 | missense | c.2257T > A | p.Trp753Arg |
| 2:25240367:AG:A | rs770968422 | frameshift | c.2256delC | p.Trp753fs |
| 2:25240367:AGA | NA | frameshift | c.2253_2256delCTT | p.Phe752fs |
| AG:A | | | c | |
| 2:25240367:AGA | NA | frameshift | c.2246_2256delGC | p.Arg749fs |
| AGAAGGGGC:A | | | CCCTTCTTCC | |
| 2:25240368:G:C | rs760959622 | missense | c.2256C > G | p.Phe752Leu |
| 2:25240368:G:GC | NA | frameshift | c.2255_2256insG | p.Phe752fs |
| 2:25240368:G:T | NA | missense | c.2256C > A | p.Phe752Leu |
| 2:25240368:GA:G | NA | frameshift | c.2255delT | p.Phe752fs |
| 2:25240368:GAA: | NA | frameshift | c.2254_2255delTT | p.Phe752fs |
| G | | | | |
| 2:25240369:A:AA | NA | frameshift | c.2242_2254dupG | p.Phe752fs |
| GAAGGGGCGATC | | | ATCGCCCCTTCT | |
| 2:25240369:A:C | rs764464925 | missense | c.2255T > G | p.Phe752Cys |
| 2:25240369:A:G | NA | missense | c.2255T > C | p.Phe752Ser |
| 2:25240369:AAG: | NA | frameshift | c.2253_2254delCT | p.Phe752fs |
| A | | | | |
| 2:25240369:AAG | NA | frameshift | c.2247_2254delCC | p.Pro750fs |
| AAGGGG:A | | | CCTTCT | |
| 2:25240370:A:AG | NA | frameshift | c.2253_2254insAA | p.Phe752fs |
| AAGGGGCTTT | | | AGCCCCTTC | |
| 2:25240370:A:C | NA | missense | c.2254T > G | p.Phe752Val |
| 2:25240370:A:G | rs776844126 | missense | c.2254T > C | p.Phe752Leu |
| 2:25240370:A:T | | missense | c.2254T > A | p.Phe752Ile |
| 2:25240370:AG:A | NA | frameshift | c.2253delC | p.Phe752fs |
| 2:25240370:AGA | NA | frameshift | c.2247_2253delCC | p.Pro750fs |
| AGGGG:A | | | CCTTC | |
| 2:25240371:G:C | rs762020470 | missense | c.2253C > G | p.Phe751Leu |
| 2:25240371:G:GA | NA | frameshift | c.2252dupT | p.Phe752fs |
| 2:25240371:G:GT | NA | frameshift | c.2252_2253insA | p.Phe751fs |
| 2:25240371:G:T | NA | missense | c.2253C > A | p.Phe751Leu |
| 2:25240371:GA:G | NA | frameshift | c.2252delT | p.Phe751fs |
| 2:25240372:A:C | rs765813304 | missense | c.2252T > G | p.Phe751Cys |
| 2:25240372:A:G | rs765813304 | missense | c.2252T > C | p.Phe751Ser |
| 2:25240373:A:AG | rs1195416744 | frameshift | c.2250dupC | p.Phe751fs |
| 2:25240373:A:C | NA | missense | c.2251T > G | p.Phe751Val |
| 2:25240373:A:G | NA | missense | c.2251T > C | p.Phe751Leu |
| 2:25240373:A:T | rs750962348 | missense | c.2251T > A | p.Phe751Ile |
| 2:25240373:AG:A | NA | frameshift | c.2250delC | p.Phe751fs |
| 2:25240374:GGG | NA | frameshift | c.2246_2249delGC | p.Arg749fs |
| GCG | | | CC | |
| 2:25240375:G:C | NA | missense | c.2249C > G | p.Pro750Arg |
| 2:25240375:G:GG | NA | frameshift | c.2230_2248dupAA | p.Pro750fs |
| GCGATCATCTCCC | | | GGAGGGAGATGAT | |
| TCCTT | | | CGCC | |
| 2:25240375:GGG | NA | inframe_indel | c.2240_2248delAT | p.Asp747_Pro750 |
| CGATCAT:G | | | GATCGCC | delinsAla |
| 2:25240376:G:A | NA | missense | c.2248C > T | p.Pro750Ser |
| 2:25240376:G:C | NA | missense | c.2248C > G | p.Pro750Ala |
| 2:25240376:G:GG | NA | frameshift | c.2235_2247dupG | p.Pro750fs |
| CGATCATCTCCC | | | GGAGATGATCGC | |
| 2:25240376:GGC: | NA | frameshift | c.2246_2247delGC | p.Arg749fs |
| G | | | | |
| 2:25240377:G:GA | NA | frameshift | c.2246_2247insT | p.Phe751fs |
| 2:25240377:G:GC | NA | frameshift | c.2246dupG | p.Phe751fs |
| 2:25240377:G:GC | NA | frameshift | c.2246_2247insAG | p.Pro750fs |
| GATCATCTCCCTC | | | CCAGAGGCCCAAG | |
| CTTGGGCCTCTGG | | | GAGGGAGATGATC | |
| CT | | | G | |
| 2:25240377:GC:G | NA | frameshift | c.2246delG | p.Arg749fs |
| 2:25240377:GCG | NA | frameshift | c.2243_2246delAT | p.Asp748fs |
| AT:G | | | CG | |
| 2:25240377:GCG | NA | inframe_indel | c.2226_2246delGC | p.Pro743_Arg749 |
| ATCATCTCCCTCCT | | | CCAAGGAGGGAGA | del |
| TGGGC:G | | | TGATCG | |
| 2:25240378:C:A | rs34843713 | missense | c.2246G > T | p.Arg749Leu |
| 2:25240378:C:G | rs34843713 | missense | c.2246G > C | p.Arg749Pro |
| 2:25240378:C:T | rs34843713 | missense | c.2246G > A | p.Arg749His |
| 2:25240378:CG:C | NA | frameshift | c.2245delC | p.Arg749fs |
| 2:25240378:CGAT | NA | frameshift | c.2241_2245delTG | p.Asp747fs |
| CA:C | | | ATC | |
| 2:25240379:G:A | rs754613602 | missense | c.2245C > T | p.Arg749Cys |
| 2:25240379:G:C | rs754613602 | missense | c.2245C > G | p.Arg749Gly |
| 2:25240379:G:GT | NA | frameshift | c.2244_2245insA | p.Arg749fs |
| 2:25240379:G:T | NA | missense | c.2245C > A | p.Arg749Ser |
| 2:25240379:GA:G | NA | frameshift | c.2244delT | p.Arg749fs |
| 2:25240379:GATC | NA | frameshift | c.2241_2244delTG | p.Asp748fs |
| A:G | | | AT | |
| 2:25240380:AT:A | NA | frameshift | c.2243delA | p.Asp748fs |
| 2:25240381:T:TC | NA | frameshift | c.2242dupG | p.Asp748fs |
| 2:25240381:T:TC | NA | frameshift | c.2224_2242dupCG | p.Asp748fs |
| ATCTCCCTCCTTG | | | GCCCAAGGAGGGA | |
| GGCCG | | | GATG | |
| 2:25240381:TC:T | NA | frameshift | c.2242delG | p.Asp748fs |
| 2:25240383:A:AT | NA | frameshift | c.2239_2240dupG | p.Asp747fs |
| C | | | A | |
| 2:25240384:T:C | NA | missense | c.2240A > G | p.Asp747Gly |
| 2:25240384:T:G | NA | missense | c.2240A > C | p.Asp747Ala |
| 2:25240384:TCTC | NA | frameshift | c.2233_2239delGA | p.Glu745fs |
| CCTC:T | | | GGGAG | |
| 2:25240385:C:A | NA | missense | c.2239G > T | p.Asp747Tyr |
| 2:25240385:CT:C | NA | frameshift | c.2238delA | p.Asp747fs |
| 2:25240385:CTCC | NA | frameshift | c.2219_2238delAT | p.Asp740fs |
| CTCCTTGGGCCGC | | | GCGCGGCCCAAGG | |
| GCAT:C | | | AGGGA | |
| 2:25240386:TC:T | NA | frameshift | c.2237delG | p.Gly746fs |
| 2:25240387:C:T | NA | missense | c.2237G > A | p.Gly746Glu |
| 2:25240388:C:A | NA | stop_gained | c.2236G > T | p.Gly746* |
| 2:25240388:C:T | rs747626849 | missense | c.2236G > A | p.Gly746Arg |
| 2:25240389:CTCC | NA | frameshift | c.2231_2234delAG | p.Lys744fs |
| T:C | | | GA | |
| 2:25240390:T:TCC | NA | frameshift | c.2232_2233dupG | p.Glu745fs |
| | | | G | |
| 2:25240390:T:TCC | NA | frameshift | c.2227_2233dupCC | p.Glu745fs |
| TTGGG | | | CAAGG | |
| 2:25240391:C:A | rs1328524162 | stop_gained | c.2233G > T | p.Glu745* |
| 2:25240392:C:CT | NA | frameshift | c.2231dupA | p.Glu745fs |
| 2:25240392:CT:C | NA | frameshift | c.2231delA | p.Lys744fs |
| 2:25240394:T:A | rs1389075270 | stop_gained | c.2230A > T | p.Lys744* |
| 2:25240394:T:C | NA | missense | c.2230A > G | p.Lys744Glu |
| 2:25240394:T:TG | NA | frameshift | c.2229dupC | p.Lys744fs |
| 2:25240394:T:TG | NA | frameshift | c.2177_2229dupGC | p.Lys744fs |
| GGCCGCGCATCAT | | | ACTGGCCGGCTCTT | |
| GCAGGAGGCGGT | | | CTTTGAGTTCTACC | |
| AGAACTCAAAGA | | | GCCTCCTGCATGAT | |
| AGAGCCGGCCAG | | | GCGCGGCCC | |
| TGC | | | | |
| 2:25240394:T:TTA | NA | frameshift | c.2229_2230insTA | p.Lys744fs |
| 2:25240394:TG:T | NA | frameshift | c.2229delC | p.Lys744fs |
| 2:25240394:TGG | NA | frameshift | c.2225_2229delGG | p.Arg742fs |
| GCC:T | | | CCC | |
| 2:25240396:G:A | rs755673156 | missense | c.2228C > T | p.Pro743Leu |
| 2:25240396:G:C | rs755673156 | missense | c.2228C > G | p.Pro743Arg |
| 2:25240396:G:GG | NA | frameshift | c.2223_2227dupGC | p.Pro743fs |
| CCGC | | | GGC | |
| 2:25240396:G:T | NA | missense | c.2228C > A | p.Pro743His |
| 2:25240397:G:A | rs1433353413 | missense | c.2227C > T | p.Pro743Ser |
| 2:25240397:G:C | rs1433353413 | missense | c.2227C > G | p.Pro743Ala |
| 2:25240397:G:GC | NA | frameshift | c.2226dupG | p.Pro743fs |
| 2:25240397:GC:G | rs778606920 | frameshift | c.2226delG | p.Lys744fs |
| 2:25240398:C:CC | rs745364706 | frameshift | c.2224_2225dupCG | p.Pro743fs |
| G | | | | |
| 2:25240398:CCG: | NA | frameshift | c.2224_2225delCG | p.Arg742fs |
| C | | | | |
| 2:25240398:CCGC | NA | frameshift | c.2222_2225delCG | p.Ala741fs |
| G:C | | | CG | |
| 2:25240398:CCGC | NA | frameshift | c.2215_2225delCA | p.His739fs |
| GCATCATG:C | | | TGATGCGCG | |
| 2:25240399:C:CG | NA | frameshift | c.2224dupC | p.Arg742fs |
| 2:25240399:C:CT | NA | frameshift | c.2224_2225insA | p.Arg742fs |
| 2:25240399:C:G | rs749282210 | missense | c.2225G > C | p.Arg742Pro |
| 2:25240399:C:T | NA | missense | c.2225G > A | p.Arg742Gln |
| 2:25240399:CG:C | NA | frameshift | c.2224delC | p.Arg742fs |
| 2:25240399:CGC | NA | frameshift | c.2220_2224delTG | p.Asp740fs |
| GCA:C | | | CGC | |
| 2:25240399:CGC | NA | frameshift | c.2215_2224delCA | p.His739fs |
| GCATCATG:C | | | TGATGCGC | |
| 2:25240399:CGC | NA | inframe_indel | c.2213_2224delTG | p.Leu738_Ala741 |
| GCATCATGCA:C | | | CATGATGCGC | del |
| 2:25240399:CGC | NA | frameshift | c.2209_2224delCT | p.Leu737fs |
| GCATCATGCAGGA | | | CCTGCATGATGCGC | |
| G:C | | | | |
| 2:25240400:G:C | NA | missense | c.2224C > G | p.Arg742Gly |
| 2:25240402:G:A | rs770568549 | missense | c.2222C > T | p.Ala741Val |
| 2:25240402:G:C | NA | missense | c.2222C > G | p.Ala741Gly |
| 2:25240402:G:GC | NA | frameshift | c.2221dupG | p.Ala741fs |
| 2:25240402:G:T | rs770568549 | missense | c.2222C > A | p.Ala741Glu |
| 2:25240403:C:CA | NA | frameshift | c.2220dupT | p.Ala741fs |
| 2:25240403:C:CA | NA | frameshift | c.2211_2220dupCC | p.Ala741fs |
| TCATGCAGG | | | TGCATGAT | |
| 2:25240403:C:G | NA | missense | c.2221G > C | p.Ala741Pro |
| 2:25240403:C:T | NA | missense | c.2221G > A | p.Ala741Thr |
| 2:25240403:CATC | NA | frameshift | c.2217_2220delTG | p.His739fs |
| A:C | | | AT | |
| 2:25240404:A:AA | NA | inframe_indel | c.2219_2220insCG | p.Asp740_Ala741 |
| CG | | | T | insVal |
| 2:25240406:C:CA | NA | frameshift | c.2217dupT | p.Asp740fs |
| 2:25240407:A:AT | NA | frameshift | c.2174- | p.His739fs |
| GCAGGAGGCGGT | | | 1_2216dupGAGGG | |
| AGAACTCAAAGA | | | CACTGGCCGGCTCT | |
| AGAGCCGGCCAG | | | TCTTTGAGTTCTACC | |
| TGCCCTC | | | GCCTCCTGCA | |
| 2:25240408:T:C | NA | missense | c.2216A > G | p.His739Arg |
| 2:25240408:T:G | rs1053660507 | missense | c.2216A > C | p.His739Pro |
| 2:25240409:G:A | NA | missense | c.2215C > T | p.His739Tyr |
| 2:25240410:CAG | NA | inframe_indel | c.2211_2213delCC | p.Leu738del |
| G:C | | | T | |
| 2:25240411:A:AG | NA | frameshift | c.2212dupC | p.Leu738fs |
| 2:25240411:A:C | rs745714537 | missense | c.2213T > G | p.Leu738Arg |
| 2:25240411:A:G | NA | missense | c.2213T > C | p.Leu738Pro |
| 2:25240411:A:T | rs745714537 | missense | c.2213T > A | p.Leu738Gln |
| 2:25240411:AG:A | NA | frameshift | c.2212delC | p.Leu738fs |
| 2:25240412:G:C | rs1374722278 | missense | c.2212C > G | p.Leu738Val |
| 2:25240412:GGA | NA | frameshift | c.2198_2211delAG | p.Glu733fs |
| GGCGGTAGAACT: | | | TTCTACCGCCTC | |
| G | | | | |
| 2:25240413:GA:G | NA | frameshift | c.2210delT | p.Leu737fs |
| 2:25240414:A:C | rs768964533 | missense | c.2210T > G | p.Leu737Arg |
| 2:25240414:A:G | NA | missense | c.2210T > C | p.Leu737Pro |
| 2:25240414:A:T | NA | missense | c.2210T > A | p.Leu737His |
| 2:25240414:AG:A | NA | frameshift | c.2209delC | p.Leu737fs |
| 2:25240415:G:A | rs1340822377 | missense | c.2209C > T | p.Leu737Phe |
| 2:25240416:GCG | NA | frameshift | c.2198_2207delAG | p.Glu733fs |
| GTAGAACT:G | | | TTCTACCG | |
| 2:25240416:GCG | NA | frameshift | c.2192_2207delTCT | p.Phe731fs |
| GTAGAACTCAAAG | | | TTGAGTTCTACCG | |
| A:G | | | | |
| 2:25240417:C:A | NA | missense | c.2207G > T | p.Arg736Leu |
| 2:25240417:C:G | NA | missense | c.2207G > C | p.Arg736Pro |
| 2:25240417:C:T | rs139293773 | missense | c.2207G > A | p.Arg736His |
| 2:25240418:G:A | rs761934754 | missense | c.2206C > T | p.Arg736Cys |
| 2:25240418:G:C | rs761934754 | missense | c.2206C > G | p.Arg736Gly |
| 2:25240418:G:T | rs761934754 | missense | c.2206C > A | p.Arg736Ser |
| 2:25240419:G:C | rs765439043 | stop_gained | c.2205C > G | p.Tyr735* |
| 2:25240419:G:GT | NA | frameshift | c.2204dupA | p.Tyr735fs |
| 2:25240419:G:T | NA | stop_gained | c.2205C > A | p.Tyr735* |
| 2:25240419:GT:G | NA | frameshift | c.2204delA | p.Tyr735fs |
| 2:25240420:T:A | NA | missense | c.2204A > T | p.Tyr735Phe |
| 2:25240420:T:C | rs147828672 | missense | c.2204A > G | p.Tyr735Cys |
| 2:25240420:T:G | rs147828672 | missense | c.2204A > C | p.Tyr735Ser |
| 2:25240420:T:TA | NA | frameshift | c.2193_2203dupCT | p.Tyr735fs |
| GAACTCAAAG | | | TTGAGTTCT | |
| 2:25240420:TAG: | | frameshift | c.2202_2203delCT | p.Phe734fs |
| T | | | | |
| 2:25240420:TAGA | NA | frameshift | c.2197_2203delGA | p.Glu733fs |
| ACTC:T | | | GTTCT | |
| 2:25240421:A:AG | NA | frameshift | c.2174- | p.Tyr735fs |
| AACTCAAAGAAG | | | 2_2202dupAGAGG | |
| AGCCGGCCAGTG | | | GCACTGGCCGGCTC | |
| CCCTCT | | | TTCTTTGAGTTC | |
| 2:25240421:A:C | NA | missense | c.2203T > G | p.Tyr735Asp |
| 2:25240421:A:G | NA | missense | c.2203T > C | p.Tyr735His |
| 2:25240421:A:T | NA | missense | c.2203T > A | p.Tyr735Asn |
| 2:25240421:AG:A | NA | frameshift | c.2202delC | p.Tyr735fs |
| 2:25240421:AGA | NA | frameshift | c.2190_2202delCTT | p.Phe731fs |
| ACTCAAAGAAG:A | | | CTTTGAGTTC | |
| 2:25240422:G:C | | missense | c.2202C > G | p.Phe734Leu |
| 2:25240422:G:GA | NA | frameshift | c.2201dupT | p.Tyr735fs |
| 2:25240422:G:GA | NA | frameshift | c.2192_2201dupTC | p.Tyr735fs |
| ACTCAAAGA | | | TTTGAGTT | |
| 2:25240422:G:GA | NA | inframe_indel | c.2201_2202insAG | p.Phe734delinsLe |
| CT | | | T | uVal |
| 2:25240422:G:GA | NA | frameshift | c.2201_2202insAT | p.Phe734fs |
| T | | | | |
| 2:25240422:G:T | rs766854284 | missense | c.2202C > A | p.Phe734Leu |
| 2:25240422:GA:G | | frameshift | c.2201delT | p.Phe734fs |
| 2:25240422:GAA | NA | frameshift | c.2189_2201delTCT | p.Leu730fs |
| CTCAAAGAAGA:G | | | TCTTTGAGTT | |
| 2:25240423:A:C | rs1215180755 | missense | c.2201T > G | p.Phe734Cys |
| 2:25240423:A:G | rs1215180755 | missense | c.2201T > C | p.Phe734Ser |
| 2:25240423:AACT: | NA | inframe_indel | c.2198_2200delAG | p.Glu733_Phe734 |
| A | | | T | delinsVal |
| 2:25240423:AACT | NA | frameshift | c.2185_2200delCG | p.Arg729fs |
| CAAAGAAGAGCC | | | GCTCTTCTTTGAGT | |
| G:A | | | | |
| 2:25240424:A:C | rs751950768 | missense | c.2200T > G | p.Phe734Val |
| 2:25240424:A:G | NA | missense | c.2200T > C | p.Phe734Leu |
| 2:25240424:A:T | NA | missense | c.2200T > A | p.Phe734Ile |
| 2:25240425:C:CT | NA | frameshift | c.2198dupA | p.Phe734fs |
| 2:25240425:CTCA | NA | frameshift | c.2188_2198delCT | p.Leu730fs |
| AAGAAGAG:C | | | CTTCTTTGA | |
| 2:25240426:T:A | NA | missense | c.2198A > T | p.Glu733Val |
| 2:25240426:T:C | rs755376933 | missense | c.2198A > G | p.Glu733Gly |
| 2:25240426:T:G | NA | missense | c.2198A > C | p.Glu733Ala |
| 2:25240426:T:TC | NA | frameshift | c.2197dupG | p.Glu733fs |
| 2:25240426:TC:T | NA | frameshift | c.2197delG | p.Glu733fs |
| 2:25240426:TCAA | NA | inframe_indel | c.2183_2197delGC | p.Gly728_Phe732 |
| AGAAGAGCCGGC: | | | CGGCTCTTGTTTG | del |
| T | | | | |
| 2:25240427:C:A | NA | stop_gained | c.2197G > T | p.Glu733* |
| 2:25240427:C:CA | | frameshift | c.2196dupT | p.Glu733fs |
| 2:25240427:C:CA | rs771906613 | frameshift | c.2195_2196dupTT | p.Glu733fs |
| A | | | | |
| 2:25240427:C:CA | NA | frameshift | c.2196_2197insCT | p.Glu733fs |
| G | | | | |
| 2:25240427:C:G | NA | missense | c.2197G > C | p.Glu733Gln |
| 2:25240427:C:T | NA | missense | c.2197G > A | p.Glu733Lys |
| 2:25240427:CA:C | NA | frameshift | c.2196delT | p.Phe732fs |
| 2:25240427:CAA: | rs774944755 | frameshift | c.2195_2196delTT | p.Phe732fs |
| C | | | | |
| 2:25240427:CAAA | NA | frameshift | c.2193_2196delCTT | p.Phe731fs |
| G:C | | | T | |
| 2:25240427:CAAA | NA | frameshift | c.2192_2196delTCT | p.Phe731fs |
| GA:C | | | TT | |
| 2:25240427:CAAA | NA | frameshift | c.2190_2196delCTT | p.Phe731fs |
| GAAG:C | | | CTTT | |
| 2:25240428:A:C | NA | missense | c.2196T > G | p.Phe732Leu |
| 2:25240428:A:T | NA | missense | c.2196T > A | p.Phe732Leu |
| 2:25240428:AAA | rs761103716 | inframe_indel | c.2193_2195delCTT | p.Phe732del |
| G:A | | | | |
| 2:25240428:AAA | NA | inframe_indel | c.2190_2195delCTT | p.Phe731_Phe73 |
| GAAG:A | | | CTT | 2del |
| 2:25240429:A:C | rs767134940 | missense | c.2195T > G | p.Phe732Cys |
| 2:25240429:A:G | NA | missense | c.2195T > C | p.Phe732Ser |
| 2:25240429:A:T | NA | missense | c.2195T > A | p.Phe732Tyr |
| 2:25240429:AAG: | NA | frameshift | c.2193_2194delCT | p.Phe732fs |
| A | | | | |
| 2:25240429:AAG | NA | frameshift | c.2190_2194delCTT | p.Phe731fs |
| AAG:A | | | CT | |
| 2:25240430:A:C | NA | missense | c.2194T > G | p.Phe732Val |
| 2:25240430:A:G | NA | missense | c.2194T > C | p.Phe732Leu |
| 2:25240430:A:T | rs149043640 | missense | c.2194T > A | p.Phe732Ile |
| 2:25240430:AG:A | NA | frameshift | c.2193delC | p.Phe732fs |
| 2:25240430:AGA | NA | frameshift | c.2186_2193delGG | p.Arg729fs |
| AGAGCC:A | | | CTCTTC | |
| 2:25240431:G:C | NA | missense | c.2193C > G | p.Phe731Leu |
| 2:25240431:G:GC | NA | frameshift | c.2192_2193insG | p.Phe731fs |
| 2:25240431:G:GT | NA | frameshift | c.2192_2193insA | p.Phe731fs |
| 2:25240431:G:GT | rs768858047 | frameshift | c.2192_2193insAA | p.Phe731fs |
| T | | | | |
| 2:25240431:G:T | NA | missense | c.2193C > A | p.Phe731Leu |
| 2:25240431:GA:G | NA | frameshift | c.2192delT | p.Phe731fs |
| 2:25240431:GAA: | NA | frameshift | c.2191_2192delTT | p.Phe731fs |
| G | | | | |
| 2:25240432:A:C | rs755585212 | missense | c.2192T > G | p.Phe731Cys |
| 2:25240432:A:G | NA | missense | c.2192T > C | p.Phe731Ser |
| 2:25240432:A:T | NA | missense | c.2192T > A | p.Phe731Tyr |
| 2:25240432:AAG: | NA | frameshift | c.2190_2191delCT | p.Phe731fs |
| A | | | | |
| 2:25240433:A:AT | NA | frameshift | c.2190_2191insA | p.Phe731fs |
| 2:25240433:A:C | NA | missense | c.2191T > G | p.Phe731Val |
| 2:25240433:A:G | rs143019657 | missense | c.2191T > C | p.Phe731Leu |
| 2:25240433:A:T | NA | missense | c.2191T > A | p.Phe731Ile |
| 2:25240433:AG:A | NA | frameshift | c.2190delC | p.Phe731fs |
| 2:25240433:AGA | NA | frameshift | c.2186_2190delGG | p.Arg729fs |
| GCC:A | | | CTC | |
| 2:25240434:G:GA | NA | frameshift | c.2186_2189dupG | p.Phe731fs |
| GCC | | | GCT | |
| 2:25240434:GA:G | NA | frameshift | c.2189delT | p.Leu730fs |
| 2:25240434:GAG | NA | frameshift | c.2182_2189delGG | p.Gly728fs |
| CCGGCCTG | | | CCGGCT | |
| 2:25240435:A:AG | NA | frameshift | c.2187_2188dupGC | p.Leu730fs |
| C | | | | |
| 2:25240435:A:G | rs749126333 | missense | c.2189T > C | p.Leu730Pro |
| 2:25240435:AG:A | NA | frameshift | c.2188delC | p.Leu730fs |
| 2:25240435:AGCC | NA | frameshift | c.2179_2188delAC | p.Thr727fs |
| GGCCAGT:A | | | TGGCCGGC | |
| 2:25240437:C:CC | NA | inframe_indel | c.2186_2187insAG | p.Arg729dup |
| CT | | | G | |
| 2:25240437:C:CC | NA | frameshift | c.2174- | p.Leu730fs |
| GGCCAGTGCCCTC | | | 9_2186dupACCTCT | |
| TGAGAGGT | | | CAGAGGGCACTGG | |
| | | | CCG | |
| 2:25240437:CCG: | NA | frameshift | c.2185_2186delCG | p.Arg729fs |
| C | | | | |
| 2:25240438:C:A | NA | missense | c.2186G > T | p.Arg729Leu |
| 2:25240438:C:CG | NA | frameshift | c.2181_2185dupTG | p.Arg729fs |
| GCCA | | | GCC | |
| 2:25240438:C:CG | NA | frameshift | c.2174- | p.Arg729fs |
| GCCAGTGCCCTCT | | | 4_2185dupTCAGA | |
| GA | | | GGGCACTGGCC | |
| 2:25240438:C:G | NA | missense | c.2186G > C | p.Arg729Pro |
| 2:25240438:C:T | rs757211277 | missense | c.2186G > A | p.Arg729Gln |
| 2:25240438:CG:C | NA | frameshift | c.2185delC | p.Arg729fs |
| 2:25240439:G:A | rs200018028 | missense | c.2185C > T | p.Arg729Trp |
| 2:25240439:G:C | rs200018028 | missense | c.2185C > G | p.Arg729Gly |
| 2:25240439:GGC | NA | frameshift | c.2181_2184delTG | p.Arg729fs |
| CA:G | | | GC | |
| 2:25240440:G:GC | NA | frameshift | c.2183dupG | p.Arg729fs |
| 2:25240440:GC:G | NA | frameshift | c.2183delG | p.Gly728fs |
| 2:25240441:C:A | NA | missense | c.2183G > T | p.Gly728Val |
| 2:25240441:C:CA | NA | frameshift | c.2182_2183insT | p.Gly728fs |
| 2:25240441:C:T | NA | missense | c.2183G > A | p.Gly728Asp |
| 2:25240442:C:A | NA | missense | c.2182G > T | p.Gly728Cys |
| 2:25240442:C:G | NA | missense | c.2182G > C | p.Gly728Arg |
| 2:25240442:C:T | NA | missense | c.2182G > A | p.Gly728Ser |
| 2:25240443:AG:A | NA | frameshift | c.2180delC | p.Thr727fs |
| 2:25240444:GT:G | NA | frameshift | c.2179delA | p.Thr727fs |
| 2:25240445:TG:T | NA | frameshift | c.2178delC | p.Thr727fs |
| 2:25240446:GC:G | NA | frameshift | c.2177delG | p.Gly726fs |
| 2:25240446:GCCC | NA | frameshift | c.2174- | p.Glu725fs |
| TCTGAGAGGTC:G | | | 10_2177delGACCT | |
| | | | CTCAGAGGG | |
| 2:25240447:C:A | rs1432383727 | missense | c.2177G > T | p.Gly726Val |
| 2:25240447:C:T | NA | missense | c.2177G > A | p.Gly726Asp |
| 2:25240448:C:A | NA | missense | c.2176G > T | p.Gly726Cys |
| 2:25240448:C:G | NA | missense | c.2176G > C | p.Gly726Arg |
| 2:25240448:CCT: | rs1175429748 | frameshift | c.2174_2175delAG | p.Glu725fs |
| C | | | | |
| 2:25240448:CCTC | NA | frameshift | c.2174- | p.Glu725fs |
| T:C | | | 2_2175delAGAG | |
| 2:25240449:CT:C | NA | frameshift | c.2174delA | p.Glu725fs |
| 2:25240451:C:A | NA | splice_acceptor | c.2174-1G > T | |
| 2:25240451:C:G | NA | splice_acceptor | c.2174-1G > C | |
| 2:25240451:C:T | rs1262354256 | splice_acceptor | c.2174-1G > A | |
| 2:25240452:T:A | NA | splice_acceptor | c.2174-2A > T | |
| 2:25240452:T:C | rs772010891 | splice_acceptor | c.2174-2A > G | |
| 2:25240452:T:G | rs772010891 | splice_acceptor | c.2174-2A > C | |
| 2:25240631:GGT | NA | frameshift | c.2173_2173+8del | p.Glu725fs |
| ACCTACC:G | | | GGTAGGTAC | |
| 2:25240633:TACC | NA | frameshift | c.2170_2173+6delT | p.Tyr724fs |
| TACCGTA:T | | | ACGGTAGGT | |
| 2:25240637:T:TA | NA | splice_donor | c.2173+2dupT | |
| 2:25240638:A:G | NA | splice_donor | c.2173+2T > C | |
| 2:25240638:AC:A | NA | splice_donor | c.2173+1delG | |
| 2:25240639:C:A | NA | splice_donor | c.2173+1G > T | |
| 2:25240639:C:G | NA | splice_donor | c.2173+1G > C | |
| 2:25240639:C:T | rs763716866 | splice_donor | c.2173+1G > A | |
| 2:25240640:C:A | | stop_gained | c.2173G > T | p.Glu725* |
| 2:25240640:C:CC | NA | stop_gained | c.2172_2173insTA | p.Tyr724_Glu725 |
| TA | | | G | insTer |
| 2:25240640:C:CG | NA | frameshift | c.2171_2172dupAC | p.Glu725fs |
| T | | | | |
| 2:25240641:G:C | NA | stop_gained | c.2172C > G | p.Tyr724* |
| 2:25240641:G:T | NA | stop_gained | c.2172C > A | p.Tyr724* |
| 2:25240642:T:C | rs1330225644 | missense | c.2171A > G | p.Tyr724Cys |
| 2:25240642:T:TCT | NA | stop_gained | c.2170_2171insGA | p.Tyr724delinsTer |
| C | | | G | Asp |
| 2:25240644:G:GA | NA | frameshift | c.2168dupT | p.Tyr724fs |
| 2:25240644:GA:G | NA | frameshift | c.2168delT | p.Leu723fs |
| 2:25240645:A:T | NA | missense | c.2168T > A | p.Leu723His |
| 2:25240645:AGG: | NA | frameshift | c.2166_2167delCC | p.Tyr724fs |
| A | | | | |
| 2:25240646:G:T | NA | missense | c.2167C > A | p.Leu723Ile |
| 2:25240647:GC:G | NA | frameshift | c.2165delG | p.Gly722fs |
| 2:25240647:GCC: | NA | frameshift | c.2164_2165delGG | p.Gly722fs |
| G | | | | |
| 2:25240648:C:T | NA | missense | c.2165G > A | p.Gly722Asp |
| 2:25240650:C:CTT | NA | frameshift | c.2161_2162dupAA | p.Gly722fs |
| 2:25240651:T:A | NA | missense | c.2162A > T | p.Lys721Met |
| 2:25240652:T:A | NA | stop_gained | c.2161A > T | p.Lys721* |
| 2:25240652:T:C | NA | missense | c.2161A > G | p.Lys721Glu |
| 2:25240652:T:TCC | NA | frameshift | c.2160_2161insTC | p.Lys721fs |
| GA | | | GG | |
| 2:25240653:GC:G | NA | frameshift | c.2159delG | p.Arg720fs |
| 2:25240654:C:A | rs1272076221 | missense | c.2159G > T | p.Arg720Leu |
| 2:25240654:C:G | NA | missense | c.2159G > C | p.Arg720Pro |
| 2:25240654:C:T | NA | missense | c.2159G > A | p.Arg720His |
| 2:25240654:CG:C | NA | frameshift | c.2158delC | p.Arg720fs |
| 2:25240654:CGA | NA | frameshift | c.2152_2158delCC | p.Pro718fs |
| GCAGG:C | | | TGCTC | |
| 2:25240655:G:A | rs1197133406 | missense | c.2158C > T | p.Arg720Cys |
| 2:25240655:G:C | NA | missense | c.2158C > G | p.Arg720Gly |
| 2:25240655:G:T | NA | missense | c.2158C > A | p.Arg720Ser |
| 2:25240657:G:GT | NA | frameshift | c.2155_2156insA | p.Ala719fs |
| 2:25240657:GCA | NA | frameshift | c.2149_2155delAA | p.Asn717fs |
| GGGTT:G | | | CCCTG | |
| 2:25240658:C:G | NA | missense | c.2155G > C | p.Ala719Pro |
| 2:25240658:C:T | NA | missense | c.2155G > A | p.Ala719Thr |
| 2:25240658:CAG | NA | frameshift | c.2139_2154delCT | p.Ser714fs |
| GGTTGACGATGG | | | CCATCGTCAACCCT | |
| AG:C | | | | |
| 2:25240659:AG:A | NA | frameshift | c.2153delC | p.Pro718fs |
| 2:25240660:G:A | NA | missense | c.2153C > T | p.Pro718Leu |
| 2:25240660:G:GG | NA | frameshift | c.2143_2152dupAT | p.Pro718fs |
| GTTGACGAT | | | CGTCAACC | |
| 2:25240661:G:A | NA | missense | c.2152C > T | p.Pro718Ser |
| 2:25240662:G:GT | NA | frameshift | c.2150dupA | p.Asn717fs |
| 2:25240662:G:GT | NA | frameshift | c.2149_2150dupAA | p.Asn717fs |
| T | | | | |
| 2:25240662:GT:G | NA | frameshift | c.2150delA | p.Asn717fs |
| 2:25240663:T:A | NA | missense | c.2150A > T | p.Asn717Ile |
| 2:25240663:T:C | NA | missense | c.2150A > G | p.Asn717Ser |
| 2:25240663:T:TT | NA | frameshift | c.2148_2149dupCA | p.Asn717fs |
| G | | | | |
| 2:25240664:TGA: | NA | frameshift | c.2147_2148delTC | p.Va1716fs |
| T | | | | |
| 2:25240666:A:C | NA | missense | c.2147T > G | p.Va1716Gly |
| 2:25240666:A:T | rs778857953 | missense | c.2147T > A | p.Va1716Asp |
| 2:25240666:AC:A | NA | frameshift | c.2146delG | p.Va1716fs |
| 2:25240667:C:A | rs1418039680 | missense | c.2146G > T | p.Va1716Phe |
| 2:25240667:C:CA | NA | frameshift | c.2145_2146insT | p.Va1716fs |
| 2:25240667:C:T | rs1418039680 | missense | c.2146G > A | p.Va1716Ile |
| 2:25240667:CG:C | NA | frameshift | c.2145delC | p.Ile715fs |
| 2:25240668:GAT: | NA | frameshift | c.2143_2144delAT | p.Ile715fs |
| G | | | | |
| 2:25240668:GAT | NA | frameshift | c.2134_2144delGA | p.Asp712fs |
| GGAGAGGTC:G | | | CCTCTCCAT | |
| 2:25240669:ATG | NA | frameshift | c.2136_2143delCC | p.Leu713fs |
| GAGAGG:A | | | TCTCCA | |
| 2:25240670:T:TG | rs1179539918 | frameshift | c.2142dupC | p.Ile715fs |
| 2:25240670:TG:T | NA | frameshift | c.2142delC | p.Ile715fs |
| 2:25240670:TGG | NA | frameshift | c.2138_2142delTCT | p.Leu713fs |
| AGA:T | | | CC | |
| 2:25240671:G:GA | NA | frameshift | c.2141_2142insT | p.Ile715fs |
| 2:25240671:G:GG | NA | frameshift | c.2140_2141dupTC | p.Ile715fs |
| A | | | | |
| 2:25240671:G:GG | NA | frameshift | c.2138_2141dupTC | p.Ile715fs |
| AGA | | | TC | |
| 2:25240671:GGA: | rs1281903798 | frameshift | c.2140_2141delTC | p.Ser714fs |
| G | | | | |
| 2:25240672:G:A | NA | missense | c.2141C > T | p.Ser714Phe |
| 2:25240672:G:C | rs367909007 | missense | c.2141C > G | p.Ser714Cys |
| 2:25240672:G:GA | NA | frameshift | c.2140dupT | p.Ser714fs |
| 2:25240672:G:T | NA | missense | c.2141C > A | p.Ser714Tyr |
| 2:25240674:GAG | NA | frameshift | c.2134_2138delGA | p.Asp712fs |
| GTCTG | | | CCT | |
| 2:25240675:A:AT | NA | frameshift | c.2137_2138insA | p.Leu713fs |
| 2:25240675:AG:A | rs777214520 | frameshift | c.2137delC | p.Leu713fs |
| 2:25240675:AGG | NA | frameshift | c.2127_2137delCT | p.Cys710fs |
| TCATTGCAG:A | | | GCAATGACC | |
| 2:25240676:G:A | rs780006700 | missense | c.2137C > T | p.Leu713Phe |
| 2:25240676:G:C | NA | missense | c.2137C > G | p.Leu713Val |
| 2:25240676:GGT: | NA | frameshift | c.2135_2136delAC | p.Asp712fs |
| G | | | | |
| 2:25240677:G:GT | NA | frameshift | c.2135dupA | p.Asp712fs |
| 2:25240677:G:GT | NA | frameshift | c.2132_2135dupAT | p.Asp712fs |
| CAT | | | GA | |
| 2:25240677:G:T | NA | missense | c.2136C > A | p.Asp712Glu |
| 2:25240677:GT:G | NA | frameshift | c.2135delA | p.Asp712fs |
| 2:25240678:T:A | NA | missense | c.2135A > T | p.Asp712Val |
| 2:25240678:T:C | NA | missense | c.2135A > G | p.Asp712Gly |
| 2:25240678:T:G | NA | missense | c.2135A > C | p.Asp712Ala |
| 2:25240678:TC:T | NA | frameshift | c.2134delG | p.Asp712fs |
| 2:25240678:TCAT | NA | frameshift | c.2118_2134delGG | p.Gly707fs |
| TGCAGGGACTGC | | | GCAGTCCCTGCAAT | |
| CC:T | | | G | |
| 2:25240680:A:AT | NA | frameshift | c.2132dupA | p.Asn711fs |
| 2:25240682:TG:T | NA | frameshift | c.2130delC | p.Cys710fs |
| 2:25240683:G:T | NA | stop_gained | c.2130C > A | p.Cys710* |
| 2:25240683:GCA | NA | frameshift | c.2117_2129delGG | p.Gly706fs |
| GGGACTGCCCC:G | | | GGCAGTCCCTG | |
| 2:25240684:C:G | rs1444323322 | missense | c.2129G > C | p.Cys710Ser |
| 2:25240684:C:T | NA | missense | c.2129G > A | p.Cys710Tyr |
| 2:25240684:CA:C | NA | frameshift | c.2128delT | p.Cys710fs |
| 2:25240685:A:G | NA | missense | c.2128T > C | p.Cys710Arg |
| 2:25240685:A:T | rs746857175 | missense | c.2128T > A | p.Cys710Ser |
| 2:25240685:AG:A | NA | frameshift | c.2127delC | p.Cys710fs |
| 2:25240687:G:A | NA | missense | c.2126C > T | p.Pro709Leu |
| 2:25240687:G:GA | NA | frameshift | c.2125_2126insT | p.Pro709fs |
| 2:25240687:GGA | NA | frameshift | c.2112_2125delGA | p.Ile705fs |
| CTGCCCCCAATCTG | | | TTGGGGGCAGTC | |
| 2:25240688:GA:G | NA | frameshift | c.2124delT | p.Cys710fs |
| 2:25240689:AC:A | NA | frameshift | c.2123delG | p.Ser708fs |
| 2:25240689:ACTG | NA | inframe_indel | c.2094_2123delGG | p.Trp698_Ser708 |
| CCCCCAATCACCA | | | GCCCATTCGATCTG | delinsCys |
| GATCGAATGGGC | | | GTGATTGGGGGCA | |
| CC:A | | | G | |
| 2:25240690:C:A | NA | missense | c.2123G > T | p.Ser708Ile |
| 2:25240690:C:T | NA | missense | c.2123G > A | p.Ser708Asn |
| 2:25240690:CT:C | NA | frameshift | c.2122delA | p.Ser708fs |
| 2:25240690:CTG: | NA | frameshift | c.2121_2122delCA | p.Pro709fs |
| C | | | | |
| 2:25240691:TG:T | NA | frameshift | c.2121delC | p.Ser708fs |
| 2:25240692:G:GC | rs1402117804 | frameshift | c.2120dupG | p.Ser708fs |
| 2:25240692:GC:G | rs762350913 | frameshift | c.2120delG | p.Gly707fs |
| 2:25240693:C:A | NA | missense | c.2120G > T | p.Gly707Val |
| 2:25240693:C:T | rs927337009 | missense | c.2120G > A | p.Gly707Asp |
| 2:25240694:C:A | NA | missense | c.2119G > T | p.Gly707Cys |
| 2:25240694:C:G | NA | missense | c.2119G > C | p.Gly707Arg |
| 2:25240694:C:T | NA | missense | c.2119G > A | p.Gly707Ser |
| 2:25240695:CCCA | NA | frameshift | c.2090_2117delAG | p.Glu697fs |
| ATCACCAGATCGA | | | TGGGGCCCATTCGA | |
| ATGGGCCCCACT: | | | TCTGGTGATTGG | |
| C | | | | |
| 2:25240696:C:A | NA | missense | c.2117G > T | p.Gly706Val |
| 2:25240696:C:T | rs777898555 | missense | c.2117G > A | p.Gly706Glu |
| 2:25240697:C:A | rs749365376 | missense | c.2116G > T | p.Gly706Trp |
| 2:25240697:c.G | NA | missense | c.2116G > C | p.Gly706Arg |
| 2:25240697:CTT | NA | missense | c.2116G > A | p.Gly706Arg |
| 2:25240697:CA:C | NA | frameshift | c.2115delT | p.Ile705fs |
| 2:25240698:AATC | NA | frameshift | c.2107_2114delCT | p.Leu703fs |
| ACCAG:A | | | GGTGAT | |
| 2:25240699:A:C | rs777037011 | missense | c.2114T > G | p.Ile705Ser |
| 2:25240699:A:G | rs777037011 | missense | c.2114T > C | p.Ile705Thr |
| 2:25240699:A:T | NA | missense | c.2114T > A | p.Ile705Asn |
| 2:25240699:AT:A | rs1411778215 | frameshift | c.2113delA | p.Ile705fs |
| 2:25240700:T:A | rs1291851173 | missense | c.2113A > T | p.Ile705Phe |
| 2:25240700:T:C | NA | missense | c.2113A > G | p.Ile705Val |
| 2:25240700:T:TC | NA | frameshift | c.2111_2112dupTG | p.Ile705fs |
| A | | | | |
| 2:25240700:TC:T | NA | frameshift | c.2112delG | p.Ile705fs |
| 2:25240700:TCA: | NA | frameshift | c.2111_2112delTG | p.Va1704fs |
| T | | | | |
| 2:25240701:CA:C | NA | frameshift | c.2111delT | p.Va1704fs |
| 2:25240702:A:C | NA | missense | c.2111T > G | p.Va1704Gly |
| 2:25240702:A:G | rs1165074698 | missense | c.2111T > C | p.Va1704Ala |
| 2:25240702:A:T | NA | missense | c.2111T > A | p.Va1704Glu |
| 2:25240702:AC:A | rs1454822561 | frameshift | c.2110delG | p.Va1704fs |
| 2:25240704:CAG: | NA | frameshift | c.2107_2108delCT | p.Leu703fs |
| C | | | | |
| 2:25240705:A:AG | NA | frameshift | c.2107dupC | p.Leu703fs |
| 2:25240705:A:C | NA | missense | c.2108T > G | p.Leu703Arg |
| 2:25240705:A:G | NA | missense | c.2108T > C | p.Leu703Pro |
| 2:25240705:A:T | NA | missense | c.2108T > A | p.Leu703Gln |
| 2:25240705:AG:A | rs765733093 | frameshift | c.2107delC | p.Leu703fs |
| 2:25240706:G:C | NA | missense | c.2107C > G | p.Leu703Val |
| 2:25240706:GAT: | NA | frameshift | c.2105_2106delAT | p.Asp702fs |
| G | | | | |
| 2:25240707:A:C | NA | missense | c.2106T > G | p.Asp702Glu |
| 2:25240707:A:T | NA | missense | c.2106T > A | p.Asp702Glu |
| 2:25240707:AT:A | NA | frameshift | c.2105delA | p.Asp702fs |
| 2:25240708:T:A | NA | missense | c.2105A > T | p.Asp702Val |
| 2:25240708:T:C | rs1400330086 | missense | c.2105A > G | p.Asp702Gly |
| 2:25240708:T:G | NA | missense | c.2105A > C | p.Asp702Ala |
| 2:25240708:TC:T | NA | frameshift | c.2104delG | p.Asp702fs |
| 2:25240708:TCGA | NA | frameshift | c.2097_2104delCC | p.Pro700fs |
| ATGGG:T | | | CATTCG | |
| 2:25240709:C:A | rs770938712 | missense | c.2104G > T | p.Asp702Tyr |
| 2:25240709:C:G | NA | missense | c.2104G > C | p.Asp702His |
| 2:25240709:C:T | rs770938712 | missense | c.2104G > A | p.Asp702Asn |
| 2:25240710:G:C | NA | missense | c.2103C > G | p.Phe701Leu |
| 2:25240710:G:T | NA | missense | c.2103C > A | p.Phe701Leu |
| 2:25240710:GA:G | NA | frameshift | c.2102delT | p.Phe701fs |
| 2:25240711:A:C | NA | missense | c.2102T > G | p.Phe701Cys |
| 2:25240711:A:G | NA | missense | c.2102T > C | p.Phe701Ser |
| 2:25240711:A:T | NA | missense | c.2102T > A | p.Phe701Tyr |
| 2:25240712:A:C | rs760042734 | missense | c.2101T > G | p.Phe701Val |
| 2:25240712:A:G | NA | missense | c.2101T > C | p.Phe701Leu |
| 2:25240712:AT:A | NA | frameshift | c.2100delA | p.Phe701fs |
| 2:25240712:ATG | NA | frameshift | c.2088_2100delGG | p.Gln696fs |
| GGCCCCACTCC:A | | | AGTGGGGCCCA | |
| 2:25240713:TG:T | rs773340419 | frameshift | c.2099delC | p.Pro700fs |
| 2:25240714:G:A | rs772368909 | missense | c.2099C > T | p.Pro700Leu |
| 2:25240714:G:C | NA | missense | c.2099C > G | p.Pro700Arg |
| 2:25240714:G:T | NA | missense | c.2099C > A | p.Pro700Gln |
| 2:25240715:G:A | rs775823075 | missense | c.2098C > T | p.Pro700Ser |
| 2:25240715:G:C | rs775823075 | missense | c.2098C > G | p.Pro700Ala |
| 2:25240715:G:T | rs775823075 | missense | c.2098C > A | p.Pro700Thr |
| 2:25240716:G:GA | NA | frameshift | c.2096_2097insT | p.Phe701fs |
| 2:25240716:G:GC | rs762965546 | frameshift | c.2096dupG | p.Phe701fs |
| 2:25240716:G:GC | NA | frameshift | c.2095_2096dupG | p.Pro700fs |
| C | | | G | |
| 2:25240716:G:GC | NA | frameshift | c.2092_2096dupTG | p.Pro700fs |
| CCCA | | | GGG | |
| 2:25240716:G:GC | NA | frameshift | c.2090_2096dupA | p.Pro700fs |
| CCCACT | | | GTGGGG | |
| 2:25240716:Gc.G | | frameshift | c.2096delG | p.Gly699fs |
| 2:25240716:GCCC | NA | frameshift | c.2092_2096delTG | p.Trp698fs |
| CA:G | | | GGG | |
| 2:25240716:GCCC | NA | frameshift | c.2090_2096delAG | p.Glu697fs |
| CACT:G | | | TGGGG | |
| 2:25240717:C:A | NA | missense | c.2096G > T | p.Gly699Val |
| 2:25240717:C:T | rs761064473 | missense | c.2096G > A | p.Gly699Asp |
| 2:25240718:C:A | NA | missense | c.2095G > T | p.Gly699Cys |
| 2:25240718:C:G | NA | missense | c.2095G > C | p.Gly699Arg |
| 2:25240718:C:T | rs763776241 | missense | c.2095G > A | p.Gly699Ser |
| 2:25240718:CCCA | NA | frameshift | c.2090_2094delAG | p.Glu697fs |
| CT:C | | | TGG | |
| 2:25240719:C:A | NA | missense | c.2094G > T | p.Trp698Cys |
| 2:25240719:C:T | rs753452266 | stop_gained | c.2094G > A | p.Trp698* |
| 2:25240720:C:G | NA | missense | c.2093G > C | p.Trp698Ser |
| 2:25240720:C:T | rs1005359774 | stop_gained | c.2093G > A | p.Trp698* |
| 2:25240720:CA:C | NA | frameshift | c.2092delT | p.Trp698fs |
| 2:25240720:CACT: | NA | inframe_indel | c.2090_2092delAG | p.Glu697_Trp698 |
| C | | | T | delinsGly |
| 2:25240721:A:G | NA | missense | c.2092T > C | p.Trp698Arg |
| 2:25240721:A:T | NA | missense | c.2092T > A | p.Trp698Arg |
| 2:25240721:AC:A | NA | frameshift | c.2091delG | p.Glu697fs |
| 2:25240723:T:TC | NA | frameshift | c.2089dupG | p.Glu697fs |
| 2:25240723:TCC:T | NA | frameshift | c.2088_2089delGG | p.Glu697fs |
| 2:25240724:C:A | rs761343150 | stop_gained | c.2089G > T | p.Glu697* |
| 2:25240726:TG:T | rs766594867 | frameshift | c.2086delC | p.Gln696fs |
| 2:25240726:TGG: | NA | frameshift | c.2085_2086delCC | p.Gln696fs |
| T | | | | |
| 2:25240726:TGG | NA | frameshift | c.2083- | p.Ile695fs |
| ATCTGGGAGGAT | | | 14_2086delCTTTAT | |
| AAAG:T | | | CCTCCCAGATCC | |
| 2:25240727:G:A | rs750325978 | stop_gained | c.2086C > T | p.Gln696* |
| 2:25240727:G:T | NA | missense | c.2086C > A | p.Gln696Lys |
| 2:25240729:A:T | rs780206885 | missense | c.2084T > A | p.Ile695Asn |
| 2:25240731:C:A | NA | splice_acceptor | c.2083-1G > T | |
| 2:25240731:C:G | NA | splice_acceptor | c.2083-1G > C | |
| 2:25240731:C:T | NA | splice_acceptor | c.2083-1G > A | |
| 2:25240732:T:A | NA | splice_acceptor | c.2083-2A > T | |
| 2:25240732:T:C | rs761987159 | splice_acceptor | c.2083-2A > G | |
| 2:25241553:TGG | NA | splice_donor | c.2059_2082+8del | p.Va1687_His694 |
| ACATACATGCTTC | | | GTCCGCAGCGTCAC | del |
| TGTGTGACGCTGC | | | ACAGAAGCATGTAT | |
| GGAC:T | | | GTCC | |
| 2:25241559:T:TA | rs751693865 | splice_donor | c.2082+2dupT | |
| 2:25241559:TACA | NA | frameshift | c.2070_2082+2del | p.Thr691fs |
| TGCTTCTGTGTG:T | | | CACACAGAAGCATG | |
| | | | T | |
| 2:25241560:A:C | rs1196735995 | splice_donor | c.2082+2T > G | |
| 2:25241560:A:G | rs1196735995 | splice_donor | c.2082+2T > C | |
| 2:25241560:A:T | NA | splice_donor | c.2082+2T > A | |
| 2:25241561:C:A | NA | splice_donor | c.2082+lG > T | |
| 2:25241561:C:CA | NA | frameshift | c.2082dupT | p.Ile695fs |
| 2:25241561:C:CA | NA | frameshift | c.2078_2082dupA | p.Ile695fs |
| TGCT | | | GCAT | |
| 2:25241561:C:G | NA | splice_donor | c.2082+1G > C | |
| 2:25241561:C:T | rs766397390 | splice_donor | c.2082+1G > A | |
| 2:25241561:CATG | NA | frameshift | c.2075_2082delAG | p.Gln692fs |
| CTTCT:C | | | AAGCAT | |
| 2:25241562:AT:A | NA | frameshift | c.2081delA | p.His694fs |
| 2:25241563:T:C | NA | missense | c.2081A > G | p.His694Arg |
| 2:25241563:T:G | NA | missense | c.2081A > C | p.His694Pro |
| 2:25241563:TG:T | NA | frameshift | c.2080delC | p.His694fs |
| 2:25241564:G:A | NA | missense | c.2080C > T | p.His694Tyr |
| 2:25241564:GCTT | NA | frameshift | c.2073_2079delAC | p.Gln692fs |
| CTGT:G | | | AGAAG | |
| 2:25241565:C:CTT | NA | inframe_indel | c.2078_2079insTA | p.Gln692_Lys693 |
| A | | | A | insAsn |
| 2:25241565:C:G | NA | missense | c.2079G > C | p.Lys693Asn |
| 2:25241565:CT:C | NA | frameshift | c.2078delA | p.Lys693fs |
| 2:25241566:T:TTC | NA | frameshift | c.2076_2077dupG | p.Lys693fs |
| | | | A | |
| 2:25241566:TTC:T | NA | frameshift | c.2076_2077delGA | p.Lys693fs |
| 2:25241567:T:A | rs1365278000 | stop_gained | c.2077A > T | p.Lys693* |
| 2:25241568:C:CT | NA | frameshift | c.2075dupA | p.Lys693fs |
| 2:25241568:CTG: | NA | frameshift | c.2074_2075delCA | p.Gln692fs |
| C | | | | |
| 2:25241569:TGTG | NA | inframe_indel | c.2063_2074delGC | p.Arg688_Thr691 |
| TGACGCTGC:T | | | AGCGTCACAC | del |
| 2:25241570:G:A | rs1278018793 | stop_gained | c.2074C > T | p.Gln692* |
| 2:25241570:G:GT | NA | frameshift | c.2073dupA | p.Gln692fs |
| 2:25241570:GT:G | NA | frameshift | c.2073delA | p.Gln692fs |
| 2:25241571:T:TG | NA | inframe_indel | c.2070_2072dupCA | p.Thr691dup |
| TG | | | C | |
| 2:25241571:TG:T | NA | frameshift | c.2072delC | p.Thr691fs |
| 2:25241572:G:A | rs1193025338 | missense | c.2072C > T | p.Thr691Ile |
| 2:25241572:G:GT | NA | frameshift | c.2071dupA | p.Thr691fs |
| 2:25241572:GT:G | NA | frameshift | c.2071delA | p.Thr691fs |
| 2:25241573:T:G | NA | missense | c.2071A > C | p.Thr691Pro |
| 2:25241573:T:TA | NA | frameshift | c.2070_2071insT | p.Thr691fs |
| 2:25241574:GA:G | NA | frameshift | c.2069delT | p.Va1690fs |
| 2:25241575:A:AC | NA | frameshift | c.2064_2068dupCA | p.Va1690fs |
| GCTG | | | GCG | |
| 2:25241575:A:C | NA | missense | c.2069T > G | p.Va1690Gly |
| 2:25241575:A:G | NA | missense | c.2069T > C | p.Va1690Ala |
| 2:25241575:A:T | NA | missense | c.2069T > A | p.Va1690Asp |
| 2:25241575:AC:A | NA | frameshift | c.2068delG | p.Va1690fs |
| 2:25241576:C:A | NA | missense | c.2068G > T | p.Va1690Phe |
| 2:25241576:C:T | rs768899154 | missense | c.2068G > A | p.Va1690Ile |
| 2:25241576:CG:C | NA | frameshift | c.2067delC | p.Ser689fs |
| 2:25241577:G:GC | NA | frameshift | c.2041_2066dupAT | p.Ser689fs |
| TGCGGACGTCCCC | | | CATGTACGTCGGGG | |
| GACGTACATGAT | | | ACGTCCGCAG | |
| 2:25241579:TG:T | NA | frameshift | c.2064delC | p.Ser689fs |
| 2:25241580:GC:G | NA | frameshift | c.2063delG | p.Arg688fs |
| 2:25241580:GCG | NA | frameshift | c.2054_2063delGG | p.Gly685fs |
| GACGTCCC:G | | | GACGTCCG | |
| 2:25241581:C:A | NA | missense | c.2063G > T | p.Arg688Leu |
| 2:25241581:C:G | NA | missense | c.2063G > C | p.Arg688Pro |
| 2:25241581:C:T | rs369713081 | missense | c.2063G > A | p.Arg688His |
| 2:25241581:CG:C | NA | frameshift | c.2062delC | p.Arg688fs |
| 2:25241582:G:A | rs1484795800 | missense | c.2062C > T | p.Arg688Cys |
| 2:25241582:G:C | rs1484795800 | missense | c.2062C > G | p.Arg688Gly |
| 2:25241582:G:T | NA | missense | c.2062C > A | p.Arg688Ser |
| 2:25241584:A:C | NA | missense | c.2060T > G | p.Va1687Gly |
| 2:25241584:A:G | NA | missense | c.2060T > C | p.Va1687Ala |
| 2:25241584:A:T | NA | missense | c.2060T > A | p.Va1687Asp |
| 2:25241585:C:A | NA | missense | c.2059G > T | p.Va1687Phe |
| 2:25241585:c.G | NA | missense | c.2059G > C | p.Va1687Leu |
| 2:25241586:G:C | NA | missense | c.2058C > G | p.Asp686Glu |
| 2:25241586:G:GT | NA | frameshift | c.2057_2058insTG | p.Va1687fs |
| CCCCGACGTACAT | | | ATCATGTACGTCGG | |
| GATCA | | | GGA | |
| 2:25241586:GT:G | NA | frameshift | c.2057delA | p.Asp686fs |
| 2:25241587:T:A | rs754764419 | missense | c.2057A > T | p.Asp686Val |
| 2:25241587:T:C | rs754764419 | missense | c.2057A > G | p.Asp686Gly |
| 2:25241587:T:G | NA | missense | c.2057A > C | p.Asp686Ala |
| 2:25241587:T:TC | NA | frameshift | c.2056dupG | p.Asp686fs |
| 2:25241587:TC:T | NA | frameshift | c.2056delG | p.Asp686fs |
| 2:25241587:TCC:T | rs1344775606 | frameshift | c.2055_2056delGG | p.Asp686fs |
| 2:25241587:TCCC | NA | frameshift | c.2046_2056delGT | p.Met682fs |
| CGACGTAC:T | | | ACGTCGGGG | |
| 2:25241588:C:A | rs767224028 | missense | c.2056G > T | p.Asp686Tyr |
| 2:25241588:C:G | NA | missense | c.2056G > C | p.Asp686His |
| 2:25241588:C:T | NA | missense | c.2056G > A | p.Asp686Asn |
| 2:25241589:CCCG | NA | frameshift | c.2038_2054delAA | p.Lys680fs |
| ACGTACATGATCT | | | GATCATGTACGTCG | |
| T:C | | | G | |
| 2:25241590:C:A | NA | missense | c.2054G > T | p.Gly685Val |
| 2:25241590:C:G | rs754037033 | missense | c.2054G > C | p.Gly685Ala |
| 2:25241590:C:T | rs754037033 | missense | c.2054G > A | p.Gly685Glu |
| 2:25241590:CCGA | NA | frameshift | c.2049_2053delCG | p.Tyr683fs |
| CG:C | | | TCG | |
| 2:25241591:C:A | rs779176507 | missense | c.2053G > T | p.Gly685Trp |
| 2:25241591:C:G | NA | missense | c.2053G > C | p.Gly685Arg |
| 2:25241591:C:T | rs779176507 | missense | c.2053G > A | p.Gly685Arg |
| 2:25241591:CG:C | NA | frameshift | c.2052delC | p.Asp686fs |
| 2:25241591:CGAC | NA | frameshift | c.2049_2052delCG | p.Tyr683fs |
| G:C | | | TC | |
| 2:25241591:CGAC | NA | frameshift | c.2048_2052delAC | p.Tyr683fs |
| GT:C | | | GTC | |
| 2:25241592:GA:G | NA | frameshift | c.2051delT | p.Va1684fs |
| 2:25241592:GAC | NA | inframe_indel | c.2046_2051delGT | p.Met682_Va1684 |
| GTAC:G | | | ACGT | delinsIle |
| 2:25241593:A:C | NA | missense | c.2051T > G | p.Va1684Gly |
| 2:25241593:A:T | NA | missense | c.2051T > A | p.Va1684Asp |
| 2:25241593:ACGT | NA | frameshift | c.2035_2050delGG | p.Gly679fs |
| ACATGATCTTCCC: | | | GAAGATCATGTACG | |
| A | | | | |
| 2:25241594:C:A | NA | missense | c.2050G > T | p.Va1684Phe |
| 2:25241594:CG:C | NA | frameshift | c.2049delC | p.Tyr683fs |
| 2:25241594:CGTA | NA | frameshift | c.2043_2049delCA | p.Ile681fs |
| CATG:C | | | TGTAC | |
| 2:25241595:G:C | NA | stop_gained | c.2049C > G | p.Tyr683* |
| 2:25241595:G:T | NA | stop_gained | c.2049C > A | p.Tyr683* |
| 2:25241595:GT:G | NA | frameshift | c.2048delA | p.Tyr683fs |
| 2:25241596:T:C | rs780495518 | missense | c.2048A > G | p.Tyr683Cys |
| 2:25241596:T:TA | NA | frameshift | c.2025_2047dupGC | p.Tyr683fs |
| CATGATCTTCCCC | | | GGCACCAGGGGAA | |
| TGGTGCCGC | | | GATCATGT | |
| 2:25241596:TACA | NA | frameshift | c.2043_2047delCA | p.Met682fs |
| TG:T | | | TGT | |
| 2:25241597:A:C | NA | missense | c.2047T > G | p.Tyr683Asp |
| 2:25241597:A:G | NA | missense | c.2047T > C | p.Tyr683His |
| 2:25241597:A:T | NA | missense | c.2047T > A | p.Tyr683Asn |
| 2:25241598:CA:C | NA | frameshift | c.2045delT | p.Met682fs |
| 2:25241598:CAT: | NA | frameshift | c.2044_2045delAT | p.Met682fs |
| C | | | | |
| 2:25241598:CATG: | NA | inframe_indel | c.2043_2045delCA | p.Ile681del |
| C | | | T | |
| 2:25241599:A:AT | NA | frameshift | c.2044dupA | p.Met682fs |
| 2:25241599:A:C | rs1324879320 | missense | c.2045T > G | p.Met682Arg |
| 2:25241599:ATGA | NA | inframe_indel | c.2030_2044delAC | p.His677_Met682 |
| TCTTCCCCTGGT:A | | | CAGGGGAAGATCA | delinsLeu |
| 2:25241600:TG:T | rs1455730245 | frameshift | c.2043delC | p.Met682fs |
| 2:25241600:TGAT | NA | frameshift | c.2024_2043delTG | p.Va1675fs |
| CTTCCCCTGGTGC | | | CGGCACCAGGGGA | |
| CGCA:T | | | AGATC | |
| 2:25241601:G:C | | missense | c.2043C > G | p.Ile681Met |
| 2:25241601:G:GT | NA | frameshift | c.2042_2043insA | p.Met682fs |
| 2:25241602:A:AT | NA | frameshift | c.2017_2041dupG | p.Ile681fs |
| CTTCCCCTGGTGC | | | GCATGGTGCGGCA | |
| CGCACCATGCC | | | CCAGGGGAAGA | |
| 2:25241602:A:G | NA | missense | c.2042T > C | p.Ile681Thr |
| 2:25241602:A:T | rs769006337 | missense | c.2042T > A | p.Ile681Asn |
| 2:25241603:T:A | NA | missense | c.2041A > T | p.Ile681Phe |
| 2:25241604:C:A | NA | missense | c.2040G > T | p.Lys680Asn |
| 2:25241604:CT:C | NA | frameshift | c.2039delA | p.Lys680fs |
| 2:25241605:T:C | NA | missense | c.2039A > G | p.Lys680Arg |
| 2:25241606:T:A | NA | stop_gained | c.2038A > T | p.Lys680* |
| 2:25241606:TC:T | rs1365479526 | frameshift | c.2037delG | p.Lys680fs |
| 2:25241608:C:A | NA | missense | c.2036G > T | p.Gly679Val |
| 2:25241608:C:T | NA | missense | c.2036G > A | p.Gly679Glu |
| 2:25241609:C:A | NA | missense | c.2035G > T | p.Gly679Trp |
| 2:25241610:CT:C | NA | frameshift | c.2033delA | p.Gln678fs |
| 2:25241611:T:TG | NA | frameshift | c.2032dupC | p.Gln678fs |
| 2:25241611:TG:T | NA | frameshift | c.2032delC | p.Gln678fs |
| 2:25241612:G:A | rs1050271318 | stop_gained | c.2032C > T | p.Gln678* |
| 2:25241614:T:C | NA | missense | c.2030A > G | p.His677Arg |
| 2:25241614:T:TG | NA | frameshift | c.2026_2029dupCG | p.His677fs |
| CCG | | | GC | |
| 2:25241615:GCC | NA | frameshift | c.2024_2028delTG | p.Va1675fs |
| GCA:G | | | CGG | |
| 2:25241616:C:CC | NA | frameshift | c.2026_2027dupCG | p.His677fs |
| G | | | | |
| 2:25241617:C:A | rs769539104 | missense | c.2027G > T | p.Arg676Leu |
| 2:25241617:C:T | rs769539104 | missense | c.2027G > A | p.Arg676Gln |
| 2:25241617:CG:C | NA | frameshift | c.2026delC | p.Arg676fs |
| 2:25241618:G:A | rs375399431 | missense | c.2026C > T | p.Arg676Trp |
| 2:25241618:G:C | NA | missense | c.2026C > G | p.Arg676Gly |
| 2:25241618:G:GC | NA | frameshift | c.2025dupG | p.Arg676fs |
| 2:25241618:G:GC | NA | frameshift | c.2021_2025dupTG | p.Arg676fs |
| ACCA | | | GTG | |
| 2:25241618:GCAC | NA | frameshift | c.2000_2025delAG | p.Glu667fs |
| CATGCCCACCGTG | | | GACTCCATCACGGT | |
| ATGGAGTCCT:G | | | GGGCATGGTG | |
| 2:25241619:CA:C | NA | frameshift | c.2024delT | p.Va1675fs |
| 2:25241620:A:AC | NA | frameshift | c.2023dupG | p.Va1675fs |
| 2:25241620:A:C | | missense | c.2024T > G | p.Va1675Gly |
| 2:25241620:A:G | | missense | c.2024T > C | p.Va1675Ala |
| 2:25241620:A:T | NA | missense | c.2024T > A | p.Va1675Glu |
| 2:25241620:AC:A | rs763843749 | frameshift | c.2023delG | p.Va1675fs |
| 2:25241621:C:A | NA | missense | c.2023G > T | p.Va1675Leu |
| 2:25241621:C:G | NA | missense | c.2023G > C | p.Va1675Leu |
| 2:25241621:C:T | rs1025238838 | missense | c.2023G > A | p.Va1675Met |
| 2:25241623:AT:A | NA | frameshift | c.2020delA | p.Met674fs |
| 2:25241623:ATGC | NA | frameshift | c.2014_2020delGT | p.Va1672fs |
| CCAC:A | | | GGGCA | |
| 2:25241624:T:C | rs1306265898 | missense | c.2020A > G | p.Met674Val |
| 2:25241624:TG:T | NA | frameshift | c.2019delC | p.Met674fs |
| 2:25241625:G:GC | NA | frameshift | c.2017_2018dupG | p.Met674fs |
| C | | | G | |
| 2:25241625:G:GC | NA | inframe_indel | c.2016_2018dupG | p.Gly673dup |
| CC | | | GG | |
| 2:25241625:G:GC | NA | frameshift | c.2015_2018dupTG | p.Met674fs |
| CCA | | | GG | |
| 2:25241625:Gc.G | NA | frameshift | c.2018delG | p.Gly673fs |
| 2:25241625:GCCC | NA | frameshift | c.2015_2018delTG | p.Va1672fs |
| A:G | | | GG | |
| 2:25241626:C:A | NA | missense | c.2018G > T | p.Gly673Val |
| 2:25241626:C:T | rs902730598 | missense | c.2018G > A | p.Gly673Asp |
| 2:25241627:C:A | NA | missense | c.2017G > T | p.Gly673Cys |
| 2:25241629:AC:A | NA | frameshift | c.2014delG | p.Va1672fs |
| 2:25241630:C:T | NA | missense | c.2014G > A | p.Va1672Met |
| 2:25241630:CCGT | NA | frameshift | c.2000_2013delAG | p.Glu667fs |
| GATGGAGTCCT:C | | | GACTCCATCACG | |
| 2:25241631:CGTG | NA | frameshift | c.2005_2012delTC | p.Ser669fs |
| ATGGA:C | | | CATCAC | |
| 2:25241632:G:A | NA | missense | c.2012C > T | p.Thr671Met |
| 2:25241632:G:GT | NA | frameshift | c.2011dupA | p.Thr671fs |
| 2:25241632:GT:G | NA | frameshift | c.2011delA | p.Thr671fs |
| 2:25241633:TGAT | NA | frameshift | c.2007_2010delCA | p.Ile670fs |
| G:T | | | TC | |
| 2:25241634:GAT: | NA | frameshift | c.2008_2009delAT | p.Ile670fs |
| G | | | | |
| 2:25241634:GAT | NA | frameshift | c.1994_2009delTG | p.Va1665fs |
| GGAGTCCTCACAC | | | TGTGAGGACTCCAT | |
| A:G | | | | |
| 2:25241635:A:AG | NA | frameshift | c.2008_2009insCC | p.Ile670fs |
| GACTCCATCACGG | | | GTGATGGAGTCC | |
| 2:25241635:A:AT | NA | frameshift | c.1974_2008dupCC | p.Ile670fs |
| GGAGTCCTCACAC | | | GCTACATTGCCTCG | |
| ACCTCCGAGGCAA | | | GAGGTGTGTGAGG | |
| TGTAGCGG | | | ACTCCA | |
| 2:25241635:AT:A | NA | frameshift | c.2008delA | p.Ile670fs |
| 2:25241635:ATG | NA | frameshift | c.2004_2008delCT | p.Ser669fs |
| GAG: A | | | CCA | |
| 2:25241636:T:A | NA | missense | c.2008A > T | p.Ile670Phe |
| 2:25241636:T:G | NA | missense | c.2008A > C | p.Ile670Leu |
| 2:25241636:T:TG | rs753637583 | frameshift | c.2007dupC | p.Ile670fs |
| 2:25241636:TG:T | NA | frameshift | c.2007delC | p.Ile670fs |
| 2:25241637:GGA: | NA | frameshift | c.2005_2006delTC | p.Ser669fs |
| G | | | | |
| 2:25241637:GGA | NA | inframe_indel | c.1998_2006delTG | p.Glu667_Ser669 |
| GTCCTCA:G | | | AGGACTC | del |
| 2:25241638:G:A | NA | missense | c.2006C > T | p.Ser669Phe |
| 2:25241638:G:T | NA | missense | c.2006C > A | p.Ser669Tyr |
| 2:25241639:AG:A | NA | frameshift | c.2004delC | p.Ser669fs |
| 2:25241641:T:A | NA | missense | c.2003A > T | p.Asp668Val |
| 2:25241641:T:TC | NA | frameshift | c.2002dupG | p.Asp668fs |
| 2:25241641:TC:T | NA | frameshift | c.2002delG | p.Asp668fs |
| 2:25241642:C:T | NA | missense | c.2002G > A | p.Asp668Asn |
| 2:25241642:CCT: | NA | frameshift | c.2000_2001delAG | p.Glu667fs |
| C | | | | |
| 2:25241643:CT:C | NA | frameshift | c.2000delA | p.Glu667fs |
| 2:25241644:T:C | NA | missense | c.2000A > G | p.Glu667Gly |
| 2:25241644:TCA: | rs1467575781 | frameshift | c.1998_1999delTG | p.Cys666fs |
| T | | | | |
| 2:25241644:TCAC | NA | frameshift | c.1996_1999delTG | p.Cys666fs |
| A:T | | | TG | |
| 2:25241645:C:A | NA | stop_gained | c.1999G > T | p.Glu667* |
| 2:25241645:C:CA | NA | frameshift | c.1998dupT | p.Glu667fs |
| 2:25241646:A:C | NA | missense | c.1998T > G | p.Cys666Trp |
| 2:25241646:A:T | NA | stop_gained | c.1998T > A | p.Cys666* |
| 2:25241647:C:A | NA | missense | c.1997G > T | p.Cys666Phe |
| 2:25241647:C:T | NA | missense | c.1997G > A | p.Cys666Tyr |
| 2:25241648:A:C | NA | missense | c.1996T > G | p.Cys666Gly |
| 2:25241649:CA:C | rs1268925454 | frameshift | c.1994delT | p.Va1665fs |
| 2:25241650:A:C | rs762503226 | missense | c.1994T > G | p.Va1665Gly |
| 2:25241650:A:G | NA | missense | c.1994T > C | p.Va1665Ala |
| 2:25241650:A:T | rs762503226 | missense | c.1994T > A | p.Va1665Glu |
| 2:25241650:AC:A | NA | frameshift | c.1993delG | p.Va1665fs |
| 2:25241651:C:A | rs766020170 | missense | c.1993G > T | p.Va1665Leu |
| 2:25241651:C:T | NA | missense | c.1993G > A | p.Va1665Met |
| 2:25241652:CT:C | NA | frameshift | c.1991delA | p.Glu664fs |
| 2:25241652:CTCC | NA | inframe_indel | c.1974_1991delCC | p.Asp658_Ser663 |
| GAGGCAATGTAG | | | GCTACATTGCCTCG | del |
| CGG:C | | | GA | |
| 2:25241653:T:C | NA | missense | c.1991A > G | p.Glu664Gly |
| 2:25241653:TC:T | NA | frameshift | c.1990delG | p.Glu664fs |
| 2:25241653:TCCG | NA | frameshift | c.1977_1990delCT | p.Tyr660fs |
| AGGCAATGTAG:T | | | ACATTGCCTCGG | |
| 2:25241654:C:A | rs1476901854 | stop_gained | c.1990G > T | p.Glu664* |
| 2:25241654:C:G | NA | missense | c.1990G > C | p.Glu664Gln |
| 2:25241656:G:A | rs553080210 | missense | c.1988C > T | p.Ser663Leu |
| 2:25241656:G:C | NA | missense | c.1988_1998G | p.Ser663Trp |
| 2:25241656:G:T | NA | stop_gained | c.1988_1998A | p.Ser663* |
| 2:25241656:GAG | NA | inframe_indel | c.1979_1987delAC | p.Tyr660_Ala662 |
| GCAATGT:G | | | ATTGCCT | del |
| 2:25241657:A:G | NA | missense | c.1987T > C | p.Ser663Pro |
| 2:25241657:AG:A | NA | frameshift | c.1986delC | p.Ser663fs |
| 2:25241658:GGC | NA | frameshift | c.1978_1985delTA | p.Tyr660fs |
| AATGTA:G | | | CATTGC | |
| 2:25241659:G:A | NA | missense | c.1985C > T | p.Ala662Val |
| 2:25241659:G:T | NA | missense | c.1985C > A | p.Ala662Asp |
| 2:25241659:GCA | NA | frameshift | c.1981_1984delAT | p.Ile661fs |
| AT:G | | | TG | |
| 2:25241660:C:A | NA | missense | c.1984G > T | p.Ala662Ser |
| 2:25241660:C:CA | NA | frameshift | c.1983dupT | p.Ala662fs |
| 2:25241660:C:G | rs759408234 | missense | c.1984G > C | p.Ala662Pro |
| 2:25241660:C:T | rs759408234 | missense | c.1984G > A | p.Ala662Thr |
| 2:25241660:CAA: | NA | frameshift | c.1982_1983delTT | p.Ile661fs |
| C | | | | |
| 2:25241661:A:AA | NA | frameshift | c.1939_1982dupCT | p.Ala662fs |
| TGTAGCGGTCCAC | | | CCTGGTGCTGAAGG | |
| CTGAATGCCCAAG | | | ACTTGGGCATTCAG | |
| TCCTTCAGCACCA | | | GTGGACCGCTACAT | |
| GGAG | | | | |
| 2:25241661:AATG | NA | frameshift | c.1979_1982delAC | p.Tyr660fs |
| T:A | | | AT | |
| 2:25241662:A:C | NA | missense | c.1982T > G | p.Ile661Ser |
| 2:25241662:A:G | rs1471917721 | missense | c.1982T > C | p.Ile661Thr |
| 2:25241662:A:T | NA | missense | c.1982T > A | p.Ile661Asn |
| 2:25241662:AT:A | NA | frameshift | c.1981delA | p.Ile661fs |
| 2:25241662:ATGT | NA | inframe_indel | c.1976_1981delGC | p.Arg659_Ile661 |
| AGC:A | | | TACA | delinsLeu |
| 2:25241664:G:C | NA | stop_gained | c.1980C > G | p.Tyr660* |
| 2:25241664:G:GT | rs1158856353 | frameshift | c.1979dupA | p.Tyr660fs |
| 2:25241665:T:A | NA | missense | c.1979A > T | p.Tyr660Phe |
| 2:25241665:T:C | rs767552800 | missense | c.1979A > G | p.Tyr660Cys |
| 2:25241665:T:G | NA | missense | c.1979A > C | p.Tyr660Ser |
| 2:25241666:A:C | NA | missense | c.1978T > G | p.Tyr660Asp |
| 2:25241666:A:G | NA | missense | c.1978T > C | p.Tyr660His |
| 2:25241666:A:T | NA | missense | c.1978T > A | p.Tyr660Asn |
| 2:25241668:C:CG | NA | frameshift | c.1975dupC | p.Arg659fs |
| 2:25241668:C:T | rs752434188 | missense | c.1976G > A | p.Arg659His |
| 2:25241668:CG:C | NA | frameshift | c.1975delC | p.Arg659fs |
| 2:25241669:G:A | rs755982635 | missense | c.1975C > T | p.Arg659Cys |
| 2:25241669:G:C | rs755982635 | missense | c.1975C > G | p.Arg659Gly |
| 2:25241671:T:A | rs146164726 | missense | c.1973A > T | p.Asp658Val |
| 2:25241671:T:TC | NA | frameshift | c.1972dupG | p.Asp658fs |
| 2:25241671:T:TCC | NA | frameshift | c.1971_1972dupG | p.Asp658fs |
| | | | G | |
| 2:25241671:TC:T | NA | frameshift | c.1972delG | p.Asp658fs |
| 2:25241671:TCCA | NA | inframe_indel | c.1961_1972delGC | p.Gly654_Va1657 |
| CCTGAATGC:T | | | ATTCAGGTGG | del |
| 2:25241672:C:A | NA | missense | c.1972G > T | p.Asp658Tyr |
| 2:25241672:C:CC | NA | frameshift | c.1970_1971dupTG | p.Asp658fs |
| A | | | | |
| 2:25241672:C:T | NA | missense | c.1972G > A | p.Asp658Asn |
| 2:25241672:CCA: | NA | frameshift | c.1970_1971delTG | p.Va1657fs |
| C | | | | |
| 2:25241672:CCAC | NA | frameshift | c.1964_1971delTTC | p.Ile655fs |
| CTGAA:C | | | AGGTG | |
| 2:25241674:A:C | NA | missense | c.1970T > G | p.Va1657Gly |
| 2:25241674:A:G | NA | missense | c.1970T > C | p.Va1657Ala |
| 2:25241674:A:T | NA | missense | c.1970T > A | p.Va1657Glu |
| 2:25241674:AC:A | NA | frameshift | c.1969delG | p.Va1657fs |
| 2:25241674:ACCT | NA | frameshift | c.1962_1969delCA | p.Ile655fs |
| GAATG:A | | | TTCAGG | |
| 2:25241674:ACCT | NA | frameshift | c.1960_1969delGG | p.Gly654fs |
| GAATGCC:A | | | CATTCAGG | |
| 2:25241675:C:A | NA | missense | c.1969G > T | p.Va1657Leu |
| 2:25241675:C:G | NA | missense | c.1969G > C | p.Va1657Leu |
| 2:25241675:C:T | rs368961181 | missense | c.1969G > A | p.Va1657Met |
| 2:25241676:CT:C | NA | frameshift | c.1967delA | p.Gln656fs |
| 2:25241677:T:G | rs1323531460 | missense | c.1967A > C | p.Gln656Pro |
| 2:25241677:TG:T | NA | frameshift | c.1966delC | p.Gln656fs |
| 2:25241678:G:A | rs758402405 | stop_gained | c.1966C > T | p.Gln656* |
| 2:25241678:GAAT | NA | frameshift | c.1955_1965delAC | p.Asp652fs |
| GCCCAAGT:G | | | TTGGGCATT | |
| 2:25241679:A:C | NA | missense | c.1965T > G | p.Ile655Met |
| 2:25241679:AATG | NA | frameshift | c.1961_1964delGC | p.Gly654fs |
| C:A | | | AT | |
| 2:25241680:A:C | NA | missense | c.1964T > G | p.Ile655Ser |
| 2:25241680:A:G | NA | missense | c.1964T > C | p.Ile655Thr |
| 2:25241680:A:T | rs780120142 | missense | c.1964T > A | p.Ile655Asn |
| 2:25241680:AT:A | NA | frameshift | c.1963delA | p.Ile655fs |
| 2:25241681:T:C | NA | missense | c.1963A > G | p.Ile655Val |
| 2:25241681:T:TG | NA | frameshift | c.1961_1962dupGC | p.Ile655fs |
| C | | | | |
| 2:25241681:T:TTT | NA | frameshift | c.1962_1963insTG | p.Ile655fs |
| CA | | | AA | |
| 2:25241681:TG:T | NA | frameshift | c.1962delC | p.Ile655fs |
| 2:25241682:G:GC | NA | frameshift | c.1961dupG | p.Ile655fs |
| 2:25241682:GC:G | NA | frameshift | c.1961delG | p.Gly654fs |
| 2:25241682:GCCC | NA | frameshift | c.1946_1961delTG | p.Va1649fs |
| AAGTCCTTCAGCA: | | | CTGAAGGACTTGG | |
| G | | | G | |
| 2:25241683:C:A | NA | missense | c.1961G > T | p.Gly654Val |
| 2:25241683:C:G | NA | missense | c.1961G > C | p.Gly654Ala |
| 2:25241683:C:T | rs1297531346 | missense | c.1961G > A | p.Gly654Asp |
| 2:25241684:C:CC | NA | frameshift | c.1959_1960insAG | p.Gly654fs |
| T | | | | |
| 2:25241684:C:T | rs1371093470 | missense | c.1960G > A | p.Gly654Ser |
| 2:25241685:C:A | rs747168987 | missense | c.1959G > T | p.Leu653Phe |
| 2:25241685:C:G | NA | missense | c.1959G > C | p.Leu653Phe |
| 2:25241685:CA:C | NA | frameshift | c.1958delT | p.Leu653fs |
| 2:25241686:A:C | NA | missense | c.1958T > G | p.Leu653Trp |
| 2:25241686:A:G | NA | missense | c.1958T > C | p.Leu653Ser |
| 2:25241686:A:T | rs1202526721 | stop_gained | c.1958T > A | p.Leu653* |
| 2:25241686:AAGT | NA | frameshift | c.1948_1957delCT | p.Leu650fs |
| CCTTCAG:A | | | GAAGGACT | |
| 2:25241687:A:C | rs1461998130 | missense | c.1957T > G | p.Leu653Val |
| 2:25241687:A:T | NA | missense | c.1957T > A | p.Leu653Met |
| 2:25241687:AG:A | NA | frameshift | c.1956delC | p.Leu653fs |
| 2:25241688:G:GT | NA | frameshift | c.1952_1955dupA | p.Asp652fs |
| CCT | | | GGA | |
| 2:25241688:G:GT | NA | frameshift | c.1949_1955dupTG | p.Leu653fs |
| CCTTCA | | | AAGGA | |
| 2:25241688:GT:G | NA | frameshift | c.1955delA | p.Asp652fs |
| 2:25241689:TC:T | NA | frameshift | c.1954delG | p.Asp652fs |
| 2:25241689:TCCT | NA | inframe_indel | c.1946_1954delTG | p.Va1649_Lys651 |
| TCAGCA:T | | | CTGAAGG | del |
| 2:25241689:TCCT | NA | frameshift | c.1945_1954delGT | p.Va1649fs |
| TCAGCAC:T | | | GCTGAAGG | |
| 2:25241691:CT:C | NA | frameshift | c.1952delA | p.Lys651fs |
| 2:25241693:T:A | NA | stop_gained | c.1951A > T | p.Lys651* |
| 2:25241693:TCA: | NA | frameshift | c.1949_1950delTG | p.Leu650fs |
| T | | | | |
| 2:25241693:TCAG | NA | frameshift | c.1946_1950delTG | p.Va1649fs |
| CA:T | | | CTG | |
| 2:25241694:C:CA | NA | frameshift | c.1949dupT | p.Lys651fs |
| 2:25241694:CA:C | NA | frameshift | c.1949delT | p.Leu650fs |
| 2:25241695:A:C | NA | missense | c.1949T > G | p.Leu650Arg |
| 2:25241695:A:G | NA | missense | c.1949T > C | p.Leu650Pro |
| 2:25241695:A:T | rs1467909574 | missense | c.1949T > A | p.Leu650Gln |
| 2:25241695:AGC | NA | frameshift | c.1939_1948delCT | p.Leu647fs |
| ACCAGGAG:A | | | CCTGGTGC | |
| 2:25241696:G:C | rs1334392444 | missense | c.1948C > G | p.Leu650Val |
| 2:25241696:GC:G | NA | frameshift | c.1947delG | p.Leu650fs |
| 2:25241696:GCA: | NA | frameshift | c.1946_1947delTG | p.Va1649fs |
| G | | | | |
| 2:25241697:CA:C | NA | frameshift | c.1946delT | p.Va1649fs |
| 2:25241698:A:C | NA | missense | c.1946T > G | p.Va1649Gly |
| 2:25241698:A:T | NA | missense | c.1946T > A | p.Va1649Glu |
| 2:25241699:C:A | NA | missense | c.1945G > T | p.Va1649Leu |
| 2:25241699:C:CA | NA | frameshift | c.1944_1945insT | p.Va1649fs |
| 2:25241699:c.G | NA | missense | c.1945G > C | p.Va1649Leu |
| 2:25241699:C:T | NA | missense | c.1945G > A | p.Va1649Met |
| 2:25241700:C:CA | NA | inframe_indel | c.1941_1943dupCC | p.Leu648dup |
| GG | | | T | |
| 2:25241700:CA:C | NA | frameshift | c.1943delT | p.Leu648fs |
| 2:25241701:A:C | NA | missense | c.1943T > G | p.Leu648Arg |
| 2:25241701:A:G | rs587777507 | missense | c.1943T > C | p.Leu648Pro |
| 2:25241701:AG:A | rs1292349089 | frameshift | c.1942delC | p.Leu648fs |
| 2:25241701:AGG | NA | splice_acceptor | c.1937- | p.Gly646_Leu648 |
| AGCCCTGCACCAG | | | 10_1942delGCTGG | delinsVal |
| C:A | | | TGCAGGGCTCC | |
| 2:25241701:AGG | NA | splice_acceptor | c.1937- | p.Gly646_Leu648 |
| AGCCCTGCACCAG | | | 11_1942delGGCTG | delinsVal |
| CC:A | | | GTGCAGGGCTCC | |
| 2:25241704:A:AG | NA | frameshift | c.1938_1939dupGC | p.Leu647fs |
| C | | | | |
| 2:25241704:A:C | rs781742148 | missense | c.1940T > G | p.Leu647Arg |
| 2:25241704:A:G | NA | missense | c.1940T > C | p.Leu647Pro |
| 2:25241704:A:T | rs781742148 | missense | c.1940T > A | p.Leu647His |
| 2:25241705:G:C | NA | missense | c.1939C > G | p.Leu647Val |
| 2:25241705:G:GC | NA | frameshift | c.1938dupG | p.Leu647fs |
| 2:25241705:GC:G | NA | frameshift | c.1938delG | p.Leu647fs |
| 2:25241707:C:T | NA | missense | c.1937G > A | p.Gly646Glu |
| 2:25241708:C:A | NA | splice_acceptor | c.1937-1G > T | |
| 2:25241708:C:G | NA | splice_acceptor | c.1937-1G > C | |
| 2:25241708:C:T | rs748440577 | splice_acceptor | c.1937-1G > A | |
| 2:25241708:CT:C | NA | splice_acceptor | c.1937-2delA | |
| 2:25241709:T:A | NA | splice_acceptor | c.1937-2A > T | |
| 2:25241709:T:C | rs770305758 | splice_acceptor | c.1937-2A > G | |
| 2:25241709:T:G | NA | splice_acceptor | c.1937-2A > C | |
| 2:25243893:CTCA | NA | splice_donor | c.1928_1936+4delT | p.Ile643_Gly646 |
| CCTGTAGCGA:C | | | CGCTACAGGTGA | delinsArg |
| 2:25243895:C:CA | NA | splice_donor | c.1936+2dupT | |
| 2:25243896:A:G | rs1190050788 | splice_donor | c.1936+2T > C | |
| 2:25243896:A:T | NA | splice_donor | c.1936+2T > A | |
| 2:25243896:AC:A | NA | splice_donor | c.1936+1delG | |
| 2:25243897:C:A | rs1238786275 | splice_donor | c.1936+1G > T | |
| 2:25243897:C:G | NA | splice_donor | c.1936+1G > C | |
| 2:25243897:C:T | rs1238786275 | splice_donor | c.1936+1G > A | |
| 2:25243898:C:A | rs1472933404 | missense | c.1936G > T | p.Gly646Trp |
| 2:25243898:C:G | NA | missense | c.1936G > C | p.Gly646Arg |
| 2:25243898:C:T | NA | missense | c.1936G > A | p.Gly646Arg |
| 2:25243898:CT:C | NA | frameshift | c.1935delA | p.Leu647fs |
| 2:25243900:G:A | rs1387749234 | missense | c.1934C > T | p.Thr645Ile |
| 2:25243900:G:GT | NA | frameshift | c.1933dupA | p.Thr645fs |
| 2:25243901:T:C | | missense | c.1933A > G | p.Thr645Ala |
| 2:25243903:G:A | NA | missense | c.1931C > T | p.Ala644Val |
| 2:25243903:GC:G | NA | frameshift | c.1930delG | p.Ala644fs |
| 2:25243904:C:G | NA | missense | c.1930G > C | p.Ala644Pro |
| 2:25243904:C:T | rs1431156021 | missense | c.1930G > A | p.Ala644Thr |
| 2:25243904:CG:C | NA | frameshift | c.1929delC | p.Ile643fs |
| 2:25243905:G:GA | NA | frameshift | c.1928dupT | p.Ala644fs |
| 2:25243905:G:GA | NA | frameshift | c.1924_1928dupG | p.Ile643fs |
| TTCC | | | GAAT | |
| 2:25243905:GA:G | NA | frameshift | c.1928delT | p.Ile643fs |
| 2:25243905:GATT | NA | frameshift | c.1912_1928delTCT | p.Ser638fs |
| CCATCAAAGAGA | | | CTCTTTGATGGAAT | |
| GA:G | | | | |
| 2:25243906:A:G | NA | missense | c.1928T > C | p.Ile643Thr |
| 2:25243906:AT:A | NA | frameshift | c.1927delA | p.Ile643fs |
| 2:25243907:T:C | NA | missense | c.1927A > G | p.Ile643Val |
| 2:25243907:T:TTC | NA | frameshift | c.1923_1926dupTG | p.Ile643fs |
| CA | | | GA | |
| 2:25243908:T:TC | NA | frameshift | c.1925dupG | p.Ile643fs |
| 2:25243908:TCC:T | NA | frameshift | c.1924_1925delGG | p.Gly642fs |
| 2:25243909:C:A | NA | missense | c.1925G > T | p.Gly642Val |
| 2:25243909:C:CC | NA | frameshift | c.1923_1924dupTG | p.Gly642fs |
| A | | | | |
| 2:25243909:C:T | NA | missense | c.1925G > A | p.Gly642Glu |
| 2:25243910:C:A | rs551213321 | stop_gained | c.1924G > T | p.Gly642* |
| 2:25243910:C:T | rs551213321 | missense | c.1924G > A | p.Gly642Arg |
| 2:25243910:CA:C | NA | frameshift | c.1923delT | p.Asp641fs |
| 2:25243911:A:T | NA | missense | c.1923T > A | p.Asp641Glu |
| 2:25243912:T:A | NA | missense | c.1922A > T | p.Asp641Val |
| 2:25243912:TCAA | NA | inframe_indel | c.1916_1921delTCT | p.Leu639_Asp641 |
| AGA:T | | | TTG | delinsHis |
| 2:25243912:TCAA | NA | frameshift | c.1909_1921delCT | p.Leu637fs |
| AGAGAGACAG:T | | | GTCTCTCTTTG | |
| 2:25243913:C:A | NA | missense | c.1921G > T | p.Asp641Tyr |
| 2:25243913:C:CA | NA | frameshift | c.1920dupT | p.Asp641fs |
| 2:25243913:C:G | NA | missense | c.1921G > C | p.Asp641His |
| 2:25243913:C:T | rs1331365627 | missense | c.1921G > A | p.Asp641Asn |
| 2:25243913:CA:C | NA | frameshift | c.1920delT | p.Phe640fs |
| 2:25243913:CAA: | rs1412485785 | frameshift | c.1919_1920delTT | p.Phe640fs |
| C | | | | |
| 2:25243913:CAAA | NA | frameshift | c.1917_1920delCTT | p.Phe640fs |
| G:C | | | T | |
| 2:25243913:CAAA | NA | frameshift | c.1916_1920delTCT | p.Leu639fs |
| GA:C | | | TT | |
| 2:25243915:A:C | NA | missense | c.1919T > G | p.Phe640Cys |
| 2:25243915:A:G | NA | missense | c.1919T > C | p.Phe640Ser |
| 2:25243915:AAG: | NA | frameshift | c.1917_1918delCT | p.Phe640fs |
| A | | | | |
| 2:25243915:AAG | NA | frameshift | c.1915_1918delCT | p.Phe640fs |
| AG:A | | | CT | |
| 2:25243915:AAG | NA | frameshift | c.1911_1918delGT | p.Ser638fs |
| AGAGAC:A | | | CTCTCT | |
| 2:25243916:A:C | NA | missense | c.1918T > G | p.Phe640Val |
| 2:25243916:A:G | NA | missense | c.1918T > C | p.Phe640Leu |
| 2:25243916:A:T | NA | missense | c.1918T > A | p.Phe640Ile |
| 2:25243917:GA:G | NA | frameshift | c.1916delT | p.Leu639fs |
| 2:25243918:A:C | NA | missense | c.1916T > G | p.Leu639Arg |
| 2:25243918:A:G | NA | missense | c.1916T > C | p.Leu639Pro |
| 2:25243918:A:T | NA | missense | c.1916T > A | p.Leu639His |
| 2:25243918:AG:A | NA | frameshift | c.1915delC | p.Leu639fs |
| 2:25243919:G:A | NA | missense | c.1915C > T | p.Leu639Phe |
| 2:25243919:G:C | NA | missense | c.1915C > G | p.Leu639Val |
| 2:25243919:G:GA | NA | frameshift | c.1911_1914dupGT | p.Leu639fs |
| GAC | | | CT | |
| 2:25243919:GA:G | NA | frameshift | c.1914delT | p.Leu639fs |
| 2:25243919:GAG | NA | frameshift | c.1911_1914delGT | p.Phe640fs |
| AC:G | | | CT | |
| 2:25243920:A:AG | NA | frameshift | c.1913dupC | p.Leu639fs |
| 2:25243921:G:A | rs1253868717 | missense | c.1913C > T | p.Ser638Phe |
| 2:25243921:G:C | rs1253868717 | missense | c.1913C > G | p.Ser638Cys |
| 2:25243921:G:T | NA | missense | c.1913C > A | p.Ser638Tyr |
| 2:25243921:GAC: | NA | frameshift | c.1911_1912delGT | p.Phe640fs |
| G | | | | |
| 2:25243922:A:C | NA | missense | c.1912T > G | p.Ser638Ala |
| 2:25243922:A:G | NA | missense | c.1912T > C | p.Ser638Pro |
| 2:25243922:AC:A | NA | frameshift | c.1911delG | p.Ser638fs |
| 2:25243923:CA:C | NA | frameshift | c.1910delT | p.Leu637fs |
| 2:25243924:A:C | rs371523368 | missense | c.1910T > G | p.Leu637Arg |
| 2:25243924:A:G | rs371523368 | missense | c.1910T > C | p.Leu637Pro |
| 2:25243924:A:T | NA | missense | c.1910T > A | p.Leu637Gln |
| 2:25243925:G:C | NA | missense | c.1909C > G | p.Leu637Val |
| 2:25243925:G:GC | NA | frameshift | c.1907_1908dupTG | p.Leu637fs |
| A | | | | |
| 2:25243925:G:GT | NA | frameshift | c.1908_1909insA | p.Leu637fs |
| 2:25243926:CA:C | NA | frameshift | c.1907delT | p.Va1636fs |
| 2:25243927:A:AC | NA | frameshift | c.1900_1906dupAT | p.Va1636fs |
| CCGGAT | | | CCGGG | |
| 2:25243927:A:C | rs929806900 | missense | c.1907T > G | p.Va1636Gly |
| 2:25243927:A:G | NA | missense | c.1907T > C | p.Va1636Ala |
| 2:25243927:A:T | NA | missense | c.1907T > A | p.Va1636Glu |
| 2:25243927:AC:A | rs778979256 | frameshift | c.1906delG | p.Va1636fs |
| 2:25243927:ACCC | NA | frameshift | c.1902_1906delCC | p.Arg635fs |
| GG:A | | | GGG | |
| 2:25243928:C:A | rs376550450 | missense | c.1906G > T | p.Va1636Leu |
| 2:25243928:C:CA | NA | frameshift | c.1905_1906insT | p.Va1636fs |
| 2:25243928:C:G | NA | missense | c.1906G > C | p.Va1636Leu |
| 2:25243928:C:T | rs376550450 | missense | c.1906G > A | p.Va1636Met |
| 2:25243930:C:A | NA | missense | c.1904G > T | p.Arg635Leu |
| 2:25243930:C:G | rs751562376 | missense | c.1904G > C | p.Arg635Pro |
| 2:25243930:C:T | rs751562376 | missense | c.1904G > A | p.Arg635Gln |
| 2:25243930:CG:C | rs1326186258 | frameshift | c.1903delC | p.Arg635fs |
| 2:25243930:CGG | NA | inframe_indel | c.1874_1903delCT | p.Pro625_Ile634 |
| ATGGGCTTCCTCT | | | GTCCCAGCTGAGAA | del |
| TCTCAGCTGGGAC | | | GAGGAAGCCCATCC | |
| AG:C | | | | |
| 2:25243931:G:A | rs144689354 | missense | c.1903C > T | p.Arg635Trp |
| 2:25243931:G:C | NA | missense | c.1903C > G | p.Arg635Gly |
| 2:25243932:G:C | NA | missense | c.1902C > G | p.Ile634Met |
| 2:25243932:G:GA | NA | frameshift | c.1894_1901dupAA | p.Arg635fs |
| TGGGCTT | | | GCCCAT | |
| 2:25243932:GA:G | NA | frameshift | c.1901delT | p.Ile634fs |
| 2:25243933:A:AT | NA | frameshift | c.1899_1900dupCA | p.Ile634fs |
| G | | | | |
| 2:25243933:A:C | NA | missense | c.1901T > G | p.Ile634Ser |
| 2:25243933:A:G | rs1390273539 | missense | c.1901T > C | p.Ile634Thr |
| 2:25243933:A:T | NA | missense | c.1901T > A | p.Ile634Asn |
| 2:25243933:AT:A | NA | frameshift | c.1900delA | p.Ile634fs |
| 2:25243934:T:A | NA | missense | c.1900A > T | p.Ile634Phe |
| 2:25243934:TG:T | NA | frameshift | c.1899delC | p.Ile634fs |
| 2:25243936:G:A | rs1446825883 | missense | c.1898C > T | p.Pro633Leu |
| 2:25243936:G:C | rs1446825883 | missense | c.1898C > G | p.Pro633Arg |
| 2:25243936:G:GG | NA | frameshift | c.1894_1897dupAA | p.Pro633fs |
| CTT | | | GC | |
| 2:25243936:G:T | NA | missense | c.1898C > A | p.Pro633His |
| 2:25243937:G:A | rs1191994998 | missense | c.1897C > T | p.Pro633Ser |
| 2:25243937:G:GC | NA | frameshift | c.1896dupG | p.Pro633fs |
| 2:25243937:G:T | NA | missense | c.1897C > A | p.Pro633Thr |
| 2:25243937:GC:G | NA | frameshift | c.1896delG | p.Lys632fs |
| 2:25243938:CT:C | rs1195836932 | frameshift | c.1895delA | p.Lys632fs |
| 2:25243939:T:TTC | NA | frameshift | c.1875_1894dupTG | p.Lys632fs |
| CTCTTCTCAGCTG | | | TCCCAGCTGAGAAG | |
| GGACA | | | AGGA | |
| 2:25243940:T:A | rs1245875087 | stop_gained | c.1894A > T | p.Lys632* |
| 2:25243940:T:G | NA | missense | c.1894A > C | p.Lys632Gln |
| 2:25243940:TC:T | NA | frameshift | c.1893delG | p.Lys632fs |
| 2:25243941:C:A | NA | missense | c.1893G > T | p.Arg631Ser |
| 2:25243941:c.G | NA | missense | c.1893G > C | p.Arg631Ser |
| 2:25243941:CCTC | NA | frameshift | c.1880_1892delCA | p.Pro627fs |
| TTCTCAGCTG:C | | | GCTGAGAAGAG | |
| 2:25243942:C:A | NA | missense | c.1892G > T | p.Arg631Met |
| 2:25243942:C:G | rs1159270298 | missense | c.1892G > C | p.Arg631Thr |
| 2:25243942:C:T | NA | missense | c.1892G > A | p.Arg631Lys |
| 2:25243943:T:A | rs1450721986 | missense | c.1891A > T | p.Arg631Trp |
| 2:25243943:T:C | NA | missense | c.1891A > G | p.Arg631Gly |
| 2:25243944:CT:C | NA | frameshift | c.1889delA | p.Lys630fs |
| 2:25243944:CTT:C | NA | frameshift | c.1888_1889delAA | p.Lys630fs |
| 2:25243945:T:TTC | NA | frameshift | c.1887_1888dupG | p.Lys630fs |
| | | | A | |
| 2:25243946:T:A | NA | stop_gained | c.1888A > T | p.Lys630* |
| 2:25243947:CT:C | NA | frameshift | c.1886delA | p.Glu629fs |
| 2:25243947:CTCA | NA | frameshift | c.1865_1886delTTT | p.Va1622fs |
| GCTGGGACAGGT | | | ACCCACCTGTCCCA | |
| GGGTAAA:C | | | GCTGA | |
| 2:25243949:C:A | NA | stop_gained | c.1885G > T | p.Glu629* |
| 2:25243949:C:CA | NA | frameshift | c.1884dupT | p.Glu629fs |
| 2:25243949:CAG: | NA | frameshift | c.1883_1884delCT | p.Ala628fs |
| C | | | | |
| 2:25243951:G:T | rs149876809 | missense | c.1883C > A | p.Ala628Asp |
| 2:25243951:GC:G | NA | frameshift | c.1882delG | p.Ala628fs |
| 2:25243953:T:TG | NA | frameshift | c.1880dupC | p.Ala628fs |
| 2:25243953:TG:T | NA | frameshift | c.1880delC | p.Pro627fs |
| 2:25243954:GGG | NA | frameshift | c.1860_1879delAA | p.Lys621fs |
| ACAGGTGGGTAA | | | AGGTTTACCCACCT | |
| ACCTTT:G | | | GTCC | |
| 2:25243956:GA:G | NA | frameshift | c.1877delT | p.Va1626fs |
| 2:25243956:GAC | NA | inframe_indel | c.1866_1877delTT | p.Tyr623_Va1626 |
| AGGTGGGTAA:G | | | ACCCACCTGT | del |
| 2:25243957:A:C | NA | missense | c.1877T > G | p.Va1626Gly |
| 2:25243957:A:T | NA | missense | c.1877T > A | p.Va1626Asp |
| 2:25243957:AC:A | NA | frameshift | c.1876delG | p.Va1626fs |
| 2:25243958:CA:C | NA | frameshift | c.1875delT | p.Va1626fs |
| 2:25243958:CAG | NA | frameshift | c.1866_1875delTT | p.Tyr623fs |
| GTGGGTAA:C | | | ACCCACCT | |
| 2:25243959:A:AG | NA | frameshift | c.1870_1874dupCC | p.Va1626fs |
| GTGG | | | ACC | |
| 2:25243959:AG:A | NA | frameshift | c.1874delC | p.Pro625fs |
| 2:25243960:G:C | NA | missense | c.1874C > G | p.Pro625Arg |
| 2:25243960:G:GG | NA | frameshift | c.1872_1873dupAC | p.Pro625fs |
| T | | | | |
| 2:25243961:G:GT | NA | frameshift | c.1872dupA | p.Pro625fs |
| 2:25243961:GT:G | rs745381125 | frameshift | c.1872delA | p.Pro625fs |
| 2:25243962:TG:T | NA | frameshift | c.1871delC | p.Pro624fs |
| 2:25243963:GGG | NA | frameshift | c.1866_1870delTT | p.Tyr623fs |
| TAA:G | | | ACC | |
| 2:25243963:GGG | rs1394353623 | inframe_indel | c.1865_1870delTTT | p.Va1622_Pro624 |
| TAAA:G | | | ACC | delinsAla |
| 2:25243964:G:GG | NA | frameshift | c.1869_1870insAT | p.Pro624fs |
| TAAACCTTTGGAG | | | GCTTAGGACCCTCC | |
| GGTCCTAAGCAT | | | AAAGGTTTAC | |
| 2:25243964:G:GG | NA | frameshift | c.1869_1870insTA | p.Pro624fs |
| TTTA | | | AAC | |
| 2:25243965:G:C | NA | stop_gained | c.1869C > G | p.Tyr623* |
| 2:25243965:G:GT | NA | frameshift | c.1868_1869insAA | p.Tyr623fs |
| T | | | | |
| 2:25243965:G:T | NA | stop_gained | c.1869C > A | p.Tyr623* |
| 2:25243965:GT:G | NA | frameshift | c.1868delA | p.Tyr623fs |
| 2:25243966:T:TA | NA | frameshift | c.1867dupT | p.Tyr623fs |
| 2:25243966:T:TA | NA | frameshift | c.1867_1868insGG | p.Tyr623fs |
| ACCC | | | GTT | |
| 2:25243966:TA:T | NA | frameshift | c.1867delT | p.Tyr623fs |
| 2:25243966:TAAA | NA | frameshift | c.1863_1867delGG | p.Va1622fs |
| CC:T | | | TTT | |
| 2:25243968:A:AA | rs1477019156 | frameshift | c.1864_1865dupGT | p.Tyr623fs |
| C | | | | |
| 2:25243968:A:AT | NA | frameshift | c.1865_1866insGT | p.Tyr623fs |
| TAC | | | AA | |
| 2:25243969:AC:A | NA | frameshift | c.1864delG | p.Va1622fs |
| 2:25243969:ACCT | NA | frameshift | c.1861_1864delAA | p.Lys621fs |
| T:A | | | GG | |
| 2:25243971:CT:C | NA | frameshift | c.1862delA | p.Lys621fs |
| 2:25243973:T:A | NA | stop_gained | c.1861A > T | p.Lys621* |
| 2:25243974:TG:T | NA | frameshift | c.1859delC | p.Pro620fs |
| 2:25243976:GA:G | NA | frameshift | c.1857delT | p.Pro620fs |
| 2:25243977:AG:A | NA | frameshift | c.1856delC | p.Pro619fs |
| 2:25243980:GT:G | NA | frameshift | c.1853delA | p.Asp618fs |
| 2:25243981:TC:T | NA | frameshift | c.1852delG | p.Asp618fs |
| 2:25243983:C:T | rs866791048 | splice_acceptor | c.1852-1G > A | |
| 2:25244151:TCA: | rs1228601968 | splice_donor | c.1851+2_1851+3del | |
| T | | | TG | |
| 2:25244152:CA:C | NA | splice_donor | c.1851+2delT | |
| 2:25244152:CACA | NA | frameshift | c.1847_1851+2del | p.Glu616fs |
| AATT:C | | | AATTTGT | |
| 2:25244153:A:C | NA | splice_donor | c.1851+2T > G | |
| 2:25244153:A:G | rs757785566 | splice_donor | c.1851+2T > C | |
| 2:25244153:A:T | rs757785566 | splice_donor | c.1851+2T > A | |
| 2:25244153:AC:A | NA | splice_donor | c.1851+1delG | |
| 2:25244153:ACAA | NA | frameshift | c.1848_1851+ldel | p.Phe617fs |
| AT:A | | | ATTTG | |
| 2:25244154:C:A | NA | splice_donor | c.1851+1G > T | |
| 2:25244154:C:G | NA | splice_donor | c.1851+1G > C | |
| 2:25244154:C:T | rs928051436 | splice_donor | c.1851+1G > A | |
| 2:25244154:CA:C | NA | frameshift | c.1851delT | p.Phe617fs |
| 2:25244157:AT:A | NA | frameshift | c.1848delA | p.Glu616fs |
| 2:25244158:T:TG | NA | frameshift | c.1847_1848insC | p.Glu616fs |
| 2:25244159:T:A | NA | missense | c.1847A > T | p.Glu616Val |
| 2:25244159:TC:T | rs1409292254 | frameshift | c.1846delG | p.Glu616fs |
| 2:25244160:C:A | rs779600073 | stop_gained | c.1846G > T | p.Glu616* |
| 2:25244161:CT:C | NA | frameshift | c.1844delA | p.Gln615fs |
| 2:25244162:TG:T | NA | frameshift | c.1843delC | p.Gln615fs |
| 2:25244162:TGGT | NA | frameshift | c.1840_1843delGA | p.Asp614fs |
| C:T | | | CC | |
| 2:25244163:G:A | rs768548187 | stop_gained | c.1843C > T | p.Gln615* |
| 2:25244163:G:GG | NA | frameshift | c.1838_1842dupAC | p.Gln615fs |
| TCGT | | | GAC | |
| 2:25244164:GT:G | NA | frameshift | c.1841delA | p.Asp614fs |
| 2:25244165:TC:T | rs757911864 | frameshift | c.1840delG | p.Asp614fs |
| 2:25244166:CG:C | NA | frameshift | c.1839delC | p.His613fs |
| 2:25244167:GT:G | rs1431450984 | frameshift | c.1838delA | p.His613fs |
| 2:25244167:GTG | NA | frameshift | c.1835_1838delAC | p.Asn612fs |
| GT:G | | | CA | |
| 2:25244167:GTG | NA | inframe_indel | c.1818_1838delGA | p.Gln606_Asn612 |
| GTTATTAGCGAAG | | | TGTTCTTCGCTAAT | del |
| AACATC:G | | | AACCA | |
| 2:25244168:T:TG | NA | frameshift | c.1837dupC | p.His613fs |
| 2:25244168:TG:T | NA | frameshift | c.1837delC | p.His613fs |
| 2:25244168:TGGT | NA | frameshift | c.1828_1837delGC | p.Ala610fs |
| TATTAGC:T | | | TAATAACC | |
| 2:25244170:G:GT | NA | frameshift | c.1834_1835dupAA | p.Asn612fs |
| T | | | | |
| 2:25244170:GT:G | NA | frameshift | c.1835delA | p.Asn612fs |
| 2:25244170:GTT: | NA | frameshift | c.1834_1835delAA | p.Asn612fs |
| G | | | | |
| 2:25244170:GTTA | NA | frameshift | c.1832_1835delAT | p.Asn611fs |
| T:G | | | AA | |
| 2:25244172:TA:T | NA | frameshift | c.1833delT | p.Asn611fs |
| 2:25244172:TATT | NA | frameshift | c.1827_1833delCG | p.Phe609fs |
| AGCG:T | | | CTAAT | |
| 2:25244172:TATT | NA | frameshift | c.1826_1833delTC | p.Phe609fs |
| AGCGA:T | | | GCTAAT | |
| 2:25244173:AT:A | NA | frameshift | c.1832delA | p.Asn611fs |
| 2:25244174:TTAG | NA | frameshift | c.1827_1831delCG | p.Phe609fs |
| CG:T | | | CTA | |
| 2:25244176:AG:A | NA | frameshift | c.1829delC | p.Ala610fs |
| 2:25244177:G:C | rs930025818 | missense | c.1829C > G | p.Ala610Gly |
| 2:25244178:C:CG | NA | frameshift | c.1827dupC | p.Ala610fs |
| 2:25244178:C:T | NA | missense | c.1828G > A | p.Ala610Thr |
| 2:25244178:CG:C | | frameshift | c.1827delC | p.Phe609fs |
| 2:25244179:GA:G | NA | frameshift | c.1826delT | p.Phe609fs |
| 2:25244181:AG:A | NA | frameshift | c.1824delC | p.Phe609fs |
| 2:25244182:G:GC | NA | frameshift | c.1823_1824insG | p.Phe608fs |
| 2:25244182:G:GT | NA | frameshift | c.1823_1824insA | p.Phe608fs |
| 2:25244182:GA:G | NA | frameshift | c.1823delT | p.Phe608fs |
| 2:25244183:AAC: | NA | frameshift | c.1821_1822delGT | p.Met607fs |
| A | | | | |
| 2:25244184:A:AC | NA | frameshift | c.1817_1821dupA | p.Phe608fs |
| ATCT | | | GATG | |
| 2:25244184:A:AC | NA | frameshift | c.1812_1821dupGC | p.Phe608fs |
| ATCTGGAGC | | | TCCAGATG | |
| 2:25244185:CATC | NA | frameshift | c.1816_1820delCA | p.Gln606fs |
| TG:C | | | GAT | |
| 2:25244188:CT:C | NA | frameshift | c.1817delA | p.Gln606fs |
| 2:25244188:CTG: | NA | frameshift | c.1816_1817delCA | p.Gln606fs |
| C | | | | |
| 2:25244189:T:TG | NA | frameshift | c.1810_1816dupCG | p.Gln606fs |
| GAGCCG | | | GCTCC | |
| 2:25244189:TG:T | NA | frameshift | c.1816delC | p.Gln606fs |
| 2:25244190:G:A | rs1396358001 | stop_gained | c.1816C > T | p.Gln606* |
| 2:25244193:GC:G | NA | frameshift | c.1812delG | p.Leu605fs |
| 2:25244195:C:G | NA | missense | c.1811G > C | p.Arg604Pro |
| 2:25244195:C:T | rs759818409 | missense | c.1811G > A | p.Arg604Gln |
| 2:25244195:CG:C | rs779782698 | frameshift | c.1810delC | p.Arg604fs |
| 2:25244196:G:A | rs1368287759 | missense | c.1810C > T | p.Arg604Trp |
| 2:25244196:G:C | NA | missense | c.1810C > G | p.Arg604Gly |
| 2:25244198:GA:G | NA | frameshift | c.1807delT | p.Ser603fs |
| 2:25244199:AG:A | NA | frameshift | c.1806delC | p.Ser603fs |
| 2:25244199:AGG: | NA | frameshift | c.1805_1806delCC | p.Pro602fs |
| A | | | | |
| 2:25244199:AGG | NA | frameshift | c.1803_1806delGC | p.Trp601fs |
| GC:A | | | CC | |
| 2:25244202:G:T | NA | missense | c.1804C > A | p.Pro602Thr |
| 2:25244202:GC:G | NA | frameshift | c.1803delG | p.Trp601fs |
| 2:25244202:GCCA | NA | frameshift | c.1799_1803delAC | p.Asp600fs |
| GT:G | | | TGG | |
| 2:25244203:C:T | rs767692203 | stop_gained | c.1803G > A | p.Trp601* |
| 2:25244203:CCA: | NA | frameshift | c.1801_1802delTG | p.Trp601fs |
| C | | | | |
| 2:25244204:C:T | rs941325374 | stop_gained | c.1802G > A | p.Trp601* |
| 2:25244204:CA:C | NA | frameshift | c.1801delT | p.Trp601fs |
| 2:25244205:A:AG | NA | frameshift | c.1800dupC | p.Trp601fs |
| 2:25244205:A:G | NA | missense | c.1801T > C | p.Trp601Arg |
| 2:25244205:AG:A | NA | frameshift | c.1800delC | p.Trp601fs |
| 2:25244206:G:GT | NA | frameshift | c.1795_1799dupG | p.Asp600fs |
| CCTC | | | AGGA | |
| 2:25244208:C:CC | NA | frameshift | c.1796_1797dupA | p.Asp600fs |
| T | | | G | |
| 2:25244209:CT:C | NA | frameshift | c.1796delA | p.Glu599fs |
| 2:25244211:C:A | NA | stop_gained | c.1795G > T | p.Glu599* |
| 2:25244211:C:CT | NA | frameshift | c.1794dupA | p.Glu599fs |
| 2:25244212:T:TC | NA | frameshift | c.1793dupG | p.Glu599fs |
| 2:25244212:TCGC | NA | inframe_indel | c.1791_1793delGC | p.Arg598del |
| T | | | G | |
| 2:25244213:C:T | rs764679587 | missense | c.1793G > A | p.Arg598Gln |
| 2:25244214:G:A | rs568207978 | stop_gained | c.1792C > T | p.Arg598* |
| 2:25244214:G:C | NA | missense | c.1792C > G | p.Arg598Gly |
| 2:25244216:CG:C | NA | frameshift | c.1789delC | p.Arg597fs |
| 2:25244216:CGCC | NA | inframe_indel | c.1781_1789delTG | p.Leu594_Arg596 |
| GCAGCA:C | | | CTGCGGC | del |
| 2:25244217:G:A | rs757492795 | missense | c.1789C > T | p.Arg597Trp |
| 2:25244217:G:GC | NA | frameshift | c.1788dupG | p.Arg597fs |
| 2:25244217:GC:G | NA | frameshift | c.1788delG | p.Arg597fs |
| 2:25244218:C:CC | NA | frameshift | c.1783_1787dupCT | p.Arg597fs |
| GCAG | | | GCG | |
| 2:25244218:C:CC | NA | frameshift | c.1787_1788insACT | p.Arg597fs |
| GCAGCAGCCCGTA | | | ACGGGCTGCTGCG | |
| GT | | | | |
| 2:25244219:C:T | rs899717364 | missense | c.1787G > A | p.Arg596Gln |
| 2:25244219:CGCA | NA | frameshift | c.1773_1786delCT | p.Tyr592fs |
| GCAGCCCGTAG:C | | | ACGGGCTGCTGC | |
| 2:25244220:G:GC | NA | frameshift | c.1785dupG | p.Arg596fs |
| 2:25244220:GC:G | NA | frameshift | c.1785delG | p.Arg596fs |
| 2:25244221:CA:C | NA | frameshift | c.1784delT | p.Leu595fs |
| 2:25244222:A:C | NA | missense | c.1784T > G | p.Leu595Arg |
| 2:25244222:A:G | rs1483256801 | missense | c.1784T > C | p.Leu595Pro |
| 2:25244223:G:GC | NA | frameshift | c.1779_1782dupGC | p.Leu595fs |
| AGC | | | TG | |
| 2:25244223:GC:G | NA | frameshift | c.1782delG | p.Leu595fs |
| 2:25244224:CA:C | NA | frameshift | c.1781delT | p.Leu594fs |
| 2:25244225:AGCC | NA | frameshift | c.1759_1780delGG | p.Gly587fs |
| CGTAGGTACCCTT | | | GCACAAGGGTACCT | |
| GTGCCC:A | | | ACGGGC | |
| 2:25244226:GCG | NA | frameshift | c.1779delG | p.Leu594fs |
| 2:25244229:C:CG | NA | frameshift | c.1773_1776dupCT | p.Gly593fs |
| TAG | | | AC | |
| 2:25244229:C:T | rs755573228 | missense | c.1777G > A | p.Gly593Arg |
| 2:25244229:CG:C | NA | frameshift | c.1776delC | p.Tyr592fs |
| 2:25244230:G:C | rs758063797 | stop_gained | c.1776C > G | p.Tyr592* |
| 2:25244230:G:GT | NA | frameshift | c.1775_1776insGA | p.Tyr592fs |
| C | | | | |
| 2:25244230:G:T | rs758063797 | stop_gained | c.1776C > A | p.Tyr592* |
| 2:25244231:TA:T | NA | frameshift | c.1774delT | p.Tyr592fs |
| 2:25244231:TAG | NA | frameshift | c.1755_1774delGT | p.Met585fs |
| GTACCCTTGTGCC | | | GCGGGCACAAGGG | |
| CGCAC:T | | | TACCT | |
| 2:25244232:A:AG | NA | frameshift | c.1773dupC | p.Tyr592fs |
| 2:25244234:G:A | NA | missense | c.1772C > T | p.Thr591Ile |
| 2:25244235:TACC | NA | frameshift | c.1764_1770delCA | p.His588fs |
| CTTG:T | | | AGGGT | |
| 2:25244235:TACC | NA | frameshift | c.1748_1770delGC | p.Cys583fs |
| CTTGTGCCCGCAC | | | TACATGTGCGGGCA | |
| ATGTAGC:T | | | CAAGGGT | |
| 2:25244236:AC:A | NA | frameshift | c.1769delG | p.Gly590fs |
| 2:25244236:ACCC | NA | frameshift | c.1763_1769delAC | p.His588fs |
| TTGT:A | | | AAGGG | |
| 2:25244239:CT:C | NA | frameshift | c.1766delA | p.Lys589fs |
| 2:25244240:T:C | rs1324043719 | missense | c.1766A > G | p.Lys589Arg |
| 2:25244240:TTG: | NA | frameshift | c.1764_1765delCA | p.His588fs |
| T | | | | |
| 2:25244240:TTGT | NA | frameshift | c.1761_1765delGC | p.His588fs |
| GC:T | | | ACA | |
| 2:25244242:GT:G | NA | frameshift | c.1763delA | p.His588fs |
| 2:25244242:GTGC | NA | frameshift | c.1753_1763delAT | p.Met585fs |
| CCGCACAT:G | | | GTGCGGGCA | |
| 2:25244243:T:TG | NA | frameshift | c.1759_1762dupG | p.His588fs |
| CCC | | | GGC | |
| 2:25244243:TG:T | NA | frameshift | c.1762delC | p.His588fs |
| 2:25244244:GC:G | rs1313980878 | frameshift | c.1761delG | p.His588fs |
| 2:25244246:C:A | NA | missense | c.1760G > T | p.Gly587Val |
| 2:25244246:C:G | NA | missense | c.1760G > C | p.Gly587Ala |
| 2:25244247:CGCA | NA | frameshift | c.1752_1758delCA | p.Tyr584fs |
| CATG:C | | | TGTGC | |
| 2:25244248:G:C | NA | missense | c.1758C > G | p.Cys586Trp |
| 2:25244248:G:GC | NA | frameshift | c.1756_1757dupTG | p.Gly587fs |
| A | | | | |
| 2:25244248:G:T | rs746498722 | stop_gained | c.1758C > A | p.Cys586* |
| 2:25244248:GCA: | NA | frameshift | c.1756_1757delTG | p.Cys586fs |
| G | | | | |
| 2:25244249:C:CA | NA | frameshift | c.1756dupT | p.Cys586fs |
| 2:25244249:C:G | NA | missense | c.1757G > C | p.Cys586Ser |
| 2:25244249:C:T | rs754506713 | missense | c.1757G > A | p.Cys586Tyr |
| 2:25244250:A:AC | rs1371954044 | frameshift | c.1755dupG | p.Cys586fs |
| 2:25244250:A:C | NA | missense | c.1756T > G | p.Cys586Gly |
| 2:25244250:A:G | NA | missense | c.1756T > C | p.Cys586Arg |
| 2:25244250:AC:A | rs1376463207 | frameshift | c.1755delG | p.Met585fs |
| 2:25244250:ACAT | NA | frameshift | c.1743_1755delGA | p.Trp581fs |
| GTAGCAGTTC:A | | | ACTGCTACATG | |
| 2:25244251:CA:C | NA | frameshift | c.1754delT | p.Met585fs |
| 2:25244251:CATG | NA | frameshift | c.1747_1754delTG | p.Cys583fs |
| TAGCA:C | | | CTACAT | |
| 2:25244251:CATG | NA | frameshift | c.1745_1754delAC | p.Asn582fs |
| TAGCAGT:C | | | TGCTACAT | |
| 2:25244252:A:AT | NA | frameshift | c.1752_1753dupCA | p.Met585fs |
| G | | | | |
| 2:25244252:AT:A | NA | frameshift | c.1753delA | p.Met585fs |
| 2:25244252:ATGT | NA | frameshift | c.1746_1753delCT | p.Cys583fs |
| AGCAG:A | | | GCTACA | |
| 2:25244253:T:C | NA | missense | c.1753A > G | p.Met585Val |
| 2:25244253:TG:T | NA | frameshift | c.1752delC | p.Tyr584fs |
| 2:25244253:TGTA | NA | frameshift | c.1740_1752delCT | p.Trp581fs |
| GCAGTTCCAG:T | | | GGAACTGCTAC | |
| 2:25244253:TGTA | NA | frameshift | c.1739_1752delCC | p.Pro580fs |
| GCAGTTCCAGG:T | | | TGGAACTGCTAC | |
| 2:25244254:G:C | NA | stop_gained | c.1752C > G | p.Tyr584* |
| 2:25244254:G:GT | NA | frameshift | c.1751dupA | p.Tyr584fs |
| 2:25244254:G:T | NA | stop_gained | c.1752C > A | p.Tyr584* |
| 2:25244254:GT:G | NA | frameshift | c.1751delA | p.Tyr584fs |
| 2:25244255:T:C | NA | missense | c.1751A > G | p.Tyr584Cys |
| 2:25244256:A:G | NA | missense | c.1750T > C | p.Tyr584His |
| 2:25244257:G:C | NA | missense | c.1749C > G | p.Cys583Trp |
| 2:25244257:G:T | NA | stop_gained | c.1749C > A | p.Cys583* |
| 2:25244257:GC:G | NA | frameshift | c.1748delG | p.Cys583fs |
| 2:25244258:C:G | NA | missense | c.1748G > C | p.Cys583Ser |
| 2:25244258:C:T | rs748082838 | missense | c.1748G > A | p.Cys583Tyr |
| 2:25244259:A:G | NA | missense | c.1747T > C | p.Cys583Arg |
| 2:25244259:A:T | NA | missense | c.1747T > A | p.Cys583Ser |
| 2:25244259:AG:A | NA | frameshift | c.1746delC | p.Cys583fs |
| 2:25244260:G:GT | NA | frameshift | c.1745dupA | p.Asn582fs |
| 2:25244260:GT:G | NA | frameshift | c.1745delA | p.Asn582fs |
| 2:25244260:GTTC | NA | frameshift | c.1735_1745delGA | p.Asp579fs |
| CAGGGGTC:G | | | CCCCTGGAA | |
| 2:25244262:TC:T | NA | frameshift | c.1743delG | p.Trp581fs |
| 2:25244263:C:A | rs769419803 | missense | c.1743G > T | p.Trp581Cys |
| 2:25244263:CCC | NA | frameshift | c.1741_1742dupTG | p.Trp581fs |
| A | | | | |
| 2:25244263:C:G | rs769419803 | missense | c.1743G > C | p.Trp581Cys |
| 2:25244263:C:T | rs769419803 | stop_gained | c.1743G > A | p.Trp581* |
| 2:25244264:C:A | NA | missense | c.1742G > T | p.Trp581Leu |
| 2:25244264:C:G | NA | missense | c.1742G > C | p.Trp581Ser |
| 2:25244264:C:T | rs1050697275 | stop_gained | c.1742G > A | p.Trp581* |
| 2:25244264:CA:C | NA | frameshift | c.1741delT | p.Trp581fs |
| 2:25244264:CAG | NA | frameshift | c.1738_1741delCC | p.Pro580fs |
| GG:C | | | CT | |
| 2:25244264:CAG | NA | inframe_indel | c.1721_1741delCA | p.Ala574_Trp581 |
| GGGTCTTCCTTAA | | | GCCATTAAGGAAG | delinsGly |
| TGGCTG:C | | | ACCCCT | |
| 2:25244265:A:AG | NA | frameshift | c.1740dupC | p.Trp581fs |
| 2:25244265:A:AG | NA | frameshift | c.1739_1740dupCC | p.Trp581fs |
| G | | | | |
| 2:25244265:A:C | rs1342803588 | missense | c.1741T > G | p.Trp581Gly |
| 2:25244265:A:G | NA | missense | c.1741T > C | p.Trp581Arg |
| 2:25244265:A:T | NA | missense | c.1741T > A | p.Trp581Arg |
| 2:25244265:AG:A | NA | frameshift | c.1740delC | p.Trp581fs |
| 2:25244266:GGG | NA | frameshift | c.1730_1739delAG | p.Lys577fs |
| GTCTTCCT:G | | | GAAGACCC | |
| 2:25244266:GGG | NA | frameshift | c.1718_1739delAG | p.Gln573fs |
| GTCTTCCTTAATG | | | GCAGCCATTAAGGA | |
| GCTGCCT:G | | | AGACCC | |
| 2:25244267:G:A | rs773048923 | missense | c.1739C > T | p.Pro580Leu |
| 2:25244267:G:T | NA | missense | c.1739C > A | p.Pro580His |
| 2:25244267:GGG | NA | frameshift | c.1735_1738delGA | p.Asp579fs |
| TC:G | | | CC | |
| 2:25244268:G:A | NA | missense | c.1738C > T | p.Pro580Ser |
| 2:25244269:GT:G | NA | frameshift | c.1736delA | p.Asp579fs |
| 2:25244269:GTCT | NA | frameshift | c.1732_1736delGA | p.Glu578fs |
| TC:G | | | AGA | |
| 2:25244270:T:A | NA | missense | c.1736A > T | p.Asp579Val |
| 2:25244270:T:C | NA | missense | c.1736A > G | p.Asp579Gly |
| 2:25244270:TCTT | NA | frameshift | c.1732_1735delGA | p.Glu578fs |
| C:T | | | AG | |
| 2:25244270:TCTT | NA | frameshift | c.1720_1735delGC | p.Ala574fs |
| CCTTAATGGCTGC: | | | AGCCATTAAGGAA | |
| T | | | G | |
| 2:25244271:C:A | NA | missense | c.1735G > T | p.Asp579Tyr |
| 2:25244271:C:CT | NA | frameshift | c.1734dupA | p.Asp579fs |
| 2:25244271:C:T | NA | missense | c.1735G > A | p.Asp579Asn |
| 2:25244271:CT:C | NA | frameshift | c.1734delA | p.Asp579fs |
| 2:25244272:T:TA | NA | frameshift | c.1733_1734insGT | p.Asp579fs |
| GAC | | | CT | |
| 2:25244272:TTC:T | NA | frameshift | c.1732_1733delGA | p.Glu578fs |
| 2:25244272:TTCC: | NA | inframe_indel | c.1731_1733delGG | p.Glu578del |
| T | | | A | |
| 2:25244273:T:C | NA | missense | c.1733A > G | p.Glu578Gly |
| 2:25244273:T:TC | NA | frameshift | c.1732dupG | p.Glu578fs |
| 2:25244273:TC:T | NA | frameshift | c.1732delG | p.Glu578fs |
| 2:25244273:TCC:T | NA | frameshift | c.1731_1732delGG | p.Glu578fs |
| 2:25244273:TCCT | NA | inframe_indel | c.1721_1732delCA | p.Ala574_Lys577 |
| TAATGGCTG:T | | | GCCATTAAGG | del |
| 2:25244274:C:A | NA | stop_gained | c.1732G > T | p.Glu578* |
| 2:25244275:C:CT | NA | frameshift | c.1730dupA | p.Glu578fs |
| 2:25244275:CTTA | NA | frameshift | c.1727_1730delTT | p.Ile576fs |
| A:C | | | AA | |
| 2:25244277:T:TA | NA | frameshift | c.1728dupT | p.Lys577fs |
| 2:25244277:TA:T | NA | frameshift | c.1728delT | p.Lys577fs |
| 2:25244278:AATG | NA | inframe_indel | c.1719_1727delGG | p.Gln573_Ile576 |
| GCTGCC:A | | | CAGCCAT | delinsHis |
| 2:25244278:AATG | NA | frameshift | c.1706_1727delCG | p.Pro569fs |
| GCTGCCTGGGCA | | | GGGGCTGCCCAGG | |
| GCCCCCG:A | | | CAGCCAT | |
| 2:25244279:A:C | rs1247716811 | missense | c.1727T > G | p.Ile576Ser |
| 2:25244279:A:G | NA | missense | c.1727T > C | p.Ile576Thr |
| 2:25244279:A:T | NA | missense | c.1727T > A | p.Ile576Asn |
| 2:25244279:AT:A | NA | frameshift | c.1726delA | p.Ile576fs |
| 2:25244279:ATG | NA | frameshift | c.1719_1726delGG | p.Gln573fs |
| GCTGCC:A | | | CAGCCA | |
| 2:25244279:ATG | NA | frameshift | c.1711_1726delGC | p.Ala571fs |
| GCTGCCTGGGCA | | | TGCCCAGGCAGCCA | |
| GC:A | | | | |
| 2:25244280:T:C | NA | missense | c.1726A > G | p.Ile576Val |
| 2:25244280:T:G | NA | missense | c.1726A > C | p.Ile576Leu |
| 2:25244280:T:TA | NA | frameshift | c.1725_1726insTT | p.Ile576fs |
| A | | | | |
| 2:25244280:TG:T | NA | frameshift | c.1725delC | p.Ile576fs |
| 2:25244280:TGGC | NA | frameshift | c.1709_1725delGG | p.Gly570fs |
| TGCCTGGGCAGCC | | | GCTGCCCAGGCAGC | |
| C:T | | | C | |
| 2:25244280:TGGC | rs770302280 | frameshift | c.1707_1725delGG | p.Gly570fs |
| TGCCTGGGCAGCC | | | GGGCTGCCCAGGC | |
| CCC:T | | | AGCC | |
| 2:25244281:GGCT | NA | inframe_indel | c.1719_1724delGG | p.Gln573_Ala575 |
| GCC:G | | | CAGC | delinsHis |
| 2:25244281:GGCT | NA | inframe_indel | c.1713_1724delTG | p.Ala572_Ala575 |
| GCCTGGGCA:G | | | CCCAGGCAGC | del |
| 2:25244281:GGCT | NA | inframe_indel | c.1707_1724delGG | p.Gly570_Ala575 |
| GCCTGGGCAGCC | | | GGGCTGCCCAGGC | del |
| CCC:G | | | AGC | |
| 2:25244282:G:A | NA | missense | c.1724C > T | p.Ala575Val |
| 2:25244282:GCTG | NA | frameshift | c.1711_1723delGC | p.Ala571fs |
| CCTGGGCAGC:G | | | TGCCCAGGCAG | |
| 2:25244282:GCTG | NA | frameshift | c.1710_1723delGG | p.Ala571fs |
| CCTGGGCAGCCIG | | | CTGCCCAGGCAG | |
| 2:25244282:GCTG | NA | inframe_indel | c.1709_1723delGG | p.Gly570_Ala574 |
| CCTGGGCAGCCC: | | | GCTGCCCAGGCAG | del |
| G | | | | |
| 2:25244282:GCTG | NA | frameshift | c.1698_1723delGG | p.Leu566fs |
| CCTGGGCAGCCCC | | | TGGGGCCGGGGGC | |
| CGGCCCCACC:G | | | TGCCCAGGCAG | |
| 2:25244283:C:G | NA | missense | c.1723G > C | p.Ala575Pro |
| 2:25244283:CT:C | NA | frameshift | c.1722delA | p.Ala575fs |
| 2:25244284:TG:T | NA | frameshift | c.1721delC | p.Ala574fs |
| 2:25244284:TGCC | rs773643031 | inframe_indel | c.1713_1721delTG | p.Ala572_Ala574 |
| TGGGCA:T | | | CCCAGGC | del |
| 2:25244284:TGCC | NA | inframe_indel | c.1710_1721delGG | p.Ala571_Ala574 |
| TGGGCAGCC:T | | | CTGCCCAGGC | del |
| 2:25244284:TGCC | NA | inframe_indel | c.1707_1721delGG | p.Gly570_Ala574 |
| TGGGCAGCCCCC: | | | GGGCTGCCCAGGC | del |
| T | | | | |
| 2:25244285:GC:G | NA | frameshift | c.1720delG | p.Ala574fs |
| 2:25244285:GCCT | NA | frameshift | c.1711_1720delGC | p.Ala571fs |
| GGGCAGC:G | | | TGCCCAGG | |
| 2:25244285:GCCT | NA | frameshift | c.1710_1720delGG | p.Ala571fs |
| GGGCAGCC:G | | | CTGCCCAGG | |
| 2:25244286:C:T | NA | missense | c.1720G > A | p.Ala574Thr |
| 2:25244286:CCTG | NA | frameshift | c.1712_1719delCT | p.Ala571fs |
| GGCAG:C | | | GCCCAG | |
| 2:25244286:CCTG | NA | frameshift | c.1706_1719delCG | p.Pro569fs |
| GGCAGCCCCCG:C | | | GGGGCTGCCCAG | |
| 2:25244287:C:A | | missense | c.1719G > T | p.Gln573His |
| 2:25244287:C:CC | NA | frameshift | c.1718_1719insTG | p.Gln573fs |
| A | | | | |
| 2:25244287:CTGG: | NA | inframe_indel | c.1716_1718delCC | p.Gln573del |
| C | | | A | |
| 2:25244287:CTGG | rs763053459 | frameshift | c.1712_1718delCT | p.Ala571fs |
| GCAG:C | | | GCCCA | |
| 2:25244288:T:C | NA | missense | c.1718A > G | p.Gln573Arg |
| 2:25244288:TG:T | NA | frameshift | c.1717delC | p.Gln573fs |
| 2:25244288:TGG | NA | frameshift | c.1708_1717delGG | p.Gly570fs |
| GCAGCCCC:T | | | GGCTGCCC | |
| 2:25244289:G:A | rs902965419 | stop_gained | c.1717C > T | p.Gln573* |
| 2:25244289:G:GG | NA | frameshift | c.1716_1717insTG | p.Gln573fs |
| GCAGCCCCA | | | GGGCTGCC | |
| 2:25244289:GGG | NA | frameshift | c.1710_1716delGG | p.Ala571fs |
| CAGCC:G | | | CTGCC | |
| 2:25244289:GGG | NA | frameshift | c.1707_1716delGG | p.Gly570fs |
| CAGCCCCC:G | | | GGGCTGCC | |
| 2:25244290:G:GG | NA | inframe_indel | c.1713_1715dupTG | p.Ala572dup |
| CA | | | C | |
| 2:25244290:GGC: | NA | frameshift | c.1714_1715delGC | p.Ala572fs |
| G | | | | |
| 2:25244290:GGC | rs1351573938 | inframe_indel | c.1713_1715delTG | p.Ala572del |
| A:G | | | C | |
| 2:25244290:GGC | NA | frameshift | c.1711_1715delGC | p.Ala571fs |
| AGC:G | | | TGC | |
| 2:25244290:GGC | NA | frameshift | c.1708_1715delGG | p.Gly570fs |
| AGCCCC:G | | | GGCTGC | |
| 2:25244291:G:T | NA | missense | c.1715C > A | p.Ala572Asp |
| 2:25244291:GCA | NA | inframe_indel | c.1709_1714delGG | p.Gly570_Ala571 |
| GCCC:G | | | GCTG | del |
| 2:25244292:C:T | NA | missense | c.1714G > A | p.Ala572Thr |
| 2:25244292:CA:C | NA | frameshift | c.1713delT | p.Ala572fs |
| 2:25244293:AG:A | NA | frameshift | c.1712delC | p.Ala571fs |
| 2:25244294:G:GC | | frameshift | c.1711dupG | p.Ala571fs |
| 2:25244294:G:T | NA | missense | c.1712C > A | p.Ala571Asp |
| 2:25244294:GCG | | frameshift | c.1711delG | p.Ala571fs |
| 2:25244295:c.G | NA | missense | c.1711G > C | p.Ala571Pro |
| 2:25244295:C:T | NA | missense | c.1711G > A | p.Ala571Thr |
| 2:25244297:C:A | NA | missense | c.1709G > T | p.Gly570Val |
| 2:25244297:C:G | NA | missense | c.1709G > C | p.Gly570Ala |
| 2:25244297:C:T | NA | missense | c.1709G > A | p.Gly570Glu |
| 2:25244298:C:T | NA | missense | c.1708G > A | p.Gly570Arg |
| 2:25244300:G:A | rs772310511 | missense | c.1706C > T | p.Pro569Leu |
| 2:25244300:G:C | NA | missense | c.1706C > G | p.Pro569Arg |
| 2:25244301:G:GC | NA | frameshift | c.1704_1705insTG | p.Pro569fs |
| A | | | | |
| 2:25244301:GC:G | NA | frameshift | c.1704delG | p.Pro569fs |
| 2:25244301:GCC: | NA | frameshift | c.1703_1704delGG | p.Gly568fs |
| G | | | | |
| 2:25244301:GCCC | NA | frameshift | c.1682_1704delAG | p.Glu561fs |
| CACCAAGAGGTCC | | | TGTGTGGACCTCTT | |
| ACACACT:G | | | GGTGGGG | |
| 2:25244302:CCCC | NA | frameshift | c.1700_1703delTG | p.Va1567fs |
| A:C | | | GG | |
| 2:25244303:C:T | NA | missense | c.1703G > A | p.Gly568Glu |
| 2:25244303:CCCA: | rs1361235617 | inframe_indel | c.1700_1702delTG | p.Va1567del |
| C | | | G | |
| 2:25244304:CCAC | NA | frameshift | c.1688_1701delTG | p.Va1563fs |
| CAAGAGGTCCA:C | | | GACCTCTTGGTG | |
| 2:25244305:CA:C | NA | frameshift | c.1700delT | p.Va1567fs |
| 2:25244306:A:AC | NA | frameshift | c.1699dupG | p.Va1567fs |
| 2:25244306:AC:A | NA | frameshift | c.1699delG | p.Va1567fs |
| 2:25244307:CCAA | NA | frameshift | c.1695_1698delCTT | p.Leu566fs |
| G:C | | | G | |
| 2:25244308:C:CA | NA | frameshift | c.1694_1697dupTC | p.Leu566fs |
| AGA | | | TT | |
| 2:25244308:CCC | NA | frameshift | c.1697_1698insTG | p.Leu566fs |
| A | | | | |
| 2:25244308:CA:C | NA | frameshift | c.1697delT | p.Leu566fs |
| 2:25244309:A:T | NA | stop_gained | c.1697T > A | p.Leu566* |
| 2:25244311:GA:G | NA | frameshift | c.1694delT | p.Leu565fs |
| 2:25244312:A:G | rs1040053651 | missense | c.1694T > C | p.Leu565Pro |
| 2:25244312:AG:A | rs1303060010 | frameshift | c.1693delC | p.Leu565fs |
| 2:25244312:AGG | NA | frameshift | c.1690_1693delGA | p.Asp564fs |
| TC:A | | | CC | |
| 2:25244314:G:GT | NA | frameshift | c.1687_1691dupGT | p.Asp564fs |
| CCAC | | | GGA | |
| 2:25244314:GT:G | NA | frameshift | c.1691delA | p.Asp564fs |
| 2:25244315:T:TC | NA | frameshift | c.1690dupG | p.Asp564fs |
| 2:25244315:TC:T | NA | frameshift | c.1690delG | p.Asp564fs |
| 2:25244315:TCC:T | NA | frameshift | c.1689_1690delGG | p.Asp564fs |
| 2:25244315:TCCA | NA | inframe_indel | c.1679_1690delTG | p.Va1560_Va1563 |
| CACACTCCA:T | | | GAGTGTGTGG | del |
| 2:25244315:TCCA | NA | frameshift | c.1669_1690delTG | p.Cys557fs |
| CACACTCCACGCA | | | CTTTTGCGTGGAGT | |
| AAAGCA:T | | | GTGTGG | |
| 2:25244316:C:CC | NA | frameshift | c.1688_1689dupTG | p.Asp564fs |
| A | | | | |
| 2:25244316:C:CC | NA | frameshift | c.1686_1689dupTG | p.Asp564fs |
| ACA | | | TG | |
| 2:25244316:CCA: | NA | frameshift | c.1688_1689delTG | p.Va1563fs |
| C | | | | |
| 2:25244317:C:CA | NA | frameshift | c.1688dupT | p.Asp564fs |
| 2:25244318:A:AC | NA | frameshift | c.1681_1687dupG | p.Va1563fs |
| ACACTC | | | AGTGTG | |
| 2:25244318:A:T | NA | missense | c.1688T > A | p.Va1563Glu |
| 2:25244318:AC:A | NA | frameshift | c.1687delG | p.Va1563fs |
| 2:25244318:ACAC | NA | frameshift | c.1677_1687delCG | p.Va1560fs |
| ACTCCACG:A | | | TGGAGTGTG | |
| 2:25244319:C:T | rs1299203502 | missense | c.1687G > A | p.Va1563Met |
| 2:25244319:CA:C | NA | frameshift | c.1686delT | p.Cys562fs |
| 2:25244320:ACAC | NA | frameshift | c.1678_1685delGT | p.Va1560fs |
| TCCAC:A | | | GGAGTG | |
| 2:25244321:C:A | rs1218552501 | missense | c.1685G > T | p.Cys562Phe |
| 2:25244321:C:G | NA | missense | c.1685G > C | p.Cys562Ser |
| 2:25244321:C:T | rs1218552501 | missense | c.1685G > A | p.Cys562Tyr |
| 2:25244321:CA:C | NA | frameshift | c.1684delT | p.Cys562fs |
| 2:25244322:A:G | rs1057520788 | missense | c.1684T > C | p.Cys562Arg |
| 2:25244322:AC:A | NA | frameshift | c.1683delG | p.Glu561fs |
| 2:25244322:ACTC | NA | frameshift | c.1671_1683delCTT | p.Phe558fs |
| CACGCAAAAG:A | | | TTGCGTGGAG | |
| 2:25244324:TC:T | NA | frameshift | c.1681delG | p.Glu561fs |
| 2:25244324:TCCA | NA | frameshift | c.1677_1681delCG | p.Cys559fs |
| CG:T | | | TGG | |
| 2:25244324:TCCA | NA | frameshift | c.1671_1681delCTT | p.Cys557fs |
| CGCAAAAG:T | | | TTGCGTGG | |
| 2:25244325:C:A | NA | stop_gained | c.1681G > T | p.Glu561* |
| 2:25244325:CCA: | NA | frameshift | c.1679_1680delTG | p.Va1560fs |
| C | | | | |
| 2:25244325:CCAC | NA | frameshift | c.1677_1680delCG | p.Cys559fs |
| G:C | | | TG | |
| 2:25244326:CA:C | NA | frameshift | c.1679delT | p.Va1560fs |
| 2:25244327:AC:A | NA | frameshift | c.1678delG | p.Va1560fs |
| 2:25244327:ACGC | NA | frameshift | c.1671_1678delCTT | p.Phe558fs |
| AAAAG:A | | | TTGCG | |
| 2:25244328:CG:C | NA | frameshift | c.1677delC | p.Cys559fs |
| 2:25244329:G:C | NA | missense | c.1677C > G | p.Cys559Trp |
| 2:25244329:G:T | NA | stop_gained | c.1677C > A | p.Cys559* |
| 2:25244329:GCA | NA | frameshift | c.1669_1676delTG | p.Cys557fs |
| AAAGCA:G | | | CHUG | |
| 2:25244329:GCA | NA | inframe_indel | c.1668_1676delGT | p.Arg556_Cys559 |
| AAAGCAC:G | | | GCTTTTG | delinsSer |
| 2:25244330:C:A | rs1313738991 | missense | c.1676G > T | p.Cys559Phe |
| 2:25244330:C:CA | NA | frameshift | c.1675dupT | p.Cys559fs |
| 2:25244330:C:T | NA | missense | c.1676G > A | p.Cys559Tyr |
| 2:25244330:CA:C | NA | frameshift | c.1675delT | p.Cys559fs |
| 2:25244330:CAA: | NA | frameshift | c.1674_1675delTT | p.Phe558fs |
| C | | | | |
| 2:25244331:A:G | NA | missense | c.1675T > C | p.Cys559Arg |
| 2:25244333:A:C | NA | missense | c.1673T > G | p.Phe558Cys |
| 2:25244333:AAG: | NA | frameshift | c.1671_1672delCT | p.Phe558fs |
| A | | | | |
| 2:25244335:G:T | NA | stop_gained | c.1671C > A | p.Cys557* |
| 2:25244336:C:T | NA | missense | c.1670G > A | p.Cys557Tyr |
| 2:25244336:CA:C | NA | frameshift | c.1669delT | p.Cys557fs |
| 2:25244336:CACC | NA | frameshift | c.1668- | p.Cys557fs |
| TGGAA:C | | | 6_1669delTTCCAG | |
| | | | GT | |
| 2:25244337:A:C | rs760791871 | missense | c.1669T > G | p.Cys557Gly |
| 2:25244337:A:G | NA | missense | c.1669T > C | p.Cys557Arg |
| 2:25244337:AC:A | rs1267683924 | frameshift | c.1668delG | p.Arg556fs |
| 2:25244337: ACC: | NA | frameshift | c.1668- | p.Arg556fs |
| A | | | 1_1668delGG | |
| 2:25244338:C:A | NA | missense | c.1668G > T | p.Arg556Ser |
| 2:25244338:C:G | NA | missense | c.1668G > C | p.Arg556Ser |
| 2:25244339:C:A | NA | splice_acceptor | c.1668-1G > T | |
| 2:25244339:C:G | NA | splice_acceptor | c.1668-1G > C | |
| 2:25244339:C:T | NA | splice_acceptor | c.1668-1G > A | |
| 2:25244339:CT:C | NA | splice_acceptor | c.1668-2delA | |
| 2:25244339:CTGG | NA | splice_acceptor | c.1668-6_1668- | |
| AA:C | | | 2delTTCCA | |
| 2:25244340:T:C | rs764270325 | splice_acceptor | c.1668-2A > G | |
| 2:25244340:T:G | NA | splice_acceptor | c.1668-2A > C | |
| 2:25244530:ACA | NA | splice_donor | c.1665_1667+9del | p.Cys555_Arg556 |
| GCCTCACCTG:A | | | CAGGTGAGGCTG | delinsTrp |
| 2:25244538:A:C | NA | splice_donor | c.1667+2T > G | |
| 2:25244538:A:G | NA | splice_donor | c.1667+2T > C | |
| 2:25244538:A:T | NA | splice_donor | c.1667+2T > A | |
| 2:25244539:C:A | rs776844136 | splice_donor | c.1667+1G > T | |
| 2:25244539:C:G | rs776844136 | splice_donor | c.1667+1G > C | |
| 2:25244539:C:T | rs776844136 | splice_donor | c.1667+1G > A | |
| 2:25244540:C:CT | NA | frameshift | c.1663_1666dupTG | p.Arg556fs |
| GCA | | | CA | |
| 2:25244540:C:G | rs1162120698 | missense | c.1667G > C | p.Arg556Thr |
| 2:25244540:CT:C | NA | frameshift | c.1666delA | p.Arg556fs |
| 2:25244541:T:A | NA | missense | c.1666A > T | p.Arg556Trp |
| 2:25244541:T:C | rs770311493 | missense | c.1666A > G | p.Arg556Gly |
| 2:25244542:G:T | NA | stop_gained | c.1665C > A | p.Cys555* |
| 2:25244543:C:CA | NA | frameshift | c.1663dupT | p.Cys555fs |
| 2:25244543:CA:C | NA | frameshift | c.1663delT | p.Cys555fs |
| 2:25244544:A:G | NA | missense | c.1663T > C | p.Cys555Arg |
| 2:25244545:G:T | NA | stop_gained | c.1662C > A | p.Cys554* |
| 2:25244546:CAG: | NA | frameshift | c.1659_1660delCT | p.Asn553fs |
| C | | | | |
| 2:25244547:A:C | NA | missense | c.1660T > G | p.Cys554Gly |
| 2:25244547:A:G | NA | missense | c.1660T > C | p.Cys554Arg |
| 2:25244547:AGT: | NA | frameshift | c.1658_1659delAC | p.Asn553fs |
| A | | | | |
| 2:25244547:AGTT | NA | frameshift | c.1656_1659delCA | p.Asn553fs |
| G:A | | | AC | |
| 2:25244548:G:C | NA | missense | c.1659C > G | p.Asn553Lys |
| 2:25244548:G:T | NA | missense | c.1659C > A | p.Asn553Lys |
| 2:25244548:GT:G | NA | frameshift | c.1658delA | p.Asn553fs |
| 2:25244548:GTT: | NA | frameshift | c.1657_1658delAA | p.Asn553fs |
| G | | | | |
| 2:25244549:T:TT | NA | frameshift | c.1657_1658insCA | p.Asn553fs |
| GTGTTG | | | ACACA | |
| 2:25244550:TG:T | NA | frameshift | c.1656delC | p.Asn552fs |
| 2:25244551:GT:G | rs1229047002 | frameshift | c.1655delA | p.Asn552fs |
| 2:25244551:GTTG | NA | frameshift | c.1652_1655delAC | p.Asn551fs |
| T:G | | | AA | |
| 2:25244554:G:GT | NA | frameshift | c.1652dupA | p.Asn551fs |
| 2:25244556:TTCC | NA | frameshift | c.1635_1650delGG | p.Va1546fs |
| GCACATGAGCACC | | | TGCTCATGTGCGGA | |
| T | | | | |
| 2:25244557:TC:T | NA | frameshift | c.1649delG | p.Gly550fs |
| 2:25244557:TCCG | NA | stop_gained | c.1647_1649delCG | p.Cys549_Gly550 |
| T | | | G | delinsTer |
| 2:25244557:TCCG | NA | frameshift | c.1637_1649delTG | p.Va1546fs |
| CACATGAGCA:T | | | CTCATGTGCGG | |
| 2:25244559:C:A | rs763336898 | stop_gained | c.1648G > T | p.Gly550* |
| 2:25244559:C:G | NA | missense | c.1648G > C | p.Gly550Arg |
| 2:25244559:C:T | rs763336898 | missense | c.1648G > A | p.Gly550Arg |
| 2:25244559:CGCA | NA | frameshift | c.1640_1647delTC | p.Leu547fs |
| CATGA:C | | | ATGTGC | |
| 2:25244560:G:C | NA | missense | c.1647C > G | p.Cys549Trp |
| 2:25244560:G:T | NA | stop_gained | c.1647C > A | p.Cys549* |
| 2:25244561:C:T | NA | missense | c.1646G > A | p.Cys549Tyr |
| 2:25244561:CA:C | NA | frameshift | c.1645delT | p.Cys549fs |
| 2:25244562:A:AC | rs1397787959 | frameshift | c.1644dupG | p.Cys549fs |
| 2:25244562:A:AC | NA | frameshift | c.1644_1645insCG | p.Cys549fs |
| G | | | | |
| 2:25244562:A:G | NA | missense | c.1645T > C | p.Cys549Arg |
| 2:25244563:C:A | NA | missense | c.1644G > T | p.Met548Ile |
| 2:25244563:C:T | rs1403215240 | missense | c.1644G > A | p.Met548Ile |
| 2:25244563:CAT: | rs1307594893 | frameshift | c.1642_1643delAT | p.Met548fs |
| C | | | | |
| 2:25244564:A:C | NA | missense | c.1643T > G | p.Met548Arg |
| 2:25244564:A:G | NA | missense | c.1643T > C | p.Met548Thr |
| 2:25244564:A:T | rs587777509 | missense | c.1643T > A | p.Met548Lys |
| 2:25244565:T:C | NA | missense | c.1642A > G | p.Met548Val |
| 2:25244566:GAG | NA | frameshift | c.1607_1640delAC | p.Tyr536fs |
| CACCTCACGGCCC | | | TGCACCATCTGCTG | |
| CCACAGCAGATG | | | TGGGGGCCGTGAG | |
| GTGCAGT:G | | | GTGCT | |
| 2:25244567:A:C | rs111848085 | missense | c.1640T > G | p.Leu547Arg |
| 2:25244567:A:G | rs111848085 | missense | c.1640T > C | p.Leu547Pro |
| 2:25244567:A:T | rs111848085 | missense | c.1640T > A | p.Leu547His |
| 2:25244568:G:A | rs759176128 | missense | c.1639C > T | p.Leu547Phe |
| 2:25244568:G:GA | NA | frameshift | c.1638_1639insT | p.Leu547fs |
| 2:25244570:AC:A | NA | frameshift | c.1636delG | p.Va1546fs |
| 2:25244573:T:TC | NA | frameshift | c.1603_1633dupTC | p.Glu545fs |
| ACGGCCCCCACAG | | | CTACTGCACCATCT | |
| CAGATGGTGCAG | | | GCTGTGGGGGCCG | |
| TAGGA | | | TG | |
| 2:25244573:TC:T | NA | frameshift | c.1633delG | p.Glu545fs |
| 2:25244574:C:A | NA | stop_gained | c.1633G > T | p.Glu545* |
| 2:25244574:C:CA | NA | frameshift | c.1632dupT | p.Glu545fs |
| 2:25244575:AC:A | NA | frameshift | c.1631delG | p.Arg544fs |
| 2:25244576:CG:C | NA | frameshift | c.1630delC | p.Arg544fs |
| 2:25244577:GGC | NA | frameshift | c.1625_1629delGG | p.Gly542fs |
| CCC:G | | | GGC | |
| 2:25244578:G:GC | rs1164367418 | frameshift | c.1628dupG | p.Arg544fs |
| 2:25244578:G:GC | NA | frameshift | c.1627_1628dupG | p.Arg544fs |
| C | | | G | |
| 2:25244578:GC:G | rs1392385564 | frameshift | c.1628delG | p.Gly543fs |
| 2:25244579:C:A | rs767226511 | missense | c.1628G > T | p.Gly543Val |
| 2:25244579:C:G | rs767226511 | missense | c.1628G > C | p.Gly543Ala |
| 2:25244579:C:T | rs767226511 | missense | c.1628G > A | p.Gly543Asp |
| 2:25244580:C:A | rs752222356 | missense | c.1627G > T | p.Gly543Cys |
| 2:25244580:C:T | NA | missense | c.1627G > A | p.Gly543Ser |
| 2:25244582:C:CC | NA | frameshift | c.1624_1625insCCC | p.Gly542fs |
| ACAGCAGATGGT | | | TACTGCACCATCTG | |
| GCAGTAGGG | | | CTGTG | |
| 2:25244584:A:T | NA | stop_gained | c.1623T > A | p.Cys541* |
| 2:25244585:CA:C | NA | frameshift | c.1621delT | p.Cys541fs |
| 2:25244586:A:AG | NA | frameshift | c.1620dupC | p.Cys541fs |
| 2:25244586:AGC | NA | frameshift | c.1610_1620delGC | p.Cys537fs |
| AGATGGTGC:A | | | ACCATCTGC | |
| 2:25244586:AGC | NA | frameshift | c.1605_1620delCT | p.Tyr536fs |
| AGATGGTGCAGT | | | ACTGCACCATCTGC | |
| AG:A | | | | |
| 2:25244587:G:T | NA | stop_gained | c.1620C > A | p.Cys540* |
| 2:25244588:C:CA | rs1438581688 | frameshift | c.1618dupT | p.Cys540fs |
| 2:25244588:CA:C | rs1366761466 | frameshift | c.1618delT | p.Cys540fs |
| 2:25244588:CAG | NA | frameshift | c.1615_1618delAT | p.Ile539fs |
| AT:C | | | CT | |
| 2:25244588:CAG | NA | frameshift | c.1608_1618delCT | p.Tyr536fs |
| ATGGTGCAG:C | | | GCACCATCT | |
| 2:25244589:AG:A | NA | frameshift | c.1617delC | p.Cys540fs |
| 2:25244589:AGAT | NA | frameshift | c.1614_1617delCA | p.Ile539fs |
| G:A | | | TC | |
| 2:25244590:GA:G | NA | frameshift | c.1616delT | p.Ile539fs |
| 2:25244592:T:TG | NA | frameshift | c.1614dupC | p.Ile539fs |
| 2:25244592:TG:T | rs1291735877 | frameshift | c.1614delC | p.Ile539fs |
| 2:25244592:TGG: | NA | frameshift | c.1613_1614delCC | p.Thr538fs |
| T | | | | |
| 2:25244593:G:GG | NA | frameshift | c.1612_1613dupAC | p.Ile539fs |
| T | | | | |
| 2:25244593:G:GT | NA | frameshift | c.1613_1614insGT | p.Ile539fs |
| GCAC | | | GCA | |
| 2:25244596:G:C | NA | missense | c.1611C > G | p.Cys537Trp |
| 2:25244596:G:GC | NA | frameshift | c.1609_1610dupTG | p.Thr538fs |
| A | | | | |
| 2:25244596:G:T | NA | stop_gained | c.1611C > A | p.Cys537* |
| 2:25244596:GC:G | NA | frameshift | c.1610delG | p.Cys537fs |
| 2:25244597:C:A | NA | missense | c.1610G > T | p.Cys537Phe |
| 2:25244597:C:T | NA | missense | c.1610G > A | p.Cys537Tyr |
| 2:25244598:A:C | NA | missense | c.1609T > G | p.Cys537Gly |
| 2:25244598:A:G | NA | missense | c.1609T > C | p.Cys537Arg |
| 2:25244598:A:T | NA | missense | c.1609T > A | p.Cys537Ser |
| 2:25244598:AG:A | NA | frameshift | c.1608delC | p.Cys537fs |
| 2:25244599:G:C | rs370376334 | stop_gained | c.1608C > G | p.Tyr536* |
| 2:25244599:G:GT | NA | frameshift | c.1607dupA | p.Tyr536fs |
| 2:25244599:G:T | NA | stop_gained | c.1608C > A | p.Tyr536* |
| 2:25244599:GT:G | rs759716976 | frameshift | c.1607delA | p.Tyr536fs |
| 2:25244600:T:TA | NA | frameshift | c.1603_1606dupTC | p.Tyr536fs |
| GGA | | | CT | |
| 2:25244600:TAG: | NA | frameshift | c.1605_1606delCT | p.Tyr536fs |
| T | | | | |
| 2:25244601:AG:A | rs767775447 | frameshift | c.1605delC | p.Tyr536fs |
| 2:25244601:AGG | NA | frameshift | c.1598_1605delAC | p.Tyr533fs |
| ACTGGT:A | | | CAGTCC | |
| 2:25244602:G:GG | NA | frameshift | c.1603_1604dupTC | p.Tyr536fs |
| A | | | | |
| 2:25244602:G:GG | NA | frameshift | c.1601_1604dupA | p.Tyr536fs |
| ACT | | | GTC | |
| 2:25244604:AC:A | NA | frameshift | c.1602delG | p.Gln534fs |
| 2:25244605:CTGG: | NA | stop_gained | c.1599_1601delCC | p.Tyr533_Gln534 |
| C | | | A | delinsTer |
| 2:25244606:TG:T | NA | frameshift | c.1600delC | p.Gln534fs |
| 2:25244607:G:A | rs1167876410 | stop_gained | c.1600C > T | p.Gln534* |
| 2:25244608:G:C | rs757057121 | stop_gained | c.1599C > G | p.Tyr533* |
| 2:25244608:G:GT | NA | frameshift | c.1576_1598dupTA | p.Gln534fs |
| AGCCGTCGTCGTC | | | CCAGTACGACGACG | |
| GTACTGGTA | | | ACGGCTA | |
| 2:25244608:G:T | rs757057121 | stop_gained | c.1599C > A | p.Tyr533* |
| 2:25244608:GT:G | NA | frameshift | c.1598delA | p.Tyr533fs |
| 2:25244609:TA:T | NA | frameshift | c.1597delT | p.Tyr533fs |
| 2:25244610:AG:A | NA | frameshift | c.1596delC | p.Tyr533fs |
| 2:25244612:C:A | NA | missense | c.1595G > T | p.Gly532Val |
| 2:25244613:C:CG | NA | frameshift | c.1592_1593dupAC | p.Gly532fs |
| T | | | | |
| 2:25244613:C:T | rs951361433 | missense | c.1594G > A | p.Gly532Ser |
| 2:25244613:CG:C | rs1329318931 | frameshift | c.1593delC | p.Asp531fs |
| 2:25244614:GT:G | NA | frameshift | c.1592delA | p.Asp531fs |
| 2:25244615:TC:T | NA | frameshift | c.1591delG | p.Asp531fs |
| 2:25244616:C:T | rs745721709 | missense | c.1591G > A | p.Asp531Asn |
| 2:25244616:CG:C | NA | frameshift | c.1590delC | p.Asp530fs |
| 2:25244617:G:GC | NA | frameshift | c.1589_1590insG | p.Asp530fs |
| 2:25244617:GTCG | NA | frameshift | c.1582_1589delTA | p.Tyr528fs |
| TCGTA:G | | | CGACGA | |
| 2:25244621:TCGT | NA | frameshift | c.1582_1585delTA | p.Tyr528fs |
| A:T | | | CG | |
| 2:25244621:TCGT | NA | frameshift | c.1567_1585delGA | p.Glu523fs |
| ACTGGTACGCACA | | | GTGTGCGTACCAGT | |
| CTC:T | | | ACG | |
| 2:25244622:C:CG | NA | frameshift | c.1584dupC | p.Asp529fs |
| 2:25244622:C:T | rs962805778 | missense | c.1585G > A | p.Asp529Asn |
| 2:25244623:G:C | NA | stop_gained | c.1584C > G | p.Tyr528* |
| 2:25244623:G:T | NA | stop_gained | c.1584C > A | p.Tyr528* |
| 2:25244624:T:TA | NA | frameshift | c.1576_1582dupTA | p.Tyr528fs |
| CTGGTA | | | CCAGT | |
| 2:25244624:TACT | NA | frameshift | c.1576_1582delTA | p.Tyr526fs |
| GGTA:T | | | CCAGT | |
| 2:25244625:A:AC | NA | frameshift | c.1581dupG | p.Tyr528fs |
| 2:25244625:AC:A | NA | frameshift | c.1581delG | p.Gln527fs |
| 2:25244625:ACTG | NA | frameshift | c.1578_1581delCC | p.Gln527fs |
| G:A | | | AG | |
| 2:25244626:C:A | NA | missense | c.1581G > T | p.Gln527His |
| 2:25244626:C:G | NA | missense | c.1581G > C | p.Gln527His |
| 2:25244626:CT:C | NA | frameshift | c.1580delA | p.Gln527fs |
| 2:25244627:T:G | rs773701621 | missense | c.1580A > C | p.Gln527Pro |
| 2:25244627:TG:T | NA | frameshift | c.1579delC | p.Gln527fs |
| 2:25244628:G:A | rs1372858208 | stop_gained | c.1579C > T | p.Gln527* |
| 2:25244628:GGT | NA | splice_acceptor | c.1555- | p.Asn519_Gln527 |
| ACGCACACTCCAG | | | 3_1578delTAGAAC | del |
| AAAGCAGTTCTA: | | | TGCTTTCTGGAGTG | |
| G | | | TGCGTAC | |
| 2:25244629:G:C | rs763384327 | stop_gained | c.1578C > G | p.Tyr526* |
| 2:25244629:G:T | NA | stop_gained | c.1578C > A | p.Tyr526* |
| 2:25244629:GTAC | NA | frameshift | c.1571_1577delGT | p.Cys524fs |
| GCAC:G | | | GCGTA | |
| 2:25244629:GTAC | NA | frameshift | c.1567_1577delGA | p.Glu523fs |
| GCACACTC:G | | | GTGTGCGTA | |
| 2:25244631:AC:A | NA | frameshift | c.1575delG | p.Tyr526fs |
| 2:25244633:GC:G | NA | frameshift | c.1573delG | p.Ala525fs |
| 2:25244633:GCA: | rs765267873 | frameshift | c.1572_1573delTG | p.Ala525fs |
| G | | | | |
| 2:25244634:C:CA | NA | frameshift | c.1572dupT | p.Ala525fs |
| 2:25244634:CACA | NA | frameshift | c.1563_1572delTCT | p.Phe521fs |
| CTCCAGA:C | | | GGAGTGT | |
| 2:25244635:A:T | NA | stop_gained | c.1572T > A | p.Cys524* |
| 2:25244637:AC:A | rs1238915745 | frameshift | c.1569delG | p.Glu523fs |
| 2:25244638:CT:C | NA | frameshift | c.1568delA | p.Glu523fs |
| 2:25244639:TC:T | NA | frameshift | c.1567delG | p.Glu523fs |
| 2:25244640:C:A | NA | stop_gained | c.1567G > T | p.Glu523* |
| 2:25244641:CA:C | NA | frameshift | c.1565delT | p.Leu522fs |
| 2:25244643:G:GA | NA | frameshift | c.1563dupT | p.Leu522fs |
| 2:25244643:GA:G | NA | frameshift | c.1563delT | p.Leu522fs |
| 2:25244647:G:GC | NA | frameshift | c.1558_1559dupTG | p.Phe521fs |
| A | | | | |
| 2:25244647:G:T | rs774696234 | stop_gained | c.1560C > A | p.Cys520* |
| 2:25244647:GC:G | NA | frameshift | c.1559delG | p.Cys520fs |
| 2:25244648:C:CA | NA | frameshift | c.1558dupT | p.Cys520fs |
| 2:25244648:CA:C | NA | frameshift | c.1558delT | p.Cys520fs |
| 2:25244648:CAGT | NA | frameshift | c.1555- | p.Asn519fs |
| TCTAGACAGCAGC | | | 24_1558delCTGAC | |
| GGGAAGGGTCAG: | | | CCTTCCCGCTGCTG | |
| C | | | TCTAGAACT | |
| 2:25244650:GT:G | NA | frameshift | c.1556delA | p.Asn519fs |
| 2:25244652:T:TC | NA | splice_acceptor | c.1555-1dupG | |
| 2:25244653:C:G | rs759936287 | splice_acceptor | c.1555-1G > C | |
| 2:25244653:C:T | NA | splice_acceptor | c.1555-1G > A | |
| 2:25244654:T:A | NA | splice_acceptor | c.1555-2A > T | |
| 2:25244654:T:C | rs767136883 | splice_acceptor | c.1555-2A > G | |
| 2:25244654:T:G | rs767136883 | splice_acceptor | c.1555-2A > C | |
| 2:25245251:A:C | rs758228404 | splice_donor | c.1554+2T > G | |
| 2:25245251:A:G | NA | splice_donor | c.1554+2T > C | |
| 2:25245251:AC:A | NA | splice_donor | c.1554+1delG | |
| 2:25245252:C:A | NA | splice_donor | c.1554+1G > T | |
| 2:25245252:C:G | NA | splice_donor | c.1554+1G > C | |
| 2:25245252:C:T | rs766110518 | splice_donor | c.1554+1G > A | |
| 2:25245253:C:CTT | NA | frameshift | c.1547_1553dupAC | p.Asn519fs |
| GCAGT | | | TGCAA | |
| 2:25245255:T:A | NA | stop_gained | c.1552A > T | p.Lys518* |
| 2:25245255:T:TG | NA | frameshift | c.1551dupC | p.Lys518fs |
| 2:25245255:T:TG | NA | frameshift | c.1550_1551dupGC | p.Lys518fs |
| C | | | | |
| 2:25245255:TG:T | NA | frameshift | c.1551delC | p.Cys517fs |
| 2:25245256:G:T | NA | stop_gained | c.1551C > A | p.Cys517* |
| 2:25245256:GC:G | NA | frameshift | c.1550delG | p.Cys517fs |
| 2:25245258:AG:A | NA | frameshift | c.1548delC | p.Cys517fs |
| 2:25245259:GT:G | rs746528594 | frameshift | c.1547delA | p.Asn516fs |
| 2:25245261:TTTG | NA | frameshift | c.1538_1545delTG | p.Met513fs |
| GCACA:T | | | TGCCAA | |
| 2:25245263:T:TG | NA | frameshift | c.1519_1543dupCC | p.Gln515fs |
| GCACATTCCTCCA | | | CCTCTTCGTTGGAG | |
| ACGAAGAGGGG | | | GAATGTGCC | |
| 2:25245263:TG:T | NA | frameshift | c.1543delC | p.Gln515fs |
| 2:25245263:TGGC | NA | frameshift | c.1531_1543delGG | p.Gly511fs |
| ACATTCCTCC:T | | | AGGAATGTGCC | |
| 2:25245263:TGGC | NA | stop_gained | c.1526_1543delTC | p.Phe509_Gln515 |
| ACATTCCTCCAAC | | | GTTGGAGGAATGT | delinsTer |
| GA:T | | | GCC | |
| 2:25245264:G:A | rs1449820788 | stop_gained | c.1543C > T | p.Gln515* |
| 2:25245265:G:T | NA | stop_gained | c.1542C > A | p.Cys514* |
| 2:25245266:CA:C | | frameshift | c.1540delT | p.Cys514fs |
| 2:25245267:AC:A | NA | frameshift | c.1539delG | p.Met513fs |
| 2:25245269:A:AT | rs780842310 | frameshift | c.1537dupA | p.Met513fs |
| 2:25245269:AT:A | NA | frameshift | c.1537delA | p.Met513fs |
| 2:25245269:ATTC | NA | frameshift | c.1524_1537delCTT | p.Phe509fs |
| CTCCAACGAAG:A | | | CGTTGGAGGAA | |
| 2:25245270:TTC:T | NA | frameshift | c.1535_1536delGA | p.Gly512fs |
| 2:25245271:T:TCC | NA | frameshift | c.1532_1535dupG | p.Met513fs |
| TC | | | AGG | |
| 2:25245272:C:CC | NA | frameshift | c.1498_1534dupCT | p.Gly512fs |
| TCCAACGAAGAG | | | CAATGTTACCCTGG | |
| GGGGTGTTCCAG | | | AACACCCCCTCTTC | |
| GGTAACATTGAG | | | GTTGGAG | |
| 2:25245274:TC:T | NA | frameshift | c.1532delG | p.Gly511fs |
| 2:25245276:C:A | NA | stop_gained | c.1531G > T | p.Gly511* |
| 2:25245276:C:CA | NA | frameshift | c.1530dupT | p.Gly511fs |
| 2:25245276:CA:C | NA | frameshift | c.1530delT | p.Gly511fs |
| 2:25245276:CAAC | NA | frameshift | c.1527_1530delCG | p.Phe509fs |
| G:C | | | TT | |
| 2:25245278:AC:A | NA | frameshift | c.1528delG | p.Va1510fs |
| 2:25245278:ACG: | NA | frameshift | c.1527_1528delCG | p.Va1510fs |
| A | | | | |
| 2:25245279:CG:C | NA | frameshift | c.1527delC | p.Phe509fs |
| 2:25245281:AAG: | NA | frameshift | c.1524_1525delCT | p.Phe509fs |
| A | | | | |
| 2:25245283:GA:G | NA | frameshift | c.1523delT | p.Leu508fs |
| 2:25245284:A:AG | NA | frameshift | c.1522dupC | p.Leu508fs |
| 2:25245284:A:G | rs1056677010 | missense | c.1523T > C | p.Leu508Pro |
| 2:25245284:AG:A | rs1162765172 | frameshift | c.1522delC | p.Leu508fs |
| 2:25245288:G:GG | NA | frameshift | c.1514_1518dupAA | p.Pro507fs |
| TGTT | | | CAC | |
| 2:25245289:GT:G | NA | frameshift | c.1517delA | p.His506fs |
| 2:25245290:T:TC | NA | frameshift | c.1516_1517insG | p.His506fs |
| 2:25245290:T:TCC | NA | frameshift | c.1516_1517insGG | p.His506fs |
| 2:25245290:TGTT | NA | frameshift | c.1512_1516delGG | p.Glu505fs |
| CC:T | | | AAC | |
| 2:25245290:TGTT | NA | frameshift | c.1510_1516delCT | p.Leu504fs |
| CCAG:T | | | GGAAC | |
| 2:25245291:GT:G | NA | frameshift | c.1515delA | p.Glu505fs |
| 2:25245293:T:TCC | NA | frameshift | c.1492_1513dupG | p.Glu505fs |
| AGGGTAACATTGA | | | GGAGCCTCAATGTT | |
| GGCTCCC | | | ACCCTGG | |
| 2:25245293:TC:T | NA | frameshift | c.1513delG | p.Glu505fs |
| 2:25245293:TCCA | NA | frameshift | c.1489_1513delTG | p.Cys497fs |
| GGGTAACATTGA | | | TGGGAGCCTCAATG | |
| GGCTCCCACA:T | | | TTACCCTGG | |
| 2:25245294:C:A | rs373860660 | stop_gained | c.1513G > T | p.Glu505* |
| 2:25245294:C:CC | NA | frameshift | c.1487_1512dupCC | p.Glu505fs |
| AGGGTAACATTGA | | | TGTGGGAGCCTCAA | |
| GGCTCCCACAGG | | | TGTTACCCTG | |
| 2:25245295:CA:C | NA | frameshift | c.1511delT | p.Leu504fs |
| 2:25245295:CAG | NA | frameshift | c.1508_1511delCC | p.Thr503fs |
| GG:C | | | CT | |
| 2:25245296:AG:A | NA | frameshift | c.1510delC | p.Leu504fs |
| 2:25245298:G:GG | NA | frameshift | c.1507_1508dupAC | p.Leu504fs |
| T | | | | |
| 2:25245298:G:GG | NA | frameshift | c.1505_1508dupTT | p.Leu504fs |
| TAA | | | AC | |
| 2:25245298:GGT: | NA | frameshift | c.1507_1508delAC | p.Thr503fs |
| G | | | | |
| 2:25245299:G:GT | NA | frameshift | c.1507dupA | p.Thr503fs |
| 2:25245299:GT:G | NA | frameshift | c.1507delA | p.Thr503fs |
| 2:25245300:T:TA | NA | frameshift | c.1506dupT | p.Thr503fs |
| 2:25245300:TA:T | NA | frameshift | c.1506delT | p.Thr503fs |
| 2:25245300:TAA: | NA | frameshift | c.1505_1506delTT | p.Va1502fs |
| T | | | | |
| 2:25245302:AC:A | NA | frameshift | c.1504delG | p.Va1502fs |
| 2:25245303:C:CA | NA | frameshift | c.1503dupT | p.Va1502fs |
| 2:25245303:CA:C | NA | frameshift | c.1503delT | p.Asn501fs |
| 2:25245303:CAT: | NA | frameshift | c.1502_1503delAT | p.Asn501fs |
| C | | | | |
| 2:25245304:A:AT | NA | frameshift | c.1501_1502dupAA | p.Asn501fs |
| T | | | | |
| 2:25245304:AT:A | NA | frameshift | c.1502delA | p.Asn501fs |
| 2:25245304:ATT: | NA | frameshift | c.1501_1502delAA | p.Asn501fs |
| A | | | | |
| 2:25245304:ATTG | NA | frameshift | c.1498_1502delCT | p.Leu500fs |
| AG:A | | | CAA | |
| 2:25245306:TGA: | NA | frameshift | c.1499_1500delTC | p.Leu500fs |
| T | | | | |
| 2:25245308:AG:A | NA | frameshift | c.1498delC | p.Leu500fs |
| 2:25245309:G:GG | NA | frameshift | c.1494_1497dupG | p.Leu500fs |
| CTC | | | AGC | |
| 2:25245311:CT:C | NA | frameshift | c.1495delA | p.Ser499fs |
| 2:25245312:TC:T | NA | frameshift | c.1494delG | p.Ser499fs |
| 2:25245314:C:CC | NA | frameshift | c.1491_1492dupTG | p.Gly498fs |
| A | | | | |
| 2:25245315:C:CA | NA | frameshift | c.1491dupT | p.Gly498fs |
| 2:25245316:A:C | NA | missense | c.1491T > G | p.Cys497Trp |
| 2:25245316:AC:A | NA | frameshift | c.1490delG | p.Cys497fs |
| 2:25245317:C:A | rs779323387 | missense | c.1490G > T | p.Cys497Phe |
| 2:25245317:C:G | NA | missense | c.1490G > C | p.Cys497Ser |
| 2:25245317:C:T | rs779323387 | missense | c.1490G > A | p.Cys497Tyr |
| 2:25245317:CA:C | NA | frameshift | c.1489delT | p.Cys497fs |
| 2:25245317:CAG | NA | splice_acceptor | c.1475- | p.Asp492_Cys497 |
| GAGATGCAGATG | | | 6_1489delTTCCAG | delinsGly |
| TCTGGAA:C | | | ACATCTGCATCTCCT | |
| 2:25245318:A:G | NA | missense | c.1489T > C | p.Cys497Arg |
| 2:25245318:A:T | NA | missense | c.1489T > A | p.Cys497Ser |
| 2:25245318:AG:A | NA | frameshift | c.1488delC | p.Cys497fs |
| 2:25245320:GA:G | NA | frameshift | c.1486delT | p.Ser496fs |
| 2:25245321:AG:A | NA | frameshift | c.1485delC | p.Ser496fs |
| 2:25245325:G:C | rs1224606813 | missense | c.1482C > G | p.Cys494Trp |
| 2:25245325:G:T | NA | stop_gained | c.1482C > A | p.Cys494* |
| 2:25245326:C:A | NA | missense | c.1481G > T | p.Cys494Phe |
| 2:25245326:C:G | rs1240736156 | missense | c.1481G > C | p.Cys494Ser |
| 2:25245326:C:T | NA | missense | c.1481G > A | p.Cys494Tyr |
| 2:25245327:A:C | NA | missense | c.1480T > G | p.Cys494Gly |
| 2:25245327:A:G | NA | missense | c.1480T > C | p.Cys494Arg |
| 2:25245327:A:T | NA | missense | c.1480T > A | p.Cys494Ser |
| 2:25245327:AG:A | NA | frameshift | c.1479delC | p.Cys494fs |
| 2:25245328:GA:G | NA | frameshift | c.1478delT | p.Ile493fs |
| 2:25245329:AT:A | NA | frameshift | c.1477delA | p.Ile493fs |
| 2:25245330:TG:T | NA | frameshift | c.1476delC | p.Asp492fs |
| 2:25245330:TGTC | NA | frameshift | c.1475- | p.Ile493fs |
| TGGAAAGCA:T | | | 10_1476delTGCTTT | |
| | | | CCAGAC | |
| 2:25245331:GT:G | NA | frameshift | c.1475delA | p.Asp492fs |
| 2:25245333:C:G | rs746402248 | splice_acceptor | c.1475-1G > C | |
| 2:25245333:C:T | NA | splice_acceptor | c.1475-1G > A | |
| 2:25245333:CT:C | NA | splice_acceptor | c.1475-2delA | |
| 2:25245333:CTGG | NA | splice_acceptor | c.1475-21_1475- | |
| AAAGCAGAGGGA | | | 2delTCCCCTCCCTCT | |
| GGGGA:C | | | GCTTTCCA | |
| 2:25246010:ACAA | NA | splice_donor | c.1472_1474+9del | p.Glu491del |
| ACTTACCCT:A | | | AGGGTAAGTTTG | |
| 2:25246015:CTTA | NA | frameshift | c.1467_1474+4del | p.Asn489fs |
| CCCTCAATG:C | | | CATTGAGGGTAA | |
| 2:25246018:A:AC | NA | splice_donor | c.1474+ldupG | |
| 2:25246018:A:C | NA | splice_donor | c.1474+2T > G | |
| 2:25246018:AC:A | rs1200077617 | splice_donor | c.1474+ldelG | |
| 2:25246018:ACCC | NA | splice_donor | c.1472_1474+ldel | p.Glu491del |
| T:A | | | AGGG | |
| 2:25246019:C:A | NA | splice_donor | c.1474+1G > T | |
| 2:25246019:C:G | NA | splice_donor | c.1474+1G > C | |
| 2:25246019:C:T | rs761608224 | splice_donor | c.1474+1G > A | |
| 2:25246022:T:TC | NA | frameshift | c.1470_1471dupTG | p.Glu491fs |
| A | | | | |
| 2:25246022:TC:T | NA | frameshift | c.1471delG | p.Glu491fs |
| 2:25246022:TCAA | NA | frameshift | c.1461_1471delCC | p.Cys487fs |
| TGTTCCGG:T | | | GGAACATTG | |
| 2:25246023:C:A | NA | stop_gained | c.1471G > T | p.Glu491* |
| 2:25246023:C:CA | NA | frameshift | c.1470dupT | p.Glu491fs |
| 2:25246023:CA:C | NA | frameshift | c.1470delT | p.Ile490fs |
| 2:25246024:A:AA | NA | frameshift | c.1469_1470insGC | p.Ile490fs |
| TGGTGCGGC | | | CGCACCAT | |
| 2:25246025:A:AT | NA | frameshift | c.1455_1468dupG | p.Ile490fs |
| GTTCCGGCACTTC | | | AAGTGCCGGAACA | |
| 2:25246026:TG:T | NA | frameshift | c.1467delC | p.Asn489fs |
| 2:25246027:GT:G | NA | frameshift | c.1466delA | p.Asn489fs |
| 2:25246028:TTCC | NA | frameshift | c.1455_1465delGA | p.Lys486fs |
| GGCACTTC:T | | | AGTGCCGGA | |
| 2:25246029:T:TC | NA | frameshift | c.1464dupG | p.Asn489fs |
| 2:25246029:TC:T | | frameshift | c.1464delG | p.Asn489fs |
| 2:25246030:CCG | NA | frameshift | c.1459_1463delTG | p.Cys487fs |
| GCA:C | | | CCG | |
| 2:25246031:C:CG | NA | frameshift | c.1462dupC | p.Arg488fs |
| 2:25246033:G:T | NA | stop_gained | c.1461C > A | p.Cys487* |
| 2:25246033:GC:G | NA | frameshift | c.1460delG | p.Cys487fs |
| 2:25246034:CA:C | NA | frameshift | c.1459delT | p.Cys487fs |
| 2:25246035:AC:A | NA | frameshift | c.1458delG | p.Lys486fs |
| 2:25246035:ACTT | NA | frameshift | c.1454_1458delAG | p.Gln485fs |
| CT:A | | | AAG | |
| 2:25246036:CT:C | NA | frameshift | c.1457delA | p.Lys486fs |
| 2:25246037:TTC:T | NA | frameshift | c.1455_1456delGA | p.Lys486fs |
| 2:25246038:T:A | NA | stop_gained | c.1456A > T | p.Lys486* |
| 2:25246038:TC:T | NA | frameshift | c.1455delG | p.Lys486fs |
| 2:25246040:TGCC | NA | frameshift | c.1443_1453delCG | p.Tyr481fs |
| GCACCTCG:T | | | AGGTGCGGC | |
| 2:25246041:G:A | NA | stop_gained | c.1453C > T | p.Gln485* |
| 2:25246041:GC:G | NA | frameshift | c.1452delG | p.Gln485fs |
| 2:25246042:C:CC | rs1131691760 | frameshift | c.1450_1451dupCG | p.Gln485fs |
| G | | | | |
| 2:25246042:CCG: | NA | frameshift | c.1450_1451delCG | p.Arg484fs |
| C | | | | |
| 2:25246044:G:GC | NA | frameshift | c.1449dupG | p.Arg484fs |
| 2:25246044:G:GC | NA | frameshift | c.1448_1449dupTG | p.Arg484fs |
| A | | | | |
| 2:25246045:CACC | NA | frameshift | c.1442_1448delAC | p.Tyr481fs |
| TCGT:C | | | GAG GT | |
| 2:25246046:AC:A | NA | frameshift | c.1447delG | p.Va1483fs |
| 2:25246046:ACC: | NA | frameshift | c.1446_1447delGG | p.Glu482fs |
| A | | | | |
| 2:25246047:CCTC | NA | frameshift | c.1430_1446delAG | p.Glu477fs |
| GTACACCAGCCGC | | | CGGCTGGTGTACGA | |
| T:C | | | G | |
| 2:25246050:C:A | rs769071767 | stop_gained | c.1444G > T | p.Glu482* |
| 2:25246050:CG:C | NA | frameshift | c.1443delC | p.Tyr481fs |
| 2:25246051:G:C | rs145465364 | stop_gained | c.1443C > G | p.Tyr481* |
| 2:25246051:G:T | NA | stop_gained | c.1443C > A | p.Tyr481* |
| 2:25246052:T:TA | rs1273047372 | frameshift | c.1441dupT | p.Tyr481fs |
| 2:25246052:TAC: | NA | frameshift | c.1440_1441delGT | p.Tyr481fs |
| T | | | | |
| 2:25246054:CACC | NA | frameshift | c.1436_1439delTG | p.Leu479fs |
| A:C | | | GT | |
| 2:25246055:A:AC | NA | frameshift | c.1438dupG | p.Va1480fs |
| 2:25246057:CA:C | NA | frameshift | c.1436delT | p.Leu479fs |
| 2:25246058:AG:A | NA | frameshift | c.1435delC | p.Leu479fs |
| 2:25246058:AGCC | NA | frameshift | c.1432_1435delCG | p.Arg478fs |
| G:A | | | GC | |
| 2:25246058:AGCC | NA | splice_acceptor | c.1430- | p.Glu477_Leu479 |
| GCTCTGCAAG:A | | | 7_1435delCTTGCA | delinsVal |
| | | | GAGCGGC | |
| 2:25246059:GC:G | NA | frameshift | c.1434delG | p.Leu479fs |
| 2:25246061:CG:C | NA | frameshift | c.1432delC | p.Arg478fs |
| 2:25246061:CGCT | NA | splice_acceptor | c.1430- | p.Glu477_Arg478 |
| CTGCAAGGGGAG | | | 19_1432delAGCTC | delinsGly |
| GAGAGCT:C | | | TCCTCCCCTTGCAG | |
| | | | AGC | |
| 2:25246062:G:GC | NA | frameshift | c.1430_1431dupA | p.Arg478fs |
| T | | | G | |
| 2:25246062:G:GC | NA | frameshift | c.1430- | p.Leu479fs |
| TCT | | | 2_1431dupAGAG | |
| 2:25246062:GC:G | NA | frameshift | c.1431delG | p.Glu477fs |
| 2:25246062:GCTC: | NA | frameshift | c.1430- | p.Glu477fs |
| G | | | 1_1431delGAG | |
| 2:25246064:T:TC | NA | splice_acceptor | c.1430-1dupG | |
| 2:25246065:C:A | rs1363411956 | splice_acceptor | c.1430-1G > T | |
| 2:25246065:C:G | NA | splice_acceptor | c.1430-1G > C | |
| 2:25246065:C:T | NA | splice_acceptor | c.1430-1G > A | |
| 2:25246066:T:A | NA | splice_acceptor | c.1430-2A > T | |
| 2:25246066:T:C | rs1307856488 | splice_acceptor | c.1430-2A > G | |
| 2:25246066:T:G | NA | splice_acceptor | c.1430-2A > C | |
| 2:25246155:ACTA | NA | splice_donor | c.1424_1429+4del | p.Thr475_Glu477 |
| CTCTTTG:A | | | CAAGAGGTAG | delinsLys |
| 2:25246157:TA:T | NA | splice_donor | c.1429+2delT | |
| 2:25246158:A:C | NA | splice_donor | c.1429+2T > G | |
| 2:25246158:A:G | rs1207237998 | splice_donor | c.1429+2T > C | |
| 2:25246158:A:T | NA | splice_donor | c.1429+2T > A | |
| 2:25246158:AC:A | rs772195614 | splice_donor | c.1429+1delG | |
| 2:25246159:C:A | NA | splice_donor | c.1429+1G > T | |
| 2:25246159:C:CC | NA | frameshift | c.1428_1429dupA | p.Arg478fs |
| T | | | G | |
| 2:25246159:C:G | NA | splice_donor | c.1429+1G > C | |
| 2:25246159:C:T | rs774112139 | splice_donor | c.1429+1G > A | |
| 2:25246159:CCT: | NA | frameshift | c.1428_1429delAG | p.Glu477fs |
| C | | | | |
| 2:25246159:CCTC | NA | frameshift | c.1423_1429delAC | p.Thr475fs |
| TTGT:C | | | AAGAG | |
| 2:25246159:CCTC | NA | frameshift | c.1422_1429delCA | p.Thr475fs |
| TTGTG:C | | | CAAGAG | |
| 2:25246160:C:A | NA | stop_gained | c.1429G > T | p.Glu477* |
| 2:25246160:CT:C | NA | frameshift | c.1428delA | p.Glu477fs |
| 2:25246162:C:CT | NA | frameshift | c.1426dupA | p.Arg476fs |
| 2:25246162:CT:C | NA | frameshift | c.1426delA | p.Arg476fs |
| 2:25246163:T:A | rs772041976 | stop_gained | c.1426A > T | p.Arg476* |
| 2:25246163:TTG: | NA | frameshift | c.1424_1425delCA | p.Thr475fs |
| T | | | | |
| 2:25246164:TG:T | NA | frameshift | c.1424delC | p.Thr475fs |
| 2:25246165:G:GT | NA | frameshift | c.1419_1423dupGC | p.Thr475fs |
| GCGC | | | GCA | |
| 2:25246165:G:GT | NA | frameshift | c.1423_1424insAA | p.Thr475fs |
| GCGCTT | | | GCGCA | |
| 2:25246166:TGC: | NA | frameshift | c.1421_1422delGC | p.Arg474fs |
| T | | | | |
| 2:25246167:GC:G | NA | frameshift | c.1421delG | p.Arg474fs |
| 2:25246167:GCG | NA | frameshift | c.1418_1421delAG | p.Glu473fs |
| CT:G | | | CG | |
| 2:25246168:C:CT | NA | frameshift | c.1420_1421insA | p.Arg474fs |
| 2:25246168:CG:C | NA | frameshift | c.1420delC | p.Arg474fs |
| 2:25246169:G:GC | NA | frameshift | c.1416_1419dupTG | p.Arg474fs |
| TCA | | | AG | |
| 2:25246170:CT:C | NA | frameshift | c.1418delA | p.Glu473fs |
| 2:25246171:TC:T | NA | frameshift | c.1417delG | p.Glu473fs |
| 2:25246172:C:A | NA | stop_gained | c.1417G > T | p.Glu473* |
| 2:25246172:C:CA | NA | frameshift | c.1416dupT | p.Glu473fs |
| 2:25246172:CA:C | NA | frameshift | c.1416delT | p.Asp472fs |
| 2:25246172:CATC | NA | frameshift | c.1413_1416delTG | p.Ile471fs |
| A:C | | | AT | |
| 2:25246172:CATC | NA | frameshift | c.1409_1416delTT | p.Ile470fs |
| AATAA:C | | | ATTGAT | |
| 2:25246174:T:TA | NA | frameshift | c.1414_1415insT | p.Asp472fs |
| 2:25246174:TC:T | rs1347154070 | frameshift | c.1414delG | p.Asp472fs |
| 2:25246175:CA:C | NA | frameshift | c.1413delT | p.Ile471fs |
| 2:25246178:T:TA | NA | frameshift | c.1410dupT | p.Ile471fs |
| 2:25246178:T:TA | NA | frameshift | c.1409_1410dupTT | p.Ile471fs |
| A | | | | |
| 2:25246178:T:TA | NA | frameshift | c.1407_1410dupG | p.Ile471fs |
| ATC | | | ATT | |
| 2:25246178:T:TA | NA | frameshift | c.1401_1410dupCA | p.Ile471fs |
| ATCTCCTTG | | | AGGAGATT | |
| 2:25246178:TA:T | NA | frameshift | c.1410delT | p.Ile471fs |
| 2:25246179:A:AT | NA | frameshift | c.1409_1410insA | p.Ile471fs |
| 2:25246179:AAT: | NA | frameshift | c.1408_1409delAT | p.Ile470fs |
| A | | | | |
| 2:25246180:A:AT | NA | frameshift | c.1407_1408dupG | p.Ile470fs |
| C | | | A | |
| 2:25246180:AT:A | rs1348892100 | frameshift | c.1408delA | p.Ile470fs |
| 2:25246180:ATC: | NA | frameshift | c.1407_1408delGA | p.Glu469fs |
| A | | | | |
| 2:25246180:ATCT | NA | frameshift | c.1392_1408delGC | p.Lys464fs |
| CCTTGACCTTGGG | | | CCAAGGTCAAGGA | |
| C:A | | | GA | |
| 2:25246182:CT:C | NA | frameshift | c.1406delA | p.Glu469fs |
| 2:25246182:CTCC | NA | frameshift | c.1397_1406delAG | p.Lys466fs |
| TTGACCT:C | | | GTCAAGGA | |
| 2:25246183:TC:T | NA | frameshift | c.1405delG | p.Glu469fs |
| 2:25246184:C:A | rs765389013 | stop_gained | c.1405G > T | p.Glu469* |
| 2:25246185:C:CT | NA | frameshift | c.1403dupA | p.Glu469fs |
| 2:25246185:CT:C | NA | frameshift | c.1403delA | p.Lys468fs |
| 2:25246187:TG:T | NA | frameshift | c.1401delC | p.Lys468fs |
| 2:25246189:AC:A | rs1360596024 | frameshift | c.1399delG | p.Va1467fs |
| 2:25246189:ACC: | NA | frameshift | c.1398_1399delGG | p.Lys466fs |
| A | | | | |
| 2:25246191:CT:C | rs775785710 | frameshift | c.1397delA | p.Lys466fs |
| 2:25246191:CTTG | NA | frameshift | c.1391_1397delAG | p.Lys464fs |
| GGCT:C | | | CCCAA | |
| 2:25246192:T:TT | NA | frameshift | c.1384_1396dupGC | p.Lys466fs |
| GGGCTTCTCCGC | | | GGAGAAGCCCA | |
| 2:25246193:T:A | NA | stop_gained | c.1396A > T | p.Lys466* |
| 2:25246193:T:TG | NA | frameshift | c.1395dupC | p.Lys466fs |
| 2:25246193:TG:T | NA | frameshift | c.1395delC | p.Lys466fs |
| 2:25246196:GCTT | NA | frameshift | c.1388_1392delAG | p.Glu463fs |
| CT:G | | | AAG | |
| 2:25246197:C:CG | NA | frameshift | c.1391_1392insTTC | p.Lys464fs |
| AGAA | | | TC | |
| 2:25246198:T:TTC | NA | frameshift | c.1389_1390dupG | p.Lys464fs |
| | | | A | |
| 2:25246198:TTC:T | rs1349621546 | frameshift | c.1389_1390delGA | p.Lys464fs |
| 2:25246199:T:A | NA | stop_gained | c.1390A > T | p.Lys464* |
| 2:25246201:T:TC | NA | frameshift | c.1387dupG | p.Glu463fs |
| 2:25246201:TC:T | NA | frameshift | c.1387delG | p.Glu463fs |
| 2:25246201:TCCG | NA | frameshift | c.1380_1387delCA | p.Ser460fs |
| CTGTG:T | | | CAGCGG | |
| 2:25246202:C:A | NA | stop_gained | c.1387G > T | p.Glu463* |
| 2:25246202:C:CA | NA | frameshift | c.1386_1387insT | p.Glu463fs |
| 2:25246203:CG:C | NA | frameshift | c.1385delC | p.Ala462fs |
| 2:25246205:C:CT | NA | frameshift | c.1364_1383dupAA | p.Ala462fs |
| GTGCTCTTCCGGG | | | AAGCCCCGGAAGA | |
| GCTTTT | | | GCACA | |
| 2:25246205:CT:C | NA | frameshift | c.1383delA | p.Ala462fs |
| 2:25246207:G:GC | NA | frameshift | c.1381_1382insG | p.Thr461fs |
| 2:25246208:TG:T | NA | frameshift | c.1380delC | p.Ser460fs |
| 2:25246209:GCT: | NA | frameshift | c.1378_1379delAG | p.Ser460fs |
| G | | | | |
| 2:25246209:GCTC | NA | frameshift | c.1375_1379delAA | p.Lys459fs |
| TT:G | | | GAG | |
| 2:25246211:T:TCT | NA | frameshift | c.1361_1377dupCC | p.Ser460fs |
| TCCGGGGCTTTTT | | | AAAAAGCCCCGGA | |
| GG | | | AG | |
| 2:25246212:CT:C | NA | frameshift | c.1376delA | p.Lys459fs |
| 2:25246213:TTCC | NA | frameshift | c.1368_1375delGC | p.Pro457fs |
| GGGGC:T | | | CCCGGA | |
| 2:25246214:T:A | rs768204905 | stop_gained | c.1375A > T | p.Lys459* |
| 2:25246214:T:TC | NA | frameshift | c.1374dupG | p.Lys459fs |
| 2:25246214:TC:T | rs886041641 | frameshift | c.1374delG | p.Lys459fs |
| 2:25246214:TCC:T | NA | frameshift | c.1373_1374delGG | p.Arg458fs |
| 2:25246216:C:CG | NA | frameshift | c.1372dupC | p.Arg458fs |
| 2:25246216:CG:C | NA | frameshift | c.1372delC | p.Arg458fs |
| 2:25246219:GGCT | NA | frameshift | c.1359_1369delAG | p.Ala454fs |
| TTTTGGCT:G | | | CCAAAAAGC | |
| 2:25246220:GC:G | NA | frameshift | c.1368delG | p.Lys456fs |
| 2:25246221:C:CT | NA | frameshift | c.1367dupA | p.Pro457fs |
| 2:25246226:T:A | NA | stop_gained | c.1363A > T | p.Lys455* |
| 2:25246226:TG:T | rs765073748 | frameshift | c.1362delC | p.Lys456fs |
| 2:25246228:G:GC | NA | frameshift | c.1360dupG | p.Ala454fs |
| 2:25246228:GC:G | NA | frameshift | c.1360delG | p.Ala454fs |
| 2:25246230:T:TG | NA | frameshift | c.1357_1358dupCC | p.Ala454fs |
| G | | | | |
| 2:25246232:G:GT | NA | frameshift | c.1356dupA | p.Pro453fs |
| 2:25246233:T:TG | NA | frameshift | c.1355dupC | p.Pro453fs |
| 2:25246235:GA:G | NA | frameshift | c.1353delT | p.Pro452fs |
| 2:25246235:GAG | NA | frameshift | c.1343_1353delAC | p.Tyr448fs |
| GTGGTGCGT:G | | | GCACCACCT | |
| 2:25246236:AG:A | NA | frameshift | c.1352delC | p.Pro451fs |
| 2:25246236:AGG | NA | frameshift | c.1340_1352delCC | p.Ala447fs |
| TGGTGCGTAGG:A | | | TACGCACCACC | |
| 2:25246236:AGG | NA | frameshift | c.1328_1352delCT | p.Pro443fs |
| TGGTGCGTAGGC | | | GAGGCAGCTGCCTA | |
| AGCTGCCTCAG:A | | | CGCACCACC | |
| 2:25246238:G:GT | NA | frameshift | c.1350dupA | p.Pro451fs |
| 2:25246238:GT:G | NA | frameshift | c.1350delA | p.Pro451fs |
| 2:25246239:TG:T | rs1287033864 | frameshift | c.1349delC | p.Pro450fs |
| 2:25246241:GT:G | NA | frameshift | c.1347delA | p.Pro450fs |
| 2:25246242:T:TG | NA | frameshift | c.1346dupC | p.Pro450fs |
| 2:25246242:TGC: | NA | frameshift | c.1345_1346delGC | p.Ala449fs |
| T | | | | |
| 2:25246244:CGTA | NA | frameshift | c.1338_1344delTG | p.Ala447fs |
| GGCA:C | | | CCTAC | |
| 2:25246245:G:C | NA | stop_gained | c.1344C > G | p.Tyr448* |
| 2:25246245:G:GT | NA | frameshift | c.1343_1344insAA | p.Tyr448fs |
| T | | | | |
| 2:25246245:G:T | rs137931376 | stop_gained | c.1344C > A | p.Tyr448* |
| 2:25246246:TA:T | NA | frameshift | c.1342delT | p.Tyr448fs |
| 2:25246246:TAG | NA | frameshift | c.1330_1342delGA | p.Glu444fs |
| GCAGCTGCCTC:T | | | GGCAGCTGCCT | |
| 2:25246247:AG:A | NA | frameshift | c.1341delC | p.Tyr448fs |
| 2:25246247:AGG | NA | frameshift | c.1335_1341delAG | p.Ala446fs |
| CAGCT:A | | | CTGCC | |
| 2:25246249:GC:G | NA | frameshift | c.1339delG | p.Ala447fs |
| 2:25246249:GCA | NA | frameshift | c.1326_1339delAC | p.Glu442fs |
| GCTGCCTCAGGT: | | | CTGAGGCAGCTG | |
| G | | | | |
| 2:25246250:C:CA | NA | frameshift | c.1335_1338dupA | p.Ala447fs |
| GCT | | | GCT | |
| 2:25246252:GCTG | NA | frameshift | c.1333_1336delGC | p.Ala445fs |
| C:G | | | AG | |
| 2:25246252:GCTG | NA | frameshift | c.1320_1336delGG | p.Trp440fs |
| CCTCAGGTTCCAC | | | TGGAACCTGAGGC | |
| C:G | | | AG | |
| 2:25246253:CTGC | NA | frameshift | c.1316_1335delTG | p.Met439fs |
| CTCAGGTTCCACC | | | TGGGTGGAACCTG | |
| CACA:C | | | AGGCA | |
| 2:25246254:TG:T | NA | frameshift | c.1334delC | p.Ala445fs |
| 2:25246259:C:A | rs1481248882 | stop_gained | c.1330G > T | p.Glu444* |
| 2:25246259:CA:C | NA | frameshift | c.1329delT | p.Glu444fs |
| 2:25246259:CAG: | NA | frameshift | c.1328_1329delCT | p.Pro443fs |
| C | | | | |
| 2:25246260:A:AG | NA | frameshift | c.1328dupC | p.Glu444fs |
| 2:25246260:AG:A | NA | frameshift | c.1328delC | p.Pro443fs |
| 2:25246262:GT:G | NA | frameshift | c.1326delA | p.Glu442fs |
| 2:25246265:C:A | rs1205125035 | stop_gained | c.1324G > T | p.Glu442* |
| 2:25246266:CA:C | NA | frameshift | c.1322delT | p.Va1441fs |
| 2:25246267:A:AC | NA | frameshift | c.1321dupG | p.Va1441fs |
| 2:25246267:AC:A | rs1440952501 | frameshift | c.1321delG | p.Va1441fs |
| 2:25246269:C:CC | NA | frameshift | c.1318_1319dupTG | p.Trp440fs |
| A | | | | |
| 2:25246269:C:T | rs866490834 | stop_gained | c.1320G > A | p.Trp440* |
| 2:25246270:C:T | rs773260349 | stop_gained | c.1319G > A | p.Trp440* |
| 2:25246270:CA:C | NA | frameshift | c.1318delT | p.Trp440fs |
| 2:25246271:A:AC | NA | frameshift | c.1317dupG | p.Trp440fs |
| 2:25246271:AC:A | NA | frameshift | c.1317delG | p.Met439fs |
| 2:25246272:CAT: | rs762868483 | frameshift | c.1315_1316delAT | p.Met439fs |
| C | | | | |
| 2:25246272:CATG | NA | frameshift | c.1307_1316delAC | p.Tyr436fs |
| TCCGTGT:C | | | ACGGACAT | |
| 2:25246273:A:AA | NA | frameshift | c.1315_1316insAT | p.Met439fs |
| T | | | | |
| 2:25246273:A:AT | NA | frameshift | c.1314_1315dupCA | p.Met439fs |
| G | | | | |
| 2:25246273:AT:A | NA | frameshift | c.1315delA | p.Met439fs |
| 2:25246274:T:TG | NA | frameshift | c.1311_1314dupG | p.Met439fs |
| TCC | | | GAC | |
| 2:25246274:TGTC | NA | frameshift | c.1311_1314delGG | p.Asp438fs |
| C:T | | | AC | |
| 2:25246275:G:GT | NA | frameshift | c.1294_1313dupTA | p.Met439fs |
| CCGTGTACACTTC | | | CAAAGAAGTGTACA | |
| TTTGTA | | | CGGA | |
| 2:25246276:T:TC | NA | frameshift | c.1312dupG | p.Asp438fs |
| 2:25246276:TC:T | | frameshift | c.1312delG | p.Asp438fs |
| 2:25246276:TCCG | NA | frameshift | c.1294_1312delTA | p.Tyr432fs |
| TGTACACTTCTTT | | | CAAAGAAGTGTACA | |
| GTA:T | | | CGG | |
| 2:25246279:GT:G | NA | frameshift | c.1309delA | p.Thr437fs |
| 2:25246281:G:C | NA | stop_gained | c.1308C > G | p.Tyr436* |
| 2:25246281:G:T | NA | stop_gained | c.1308C > A | p.Tyr436* |
| 2:25246282:T:TA | NA | frameshift | c.1306dupT | p.Tyr436fs |
| 2:25246282:TA:T | NA | frameshift | c.1306delT | p.Tyr436fs |
| 2:25246282:TAC: | NA | frameshift | c.1305_1306delGT | p.Tyr436fs |
| T | | | | |
| 2:25246283:ACAC | NA | frameshift | c.1301_1305delAA | p.Glu434fs |
| TT:A | | | GTG | |
| 2:25246283:ACAC | NA | frameshift | c.1295_1305delAC | p.Tyr432fs |
| TTCTTTGT:A | | | AAAGAAGTG | |
| 2:25246284:CACT | NA | frameshift | c.1291_1304delCC | p.Pro431fs |
| TCTTTGTAGGG:C | | | CTACAAAGAAGT | |
| 2:25246285:A:AC | NA | frameshift | c.1303dupG | p.Va1435fs |
| 2:25246285:A:AC | NA | frameshift | c.1302_1303dupA | p.Va1435fs |
| T | | | G | |
| 2:25246286:CT:C | NA | frameshift | c.1302delA | p.Va1435fs |
| 2:25246287:T:TTC | NA | frameshift | c.1294_1301dupTA | p.Glu434fs |
| TTTGTA | | | CAAAGA | |
| 2:25246287:TTC:T | NA | frameshift | c.1300_1301delGA | p.Glu434fs |
| 2:25246288:TC:T | NA | frameshift | c.1300delG | p.Glu434fs |
| 2:25246289:C:A | NA | stop_gained | c.1300G > T | p.Glu434* |
| 2:25246289:CT:C | NA | frameshift | c.1299delA | p.Glu434fs |
| 2:25246291:T:TT | NA | frameshift | c.1296_1297dupCA | p.Lys433fs |
| G | | | | |
| 2:25246292:T:A | NA | stop_gained | c.1297A > T | p.Lys433* |
| 2:25246292:T:TG | NA | frameshift | c.1293_1296dupCT | p.Lys433fs |
| TAG | | | AC | |
| 2:25246292:TGTA | NA | frameshift | c.1293_1296delCT | p.Tyr432fs |
| G:T | | | AC | |
| 2:25246293:G:C | NA | stop_gained | c.1296C > G | p.Tyr432* |
| 2:25246293:G:GT | NA | stop_gained | c.1287_1295dupG | p.Pro431_Tyr432 |
| AGGGATTC | | | AATCCCTA | insTerAsnPro |
| 2:25246293:G:T | NA | stop_gained | c.1296C > A | p.Tyr432* |
| 2:25246294:TA:T | NA | frameshift | c.1294delT | p.Tyr432fs |
| 2:25246295:AG:A | NA | frameshift | c.1293delC | p.Tyr432fs |
| 2:25246295:AGG: | NA | frameshift | c.1292_1293delCC | p.Pro431fs |
| A | | | | |
| 2:25246297:G:GC | NA | frameshift | c.1291_1292insG | p.Pro431fs |
| 2:25246297:G:GG | NA | frameshift | c.1290_1291dupTC | p.Pro431fs |
| A | | | | |
| 2:25246298:G:GA | NA | frameshift | c.1290dupT | p.Pro431fs |
| 2:25246298:GA:G | NA | frameshift | c.1290delT | p.Tyr432fs |
| 2:25246300:T:TTC | NA | frameshift | c.1284_1288dupG | p.Asn430fs |
| TTC | | | AAGA | |
| 2:25246300:TTCT | NA | frameshift | c.1284_1288delGA | p.Lys429fs |
| TC:T | | | AGA | |
| 2:25246303:TTC:T | NA | frameshift | c.1284_1285delGA | p.Lys429fs |
| 2:25246304:T:A | NA | stop_gained | c.1285A > T | p.Lys429* |
| 2:25246304:TCTC | NA | frameshift | c.1280- | p.Lys429fs |
| TTCTGG:T | | | 4_1284delCCAGAA | |
| | | | GAG | |
| 2:25246305:CT:C | rs751049400 | frameshift | c.1283delA | p.Glu428fs |
| 2:25246306:TC:T | NA | frameshift | c.1282delG | p.Glu428fs |
| 2:25246307:C:A | NA | stop_gained | c.1282G > T | p.Glu428* |
| 2:25246307:C:CT | NA | frameshift | c.1281dupA | p.Glu428fs |
| 2:25246307:CT:C | NA | frameshift | c.1281delA | p.Glu428fs |
| 2:25246618:A:G | NA | splice_donor | c.1279+2T > C | |
| 2:25246619:C:A | NA | splice_donor | c.1279+1G > T | |
| 2:25246619:C:G | NA | splice_donor | c.1279+1G > C | |
| 2:25246619:C:T | rs374440649 | splice_donor | c.1279+1G > A | |
| 2:25246620:C:A | NA | stop_gained | c.1279G > T | p.Glu427* |
| 2:25246620:CT:C | NA | frameshift | c.1278delA | p.Glu427fs |
| 2:25246623:C:A | NA | stop_gained | c.1276G > T | p.Glu426* |
| 2:25246623:CT:C | NA | frameshift | c.1275delA | p.Glu426fs |
| 2:25246623:CTG: | NA | frameshift | c.1274_1275delCA | p.Pro425fs |
| C | | | | |
| 2:25246624:TG:T | NA | frameshift | c.1274delC | p.Pro425fs |
| 2:25246630:CT:C | NA | frameshift | c.1268delA | p.Glu423fs |
| 2:25246632:C:A | NA | stop_gained | c.1267G > T | p.Glu423* |
| 2:25246632:C:CC | NA | frameshift | c.1265_1266dupTG | p.Glu423fs |
| A | | | | |
| 2:25246632:C:CT | NA | frameshift | c.1266_1267insCCT | p.Glu423fs |
| AGG | | | A | |
| 2:25246633:CAG | NA | frameshift | c.1256_1265delCT | p.Pro419fs |
| GCCCTTAG:C | | | AAGGGCCT | |
| 2:25246634:AG:A | rs752298245 | frameshift | c.1264delC | p.Leu422fs |
| 2:25246636:GC:G | NA | frameshift | c.1262delG | p.Gly421fs |
| 2:25246636:GCCC | NA | frameshift | c.1259_1262delAG | p.Lys420fs |
| T:G | | | GG | |
| 2:25246637:CCCT | NA | frameshift | c.1219_1261delAT | p.Ile407fs |
| TAGGGCCAGAAG | | | TGAATGGGCCCTGG | |
| GCTGGAAGCCCCC | | | GGGGCTTCCAGCCT | |
| CAGGGCCCATTCA | | | TCTGGCCCTAAGG | |
| AT:C | | | | |
| 2:25246639:CT:C | NA | frameshift | c.1259delA | p.Lys420fs |
| 2:25246640:TTAG | NA | stop_gained | c.1250_1258delCT | p.Ser417_Lys420 |
| GGCCAG:T | | | GGCCCTA | delinsTer |
| 2:25246640:TTAG | NA | frameshift | c.1249_1258delTCT | p.Ser417fs |
| GGCCAGA:T | | | GGCCCTA | |
| 2:25246641:T:A | NA | stop_gained | c.1258A > T | p.Lys420* |
| 2:25246641:T:TA | NA | frameshift | c.1257dupT | p.Lys420fs |
| 2:25246641:TA:T | NA | frameshift | c.1257delT | p.Lys420fs |
| 2:25246642:A:AG | NA | frameshift | c.1256dupC | p.Lys420fs |
| 2:25246642:AG:A | NA | frameshift | c.1256delC | p.Pro419fs |
| 2:25246645:GC:G | NA | frameshift | c.1253delG | p.Gly418fs |
| 2:25246645:GCCA | NA | frameshift | c.1235_1253delGG | p.Gly412fs |
| GAAGGCTGGAAG | | | GGCTTCCAGCCTTC | |
| CCCC:G | | | TGG | |
| 2:25246647:C:CA | NA | frameshift | c.1251dupT | p.Gly418fs |
| 2:25246647:CAG: | NA | frameshift | c.1250_1251delCT | p.Ser417fs |
| C | | | | |
| 2:25246648:A:AG | NA | stop_gained | c.1250_1251insAG | p.Ser417_Gly418 |
| CCTTCTGGCCCTA | | | GCCCTTAGGGCCAG | insGlyProTerGly |
| AGGGCCT | | | AAGGC | GlnLysAla |
| 2:25246648:AG:A | NA | frameshift | c.1250delC | p.Ser417fs |
| 2:25246649:G:GT | NA | frameshift | c.1249_1250insA | p.Ser417fs |
| 2:25246649:GA:G | NA | frameshift | c.1249delT | p.Ser417fs |
| 2:25246651:AG:A | NA | frameshift | c.1247delC | p.Pro416fs |
| 2:25246653:GC:G | NA | frameshift | c.1245delG | p.Gln415fs |
| 2:25246653:GCTG | NA | frameshift | c.1223_1245delAA | p.Glu408fs |
| GAAGCCCCCCAG | | | TGGGCCCTGGGGG | |
| GGCCCATT:G | | | GCTTCCAG | |
| 2:25246654:C:CT | NA | frameshift | c.1244dupA | p.Pro416fs |
| 2:25246654:CTGG | NA | frameshift | c.1217_1244delTG | p.Met406fs |
| AAGCCCCCCAGG | | | ATTGAATGGGCCCT | |
| GCCCATTCAATCA: | | | GGGGGGCTTCCA | |
| C | | | | |
| 2:25246655:TG:T | NA | frameshift | c.1243delC | p.Gln415fs |
| 2:25246656:G:A | rs754223052 | stop_gained | c.1243C > T | p.Gln415* |
| 2:25246656:G:GG | NA | frameshift | c.1241_1242dupTC | p.Gln415fs |
| A | | | | |
| 2:25246657:G:C | NA | missense | c.1242C > G | p.Phe414Leu |
| 2:25246657:GA:G | NA | frameshift | c.1241delT | p.Phe414fs |
| 2:25246657:GAA | NA | frameshift | c.1222_1241delGA | p.Glu408fs |
| GCCCCCCAGGGCC | | | ATGGGCCCTGGGG | |
| CATTC:G | | | GGCTT | |
| 2:25246658:A:C | NA | missense | c.1241T > G | p.Phe414Cys |
| 2:25246658:A:G | rs1258189576 | missense | c.1241T > C | p.Phe414Ser |
| 2:25246658:AAG | NA | frameshift | c.1233_1240delGG | p.Gly412fs |
| CCCCCC:A | | | GGGGCT | |
| 2:25246658:AAG | NA | frameshift | c.1227_1240delGG | p.Trp409fs |
| CCCCCCAGGGCC: | | | CCCTGGGGGGCT | |
| A | | | | |
| 2:25246659:A:C | NA | missense | c.1240T > G | p.Phe414Val |
| 2:25246659:A:G | NA | missense | c.1240T > C | p.Phe414Leu |
| 2:25246659:A:T | rs1195155939 | missense | c.1240T > A | p.Phe414Ile |
| 2:25246659:AG:A | | frameshift | c.1239delC | p.Phe414fs |
| 2:25246659:AGC: | NA | frameshift | c.1238_1239delGC | p.Gly413fs |
| A | | | | |
| 2:25246660:G:GC | rs1234388246 | frameshift | c.1238dupG | p.Phe414fs |
| 2:25246660:G:GC | NA | frameshift | c.1234_1238dupG | p.Phe414fs |
| CCCC | | | GGGG | |
| 2:25246660:GCIG | rs755744291 | frameshift | c.1238delG | p.Gly413fs |
| 2:25246663:CCC | NA | frameshift | c.1232_1235dupTG | p.Phe414fs |
| CCA | | | GG | |
| 2:25246667:A:AG | NA | frameshift | c.1231dupC | p.Leu411fs |
| 2:25246667:A:AG | NA | frameshift | c.1209_1231dupCA | p.Leu411fs |
| GGCCCATTCAATC | | | AGCCCATGATTGAA | |
| ATGGGCTTG | | | TGGGCCC | |
| 2:25246667:AG:A | NA | frameshift | c.1231delC | p.Leu411fs |
| 2:25246670:GC:G | NA | frameshift | c.1228delG | p.Ala410fs |
| 2:25246672:C:T | | stop_gained | c.1227G > A | p.Trp409* |
| 2:25246672:CCAT | rs778152015 | frameshift | c.1223_1226delAA | p.Glu408fs |
| T:C | | | TG | |
| 2:25246673:C:CA | NA | frameshift | c.1225dupT | p.Trp409fs |
| 2:25246673:C:T | NA | stop_gained | c.1226G > A | p.Trp409* |
| 2:25246674:AT:A | NA | frameshift | c.1224delA | p.Glu408fs |
| 2:25246676:T:TC | NA | frameshift | c.1222dupG | p.Glu408fs |
| 2:25246677:C:A | NA | stop_gained | c.1222G > T | p.Glu408* |
| 2:25246677:C:CA | NA | frameshift | c.1221dupT | p.Glu408fs |
| 2:25246679:A:AT | NA | frameshift | c.1219_1220insGG | p.Ile407fs |
| GGGCCC | | | GCCCA | |
| 2:25246681:C:CA | NA | frameshift | c.1217_1218insTT | p.Met406fs |
| A | | | | |
| 2:25246681:CATG | NA | frameshift | c.1207_1217delAA | p.Asn403fs |
| GGCTTGTT:C | | | CAAGCCCAT | |
| 2:25246683:TG:T | NA | frameshift | c.1215delC | p.Met406fs |
| 2:25246686:GC:G | NA | frameshift | c.1212delG | p.Lys404fs |
| 2:25246687:C:CT | NA | frameshift | c.1211dupA | p.Pro405fs |
| 2:25246689:T:A | NA | stop_gained | c.1210A > T | p.Lys404* |
| 2:25246689:T:TG | | frameshift | c.1209dupC | p.Lys404fs |
| 2:25246689:TG:T | rs771398281 | frameshift | c.1209delC | p.Asn403fs |
| 2:25246690:GT:G | NA | frameshift | c.1208delA | p.Asn403fs |
| 2:25246691:T:TTC | NA | frameshift | c.1167_1207dupCA | p.Asn403fs |
| TGCACCTCCACGG | | | GCGATGAGAGTGA | |
| CCTTGGCAGTGTC | | | CACTGCCAAGGCCG | |
| ACTCTCATCGCTG | | | TGGAGGTGCAGA | |
| 2:25246691:TTCT | NA | frameshift | c.1188_1207delCA | p.Lys397fs |
| GCACCTCCACGGC | | | AGGCCGTGGAGGT | |
| CTTG:T | | | GCAGA | |
| 2:25246692:TC:T | NA | frameshift | c.1206delG | p.Asn403fs |
| 2:25246693:CT:C | NA | frameshift | c.1205delA | p.Gln402fs |
| 2:25246694:TGCA | NA | frameshift | c.1188_1204delCA | p.Lys397fs |
| CCTCCACGGCCTT | | | AGGCCGTGGAGGT | |
| G:T | | | GC | |
| 2:25246695:G:A | rs796065342 | stop_gained | c.1204C > T | p.Gln402* |
| 2:25246696:C:CA | NA | frameshift | c.1199_1202dupA | p.Gln402fs |
| CCT | | | GGT | |
| 2:25246699:CT:C | NA | frameshift | c.1199delA | p.Glu400fs |
| 2:25246699:CTCC | NA | frameshift | c.1189_1199delAA | p.Lys397fs |
| ACGGCCTT:C | | | GGCCGTGGA | |
| 2:25246700:T:TC | NA | frameshift | c.1198dupG | p.Glu400fs |
| 2:25246700:TC:T | NA | frameshift | c.1198delG | p.Glu400fs |
| 2:25246700:TCC:T | NA | frameshift | c.1197_1198delGG | p.Glu400fs |
| 2:25246701:C:A | rs751109858 | stop_gained | c.1198G > T | p.Glu400* |
| 2:25246701:C:CC | NA | frameshift | c.1196_1197dupTG | p.Glu400fs |
| A | | | | |
| 2:25246701:CCAC | NA | frameshift | c.1190_1197delAG | p.Lys397fs |
| GGCCT:C | | | GCCGTG | |
| 2:25246703:AC:A | NA | frameshift | c.1195delG | p.Va1399fs |
| 2:25246703:ACG | NA | frameshift | c.1188_1195delCA | p.Lys397fs |
| GCCTTG:A | | | AGGCCG | |
| 2:25246704:C:CG | NA | frameshift | c.1194_1195insCC | p.Va1399fs |
| TGG | | | AC | |
| 2:25246704:CG:C | NA | frameshift | c.1194delC | p.Va1399fs |
| 2:25246705:GGC | NA | frameshift | c.1171_1193delGA | p.Asp391fs |
| CTTGGCAGTGTCA | | | TGAGAGTGACACTG | |
| CTCTCATC:G | | | CCAAGGC | |
| 2:25246706:GC:G | NA | frameshift | c.1192delG | p.Ala398fs |
| 2:25246706:GCCT | NA | frameshift | c.1189_1192delAA | p.Lys397fs |
| T:G | | | GG | |
| 2:25246710:T:A | NA | stop_gained | c.1189A > T | p.Lys397* |
| 2:25246710:TG:T | NA | frameshift | c.1188delC | p.Lys397fs |
| 2:25246710:TGGC | NA | frameshift | c.1185_1188delTG | p.Ala396fs |
| A:T | | | CC | |
| 2:25246712:GC:G | NA | frameshift | c.1186delG | p.Ala396fs |
| 2:25246713:C:CA | rs1462007191 | frameshift | c.1185dupT | p.Ala396fs |
| 2:25246714:A:AG | NA | frameshift | c.1183_1184dupAC | p.Ala396fs |
| T | | | | |
| 2:25246714:AG:A | NA | frameshift | c.1184delC | p.Thr395fs |
| 2:25246714:AGT: | NA | frameshift | c.1183_1184delAC | p.Thr395fs |
| A | | | | |
| 2:25246715:G:GT | NA | frameshift | c.1183dupA | p.Thr395fs |
| 2:25246717:G:GT | NA | frameshift | c.1181dupA | p.Asp394fs |
| 2:25246718:T:TC | NA | frameshift | c.1180_1181insGA | p.Asp394fs |
| ACTCTCATCGCTC | | | GCGATGAGAGTG | |
| 2:25246718:T:TC | NA | frameshift | c.1161_1180dupCC | p.Asp394fs |
| ACTCTCATCGCTG | | | ACGACAGCGATGA | |
| TCGTGG | | | GAGTG | |
| 2:25246718:T:TC | NA | frameshift | c.1180_1181insGC | p.Asp394fs |
| ACTCTCATCGCTG | | | AGTCCCGGTGTGCC | |
| TCGTGGCACACCG | | | ACGACAGCGATGA | |
| GGACTGC | | | GAGTG | |
| 2:25246718:TCA: | NA | frameshift | c.1179_1180delTG | p.Ser393fs |
| T | | | | |
| 2:25246719:C:CA | NA | frameshift | c.1179dupT | p.Asp394fs |
| 2:25246719:CA:C | rs1224468033 | frameshift | c.1179delT | p.Ser393fs |
| 2:25246720:ACT: | NA | frameshift | c.1177_1178delAG | p.Ser393fs |
| A | | | | |
| 2:25246721:C:CG | NA | frameshift | c.1177_1178insTC | p.Ser393fs |
| A | | | | |
| 2:25246721:C:CT | NA | frameshift | c.1177dupA | p.Ser393fs |
| 2:25246721:CT:C | NA | frameshift | c.1177delA | p.Ser393fs |
| 2:25246722:TCTC | NA | frameshift | c.1170_1176delCG | p.Ser390fs |
| ATCG:T | | | ATGAG | |
| 2:25246723:C:CT | NA | frameshift | c.1175dupA | p.Ser393fs |
| 2:25246724:T:TC | NA | frameshift | c.1174_1175insCG | p.Glu392fs |
| ATCGCG | | | CGATG | |
| 2:25246725:C:A | rs1472367159 | stop_gained | c.1174G > T | p.Glu392* |
| 2:25246726:AT:A | NA | frameshift | c.1172delA | p.Asp391fs |
| 2:25246730:C:CT | NA | frameshift | c.1167_1168dupCA | p.Ser390fs |
| G | | | | |
| 2:25246730:C:G | NA | missense | c.1169G > C | p.Ser390Thr |
| 2:25246731:TG:T | NA | frameshift | c.1167delC | p.Asp389fs |
| 2:25246732:G:GT | NA | frameshift | c.1166dupA | p.Asp389fs |
| 2:25246733:TCGT | NA | frameshift | c.1150_1165delTTC | p.Phe384fs |
| GGCACACCGGGA | | | CCGGTGTGCCACG | |
| A:T | | | | |
| 2:25246735:G:GT | NA | frameshift | c.1163dupA | p.His388fs |
| 2:25246736:TG:T | NA | frameshift | c.1162delC | p.His388fs |
| 2:25246738:G:T | rs1364266797 | stop_gained | c.1161C > A | p.Cys387* |
| 2:25246740:AC:A | NA | frameshift | c.1158delG | p.Cys387fs |
| 2:25246740:ACAC | NA | frameshift | c.1145_1158delAG | p.Lys382fs |
| CGGGAACAGCT:A | | | CTGTTCCCGGTG | |
| 2:25246742:A:AC | NA | frameshift | c.1156dupG | p.Va1386fs |
| 2:25246742:A:AC | NA | frameshift | c.1149_1156dupGT | p.Va1386fs |
| CGGGAAC | | | TCCCGG | |
| 2:25246742:AC:A | NA | frameshift | c.1156delG | p.Va1386fs |
| 2:25246744:C:CG | NA | frameshift | c.1154dupC | p.Va1386fs |
| 2:25246744:C:CG | NA | frameshift | c.1148_1154dupTG | p.Cys387fs |
| GGAACA | | | TTCCC | |
| 2:25246744:CG:C | rs779418049 | frameshift | c.1154delC | p.Pro385fs |
| 2:25246744:CGG: | NA | frameshift | c.1153_1154delCC | p.Pro385fs |
| C | | | | |
| 2:25246744:CGG | NA | frameshift | c.1151_1154delTC | p.Phe384fs |
| GA:C | | | CC | |
| 2:25246744:CGG | NA | inframe_indel | c.1128_1154delCA | p.Ser377_Pro385 |
| GAACAGCTTCCCC | | | GCAGCCGCGCGGG | del |
| GCGCGGCTGCTG: | | | GAAGCTGTTCCC | |
| C | | | | |
| 2:25246745:GGG | NA | frameshift | c.1138_1153delGC | p.Ala380fs |
| AACAGCTTCCCCG | | | GGGGAAGCTGTTCC | |
| C:G | | | | |
| 2:25246747:G:GA | | frameshift | c.1151dupT | p.Va1386fs |
| 2:25246747:G:GT | rs1196506372 | frameshift | c.1151_1152insA | p.Phe384fs |
| 2:25246747:GA:G | NA | frameshift | c.1151delT | p.Phe384fs |
| 2:25246747:GAA | NA | inframe_indel | c.1125_1151delGG | p.Ala376_Phe384 |
| CAGCTTCCCCGCG | | | CCAGCAGCCGCGC | del |
| CGGCTGCTGGCC: | | | GGGGAAGCTGTT | |
| G | | | | |
| 2:25246750:C:CA | NA | frameshift | c.1148_1149insTT | p.Phe384fs |
| A | | | | |
| 2:25246750:CA:C | NA | frameshift | c.1148delT | p.Leu383fs |
| 2:25246751:A:AA | NA | frameshift | c.1147_1148insCT | p.Leu383fs |
| G | | | | |
| 2:25246751:AGCT | NA | frameshift | c.1143_1147delGA | p.Lys382fs |
| TC:A | | | AGC | |
| 2:25246751:AGCT | NA | frameshift | c.1140_1147delGG | p.Gly381fs |
| TCCCC:A | | | GGAAGC | |
| 2:25246753:CT:C | NA | frameshift | c.1145delA | p.Lys382fs |
| 2:25246755:T:A | NA | stop_gained | c.1144A > T | p.Lys382* |
| 2:25246755:TC:T | rs745939351 | frameshift | c.1143delG | p.Lys382fs |
| 2:25246758:CCG: | NA | frameshift | c.1139_1140delCG | p.Ala380fs |
| C | | | | |
| 2:25246758:CCGC | NA | frameshift | c.1127_1140delCC | p.Ala376fs |
| GCGGCTGCTGG:C | | | AGCAGCCGCGCG | |
| 2:25246759:CG:C | NA | frameshift | c.1139delC | p.Ala380fs |
| 2:25246760:G:T | NA | missense | c.1139C > A | p.Ala380Glu |
| 2:25246761:C:CA | NA | frameshift | c.1137_1138insT | p.Ala380fs |
| 2:25246761:C:G | NA | missense | c.1138G > C | p.Ala380Pro |
| 2:25246761:CGC | NA | splice_acceptor | c.1123- | p.Va1375_Ala380 |
| GGCTGCTGGCCAC | | | 2_1137delAGGTGG | del |
| CT:C | | | CCAGCAGCCGC | |
| 2:25246762:G:GC | NA | frameshift | c.1136dupG | p.Ala380fs |
| 2:25246762:GCG | NA | frameshift | c.1129_1136delAG | p.Ser377fs |
| GCTGCT:G | | | CAGCCG | |
| 2:25246763:C:A | NA | missense | c.1136G > T | p.Arg379Leu |
| 2:25246763:C:CG | NA | frameshift | c.1135dupC | p.Arg379fs |
| 2:25246763:C:G | NA | missense | c.1136G > C | p.Arg379Pro |
| 2:25246763:C:T | rs1036696061 | missense | c.1136G > A | p.Arg379His |
| 2:25246763:CG:C | NA | frameshift | c.1135delC | p.Arg379fs |
| 2:25246764:G:A | rs754459010 | missense | c.1135C > T | p.Arg379Cys |
| 2:25246764:G:C | NA | missense | c.1135C > G | p.Arg379Gly |
| 2:25246764:G:T | NA | missense | c.1135C > A | p.Arg379Ser |
| 2:25246765:G:T | NA | missense | c.1134C > A | p.Ser378Arg |
| 2:25246765:GCTG | NA | frameshift | c.1123_1133delGT | p.Va1375fs |
| CTGGCCAC:G | | | GGCCAGCAG | |
| 2:25246766:CT:C | NA | frameshift | c.1132delA | p.Ser378fs |
| 2:25246766:CTGC | NA | frameshift | c.1129_1132delAG | p.Ser377fs |
| T:C | | | CA | |
| 2:25246767:TG:T | NA | frameshift | c.1131delC | p.Ser377fs |
| 2:25246768:GC:G | NA | frameshift | c.1130delG | p.Ser377fs |
| 2:25246770:TG:T | NA | frameshift | c.1128delC | p.Ser377fs |
| 2:25246770:TGGC | NA | splice_acceptor | c.1123- | p.Va1375_Ser377 |
| CACCTGGA:T | | | 5_1128delTCCAGG | del |
| | | | TGGCC | |
| 2:25246772:G:A | NA | missense | c.1127C > T | p.Ala376Val |
| 2:25246772:G:T | NA | missense | c.1127C > A | p.Ala376Asp |
| 2:25246772:GCG | rs1383888539 | frameshift | c.1126delG | p.Ala376fs |
| 2:25246773:C:G | NA | missense | c.1126G > C | p.Ala376Pro |
| 2:25246773:C:T | rs1446942650 | missense | c.1126G > A | p.Ala376Thr |
| 2:25246773:CCA: | NA | frameshift | c.1124_1125delTG | p.Va1375fs |
| C | | | | |
| 2:25246773:CCAC | NA | splice_acceptor | c.1123- | p.Va1375_Ala376 |
| CTGGAGG:C | | | 7_1125delCCTCCA | del |
| | | | GGTG | |
| 2:25246773:CCAC | NA | splice_acceptor | c.1123- | p.Va1375_Ala376 |
| CTGGAGGGTGA:C | | | 11_1125delTCACCC | del |
| | | | TCCAGGTG | |
| 2:25246775:A:C | NA | missense | c.1124T > G | p.Va1375Gly |
| 2:25246775:AC:A | NA | frameshift | c.1123delG | p.Va1375fs |
| 2:25246776:CCTG | NA | splice_acceptor | c.1123-13_1123- | |
| GAGGGTGACA:C | | | 1delTGTCACCCTCC | |
| | | | AG | |
| 2:25246777:C:A | rs781118037 | splice_acceptor | c.1123-1G > T | |
| 2:25246777:C:G | NA | splice_acceptor | c.1123-1G > C | |
| 2:25246777:C:T | rs781118037 | splice_acceptor | c.1123-1G > A | |
| 2:25246777:CT:C | NA | splice_acceptor | c.1123-2delA | |
| 2:25246777:CTGG | NA | splice_acceptor | c.1123-11_1123- | |
| AGGGTGA:C | | | 2delTCACCCTCCA | |
| 2:25246777:CTGG | NA | splice_acceptor | c.1123-13_1123- | |
| AGGGTGACA:C | | | 2delTGTCACCCTCC | |
| | | | A | |
| 2:25246778:T:A | NA | splice_acceptor | c.1123-2A > T | |
| 2:25246778:T:C | rs752605931 | splice_acceptor | c.1123-2A > G | |
| 2:25246778:T:G | | splice_acceptor | c.1123-2A > C | |
| 2:25247048:CA:C | NA | splice_donor | c.1122+2delT | |
| 2:25247049:A:C | NA | splice_donor | c.1122+2T > G | |
| 2:25247049:A:G | NA | splice_donor | c.1122+2T > C | |
| 2:25247049:A:T | NA | splice_donor | c.1122+2T > A | |
| 2:25247049:AC:A | NA | splice_donor | c.1122+ldelG | |
| 2:25247050:C:G | NA | splice_donor | c.1122+1G > C | |
| 2:25247050:C:T | rs747220514 | splice_donor | c.1122+1G > A | |
| 2:25247051:CT:C | NA | frameshift | c.1121delA | p.Gln374fs |
| 2:25247052:T:A | NA | missense | c.1121A > T | p.Gln374Leu |
| 2:25247052:TG:T | NA | frameshift | c.1120delC | p.Gln374fs |
| 2:25247053:G:A | rs369109129 | stop_gained | c.1120C > T | p.Gln374* |
| 2:25247054:C:CT | NA | frameshift | c.1118_1119insA | p.Gln374fs |
| 2:25247055:A:C | NA | missense | c.1118T > G | p.Leu373Arg |
| 2:25247055:A:G | NA | missense | c.1118T > C | p.Leu373Pro |
| 2:25247055:A:T | rs748329208 | missense | c.1118T > A | p.Leu373Gln |
| 2:25247055:AG:A | NA | frameshift | c.1117delC | p.Leu373fs |
| 2:25247055:AGG | NA | frameshift | c.1105_1117delAT | p.Ile369fs |
| ACCTCGTAGAT:A | | | CTACGAGGTCC | |
| 2:25247056:G:C | NA | missense | c.1117C > G | p.Leu373Val |
| 2:25247057:G:GA | NA | frameshift | c.1115dupT | p.Leu373fs |
| 2:25247058:A:T | rs773722655 | missense | c.1115T > A | p.Va1372Asp |
| 2:25247058:AC:A | NA | frameshift | c.1114delG | p.Va1372fs |
| 2:25247058:ACCT: | NA | inframe_indel | c.1112_1114delAG | p.Glu371del |
| A | | | G | |
| 2:25247059:C:G | NA | missense | c.1114G > C | p.Va1372Leu |
| 2:25247059:C:T | rs371677904 | missense | c.1114G > A | p.Va1372Ile |
| 2:25247060:CT:C | NA | frameshift | c.1112delA | p.Glu371fs |
| 2:25247061:TC:T | NA | frameshift | c.1111delG | p.Glu371fs |
| 2:25247061:TCGT | NA | frameshift | c.1108_1111delTA | p.Tyr370fs |
| A:T | | | CG | |
| 2:25247062:C:A | rs1290621612 | stop_gained | c.1111G > T | p.Glu371* |
| 2:25247062:C:T | rs1290621612 | missense | c.1111G > A | p.Glu371Lys |
| 2:25247063:G:C | NA | stop_gained | c.1110C > G | p.Tyr370* |
| 2:25247063:G:T | NA | stop_gained | c.1110C > A | p.Tyr370* |
| 2:25247064:T:G | NA | missense | c.1109A > C | p.Tyr370Ser |
| 2:25247064:T:TA | NA | frameshift | c.1108dupT | p.Tyr370fs |
| 2:25247065:AG:A | NA | frameshift | c.1107delC | p.Tyr370fs |
| 2:25247065:AGAT | NA | frameshift | c.1103_1107delCC | p.Ala368fs |
| GG:A | | | ATC | |
| 2:25247066:G:GA | NA | inframe_indel | c.1092_1106dupGT | p.Met364_Ala368 |
| TGGCTTTGCGGTA | | | ACCGCAAAGCCAT | dup |
| C | | | | |
| 2:25247066:GAT | NA | inframe_indel | c.1092_1106delGT | p.Met364_Ala368 |
| GGCTTTGCGGTAC: | | | ACCGCAAAGCCAT | del |
| G | | | | |
| 2:25247067:A:C | NA | missense | c.1106T > G | p.Ile369Ser |
| 2:25247067:A:T | rs773941479 | missense | c.1106T > A | p.Ile369Asn |
| 2:25247068:T:A | NA | missense | c.1105A > T | p.Ile369Phe |
| 2:25247068:TG:T | NA | frameshift | c.1104delC | p.Ile369fs |
| 2:25247070:G:A | rs759087082 | missense | c.1103C > T | p.Ala368Val |
| 2:25247070:G:GC | NA | frameshift | c.1102dupG | p.Ala368fs |
| 2:25247070:G:T | NA | missense | c.1103C > A | p.Ala368Asp |
| 2:25247071:C:G | NA | missense | c.1102G > C | p.Ala368Pro |
| 2:25247071:C:T | rs1347653303 | missense | c.1102G > A | p.Ala368Thr |
| 2:25247071:CT:C | NA | frameshift | c.110ldelA | p.Ala368fs |
| 2:25247072:T:TTT | NA | inframe_indel | c.1080_1100dupCA | p.Asn360_Arg366 |
| GCGGTACATGGG | | | AGCAGCCCATGTAC | dup |
| CTGCTTG | | | CGCAA | |
| 2:25247072:TTTG | NA | frameshift | c.1088_1100delCC | p.Pro363fs |
| CGGTACATGG:T | | | ATGTACCGCAA | |
| 2:25247074:TG:T | NA | frameshift | c.1098delC | p.Ala368fs |
| 2:25247076:C:A | rs767236033 | missense | c.1097G > T | p.Arg366Leu |
| 2:25247076:C:CG | NA | frameshift | c.1093_1096dupTA | p.Arg366fs |
| GTA | | | CC | |
| 2:25247076:C:G | NA | missense | c.1097G > C | p.Arg366Pro |
| 2:25247076:C:T | rs767236033 | missense | c.1097G > A | p.Arg366His |
| 2:25247076:CG:C | NA | frameshift | c.1096delC | p.Arg366fs |
| 2:25247076:CGGT | NA | frameshift | c.1093_1096delTA | p.Tyr365fs |
| A:C | | | CC | |
| 2:25247077:G:A | NA | missense | c.1096C > T | p.Arg366Cys |
| 2:25247077:G:C | NA | missense | c.1096C > G | p.Arg366Gly |
| 2:25247077:G:T | NA | missense | c.1096C > A | p.Arg366Ser |
| 2:25247078:G:C | NA | stop_gained | c.1095C > G | p.Tyr365* |
| 2:25247078:G:T | NA | stop_gained | c.1095C > A | p.Tyr365* |
| 2:25247079:T:C | rs144062658 | missense | c.1094A > G | p.Tyr365Cys |
| 2:25247079:T:G | NA | missense | c.1094A > C | p.Tyr365Ser |
| 2:25247079:T:TA | NA | frameshift | c.1092_1093dupGT | p.Tyr365fs |
| C | | | | |
| 2:25247079:TA:T | NA | frameshift | c.1093delT | p.Tyr365fs |
| 2:25247080:A:G | rs1315257786 | missense | c.1093T > C | p.Tyr365His |
| 2:25247080:AC:A | NA | frameshift | c.1092delG | p.Met364fs |
| 2:25247080:ACAT | NA | frameshift | c.1088_1092delCC | p.Pro363fs |
| GG:A | | | ATG | |
| 2:25247083:TG:T | NA | frameshift | c.1089delC | p.Met364fs |
| 2:25247085:G:GG | NA | frameshift | c.1086_1087dupGC | p.Pro363fs |
| C | | | | |
| 2:25247088:T:G | NA | missense | c.1085A > C | p.Gln362Pro |
| 2:25247088:TG:T | rs749080312 | frameshift | c.1084delC | p.Gln362fs |
| 2:25247089:G:A | rs1490273086 | stop_gained | c.1084C > T | p.Gln362* |
| 2:25247089:G:GC | NA | frameshift | c.1083dupG | p.Gln362fs |
| 2:25247089:Gc.G | NA | frameshift | c.10083delG | p.Lys361fs |
| 2:25247090:CT:C | NA | frameshift | c.1082delA | p.Lys361fs |
| 2:25247090:CTTG: | NA | inframe_indel | c.1080_1082delCA | p.Asn360del |
| C | | | A | |
| 2:25247090:CTTG | NA | frameshift | c.1076_1082delAC | p.Tyr359fs |
| TTGT:C | | | AACAA | |
| 2:25247092:T:A | NA | stop_gained | c.1081A > T | p.Lys361* |
| 2:25247092:T:C | NA | missense | c.1081A > G | p.Lys361Glu |
| 2:25247093:G:GT | NA | frameshift | c.1079dupA | p.Asn360fs |
| 2:25247093:G:GT | NA | frameshift | c.1078_1079dupAA | p.Asn360fs |
| T | | | | |
| 2:25247094:T:TT | NA | frameshift | c.1075_1078dupTA | p.Asn360fs |
| GTA | | | CA | |
| 2:25247095:TG:T | NA | frameshift | c.1077delC | p.Tyr359fs |
| 2:25247096:G:C | NA | stop_gained | c.1077C > G | p.Tyr359* |
| 2:25247096:G:GT | NA | frameshift | c.1076dupA | p.Tyr359fs |
| 2:25247096:G:T | NA | stop_gained | c.1077C > A | p.Tyr359* |
| 2:25247098:A:T | NA | missense | c.1075T > A | p.Tyr359Asn |
| 2:25247098:AC:A | NA | frameshift | c.1074delG | p.Tyr359fs |
| 2:25247099:CG:C | NA | frameshift | c.1073delC | p.Thr358fs |
| 2:25247100:G:A | rs1191462913 | missense | c.1073C > T | p.Thr358Met |
| 2:25247100:G:T | NA | missense | c.1073C > A | p.Thr358Lys |
| 2:25247100:GT:G | NA | frameshift | c.1072delA | p.Thr358fs |
| 2:25247103:GC:G | NA | frameshift | c.1069delG | p.Ala357fs |
| 2:25247104:C:T | NA | missense | c.1069G > A | p.Ala357Thr |
| 2:25247105:CT:C | NA | frameshift | c.1067delA | p.Gln356fs |
| 2:25247106:TG:T | NA | frameshift | c.1066delC | p.Gln356fs |
| 2:25247106:TGGT | NA | inframe_indel | c.1055_1066delGT | p.Ser352_Gln356 |
| GGAACGCAC:T | | | GCGTTCCACC | delinsLys |
| 2:25247107:G:A | rs764062059 | stop_gained | c.1066C > T | p.Gln356* |
| 2:25247107:GGT | NA | frameshift | c.1056_1065delTG | p.Ala353fs |
| GGAACGCA:G | | | CGTTCCAC | |
| 2:25247109:T:TG | NA | frameshift | c.1063dupC | p.His355fs |
| 2:25247109:T:TG | NA | frameshift | c.1062_1063dupCC | p.His355fs |
| G | | | | |
| 2:25247109:TG:T | rs1274074419 | frameshift | c.1063delC | p.His355fs |
| 2:25247109:TGG | NA | frameshift | c.1051_1063delTG | p.Cys351fs |
| AACGCACTGCA:T | | | CAGTGCGTTCC | |
| 2:25247111:G:GA | NA | frameshift | c.1061_1062insTTA | p.His355fs |
| ACGCACTAA | | | GTGCGTT | |
| 2:25247111:GA:G | NA | frameshift | c.1061delT | p.Phe354fs |
| 2:25247111:GAA | NA | inframe_indel | c.1050_1061delTT | p.Cys351_Phe354 |
| CGCACTGCAA:G | | | GCAGTGCGTT | del |
| 2:25247112:A:C | NA | missense | c.1061T > G | p.Phe354Cys |
| 2:25247112:A:G | NA | missense | c.1061T > C | p.Phe354Ser |
| 2:25247112:AAC | NA | frameshift | c.1057_1060delGC | p.Ala353fs |
| GC:A | | | GT | |
| 2:25247113:A:AC | NA | frameshift | c.1058_1059dupCG | p.Phe354fs |
| G | | | | |
| 2:25247113:A:C | rs1161125109 | missense | c.1060T > G | p.Phe354Val |
| 2:25247113:AC:A | NA | frameshift | c.1059delG | p.Phe354fs |
| 2:25247114:CG:C | NA | frameshift | c.1058delC | p.Ala353fs |
| 2:25247115:G:A | rs1326972729 | missense | c.1058C > T | p.Ala353Val |
| 2:25247115:G:GC | NA | frameshift | c.1056_1057dupTG | p.Ala353fs |
| A | | | | |
| 2:25247115:GC:G | NA | frameshift | c.1057delG | p.Ala353fs |
| 2:25247116:CA:C | NA | frameshift | c.1056delT | p.Ser352fs |
| 2:25247117:AC:A | NA | frameshift | c.1055delG | p.Ser352fs |
| 2:25247117:ACT: | NA | frameshift | c.1054_1055delAG | p.Ser352fs |
| A | | | | |
| 2:25247118:C:T | rs139053291 | missense | c.1055G > A | p.Ser352Asn |
| 2:25247118:CT:C | NA | frameshift | c.1054delA | p.Ser352fs |
| 2:25247119:T:C | NA | missense | c.1054A > G | p.Ser352Gly |
| 2:25247119:T:TG | NA | frameshift | c.1031_1053dupTG | p.Ser352fs |
| CAAAACGAGCTCA | | | ATGCCGCTGAGCTC | |
| GCGGCATCA | | | GTTTTGC | |
| 2:25247119:TG:T | rs761025051 | frameshift | c.1053delC | p.Cys351fs |
| 2:25247120:G:T | NA | stop_gained | c.1053C > A | p.Cys351* |
| 2:25247120:GC:G | NA | frameshift | c.1052delG | p.Cys351fs |
| 2:25247121:C:CA | NA | frameshift | c.1051dupT | p.Cys351fs |
| 2:25247121:CA:C | | frameshift | c.1051delT | p.Cys351fs |
| 2:25247121:CAA: | NA | frameshift | c.1050_1051delTT | p.Phe350fs |
| C | | | | |
| 2:25247122:AAAA | NA | frameshift | c.1047_1050delGT | p.Phe350fs |
| C:A | | | TT | |
| 2:25247124:A:G | NA | missense | c.1049T > C | p.Phe350Ser |
| 2:25247125:AC:A | NA | frameshift | c.1047delG | p.Cys351fs |
| 2:25247127:G:A | NA | missense | c.1046C > T | p.Ser349Leu |
| 2:25247127:G:GA | NA | frameshift | c.1045dupT | p.Ser349fs |
| 2:25247127:G:T | rs1297885007 | stop_gained | c.1046C > A | p.Ser349* |
| 2:25247129:GC:G | NA | frameshift | c.1043delG | p.Ser348fs |
| 2:25247130:C:T | NA | missense | c.1043G > A | p.Ser348Asn |
| 2:25247130:CTCA: | NA | inframe_indel | c.1040_1042delTG | p.Leu347_Ser348 |
| C | | | A | delinsArg |
| 2:25247130:CTCA | NA | inframe_indel | c.1034_1042delTG | p.Met345_Leu347 |
| GCGGCA:C | | | CCGCTGA | del |
| 2:25247131:T:A | NA | missense | c.1042A > T | p.Ser348Cys |
| 2:25247131:T:TC | NA | frameshift | c.1038_1041dupGC | p.Ser348fs |
| AGC | | | TG | |
| 2:25247132:C:CA | NA | frameshift | c.1040dupT | p.Ser348fs |
| 2:25247133:A:C | NA | missense | c.1040T > G | p.Leu347Arg |
| 2:25247133:A:G | rs1474463141 | missense | c.1040T > C | p.Leu347Pro |
| 2:25247133:A:T | NA | missense | c.1040T > A | p.Leu347Gln |
| 2:25247133:AG:A | NA | frameshift | c.1039delC | p.Leu347fs |
| 2:25247133:AGC | NA | inframe_indel | c.1037_1039delCG | p.Pro346del |
| G:A | | | C | |
| 2:25247135:CG:C | NA | frameshift | c.1037delC | p.Pro346fs |
| 2:25247136:G:A | rs1308281604 | missense | c.1037C > T | p.Pro346Leu |
| 2:25247136:G:GG | NA | frameshift | c.1032_1036dupG | p.Pro346fs |
| CATC | | | ATGC | |
| 2:25247137:G:GC | NA | frameshift | c.1035dupG | p.Pro346fs |
| 2:25247137:G:T | NA | missense | c.1036C > A | p.Pro346Thr |
| 2:25247138:CA:C | NA | frameshift | c.1034delT | p.Met345fs |
| 2:25247139:A:AC | NA | frameshift | c.1033_1034insG | p.Met345fs |
| 2:25247139:AT:A | NA | frameshift | c.1033delA | p.Met345fs |
| 2:25247139:ATCA | NA | frameshift | c.1021_1033delGT | p.Va1341fs |
| GCTTCTCAAC:A | | | TGAGAAGCTGA | |
| 2:25247141:C:CA | NA | frameshift | c.1031_1032insAT | p.Met345fs |
| T | | | | |
| 2:25247141:CA:C | NA | frameshift | c.1031delT | p.Leu344fs |
| 2:25247141:CAGC | NA | frameshift | c.1016_1031delTG | p.Va1339fs |
| TTCTCAACACACA: | | | TGTGTTGAGAAGCT | |
| C | | | | |
| 2:25247142:A:C | rs749817324 | missense | c.1031T > G | p.Leu344Arg |
| 2:25247142:A:G | rs749817324 | missense | c.1031T > C | p.Leu344Pro |
| 2:25247142:A:T | rs749817324 | missense | c.1031T > A | p.Leu344Gln |
| 2:25247142:AG:A | NA | frameshift | c.1030delC | p.Leu344fs |
| 2:25247143:G:T | NA | missense | c.1030C > A | p.Leu344Met |
| 2:25247144:CT:C | NA | frameshift | c.1028delA | p.Lys343fs |
| 2:25247145:T:C | NA | missense | c.1028A > G | p.Lys343Arg |
| 2:25247146:T:A | NA | stop_gained | c.1027A > T | p.Lys343* |
| 2:25247146:T:TG | NA | frameshift | c.1026_1027insCTT | p.Lys343fs |
| TCCCCCCAGGTGT | | | CTCAACACACACCT | |
| GTGTTGAGAAG | | | GGGGGGAC | |
| 2:25247147:CT:C | NA | frameshift | c.1025delA | p.Glu342fs |
| 2:25247148:TC:T | NA | frameshift | c.1024delG | p.Glu342fs |
| 2:25247149:C:A | NA | stop_gained | c.1024G > T | p.Glu342* |
| 2:25247149:CAAC | NA | frameshift | c.1020_1023delTG | p.Cys340fs |
| A:C | | | TT | |
| 2:25247150:A:AA | rs768966400 | frameshift | c.1021_1022dupGT | p.Glu342fs |
| C | | | | |
| 2:25247150:AAC: | rs1201885886 | frameshift | c.1021_1022delGT | p.Va1341fs |
| A | | | | |
| 2:25247150:AACA | NA | frameshift | c.1019_1022delGT | p.Cys340fs |
| C:A | | | GT | |
| 2:25247151:A:AC | NA | frameshift | c.1015_1021dupGT | p.Va1341fs |
| ACACAC | | | GTGTG | |
| 2:25247151:AC:A | NA | frameshift | c.1021delG | p.Va1341fs |
| 2:25247151:ACAC | NA | frameshift | c.1015- | p.Va1339fs |
| ACACCT:A | | | 2_1021delAGGTGT | |
| | | | GTG | |
| 2:25247152:CACA | NA | splice_accepto | c.1015- | p.Va1339_Va1341d |
| CACCTGGGGG:C | | r | 7_1020delCCCCCA | el |
| | | | GGTGTGT | |
| 2:25247153:A:T | NA | stop_gained | c.1020T > A | p.Cys340* |
| 2:25247153:AC:A | NA | frameshift | c.1019delG | p.Cys340fs |
| 2:25247153:ACAC | NA | frameshift | c.1015_1019delGT | p.Va1339fs |
| AC:A | | | GTG | |
| 2:25247156:C:CA | NA | frameshift | c.1016dupT | p.Cys340fs |
| 2:25247156:C:CA | NA | frameshift | c.1015- | p.Cys340fs |
| CCT | | | 2_1016dupAGGT | |
| 2:25247156:CA:C | NA | frameshift | c.1016delT | p.Va1339fs |
| 2:25247156:CACC | NA | frameshift | c.1015- | p.Va1341fs |
| TG:C | | | 3_1016delCAGGT | |
| 2:25247156:CACC | NA | frameshift | c.1015- | p.Va1341fs |
| TGGGGGG:C | | | 8_1016delCCCCCC | |
| | | | AGGT | |
| 2:25247157:A:AC | rs773201456 | frameshift | c.1015dupG | p.Va1339fs |
| 2:25247157:A:C | rs1221845015 | missense | c.1016T > G | p.Va1339Gly |
| 2:25247157:AC:A | rs34203089 | frameshift | c.1015delG | p.Va1339fs |
| 2:25247159:C:A | rs1458692279 | splice_acceptor | c.1015-1G > T | |
| 2:25247159:C:G | NA | splice_acceptor | c.1015-1G > C | |
| 2:25247159:C:T | NA | splice_acceptor | c.1015-1G > A | |
| 2:25247159:CT:C | rs762753119 | splice_acceptor | c.1015-2delA | |
| 2:25247160:T:A | rs920946076 | splice_acceptor | c.1015-2A > T | |
| 2:25247160:T:C | rs920946076 | splice_acceptor | c.1015-2A > G | |
| 2:25247160:T:G | NA | splice_acceptor | c.1015-2A > C | |
| 2:25247588:T:TA | NA | splice_donor | c.1014+2dupT | |
| 2:25247588:TACC | NA | frameshift | c.1014_1014+2del | p.Va1339fs |
| T | | | GGT | |
| 2:25247589:A:C | rs1258308759 | splice_donor | c.1014+2T > G | |
| 2:25247589:A:G | NA | splice_donor | c.1014+2T > C | |
| 2:25247589:A:T | NA | splice_donor | c.1014+2T > A | |
| 2:25247589:AC:A | NA | splice_donor | c.1014+1delG | |
| 2:25247589:ACCA | NA | frameshift | c.1010_1014+1del | p.Ser337fs |
| CTG:A | | | CAGTGG | |
| 2:25247590:C:A | NA | splice_donor | c.1014+1G > T | |
| 2:25247590:C:G | NA | splice_donor | c.1014+1G > C | |
| 2:25247590:C:T | NA | splice_donor | c.1014+1G > A | |
| 2:25247590:CCA: | NA | frameshift | c.1013_1014delTG | p.Va1338fs |
| C | | | | |
| 2:25247591:CA:C | NA | frameshift | c.1013delT | p.Va1338fs |
| 2:25247591:CACT | NA | frameshift | c.1007_1013delTCT | p.Phe336fs |
| GAGA:C | | | CAGT | |
| 2:25247593:C:CT | NA | frameshift | c.986_1011dupTGT | p.Va1338fs |
| GAGAATTTGCCGT | | | GGTTCGGAGACGG | |
| CTCCGAACCACA | | | CAAATTCTCA | |
| 2:25247593:C:T | NA | missense | c.1012G > A | p.Va1338Met |
| 2:25247594:T:TG | NA | frameshift | c.1009_1010dupTC | p.Va1338fs |
| A | | | | |
| 2:25247594:TG:T | rs1212817360 | frameshift | c.1010delC | p.Ser337fs |
| 2:25247595:G:A | rs773208295 | missense | c.1010C > T | p.Ser337Leu |
| 2:25247595:G:C | rs773208295 | stop_gained | c.1010C > G | p.Ser337* |
| 2:25247595:G:T | rs773208295 | stop_gained | c.1010C > A | p.Ser337* |
| 2:25247595:GAG | NA | inframe_indel | c.1007_1009delTCT | p.Phe336del |
| A:G | | | | |
| 2:25247596:A:G | NA | missense | c.1009T > C | p.Ser337Pro |
| 2:25247597:G:GA | NA | frameshift | c.1007_1008insAT | p.Phe336fs |
| T | | | | |
| 2:25247597:GA:G | NA | frameshift | c.1007delT | p.Phe336fs |
| 2:25247597:GAA: | NA | frameshift | c.1006_1007delTT | p.Phe336fs |
| G | | | | |
| 2:25247597:GAAT | NA | frameshift | c.1004_1007delAA | p.Lys335fs |
| T:G | | | TT | |
| 2:25247598:A:AA | NA | stop_gained | c.1004_1006dupAA | p.Lys335_Phe336 |
| TT | | | T | insTer |
| 2:25247598:AATT | NA | frameshift | c.994_1006delGGA | p.Gly332fs |
| TGCCGTCTCC:A | | | GACGGCAAAT | |
| 2:25247599:AT:A | NA | frameshift | c.1005delA | p.Lys335fs |
| 2:25247600:TTTG | NA | frameshift | c.982_1004delGTC | p.Va1328fs |
| CCGTCTCCGAACC | | | ATGTGGTTCGGAGA | |
| ACATGAC:T | | | CGGCAA | |
| 2:25247602:T:A | NA | stop_gained | c.1003A > T | p.Lys335* |
| 2:25247603:GC:G | NA | frameshift | c.1001delG | p.Gly334fs |
| 2:25247605:C:CT | NA | frameshift | c.999_1000insA | p.Gly334fs |
| 2:25247605:C:T | rs1024296111 | missense | c.1000G > A | p.Gly334Ser |
| 2:25247605:CG:C | NA | frameshift | c.999delC | p.Asp333fs |
| 2:25247605:CGTC | NA | frameshift | c.996_999delAGAC | p.Asp333fs |
| T:C | | | | |
| 2:25247606:GTC: | NA | frameshift | c.997_998delGA | p.Asp333fs |
| G | | | | |
| 2:25247607:T:A | NA | missense | c.998A > T | p.Asp333Val |
| 2:25247608:C:CT | NA | frameshift | c.993_996dupCGG | p.Asp333fs |
| CCG | | | A | |
| 2:25247608:CTCC | NA | frameshift | c.992_996delTCGG | p.Phe331fs |
| GA:C | | | A | |
| 2:25247609:TCCG | NA | frameshift | c.974_995delCCCG | p.Thr325fs |
| AACCACATGACCC | | | CTGGGTCATGTGGT | |
| AGCGGG:T | | | TCGG | |
| 2:25247610:C:A | NA | missense | c.995G > T | p.Gly332Val |
| 2:25247610:C:T | rs751360082 | missense | c.995G > A | p.Gly332Glu |
| 2:25247611:C:A | rs760854242 | stop_gained | c.994G > T | p.Gly332* |
| 2:25247611:C:T | rs760854242 | missense | c.994G > A | p.Gly332Arg |
| 2:25247611:CG:C | NA | frameshift | c.993delC | p.Phe331fs |
| 2:25247612:GA:G | NA | frameshift | c.992delT | p.Phe331fs |
| 2:25247613:AACC: | rs755832478 | inframe_indel | c.989_991delGGT | p.Trp330del |
| A | | | | |
| 2:25247614:A:C | rs753896945 | missense | c.991T > G | p.Phe331Val |
| 2:25247614:A:T | NA | missense | c.991T > A | p.Phe331Ile |
| 2:25247614:AC:A | rs777388989 | frameshift | c.990delG | p.Trp330fs |
| 2:25247615:C:A | NA | missense | c.990G > T | p.Trp330Cys |
| 2:25247615:C:T | rs757340349 | stop_gained | c.990G > A | p.Trp330* |
| 2:25247615:CCAC | NA | inframe_indel | c.984_989delCATG | p.Met329_Trp330 |
| ATG:C | | | TG | del |
| 2:25247615:CCAC | NA | frameshift | c.979_989delTGGG | p.Trp327fs |
| ATGACCCA:C | | | TCATGTG | |
| 2:25247616:C:A | NA | missense | c.989G > T | p.Trp330Leu |
| 2:25247616:C:T | rs1479473205 | stop_gained | c.989G > A | p.Trp330* |
| 2:25247616:CACA | NA | frameshift | c.984_988delCATG | p.Met329fs |
| TG:C | | | T | |
| 2:25247617:ACAT | NA | frameshift | c.978_987delCTGG | p.Trp327fs |
| GACCCAG:A | | | GTCATG | |
| 2:25247618:C:CA | NA | frameshift | c.986dupT | p.Met329fs |
| 2:25247618:C:T | NA | missense | c.987G > A | p.Met329Ile |
| 2:25247619:A:G | NA | missense | c.986T > C | p.Met329Thr |
| 2:25247619:AT:A | NA | frameshift | c.985delA | p.Met329fs |
| 2:25247621:GA:G | NA | frameshift | c.983delT | p.Va1328fs |
| 2:25247622:A:AC | NA | frameshift | c.982dupG | p.Va1328fs |
| 2:25247622:A:C | NA | missense | c.983T > G | p.Va1328Gly |
| 2:25247622:A:G | NA | missense | c.983T > C | p.Va1328Ala |
| 2:25247622:A:T | NA | missense | c.983T > A | p.Va1328Asp |
| 2:25247622:ACCC | NA | frameshift | c.972_982delCACC | p.Thr325fs |
| AGCGGGTG:A | | | CGCTGGG | |
| 2:25247623:C:A | NA | missense | c.982G > T | p.Va1328Phe |
| 2:25247624:C:T | NA | stop_gained | c.981G > A | p.Trp327* |
| 2:25247625:C:A | NA | missense | c.980G > T | p.Trp327Leu |
| 2:25247625:C:CA | NA | frameshift | c.979dupT | p.Trp327fs |
| 2:25247625:C:T | rs750966422 | stop_gained | c.980G > A | p.Trp327* |
| 2:25247625:CAGC | NA | frameshift | c.966_979delTGAA | p.Glu323fs |
| GGGTGCCTTCA:C | | | GGCACCCGCT | |
| 2:25247626:AG:A | NA | frameshift | c.978delC | p.Trp327fs |
| 2:25247627:GC:G | NA | frameshift | c.977delG | p.Arg326fs |
| 2:25247627:GCG | NA | frameshift | c.971_977delGCAC | p.Gly324fs |
| GGTGC:G | | | CCG | |
| 2:25247628:C:A | rs758881009 | missense | c.977G > T | p.Arg326Leu |
| 2:25247628:C:CA | NA | frameshift | c.976_977insAGCG | p.Arg326fs |
| GCTGAAGGCACCC | | | GGTGCCTTCAGCT | |
| GCT | | | | |
| 2:25247628:C:CG | NA | frameshift | c.976dupC | p.Arg326fs |
| 2:25247628:C:G | NA | missense | c.977G > C | p.Arg326Pro |
| 2:25247628:C:T | rs758881009 | missense | c.977G > A | p.Arg326His |
| 2:25247628:CG:C | NA | frameshift | c.976delC | p.Arg326fs |
| 2:25247629:G:A | rs747448117 | missense | c.976C > T | p.Arg326Cys |
| 2:25247629:G:C | NA | missense | c.976C > G | p.Arg326Gly |
| 2:25247629:G:T | rs747448117 | missense | c.976C > A | p.Arg326Ser |
| 2:25247631:GT:G | NA | frameshift | c.973delA | p.Thr325fs |
| 2:25247631:GTGC | NA | frameshift | c.964_973delGCTG | p.Ala322fs |
| CTTCAGC:G | | | AAGGCA | |
| 2:25247632:T:TG | NA | frameshift | c.972dupC | p.Thr325fs |
| 2:25247632:TG:T | NA | frameshift | c.972delC | p.Thr325fs |
| 2:25247633:G:GC | NA | frameshift | c.971dupG | p.Thr325fs |
| 2:25247633:GC:G | NA | frameshift | c.971delG | p.Gly324fs |
| 2:25247634:C:A | NA | missense | c.971G > T | p.Gly324Val |
| 2:25247637:TCAG | NA | inframe_indel | c.947_967delCGGG | p.Thr316_Glu323 |
| CTGCTCGGCTCCG | | | CCGGAGCCGAGCA | delinsLys |
| GCCCG:T | | | GCTG | |
| 2:25247638:C:A | NA | stop_gained | c.967G > T | p.Glu323* |
| 2:25247638:CA:C | NA | frameshift | c.966delT | p.Glu323fs |
| 2:25247639:AG:A | NA | frameshift | c.965delC | p.Ala322fs |
| 2:25247640:GCTG | NA | frameshift | c.954_964delGAGC | p.Ser319fs |
| CTCGGCTC:G | | | CGAGCAG | |
| 2:25247641:C:CT | NA | frameshift | c.963dupA | p.Ala322fs |
| 2:25247641:CT:C | NA | frameshift | c.963delA | p.Ala322fs |
| 2:25247642:TG:T | NA | frameshift | c.962delC | p.Ala321fs |
| 2:25247644:C:CT | NA | frameshift | c.960_961insACCG | p.Ala321fs |
| CGGT | | | A | |
| 2:25247646:C:T | NA | missense | c.959G > A | p.Arg320Gln |
| 2:25247646:CG:C | NA | frameshift | c.958delC | p.Arg320fs |
| 2:25247647:G:A | rs778270132 | stop_gained | c.958C > T | p.Arg320* |
| 2:25247648:G:T | NA | missense | c.957C > A | p.Ser319Arg |
| 2:25247649:CT:C | NA | frameshift | c.955delA | p.Ser319fs |
| 2:25247650:T:TCC | NA | frameshift | c.950_954dupGCC | p.Ser319fs |
| GGC | | | GG | |
| 2:25247650:TC:T | NA | frameshift | c.954delG | p.Ser319fs |
| 2:25247652:C:CG | NA | frameshift | c.952dupC | p.Arg318fs |
| 2:25247652:CG:C | NA | frameshift | c.952delC | p.Arg318fs |
| 2:25247652:CGG: | NA | frameshift | c.951_952delCC | p.Arg318fs |
| C | | | | |
| 2:25247654:G:GC | NA | frameshift | c.950dupG | p.Arg318fs |
| 2:25247654:GC:G | NA | frameshift | c.950delG | p.Gly317fs |
| 2:25247654:GCCC | NA | frameshift | c.946_950delACGG | p.Thr316fs |
| GT:G | | | G | |
| 2:25247655:CCCG | NA | frameshift | c.943_949delATGA | p.Met315fs |
| TCAT:C | | | CGG | |
| 2:25247658:GT:G | NA | frameshift | c.946delA | p.Thr316fs |
| 2:25247661:AT:A | NA | frameshift | c.943delA | p.Met315fs |
| 2:25247661:ATCC | NA | frameshift | c.939_943delGTGG | p.Trp313fs |
| AC:A | | | A | |
| 2:25247662:TC:T | NA | frameshift | c.942delG | p.Trp314fs |
| 2:25247662:TCC:T | NA | frameshift | c.941_942delGG | p.Trp314fs |
| 2:25247662:TCCA | NA | frameshift | c.936_942delTTGG | p.Trp313fs |
| CCAA:T | | | TGG | |
| 2:25247662:TCCA | NA | frameshift | c.929_942delTTGT | p.Ile310fs |
| CCAAGACACAA:T | | | GTCTTGGTGG | |
| 2:25247663:C:T | rs772972939 | stop_gained | c.942G > A | p.Trp314* |
| 2:25247663:CCA: | NA | frameshift | c.940_941delTG | p.Trp314fs |
| C | | | | |
| 2:25247663:CCAC | NA | frameshift | c.934_941delTCTT | p.Ser312fs |
| CAAGA:C | | | GGTG | |
| 2:25247664:C:T | rs749225216 | stop_gained | c.941G > A | p.Trp314* |
| 2:25247664:CA:C | NA | frameshift | c.940delT | p.Trp314fs |
| 2:25247665:A:AC | NA | frameshift | c.939dupG | p.Trp314fs |
| 2:25247665:A:AC | NA | frameshift | c.938_939dupGG | p.Trp314fs |
| C | | | | |
| 2:25247665:AC:A | NA | frameshift | c.939delG | p.Trp313fs |
| 2:25247666:C:T | rs770813937 | stop_gained | c.939G > A | p.Trp313* |
| 2:25247667:C:CA | NA | frameshift | c.937dupT | p.Trp313fs |
| 2:25247667:C:T | rs1204478457 | stop_gained | c.938G > A | p.Trp313* |
| 2:25247667:CA:C | NA | frameshift | c.937delT | p.Trp313fs |
| 2:25247670:G:A | NA | missense | c.935C > T | p.Ser312Phe |
| 2:25247670:G:T | NA | missense | c.935C > A | p.Ser312Tyr |
| 2:25247670:GA:G | NA | frameshift | c.934delT | p.Ser312fs |
| 2:25247670:GAC: | NA | frameshift | c.933_934delGT | p.Ser312fs |
| G | | | | |
| 2:25247671:AC:A | NA | frameshift | c.933delG | p.Ser312fs |
| 2:25247672:C:CA | NA | frameshift | c.932dupT | p.Ser312fs |
| 2:25247672:CA:C | NA | frameshift | c.932delT | p.Va1311fs |
| 2:25247673:A:C | NA | missense | c.932T > G | p.Va1311Gly |
| 2:25247674:C:A | NA | missense | c.931G > T | p.Va1311Leu |
| 2:25247674:C:CA | NA | frameshift | c.930dupT | p.Va1311fs |
| 2:25247674:C:CA | NA | frameshift | c.929_930dupTT | p.Va1311fs |
| A | | | | |
| 2:25247674:CA:C | NA | frameshift | c.930delT | p.Ile310fs |
| 2:25247675:A:C | NA | missense | c.930T > G | p.Ile310Met |
| 2:25247676:A:C | NA | missense | c.929T > G | p.Ile310Ser |
| 2:25247676:A:G | NA | missense | c.929T > C | p.Ile310Thr |
| 2:25247676:A:T | rs587777508 | missense | c.929T > A | p.Ile310Asn |
| 2:25247676:AT:A | NA | frameshift | c.928delA | p.Ile310fs |
| 2:25247677:T:A | rs774128516 | missense | c.928A > T | p.Ile310Phe |
| 2:25247677:T:G | NA | missense | c.928A > C | p.Ile310Leu |
| 2:25247679:c.G | | missense | c.926G > C | p.Arg309Pro |
| 2:25247679:CG:C | NA | frameshift | c.925delC | p.Arg309fs |
| 2:25247680:G:A | NA | missense | c.925C > T | p.Arg309Cys |
| 2:25247680:G:C | NA | missense | c.925C > G | p.Arg309Gly |
| 2:25247680:G:GG | NA | frameshift | c.923_924dupGC | p.Arg309fs |
| C | | | | |
| 2:25247681:GC:G | NA | frameshift | c.923delG | p.Gly308fs |
| 2:25247682:C:A | NA | missense | c.923G > T | p.Gly308Val |
| 2:25247682:C:T | NA | missense | c.923G > A | p.Gly308Asp |
| 2:25247683:C:CT | NA | frameshift | c.921dupA | p.Gly308fs |
| 2:25247683:C:G | NA | missense | c.922G > C | p.Gly308Arg |
| 2:25247683:C:T | NA | missense | c.922G > A | p.Gly308Ser |
| 2:25247683:CT:C | NA | frameshift | c.921delA | p.Gly308fs |
| 2:25247684:TG:T | NA | frameshift | c.920delC | p.Pro307fs |
| 2:25247684:TGGC | NA | frameshift | c.907_920delTTCTC | p.Phe303fs |
| CACCAGGAGAA:T | | | CTGGTGGCC | |
| 2:25247685:G:A | rs759380437 | missense | c.920C > T | p.Pro307Leu |
| 2:25247685:G:C | rs759380437 | missense | c.920C > G | p.Pro307Arg |
| 2:25247685:G:T | rs759380437 | missense | c.920C > A | p.Pro307Gln |
| 2:25247686:G:A | NA | missense | c.919C > T | p.Pro307Ser |
| 2:25247686:G:C | NA | missense | c.919C > G | p.Pro307Ala |
| 2:25247686:G:T | NA | missense | c.919C > A | p.Pro307Thr |
| 2:25247686:GC:G | NA | frameshift | c.918delG | p.Trp306fs |
| 2:25247686:GCC: | NA | frameshift | c.917_918delGG | p.Trp306fs |
| G | | | | |
| 2:25247687:C:A | rs776841024 | missense | c.918G > T | p.Trp306Cys |
| 2:25247687:C:CC | NA | frameshift | c.916_917dupTG | p.Trp306fs |
| A | | | | |
| 2:25247687:C:G | NA | missense | c.918G > C | p.Trp306Cys |
| 2:25247687:C:T | rs776841024 | stop_gained | c.918G > A | p.Trp306* |
| 2:25247688:C:A | NA | missense | c.917G > T | p.Trp306Leu |
| 2:25247688:C:G | NA | missense | c.917G > C | p.Trp306Ser |
| 2:25247688:C:T | rs1465829182 | stop_gained | c.917G > A | p.Trp306* |
| 2:25247688:CA:C | NA | frameshift | c.916delT | p.Trp306fs |
| 2:25247689:A:C | NA | missense | c.916T > G | p.Trp306Gly |
| 2:25247689:AC:A | NA | frameshift | c.915delG | p.Trp305fs |
| 2:25247689:ACCA | NA | frameshift | c.912_915delCTGG | p.Trp305fs |
| G:A | | | | |
| 2:25247690:C:A | NA | missense | c.915G > T | p.Trp305Cys |
| 2:25247690:C:T | rs761839006 | stop_gained | c.915G > A | p.Trp305* |
| 2:25247691:C:CA | NA | frameshift | c.907_913dupTTCT | p.Trp305fs |
| GGAGAA | | | CCT | |
| 2:25247691:C:G | NA | missense | c.914G > C | p.Trp305Ser |
| 2:25247691:C:T | rs765341003 | stop_gained | c.914G > A | p.Trp305* |
| 2:25247691:CA:C | NA | frameshift | c.913delT | p.Trp305fs |
| 2:25247691:CAG: | NA | frameshift | c.912_913delCT | p.Trp305fs |
| C | | | | |
| 2:25247692:A:G | NA | missense | c.913T > C | p.Trp305Arg |
| 2:25247693:G:GA | NA | frameshift | c.911_912insT | p.Trp305fs |
| 2:25247693:GGA: | NA | frameshift | c.910_911delTC | p.Ser304fs |
| G | | | | |
| 2:25247694:G:A | NA | missense | c.911C > T | p.Ser304Phe |
| 2:25247694:GA:G | NA | frameshift | c.910delT | p.Ser304fs |
| 2:25247695:AG:A | NA | frameshift | c.909delC | p.Ser304fs |
| 2:25247696:G:C | NA | missense | c.909C > G | p.Phe303Leu |
| 2:25247696:GA:G | NA | frameshift | c.908delT | p.Phe303fs |
| 2:25247699:GC:G | NA | frameshift | c.905delG | p.Gly302fs |
| 2:25247700:C:T | NA | missense | c.905G > A | p.Gly302Asp |
| 2:25247701:C:A | NA | missense | c.904G > T | p.Gly302Cys |
| 2:25247701:C:T | rs377670596 | missense | c.904G > A | p.Gly302Ser |
| 2:25247703:C:T | NA | missense | c.902G > A | p.Arg301Gln |
| 2:25247703:CG:C | NA | frameshift | c.901delC | p.Arg301fs |
| 2:25247704:G:A | NA | missense | c.901C > T | p.Arg301Trp |
| 2:25247704:GC:G | NA | frameshift | c.900delG | p.Arg301fs |
| 2:25247704:GCA | NA | frameshift | c.891_900delGGGG | p.Trp297fs |
| GTTTCCCC:G | | | AAACTG | |
| 2:25247705:CA:C | NA | frameshift | c.899delT | p.Leu300fs |
| 2:25247706:A:T | NA | missense | c.899T > A | p.Leu300Gln |
| 2:25247707:G:T | NA | missense | c.898C > A | p.Leu300Met |
| 2:25247707:GT:G | | frameshift | c.897delA | p.Lys299fs |
| 2:25247708:T:A | NA | missense | c.897A > T | p.Lys299Asn |
| 2:25247708:T:G | NA | missense | c.897A > C | p.Lys299Asn |
| 2:25247709:T:G | NA | missense | c.896A > C | p.Lys299Thr |
| 2:25247710:T:A | rs766858016 | stop_gained | c.895A > T | p.Lys299* |
| 2:25247710:T:C | NA | missense | c.895A > G | p.Lys299Glu |
| 2:25247710:T:G | rs766858016 | missense | c.895A > C | p.Lys299Gln |
| 2:25247710:T:TC | rs1157280960 | frameshift | c.894dupG | p.Lys299fs |
| 2:25247710:TC:T | NA | frameshift | c.894delG | p.Lys299fs |
| 2:25247712:C:T | rs1410828051 | missense | c.893G > A | p.Gly298Glu |
| 2:25247713:C:A | rs797044904 | missense | c.892G > T | p.Gly298Trp |
| 2:25247713:C:G | NA | missense | c.892G > C | p.Gly298Arg |
| 2:25247713:C:T | rs797044904 | missense | c.892G > A | p.Gly298Arg |
| 2:25247714:C:A | NA | missense | c.891G > T | p.Trp297Cys |
| 2:25247714:C:G | NA | missense | c.891G > C | p.Trp297Cys |
| 2:25247714:C:T | rs1442214131 | stop_gained | c.891G > A | p.Trp297* |
| 2:25247715:C:G | NA | missense | c.890G > C | p.Trp297Ser |
| 2:25247715:C:T | rs944608317 | stop_gained | c.890G > A | p.Trp297* |
| 2:25247716:A:AC | NA | frameshift | c.888dupG | p.Trp297fs |
| 2:25247716:A:C | rs751916447 | missense | c.889T > G | p.Trp297Gly |
| 2:25247716:A:G | rs751916447 | missense | c.889T > C | p.Trp297Arg |
| 2:25247716:A:T | NA | missense | c.889T > A | p.Trp297Arg |
| 2:25247716:AC:A | NA | frameshift | c.888delG | p.Trp297fs |
| 2:25247716:ACAC | NA | frameshift | c.879_888delGGAG | p.Glu294fs |
| CAGCTCC:A | | | CTGGTG | |
| 2:25247716:ACAC | NA | frameshift | c.878_888delGGGA | p.Gly293fs |
| CAGCTCCC:A | | | GCTGGTG | |
| 2:25247718:A:AC | NA | frameshift | c.886dupG | p.Va1296fs |
| 2:25247718:A:C | rs1402705749 | missense | c.887T > G | p.Va1296Gly |
| 2:25247718:A:T | rs1402705749 | missense | c.887T > A | p.Va1296Glu |
| 2:25247718:AC:A | NA | frameshift | c.886delG | p.Va1296fs |
| 2:25247719:C:A | NA | missense | c.886G > T | p.Va1296Leu |
| 2:25247719:C:G | rs755398725 | missense | c.886G > C | p.Va1296Leu |
| 2:25247719:C:T | rs755398725 | missense | c.886G > A | p.Va1296Met |
| 2:25247721:A:AG | NA | frameshift | c.882_883dupGC | p.Leu295fs |
| C | | | | |
| 2:25247721:A:C | NA | missense | c.884T > G | p.Leu295Arg |
| 2:25247721:A:G | NA | missense | c.884T > C | p.Leu295Pro |
| 2:25247721:A:T | NA | missense | c.884T > A | p.Leu295Gln |
| 2:25247721:AG:A | NA | frameshift | c.883delC | p.Leu295fs |
| 2:25247722:G:C | rs780953840 | missense | c.883C > G | p.Leu295Val |
| 2:25247722:G:GA | NA | frameshift | c.882_883insT | p.Leu295fs |
| 2:25247722:G:T | NA | missense | c.883C > A | p.Leu295Met |
| 2:25247722:GCTC | NA | frameshift | c.879_882delGGAG | p.Glu294fs |
| C:G | | | | |
| 2:25247724:T:C | NA | missense | c.881A > G | p.Glu294Gly |
| 2:25247724:TC:T | NA | frameshift | c.880delG | p.Glu294fs |
| 2:25247725:C:A | rs755769867 | stop_gained | c.880G > T | p.Glu294* |
| 2:25247725:CCCC | NA | frameshift | c.875_879delTTGG | p.Ile292fs |
| AA:C | | | G | |
| 2:25247726:C:CC | NA | inframe_indel | c.873_878dupCATT | p.Gly293_Glu294 |
| CAATG | | | GG | insIleGly |
| 2:25247727:C:A | NA | missense | c.878G > T | p.Gly293Val |
| 2:25247727:C:T | NA | missense | c.878G > A | p.Gly293Glu |
| 2:25247727:CCAA | NA | inframe_indel | c.869_877delTTGG | p.Phe290_Gly293 |
| TGCCAA:C | | | CATTG | delinsTrp |
| 2:25247728:C:A | NA | missense | c.877G > T | p.Gly293Trp |
| 2:25247728:C:CA | NA | frameshift | c.876dupT | p.Gly293fs |
| 2:25247728:C:CA | NA | frameshift | c.856- | p.Gly293fs |
| ATGCCAAAGCCCC | | | 20_876dupTCTGCT | |
| GGCCGTCCTGGA | | | CCTTGGGGCTCCAG | |
| GCCCCAAGGAGC | | | GACGGCCGGGGCT | |
| AGA | | | TTGGCATT | |
| 2:25247728:C:G | NA | missense | c.877G > C | p.Gly293Arg |
| 2:25247728:C:T | NA | missense | c.877G > A | p.Gly293Arg |
| 2:25247728:CA:C | rs1271476754 | frameshift | c.876delT | p.Ile292fs |
| 2:25247730:A:G | rs777306476 | missense | c.875T > C | p.Ile292Thr |
| 2:25247730:AT:A | NA | frameshift | c.874delA | p.Ile292fs |
| 2:25247730:ATG: | NA | frameshift | c.873_874delCA | p.Ile292fs |
| A | | | | |
| 2:25247734:CA:C | NA | frameshift | c.870delT | p.Phe290fs |
| 2:25247735:A:AC | NA | frameshift | c.869_870insG | p.Phe290fs |
| 2:25247736:A:AA | NA | frameshift | c.856- | p.Phe290fs |
| GCCCCGGCCGTCC | | | 4_868dupCCAGGA | |
| TGG | | | CGGCCGGGGCT | |
| 2:25247736:A:C | NA | missense | c.869T > G | p.Phe290Cys |
| 2:25247736:A:G | NA | missense | c.869T > C | p.Phe290Ser |
| 2:25247737:A:C | NA | missense | c.868T > G | p.Phe290Val |
| 2:25247737:A:T | NA | missense | c.868T > A | p.Phe290Ile |
| 2:25247737:AG:A | rs746332586 | frameshift | c.867delC | p.Phe290fs |
| 2:25247738:G:GC | NA | frameshift | c.866dupG | p.Phe290fs |
| 2:25247738:GC:G | NA | frameshift | c.866delG | p.Gly289fs |
| 2:25247738:GCC: | NA | frameshift | c.865_866delGG | p.Gly289fs |
| G | | | | |
| 2:25247741:C:CA | NA | frameshift | c.863_864insT | p.Phe290fs |
| 2:25247742:CGG: | NA | frameshift | c.861_862delCC | p.Arg288fs |
| C | | | | |
| 2:25247743:GGC | NA | splice_acceptor | c.856- | p.Asp286_Arg288 |
| CGTCCTGGA:G | | | 5_861delTCCAGGA | del |
| | | | CGGC | |
| 2:25247744:GC:G | NA | frameshift | c.860delG | p.Gly287fs |
| 2:25247746:CG:C | NA | frameshift | c.858delC | p.Asp286fs |
| 2:25247748:TC:T | NA | frameshift | c.856delG | p.Asp286fs |
| 2:25247749:C:CC | NA | splice_acceptor | c.856-4_856- | |
| TGG | | | 1dupCCAG | |
| 2:25247750:C:A | rs1179790183 | splice_acceptor | c.856-1G > T | |
| 2:25247750:C:G | NA | splice_acceptor | c.856-1G > C | |
| 2:25247750:C:T | rs1179790183 | splice_acceptor | c.856-1G > A | |
| 2:25247750:CTGG | NA | splice_acceptor | c.856-5_856- | |
| A:C | | | 2delTCCA | |
| 2:25247751:T:A | NA | splice_acceptor | c.856-2A > T | |
| 2:25247751:T:C | rs1234716262 | splice_acceptor | c.856-2A > G | |
| 2:25247751:T:G | rs1234716262 | splice_acceptor | c.856-2A > C | |
| 2:25248034:CACC | NA | frameshift | c.851_855+2delAC | p.Tyr284fs |
| TCGT:C | | | GAGGT | |
| 2:25248035:A:C | NA | splice_donor | c.855+2T > G | |
| 2:25248035:A:G | rs745824601 | splice_donor | c.855+2T > C | |
| 2:25248035:AC:A | NA | splice_donor | c.855+1delG | |
| 2:25248036:C:A | rs772041639 | splice_donor | c.855+1G > T | |
| 2:25248036:C:G | NA | splice_donor | c.855+1G > C | |
| 2:25248036:C:T | rs772041639 | splice_donor | c.855+1G > A | |
| 2:25248037:CT:C | NA | frameshift | c.854delA | p.Glu285fs |
| 2:25248039:C:A | rs201882909 | stop_gained | c.853G > T | p.Glu285* |
| 2:25248039:CG:C | NA | frameshift | c.852delC | p.Tyr284fs |
| 2:25248040:G:C | NA | stop_gained | c.852C > G | p.Tyr284* |
| 2:25248040:G:T | NA | stop_gained | c.852C > A | p.Tyr284* |
| 2:25248040:GT:G | NA | frameshift | c.851delA | p.Tyr284fs |
| 2:25248041:TA:T | NA | frameshift | c.850delT | p.Tyr284fs |
| 2:25248041:TAC: | NA | frameshift | c.849_850delGT | p.Tyr284fs |
| T | | | | |
| 2:25248042:A:AC | NA | frameshift | c.842_849dupAGC | p.Tyr284fs |
| TCTGGCT | | | CAGAG | |
| 2:25248044:T:TCT | NA | frameshift | c.834_847dupCGA | p.Glu283fs |
| GGCTCGTCATCG | | | TGACGAGCCAG | |
| 2:25248044:TC:T | NA | frameshift | c.847delG | p.Glu283fs |
| 2:25248045:C:A | NA | stop_gained | c.847G > T | p.Glu283* |
| 2:25248045:C:CC | NA | frameshift | c.846_847insTG | p.Glu283fs |
| A | | | | |
| 2:25248045:C:CT | rs747075883 | frameshift | c.846dupA | p.Glu283fs |
| 2:25248049:C:CT | NA | frameshift | c.839_842dupACG | p.Pro282fs |
| CGT | | | A | |
| 2:25248050:TC:T | NA | frameshift | c.841delG | p.Glu281fs |
| 2:25248051:C:A | rs773312511 | stop_gained | c.841G > T | p.Glu281* |
| 2:25248051:CG:C | NA | frameshift | c.840delC | p.Asp280fs |
| 2:25248052:GT:G | NA | frameshift | c.839delA | p.Asp280fs |
| 2:25248053:TCAT | NA | frameshift | c.811_838delGACA | p.Asp271fs |
| CGCCTGCTTTGGT | | | AGAATGCCACCAAA | |
| GGCATTCTTGTC:T | | | GCAGGCGATG | |
| 2:25248056:TC:T | NA | frameshift | c.835delG | p.Asp279fs |
| 2:25248062:GCTT | NA | frameshift | c.817_829delAATG | p.Asn273fs |
| TGGTGGCATT:G | | | CCACCAAAG | |
| 2:25248063:C:CT | NA | frameshift | c.828dupA | p.Ala277fs |
| 2:25248063:CT:C | NA | frameshift | c.828delA | p.Ala277fs |
| 2:25248063:CTTT | NA | frameshift | c.824_828delCCAA | p.Thr275fs |
| GG:C | | | A | |
| 2:25248066:T:A | NA | stop_gained | c.826A > T | p.Lys276* |
| 2:25248066:TG:T | NA | frameshift | c.825delC | p.Ala277fs |
| 2:25248068:G:GT | NA | frameshift | c.816_823dupGAA | p.Thr275fs |
| GGCATTC | | | TGCCA | |
| 2:25248068:GT:G | NA | frameshift | c.823delA | p.Thr275fs |
| 2:25248069:T:TG | NA | frameshift | c.797_822dupGGT | p.Thr275fs |
| GCATTCTTGTCCC | | | CCGATGCTGGGGA | |
| CAGCATCGGACC | | | CAAGAATGCC | |
| 2:25248069:TG:T | NA | frameshift | c.822delC | p.Thr275fs |
| 2:25248071:GC:G | NA | frameshift | c.820delG | p.Ala274fs |
| 2:25248072:CA:C | NA | frameshift | c.819delT | p.Asn273fs |
| 2:25248073:AT:A | NA | frameshift | c.818delA | p.Asn273fs |
| 2:25248075:TC:T | NA | frameshift | c.816delG | p.Asn273fs |
| 2:25248076:CT:C | NA | frameshift | c.815delA | p.Lys272fs |
| 2:25248078:T:A | NA | stop_gained | c.814A > T | p.Lys272* |
| 2:25248079:GT:G | NA | frameshift | c.812delA | p.Asp271fs |
| 2:25248080:T:TC | NA | frameshift | c.811dupG | p.Asp271fs |
| 2:25248080:TC:T | NA | frameshift | c.811delG | p.Asp271fs |
| 2:25248080:TCCC | NA | frameshift | c.799_811delTCCG | p.Ser267fs |
| CAGCATCGGA:T | | | ATGCTGGGG | |
| 2:25248083:CCAG | NA | frameshift | c.784_808delCCTG | p.Pro262fs |
| CATCGGACCCCAC | | | AGCCCGTGGGGTCC | |
| GGGCTCAGG:C | | | GATGCTG | |
| 2:25248085:AG:A | NA | frameshift | c.806delC | p.Ala269fs |
| 2:25248085:AGC | NA | frameshift | c.785_806delCTGA | p.Pro262fs |
| ATCGGACCCCACG | | | GCCCGTGGGGTCC | |
| GGCTCAG:A | | | GATGC | |
| 2:25248086:G:GC | NA | frameshift | c.805dupG | p.Ala269fs |
| 2:25248087:C:CA | NA | frameshift | c.804dupT | p.Ala269fs |
| 2:25248087:C:CT | NA | frameshift | c.804_805insA | p.Ala269fs |
| 2:25248088:ATC: | NA | frameshift | c.802_803delGA | p.Asp268fs |
| A | | | | |
| 2:25248088:ATCG | NA | frameshift | c.796_803delGGGT | p.Gly266fs |
| GACCC:A | | | CCGA | |
| 2:25248089:T:TC | NA | frameshift | c.802dupG | p.Asp268fs |
| 2:25248090:C:CG | NA | frameshift | c.801dupC | p.Asp268fs |
| 2:25248090:CG:C | NA | frameshift | c.801delC | p.Asp268fs |
| 2:25248092:G:GA | NA | frameshift | c.799dupT | p.Ser267fs |
| 2:25248092:GA:G | NA | frameshift | c.799delT | p.Ser267fs |
| 2:25248092:GAC: | NA | frameshift | c.798_799delGT | p.Ser267fs |
| G | | | | |
| 2:25248092:GACC | NA | frameshift | c.795_799delGGGG | p.Gly266fs |
| CCG | | | T | |
| 2:25248093:A:AC | NA | frameshift | c.798dupG | p.Ser267fs |
| 2:25248093:AC:A | NA | frameshift | c.798delG | p.Ser267fs |
| 2:25248096:CCAC | NA | frameshift | c.788_795delAGCC | p.Glu263fs |
| GGGCT:C | | | CGTG | |
| 2:25248098:AC:A | NA | frameshift | c.793delG | p.Va1265fs |
| 2:25248098:ACG | NA | frameshift | c.789_793delGCCC | p.Glu263fs |
| GGC:A | | | G | |
| 2:25248098:ACG | NA | frameshift | c.784_793delCCTG | p.Pro262fs |
| GGCTCAGG:A | | | AGCCCG | |
| 2:25248099:CG:C | NA | frameshift | c.792delC | p.Va1265fs |
| 2:25248099:CGG | NA | frameshift | c.785_792delCTGA | p.Pro262fs |
| GCTCAG:C | | | GCCC | |
| 2:25248100:G:GT | NA | frameshift | c.791_792insA | p.Va1265fs |
| 2:25248102:G:GC | NA | frameshift | c.788_789dupAG | p.Pro264fs |
| T | | | | |
| 2:25248102:GC:G | NA | frameshift | c.789delG | p.Glu263fs |
| 2:25248103:C:CT | NA | frameshift | c.776_788dupCTAC | p.Glu263fs |
| CAGGCGTGGTAG | | | CACGCCTGA | |
| 2:25248103:CT:C | NA | frameshift | c.788delA | p.Glu263fs |
| 2:25248104:TC:T | NA | frameshift | c.787delG | p.Glu263fs |
| 2:25248105:C:A | NA | stop_gained | c.787G > T | p.Glu263* |
| 2:25248105:CAG: | NA | frameshift | c.785_786delCT | p.Pro262fs |
| C | | | | |
| 2:25248106:AG:A | NA | frameshift | c.785delC | p.Pro262fs |
| 2:25248110:G:GT | NA | frameshift | c.781dupA | p.Thr261fs |
| 2:25248110:GT:G | NA | frameshift | c.781delA | p.Thr261fs |
| 2:25248111:T:TG | NA | frameshift | c.780dupC | p.Thr261fs |
| 2:25248111:TG:T | NA | frameshift | c.780delC | p.Thr261fs |
| 2:25248111:TGGT | NA | frameshift | c.777_780delTACC | p.Thr260fs |
| A:T | | | | |
| 2:25248113:GT:G | NA | frameshift | c.778delA | p.Thr260fs |
| 2:25248113:GTA: | NA | frameshift | c.777_778delTA | p.Thr260fs |
| G | | | | |
| 2:25248116:G:GC | NA | frameshift | c.775dupG | p.Ala259fs |
| 2:25248116:GC:G | NA | frameshift | c.775delG | p.Ala259fs |
| 2:25248117:C:CTT | NA | stop_gained | c.774_775insTAA | p.Val258_Ala259 |
| A | | | | insTer |
| 2:25248117:CCA: | NA | frameshift | c.773_774delTG | p.Val258fs |
| C | | | | |
| 2:25248118:CA:C | NA | frameshift | c.773delT | p.Val258fs |
| 2:25248119:AC:A | NA | frameshift | c.772delG | p.Val258fs |
| 2:25248120:CA:C | NA | frameshift | c.771delT | p.Val258fs |
| 2:25248120:CAGT | NA | frameshift | c.768_771delCACT | p.Thr257fs |
| G:C | | | | |
| 2:25248120:CAGT | NA | frameshift | c.761_771delCATC | p.Ala254fs |
| GGGGGATG:C | | | CCCCACT | |
| 2:25248121:A:AC | NA | frameshift | c.770_771insGG | p.Ala259fs |
| C | | | | |
| 2:25248121:A:AG | NA | frameshift | c.769_770dupAC | p.Val258fs |
| T | | | | |
| 2:25248121:AG:A | NA | frameshift | c.770delC | p.Thr257fs |
| 2:25248121:AGT: | NA | frameshift | c.769_770delAC | p.Thr257fs |
| A | | | | |
| 2:25248121:AGT | NA | frameshift | c.767_770delCCAC | p.Pro256fs |
| GG:A | | | | |
| 2:25248121:AGT | NA | frameshift | c.764_770delCCCC | p.Ser255fs |
| GGGGG:A | | | CAC | |
| 2:25248122:GT:G | NA | frameshift | c.769delA | p.Thr257fs |
| 2:25248122:GTG | NA | frameshift | c.763_769delTCCC | p.Ser255fs |
| GGGGA:G | | | CCA | |
| 2:25248123:T:TG | NA | frameshift | c.768dupC | p.Thr257fs |
| 2:25248123:TG:T | rs776931901 | frameshift | c.768delC | p.Thr257fs |
| 2:25248128:G:GC | NA | frameshift | c.763_764insG | p.Ser255fs |
| 2:25248129:ATGC | NA | frameshift | c.752_762delCTGA | p.Thr251fs |
| GGGGTCAG:A | | | CCCCGCA | |
| 2:25248130:TG:T | NA | frameshift | c.761delC | p.Ala254fs |
| 2:25248132:C:CG | NA | frameshift | c.759dupC | p.Ala254fs |
| 2:25248132:C:CG | NA | frameshift | c.758_759dupCC | p.Ala254fs |
| G | | | | |
| 2:25248132:CG:C | NA | frameshift | c.759delC | p.Ala254fs |
| 2:25248132:CGG | NA | frameshift | c.725_759delCCAG | p.Ala242fs |
| GGTCAGTGGGCT | | | CCCTCCTGCTGTGC | |
| GCTGCACAGCAG | | | AGCAGCCCACTGAC | |
| GAGGGCTGG:C | | | CCC | |
| 2:25248136:GT:G | NA | frameshift | c.755delA | p.Asp252fs |
| 2:25248138:C:CA | NA | frameshift | c.753dupT | p.Asp252fs |
| 2:25248139:AG:A | NA | frameshift | c.752delC | p.Thr251fs |
| 2:25248140:GT:G | NA | frameshift | c.751delA | p.Thr251fs |
| 2:25248141:TG:T | rs749072422 | frameshift | c.750delC | p.Thr251fs |
| 2:25248141:TGG | NA | frameshift | c.738_750delTGTG | p.Val247fs |
| GCTGCTGCACA:T | | | CAGCAGCCC | |
| 2:25248141:TGG | NA | frameshift | c.731_750delCTCC | p.Pro244fs |
| GCTGCTGCACAGC | | | TGCTGTGCAGCAGC | |
| AGGAG:T | | | CC | |
| 2:25248145:CT:C | NA | frameshift | c.746delA | p.Gln249fs |
| 2:25248146:TG:T | NA | frameshift | c.745delC | p.Gln249fs |
| 2:25248146:TGCT | NA | frameshift | c.730_745delCCTC | p.Pro244fs |
| GCACAGCAGGAG | | | CTGCTGTGCAGC | |
| G:T | | | | |
| 2:25248147:G:A | rs759747476 | stop_gained | c.745C > T | p.Gln249* |
| 2:25248147:G:T | NA | missense | c.745C > A | p.Gln249Lys |
| 2:25248149:TGCA | NA | frameshift | c.738_742delTGTG | p.Val247fs |
| CA:T | | | c | |
| 2:25248149:TGCA | NA | frameshift | c.729_742delCCCT | p.Ser243fs |
| CAGCAGGAGGG:T | | | CCTGCTGTGC | |
| 2:25248149:TGCA | NA | frameshift | c.726_742delCAGC | p.Ser243fs |
| CAGCAGGAGGGC | | | CCTCCTGCTGTGC | |
| TG:T | | | | |
| 2:25248150:G:A | rs772648943 | stop_gained | c.742C > T | p.Gln248* |
| 2:25248150:G:GC | NA | frameshift | c.740_741dupTG | p.Gln248fs |
| A | | | | |
| 2:25248150:GCAC | NA | frameshift | c.716_741delTGGA | p.Val239fs |
| AGCAGGAGGGCT | | | GGAGGCCAGCCCTC | |
| GGCCTCCTCCA:G | | | CTGCTGTG | |
| 2:25248152:A:AG | NA | frameshift | c.739_740insC | p.Val247fs |
| 2:25248152:AC:A | NA | frameshift | c.739delG | p.Val247fs |
| 2:25248154:A:AG | NA | frameshift | c.737dupC | p.Val247fs |
| 2:25248154:AG:A | NA | frameshift | c.737delC | p.Ala246fs |
| 2:25248154:AGC | NA | frameshift | c.731_737delCTCC | p.Pro244fs |
| AGGAG:A | | | TGC | |
| 2:25248155:GC:G | NA | frameshift | c.736delG | p.Ala246fs |
| 2:25248156:C:CA | NA | frameshift | c.735_736insTT | p.Ala246fs |
| A | | | | |
| 2:25248156:CAG | NA | frameshift | c.725_735delCCAG | p.Ala242fs |
| GAGGGCTGG:C | | | CCCTCCT | |
| 2:25248156:CAG | NA | frameshift | c.719_735delAGGA | p.Glu240fs |
| GAGGGCTGGCCT | | | GGCCAGCCCTCCT | |
| CCT:C | | | | |
| 2:25248157:AG:A | rs1467732928 | frameshift | c.734delC | p.Pro245fs |
| 2:25248157:AGG | NA | frameshift | c.730_734delCCTC | p.Pro244fs |
| AGG:A | | | C | |
| 2:25248157:AGG | NA | frameshift | c.719_734delAGGA | p.Glu240fs |
| AGGGCTGGCCTCC | | | GGCCAGCCCTCC | |
| T:A | | | | |
| 2:25248158:G:GA | NA | frameshift | c.733_734insT | p.Pro245fs |
| 2:25248160:A:AG | NA | frameshift | c.731dupC | p.Pro245fs |
| 2:25248160:A:AG | NA | frameshift | c.728_731dupGCC | p.Ala246fs |
| GGC | | | C | |
| 2:25248160:AG:A | NA | frameshift | c.731delC | p.Pro244fs |
| 2:25248160:AGG | NA | frameshift | c.727_731delAGCC | p.Pro244fs |
| GCT:A | | | C | |
| 2:25248161:G:GG | NA | frameshift | c.702_730dupGGA | p.Pro244fs |
| GCTGGCCTCCTCC | | | GTCTCAGAAGGTG | |
| ACCTTCTGAGACT | | | GAGGAGGCCAGCC | |
| CC | | | | |
| 2:25248161:GGG | rs767439400 | frameshift | c.727_730delAGCC | p.Ser243fs |
| CT:G | | | | |
| 2:25248162:G:GA | NA | frameshift | c.729_730insAGGA | p.Pro244fs |
| GGCCAGCCCTCCT | | | GGGCTGGCCT | |
| 2:25248162:GGC: | NA | frameshift | c.728_729delGC | p.Ser243fs |
| G | | | | |
| 2:25248163:G:GC | NA | frameshift | c.704_728dupAGT | p.Ser243fs |
| TGGCCTCCTCCAC | | | CTCAGAAGGTGGA | |
| CTTCTGAGACT | | | GGAGGCCAG | |
| 2:25248163:GC:G | NA | frameshift | c.728delG | p.Ser243fs |
| 2:25248163:GCTG | NA | frameshift | c.722_728delAGGC | p.Glu241fs |
| GCCT:G | | | CAG | |
| 2:25248163:GCTG | NA | frameshift | c.721_728delGAGG | p.Glu241fs |
| GCCTC:G | | | CCAG | |
| 2:25248164:CT:C | NA | frameshift | c.727delA | p.Ser243fs |
| 2:25248165:T:TG | NA | frameshift | c.726dupC | p.Ser243fs |
| 2:25248165:TG:T | NA | frameshift | c.726delC | p.Ser243fs |
| 2:25248166:GGC | NA | frameshift | c.721_725delGAGG | p.Glu241fs |
| CTC:G | | | C | |
| 2:25248166:GGC | NA | frameshift | c.718_725delGAGG | p.Glu240fs |
| CTCCTCG | | | AGGC | |
| 2:25248167:G:GA | NA | frameshift | c.724_725insGCTG | p.Ala242fs |
| GGAGGCCAGC | | | GCCTCCT | |
| 2:25248167:G:GA | NA | frameshift | c.724_725insGCTG | p.Ala242fs |
| GTAGGCCAGC | | | GCCTACT | |
| 2:25248167:G:GC | NA | frameshift | c.682_724dupGAA | p.Ala242fs |
| CTCCTCCACCTTCT | | | GAAAACCAGGGGC | |
| GAGACTCCCCGG | | | CCGGGGAGTCTCA | |
| GCCCCTGGTTTTC | | | GAAGGTGGAGGAG | |
| TTC | | | G | |
| 2:25248167:GC:G | NA | frameshift | c.724delG | p.Ala242fs |
| 2:25248167:GCC: | NA | frameshift | c.723_724delGG | p.Glu241fs |
| G | | | | |
| 2:25248167:GCCT | NA | frameshift | c.718_724delGAGG | p.Glu240fs |
| CCTC:G | | | AGG | |
| 2:25248169:C:CA | NA | frameshift | c.722_723insTCCT | p.Glu241fs |
| GGA | | | | |
| 2:25248171:C:A | NA | stop_gained | c.721G > T | p.Glu241* |
| 2:25248171:CCT: | NA | frameshift | c.719_720delAG | p.Glu240fs |
| C | | | | |
| 2:25248174:C:A | NA | stop_gained | c.718G > T | p.Glu240* |
| 2:25248175:CACC | NA | frameshift | c.707_716delCTCA | p.Ser236fs |
| TTCTGAG:C | | | GAAGGT | |
| 2:25248176:A:AC | NA | frameshift | c.711_715dupGAA | p.Val239fs |
| CTTC | | | GG | |
| 2:25248176:AC:A | NA | frameshift | c.715delG | p.Val239fs |
| 2:25248176:ACCT | NA | frameshift | c.702_715delGGAG | p.Glu235fs |
| TCTGAGACTCC:A | | | TCTCAGAAGG | |
| 2:25248178:C:CT | NA | frameshift | c.713dupA | p.Val239fs |
| 2:25248178:CT:C | NA | frameshift | c.713delA | p.Lys238fs |
| 2:25248179:TTC:T | NA | frameshift | c.711_712delGA | p.Lys238fs |
| 2:25248179:TTCT | NA | frameshift | c.705_712delGTCT | p.Ser236fs |
| GAGAC:T | | | CAGA | |
| 2:25248180:T:A | NA | stop_gained | c.712A > T | p.Lys238* |
| 2:25248180:TC:T | NA | frameshift | c.711delG | p.Lys238fs |
| 2:25248183:G:A | NA | stop_gained | c.709C > T | p.Gln237* |
| 2:25248183:G:GA | NA | frameshift | c.708dupT | p.Gln237fs |
| 2:25248183:GA:G | NA | frameshift | c.708delT | p.Gln237fs |
| 2:25248183:GAG | NA | frameshift | c.683_708delAAGA | p.Glu228fs |
| ACTCCCCGGGCCC | | | AAACCAGGGGCCC | |
| CTGGTTTTCTTTG | | | GGGGAGTCT | |
| 2:25248186:AC:A | NA | frameshift | c.705delG | p.Glu235fs |
| 2:25248187:C:CT | NA | frameshift | c.704dupA | p.Ser236fs |
| 2:25248187:CT:C | NA | frameshift | c.704delA | p.Glu235fs |
| 2:25248188:T:TC | rs1301352218 | frameshift | c.703dupG | p.Glu235fs |
| 2:25248188:TC:T | NA | frameshift | c.703delG | p.Glu235fs |
| 2:25248189:C:A | NA | stop_gained | c.703G > T | p.Glu235* |
| 2:25248191:C:CT | NA | frameshift | c.700_701insA | p.Gly234fs |
| 2:25248192:C:CG | NA | frameshift | c.699dupC | p.Gly234fs |
| 2:25248192:C:CG | NA | frameshift | c.698_699dupCC | p.Gly234fs |
| G | | | | |
| 2:25248192:CG:C | | frameshift | c.699delC | p.Glu235fs |
| 2:25248195:G:GC | NA | frameshift | c.696dupG | p.Pro233fs |
| 2:25248195:G:GC | NA | frameshift | c.689_696dupACC | p.Pro233fs |
| CCCTGGT | | | AGGGG | |
| 2:25248195:GC:G | rs763740173 | frameshift | c.696delG | p.Glu235fs |
| 2:25248195:GCC: | NA | frameshift | c.695_696delGG | p.Gly232fs |
| G | | | | |
| 2:25248196:C:CG | NA | frameshift | c.695_696insC | p.Pro233fs |
| 2:25248199:CT:C | | frameshift | c.692delA | p.Gln231fs |
| 2:25248200:T:TG | NA | frameshift | c.691dupC | p.Gln231fs |
| 2:25248200:T:TG | NA | frameshift | c.691_692insGGGG | p.Gln231fs |
| GTTTTCTTCCCCC | | | GAAGAAAACC | |
| 2:25248200:TG:T | NA | frameshift | c.691delC | p.Gln231fs |
| 2:25248201:G:A | NA | stop_gained | c.691C > T | p.Gln231* |
| 2:25248202:GT:G | NA | frameshift | c.689delA | p.Asn230fs |
| 2:25248203:TTTT | NA | frameshift | c.685_688delGAAA | p.Glu229fs |
| C:T | | | | |
| 2:25248206:TC:T | NA | frameshift | c.685delG | p.Glu229fs |
| 2:25248207:C:A | NA | stop_gained | c.685G > T | p.Glu229* |
| 2:25248207:CT:C | NA | frameshift | c.684delA | p.Glu229fs |
| 2:25248209:TC:T | NA | frameshift | c.682delG | p.Glu228fs |
| 2:25248210:C:A | NA | stop_gained | c.682G > T | p.Glu228* |
| 2:25248211:CA:C | NA | frameshift | c.680delT | p.Val227fs |
| 2:25248213:C:CA | NA | frameshift | c.675_678dupTGCT | p.Val227fs |
| GCA | | | | |
| 2:25248213:CA:C | NA | frameshift | c.678delT | p.Val227fs |
| 2:25248214:AG:A | NA | frameshift | c.677delC | p.Ala226fs |
| 2:25248215:G:GC | NA | frameshift | c.676dupG | p.Ala226fs |
| 2:25248215:GC:G | NA | frameshift | c.676delG | p.Ala226fs |
| 2:25248216:C:CA | NA | frameshift | c.675dupT | p.Ala226fs |
| 2:25248216:CA:C | NA | frameshift | c.675delT | p.Asn225fs |
| 2:25248217:AT:A | rs1306866372 | frameshift | c.674delA | p.Asn225fs |
| 2:25248219:TC:T | NA | frameshift | c.672delG | p.Met224fs |
| 2:25248220:CA:C | NA | frameshift | c.671delT | p.Met224fs |
| 2:25248221:ATTC | NA | frameshift | c.667_670delGGAA | p.Gly223fs |
| C:A | | | | |
| 2:25274943:C:A | NA | stop_gained | c.637G > T | p.Glu213* |
| 2:25274945:CT:C | NA | frameshift | c.634delA | p.Arg212fs |
| 2:25274950:C:T | NA | stop_gained | c.630G > A | p.Trp210* |
| 2:25274951:C:T | NA | stop_gained | c.629G > A | p.Trp210* |
| 2:25274988:T:TG | NA | frameshift | c.542_591dupGTC | p.Ile198fs |
| TAGTAGGGGTCCC | | | AGCGGCCCATGCCG | |
| CCGCCTGGAAGG | | | AGGCTCACCTTCCA | |
| TGAGCCTCGGCAT | | | GGCGGGGGACCCC | |
| GGGCCGCTGAC | | | TACTAC | |
| 2:25275009:G:A | NA | stop_gained | c.571C > T | p.Gln191* |
| 2:25275010:GAA | NA | frameshift | c.563_569delTCAC | p.Leu188fs |
| GGTGA:G | | | CTT | |
| 2:25275021:T:TCC | NA | frameshift | c.558_559insCTGA | p.Arg187fs |
| GTCAG | | | CGG | |
| 2:25275022:C:CG | NA | frameshift | c.557dupC | p.Arg187fs |
| 2:25275036:G:A | NA | stop_gained | c.544C > T | p.Gln182* |
| 2:25275050:TC:T | NA | frameshift | c.529delG | p.Glu177fs |
| 2:25275056:CCCA | NA | frameshift | c.516_523delTGGC | p.Gly173fs |
| AGCCA:C | | | TTGG | |
| 2:25275064:A:AC | NA | frameshift | c.515dupG | p.Gly173fs |
| 2:25275069:G:GT | NA | frameshift | c.510_511insA | p.Arg171fs |
| 2:25275074:CG:C | rs1458590320 | frameshift | c.505delC | p.Arg169fs |
| 2:25275076:GC:G | NA | frameshift | c.503delG | p.Gly168fs |
| 2:25275498:G:C | NA | splice_donor | c.492+2C > G | |
| 2:25275512:G:GG | NA | frameshift | c.472_479dupATC | p.Met161fs |
| ATTCGAT | | | GAATC | |
| 2:25275541:T:A | NA | stop_gained | c.451A > T | p.Lys151* |
| 2:25275545:T:C | NA | splice_acceptor | c.449-2A > G | |
| 2:25282440:C:T | NA | splice_donor | c.448+1G > A | |
| 2:25282461:CG:C | NA | frameshift | c.427delC | p.Arg143fs |
| 2:25282462:G:A | rs1352259738 | stop_gained | c.427C > T | p.Arg143* |
| 2:25282463:GCG | NA | frameshift | c.425delG | p.Gly142fs |
| 2:25282486:CA:C | NA | frameshift | c.402delT | p.Asn134fs |
| 2:25282487:AT:A | NA | frameshift | c.401delA | p.Asn134fs |
| 2:25282503:G:T | NA | stop_gained | c.386C > A | p.Ser129* |
| 2:25282538:CT:C | NA | frameshift | c.350delA | p.Glu117fs |
| 2:25282544:T:TG | NA | frameshift | c.344_345insTCTG | p.Ala116fs |
| GGGGGCAGAAGG | | | CTGGGGCCCCGCCC | |
| GCGGGGCCCCAG | | | TTCTGCCCCCC | |
| CAGA | | | | |
| 2:25282544:TG:T | NA | frameshift | c.344delC | p.Pro115fs |
| 2:25282548:GCCC | NA | frameshift | c.337_340delGGGG | p.Gly113fs |
| C:G | | | | |
| 2:25282551:C:CA | NA | frameshift | c.337_338insCCCTT | p.Gly113fs |
| GAAGGG | | | CT | |
| 2:25282561:GC:G | NA | frameshift | c.327delG | p.Gln110fs |
| 2:25282576:TC:T | NA | frameshift | c.312delG | p.Ser105fs |
| 2:25282584:T:TG | NA | frameshift | c.304_305insC | p.Glu102fs |
| 2:25282590:TG:T | NA | frameshift | c.298delC | p.Gln100fs |
| 2:25282591:G:A | NA | stop_gained | c.298C > T | p.Gln100* |
| 2:25282597:C:A | NA | stop_gained | c.292G > T | p.Glu98* |
| 2:25282610:CA:C | NA | frameshift | c.278delT | p.Leu93fs |
| 2:25282634:TG:T | NA | frameshift | c.254delC | p.Ser85fs |
| 2:25282635:G:C | NA | stop_gained | c.254C > G | p.Ser85* |
| 2:25282642:CTGA | NA | frameshift | c.230_246delCCAT | p.Ser77fs |
| GTCCTGGGCCATG | | | GGCCCAGGACTCA | |
| G:C | | | | |
| 2:25282644:G:C | NA | stop_gained | c.245C > G | p.Ser82* |
| 2:25282649:C:CC | NA | frameshift | c.239_240insTGGA | p.Gln80fs |
| CATCCA | | | TGG | |
| 2:25282651:G:A | NA | stop_gained | c.238C > T | p.Gln80* |
| 2:25282661:TG:T | NA | frameshift | c.227delC | p.Pro76fs |
| 2:25282682:AGG: | NA | frameshift | c.205_206delCC | p.Pro69fs |
| A | | | | |
| 2:25282690:T:A | NA | stop_gained | c.199A > T | p.Lys67* |
| 2:25300139:C:CG | rs769876640 | frameshift | c.176dupC | p.Val60fs |
| 2:25300231:G:A | NA | stop_gained | c.85C > T | p.Gln29* |
| 2:25313912:C:T | NA | splice_donor | c.72+1G > A | |
| 2:25313936:C:CA | NA | frameshift | c.48_49insACGCAG | p.Ala17fs |
| GCTCTGCTGCGT | | | CAGAGCT | |
| 2:25313941:G:GC | NA | stop_gained | c.43_44insGAG | p.Ser15delinsTer |
| TC | | | | Ala |
| 2:25313949:G:GC | NA | frameshift | c.35_36insTCCTCCC | p.Ser13fs |
| GGAGCGGGAGGA | | | GCTCCG | |
| 2:25313949:G:GC | NA | frameshift | c.35_36insGCCCGC | p.Ser13fs |
| GGAGCGGGC | | | TCCG | |
| 2:25313953:TC:T | rs1213222712 | frameshift | c.31delG | p.Asp11fs |
| 2:25313957:C:CG | NA | frameshift | c.27dupC | p.Gly10fs |
| 2:25313957:CG:C | NA | frameshift | c.27delC | p.Asp11fs |
| 2:25313968:GA:G | NA | frameshift | c.16delT | p.Ser6fs |
| 2:25313976:G:GC | NA | frameshift | c.8_9insTGGAGGG | p.Met4fs |
| CCTCCA | | | | |
| 2:25314163:T:A | NA | splice_acceptor | c.-177-2A > T | |
| 2:25314163:T:G | NA | splice_acceptor | c.-177-2A > C | |

Representative ASXL1 somatic mutations include:

| | | | | |
|---|---|---|---|---|
| 20:32366382:A:G | NA | splice_acceptor | c.58-2A > G | |
| 20:32369013:A:G | NA | splice_acceptor | c.144-2A > G | |
| 20:32429899:A:G | NA | splice_acceptor | c.566-2A > G | |
| 20:32431680:G:A | NA | splice_donor | c.979+1G > A | |
| 20:32432900:C:T | NA | stop_gained | c.1000 > T | p.Gln334* |
| 20:32432930:GA:G | NA | frameshift | c.1034delA | p.Lys345fs |
| 20:32432978:G:T | NA | stop_gained | c.1078G > T | p.Gly360* |
| 20:32432981:C:T | NA | stop_gained | c.1081 > T | p.Gln361* |
| 20:32432986:G:A | NA | splice_donor | c.1085+1G > A | |
| 20:32433283:G:A | NA | splice_acceptor | c.1086-1G > A | |
| 20:32433283:G:C | NA | splice_acceptor | c.1086-1G > C | |
| 20:32433283:G:T | rs777661872 | splice_acceptor | c.1086-1G > T | |
| 20:32433297:A:T | NA | stop_gained | c.1099A > T | p.Lys367* |
| 20:32433310:T:A | NA | stop_gained | c.1112T > A | p.Leu371* |
| 20:32433312:C:T | rs199846284 | stop_gained | c.1114 > T | p.Gln372* |
| 20:32433315:C:T | rs1427299519 | stop_gained | c.1117 > T | p.Gln373* |
| 20:32433322:TG:T | NA | frameshift | c.1127delG | p.Gly376fs |
| 20:32433327:C:T | rs1386196069 | stop_gained | c.1129 > T | p.Gln377* |
| 20:32433330:G:T | NA | stop_gained | c.1132G > T | p.Glu378* |
| 20:32433333:G:GA | NA | frameshift | c.1136dupA | p.Ala380fs |
| 20:32433333:G:T | NA | stop_gained | c.1135G > T | p.Glu379* |
| 20:32433339:G:T | NA | stop_gained | c.1141G > T | p.Glu381* |
| 20:32433344:CAAAAGT | NA | frameshift | c.1147_1163 | p.Lys383fs |
| GGCTTGTGTGT:C | | | delAAAAGTG | |
| | | | GCTTGTGTG | |
| | | | T | |
| 20:32433345:A:T | NA | stop_gained | c.1147A > T | p.Lys383* |
| 20:32433354:TTG:T | rs886043994 | frameshift | c.1162_1163 | p.Va1388fs |
| | | | delGT | |
| 20:32433355:T:A | rs772584710 | stop_gained | c.1157T > A | p.Leu386* |
| 20:32433355:TGTGTG:T | NA | frameshift | c.1158_1162 | p.Leu386fs |
| | | | delGTGTG | |
| 20:32433356:G:GT | NA | frameshift | c.1159dupT | p.Cys387fs |
| 20:32433359:T:A | NA | stop_gained | c.1161T > A | p.Cys387* |
| 20:32433359:T:TGTCC | NA | frameshift | c.1162_1165 | p.Pro389fs |
| | | | dupGTCC | |
| 20:32433361:T:TC | NA | frameshift | c.1166dupC | p.Gly390fs |
| 20:32433366:G:T | rs1l90212499 | stop_gained | c.1168G > T | p.Gly390* |
| 20:32433369:G:T | NA | stop_gained | c.1171G > T | p.Glu391* |
| 20:32433372:TCAGTGC | NA | frameshift | c.1178_1188 | p.Va1393fs |
| GTATA:T | | | delTGCGTAT | |
| | | | ACAG | |
| 20:32433373:C:CA | NA | frameshift | c.1176dupA | p.Va1393fs |
| 20:32433373:C:G | NA | stop_gained | c.1175 > G | p.Ser392* |
| 20:32433379:G:GT | NA | frameshift | c.1182dupT | p.Ile395fs |
| 20:32433380:T:TA | NA | frameshift | c.1183dupA | p.Ile395fs |
| 20:32433381:A:AT | NA | frameshift | c.1184dupT | p.Gln396fs |
| 20:32433384:C:T | NA | stop_gained | c.1186C > T | p.Gln396* |
| 20:32433396:G:GC | NA | frameshift | c.1200dupC | p.Thr401fs |
| 20:32433408:C:CGAGAT | NA | frameshift | c.1212_1218 | p.His407fs |
| GG | | | dupAGATGG | |
| | | | G | |
| 20:32433408:C:T | rs373145711 | stop_gained | c.1210 > T | p.Arg404* |
| 20:32433415:GGCATTTT | NA | frameshift | c.1220_1233 | p.His407fs |
| AAGAAAC:G | | | delATTTTAA | |
| | | | GAAACGC | |
| 20:32433417:CATTTTAA | NA | frameshift | c.1220_1236 | p.His407fs |
| GAAACGCTCT:C | | | delATTTTAA | |
| | | | GAAACGCTC | |
| | | | T | |
| 20:32433418:A:AT | NA | frameshift | c.1224dupT | p.Lys409fs |
| 20:32433418:ATT:A | NA | frameshift | c.1223_1224 | p.Phe408fs |
| | | | delTT | |
| 20:32433422:T:TA | NA | frameshift | c.1226dupA | p.Lys410fs |
| 20:32433423:A:T | NA | stop_gained | c.1225A > T | p.Lys409* |
| 20:32433425:G:GA | NA | frameshift | c.1230dupA | p.Arg411fs |
| 20:32433426:A:T | NA | stop_gained | c.1228A > T | p.Lys410* |
| 20:32433430:GCT:G | NA | frameshift | c.1236_1237 | p.Arg413fs |
| | | | delTC | |
| 20:32433433:C:CT | NA | frameshift | c.1236dupT | p.Arg413fs |
| 20:32433434:TCGGCCA | NA | frameshift | c.1237_1244 | p.Arg413fs |
| GA:T | | | delCGGCCAG | |
| | | | A | |
| 20:32433441:G:GA | NA | frameshift | c.1244dupA | p.Asp415fs |
| 20:32433441:GAT:G | NA | frameshift | c.1244_1245 | p.Asp415fs |
| | | | delAT | |
| 20:32433447:C:T | rs375215583 | stop_gained | c.1249delT | p.Arg417* |
| 20:32433451:CCA:C | NA | frameshift | c.1254_1255 | p.Arg419fs |
| | | | delCA | |
| 20:32433453:A:T | NA | stop_gained | c.1255A > T | p.Arg419* |
| 20:32433464:G:GA | NA | frameshift | c.1268dupA | p.Asn423fs |
| 20:32433468:CTG:C | rs1269374749 | frameshift | c.1272_1273 | p.Tyr425fs |
| | | | delGT | |
| 20:32433468:CTGTACA | NA | frameshift | c.1271_1278 | p.Leu424fs |
| AA:C | | | delTGTACAA | |
| | | | A | |
| 20:32433472:AC:A | rs755250567 | frameshift | c.1275delC | p.Tyr425fs |
| 20:32433473:C:CA | rs1187950456, | frameshift | c.1281dupA | p.Gln428fs |
| | rs886042532 | | | |
| 20:32433473:C:G | NA | stop_gained | c.1275delG | p.Tyr425* |
| 20:32433474:A:T | NA | stop_gained | c.1276A > T | p.Lys426* |
| 20:32433477:A:T | NA | stop_gained | c.1279A > T | p.Lys427* |
| 20:32433479:A:AG | NA | frameshift | c.1281_1282 | p.Gln428fs |
| | | | insG | |
| 20:32433480:C:CA | NA | frameshift | c.1283dupA | p.Glu429fs |
| 20:32433480:C:T | rs886041975 | stop_gained | c.1282delT | p.Gln428* |
| 20:32433480:CAG:C | NA | frameshift | c.1283_1284 | p.Gln428fs |
| | | | delAG | |
| 20:32433482:GGAGTCA | NA | frameshift | c.1287_1366 | p.Glu429fs |
| GAACAAGCAGGGGTTGC | | | delGTCAGAA | |
| TAAGGATGCAAAATCTG | | | CAAGCAGGG | |
| TGGCCTCAGATGTTCCCC | | | GTTGCTAAG | |
| TCTACAAGGATGGGGAG | | | GATGCAAAA | |
| GCTAA:G | | | TCTGTGGCC | |
| | | | TCAGATGTTC | |
| | | | CCCTCTACAA | |
| | | | GGATGGGGA | |
| | | | GGCTAAGA | |
| 20:32433483:G:T | NA | stop_gained | c.1285G > T | p.Glu429* |
| 20:32433486:TC:T | NA | frameshift | c.1289delC | p.Ser430fs |
| 20:32433487:C:A | NA | stop_gained | c.1289delA | p.Ser430* |
| 20:32433487:C:G | NA | stop_gained | c.1289delG | p.Ser430* |
| 20:32433489:G:T | NA | stop_gained | c.1291G > T | p.Glu431* |
| 20:32433492:C:T | rs764325672 | stop_gained | c.1294delT | p.Gln432* |
| 20:32433495:G:GC | NA | frameshift | c.1298dupC | p.Gly434fs |
| 20:32433508:AGGATGC | NA | frameshift | c.1313_1380 | p.Asp438fs |
| AAAATCTGTGGCCTCAG | | | delATGCAAA | |
| ATGTTCCCCTCTACAAGG | | | ATCTGTGGC | |
| ATGGGGAGGCTAAGACT | | | CTCAGATGTT | |
| GACCCAGCAG:A | | | CCCCTCTACA | |
| | | | AGGATGGGG | |
| | | | AGGCTAAGA | |
| | | | CTGACCCAG | |
| | | | CAGGG | |
| 20:32433528:TC:T | NA | frameshift | c.1331delC | p.Ser444fs |
| 20:32433529:C:A | rs373126831 | stop_gained | c.1331C > A | p.Ser444* |
| 20:32433529:C:G | rs373126831 | stop_gained | c.1331C > G | p.Ser444* |
| 20:32433540:C:CATCCT | NA | frameshift | c.1342_1343 | p.Leu448fs |
| TGTAGAGGGGAACA | | | insATCCTTGT | |
| | | | AGAGGGGA | |
| | | | ACA | |
| 20:32433542:C:CCCCAT | NA | frameshift | c.1344_1345 | p.Tyr449fs |
| CCTTG | | | insCCCATCCT | |
| | | | TG | |
| 20:32433544:AC:A | | frameshift | c.1347delC | p.Tyr449fs |
| 20:32433544:ACAAGGA | NA | frameshift | c.1348_1361 | p.Lys450fs |
| TGGGGAGG:A | | | delAAGGATG | |
| | | | GGGAGGC | |
| 20:32433545:C:A | NA | stop_gained | c.1347C > A | p.Tyr449* |
| 20:32433545:C:G | NA | stop_gained | c.1347C > G | p.Tyr449* |
| 20:32433551:TGG:T | NA | frameshift | c.1356_1357 | p.Glu453fs |
| | | | delGG | |
| 20:32433552:G:GTCCC | NA | frameshift | c.1354_1355 | p.Gly452fs |
| | | | insTCCC | |
| 20:32433555:G:T | NA | stop_gained | c.1357G > T | p.Glu453* |
| 20:32433556:AGGCTAA | NA | frameshift | c.1363_1382 | p.Lys455fs |
| GACTGACCCAGCAG:A | | | delAAGACTG | |
| | | | ACCCAGCAG | |
| | | | GGCT | |
| 20:32433559:C:CT | NA | frameshift | c.1362dupT | p.Lys455fs |
| 20:32433560:T:TA | NA | frameshift | c.1364dupA | p.Thr456fs |
| 20:32433561:A:T | rs1292471399 | stop_gained | c.1363A > T | p.Lys455* |
| 20:32433566:TG:T | NA | frameshift | c.1369delG | p.Asp457fs |
| 20:32433567:GACCC:G | NA | frameshift | c.1371_1374 | p.Asp457fs |
| | | | delCCCA | |
| 20:32433568:AC:A | NA | frameshift | c.1373delC | p.Pro458fs |
| 20:32433568:ACC:A | NA | frameshift | c.1372_1373 | p.Pro458fs |
| | | | delCC | |
| 20:32433572:AGCAGG: | NA | frameshift | c.1377_1381 | p.Gly460fs |
| A | | | delAGGGC | |
| 20:32433575:A:AG | NA | frameshift | c.1380dupG | p.Leu461fs |
| 20:32433578:GC:G | NA | frameshift | c.1381delC | p.Leu461fs |
| 20:32433579:C:CT | NA | frameshift | c.1382dupT | p.Ser462fs |
| 20:32433580:TGA:T | NA | frameshift | c.1384_1385 | p.Ser462fs |
| | | | delAG | |
| 20:32433583:G:GC | NA | frameshift | c.1386dupC | p.Ser463fs |
| 20:32433584:CAG:C | NA | frameshift | c.1387_1388 | p.His465fs |
| | | | delAG | |
| 20:32433593:T:TA | NA | frameshift | c.1395_1396 | p.Leu466fs |
| | | | insA | |
| 20:32433601:GCACATC | NA | frameshift | c.1405_1412 | p.Thr469fs |
| CT:G | | | delACATCCT | |
| | | | C | |
| 20:32433606:T:TC | NA | frameshift | c.1410dupC | p.Ser471fs |
| 20:32433613:CAGCACC | NA | frameshift | c.1418_1431 | p.Ala473fs |
| CGACCTGG:C | | | delCACCCGA | |
| | | | CCTGGAG | |
| 20:32433616:C:CA | NA | frameshift | c.1419dupA | p.Pro474fs |
| 20:32433620:CGACCT:C | NA | frameshift | c.1424_1428 | p.Asp475fs |
| | | | delACCTG | |
| 20:32433625:T:TG | NA | frameshift | c.1429dupG | p.Glu477fs |
| 20:32433627:G:T | rs141346625 | stop_gained | c.1429G > T | p.Glu477* |
| 20:32433636:G:T | rs545224250 | stop_gained | c.1438G > T | p.Glu480* |
| 20:32433639:TTC:T | NA | frameshift | c.1442_1443 | p.Phe481fs |
| | | | delTC | |
| 20:32433643:CAGTTG:C | NA | frameshift | c.1449_1453 | p.Glu484fs |
| | | | delTGAGT | |
| 20:32433646:T:TG | NA | frameshift | c.1448_1449 | p.Glu484fs |
| | | | insG | |
| 20:32433648:G:T | NA | stop_gained | c.1450G > T | p.Glu484* |
| 20:32433666:ATCCAGG | NA | frameshift | c.1470_1489 | p.Gln491fs |
| CTGAGCCAGACAAC:A | | | delCCAGGCT | |
| | | | GAGCCAGAC | |
| | | | AACT | |
| 20:32433669:C:T | rs1335820343 | stop_gained | c.1471C > T | p.Gln491* |
| 20:32433675:G:T | rs1434942289 | stop_gained | c.1477G > T | p.Glu493* |
| 20:32433686:C:CT | NA | frameshift | c.1490dupT | p.Leu497fs |
| 20:32433688:T:TG | NA | frameshift | c.1492dupG | p.Ala498fs |
| 20:32433693:C:CG | NA | frameshift | c.1496dupG | p.Ala500fs |
| 20:32433694:G:GT | NA | frameshift | c.1497dupT | p.Ala500fs |
| 20:32433696:G:GC | NA | frameshift | c.1500dupC | p.Ser501fs |
| 20:32433699:T:TCTGC | NA | frameshift | c.1502_1505 | p.Ser503fs |
| | | | dupCTGC | |
| 20:32433702:G:GC | NA | frameshift | c.1505dupC | p.Ser503fs |
| 20:32433703:C:CT | NA | frameshift | c.1505_1506 | p.Ser503fs |
| | | | insT | |
| 20:32433713:C:CA | NA | frameshift | c.1516dupA | p.Arg506fs |
| 20:32433713:CAG:C | rs1377319529 | frameshift | c.1517_1518 | p.Arg506fs |
| | | | delGA | |
| 20:32433714:A:T | NA | stop_gained | c.1516A > T | p.Arg506* |
| 20:32433732:C:T | rs757832294 | stop_gained | c.1534C > T | p.Gln512* |
| 20:32433734:GGAAACT | NA | frameshift | c.1538_1545 | p.Glu513fs |
| GT:G | | | delAAACTGT | |
| | | | G | |
| 20:32433735:G:GA | NA | frameshift | c.1540dupA | p.Thr514fs |
| 20:32433735:G:T | rs763708711 | stop_gained | c.1537G > T | p.Glu513* |
| 20:32433739:CTG:C | rs886041485, | frameshift | c.1544_1545 | p.Va1515fs |
| | rs777537805 | | delTG | |
| 20:32433746:T:TCAGGA | NA | frameshift | c.1550_1556 | p.Lys520fs |
| AC | | | dupAGGAAC | |
| | | | c | |
| 20:32433747:C:T | rs755464186 | stop_gained | c.1549C > T | p.Gln517* |
| 20:32433750:G:T | NA | stop_gained | c.1552G > T | p.Glu518* |
| 20:32433762:C:T | rs772017757 | stop_gained | c.1564C > T | p.Gln522* |
| 20:32433764:G:GA | NA | frameshift | c.1568dupA | p.Arg524fs |
| 20:32433765:A:T | NA | stop_gained | c.1567A > T | p.Lys523* |
| 20:32433765:AAG:A | NA | frameshift | c.1570_1571 | p.Arg524fs |
| | | | delAG | |
| 20:32433770:G:GA | NA | frameshift | c.1575dupA | p.Ser526fs |
| 20:32433774:T:TC | NA | frameshift | c.1578dupC | p.Phe527fs |
| 20:32433780:G:T | rs746873290 | stop_gained | c.1582G > T | p.Glu528* |
| 20:32433783:C:T | rs770914619 | stop_gained | c.1585C > T | p.Gln529* |
| 20:32433786:G:GC | NA | frameshift | c.1589dupC | p.Ala531fs |
| 20:32433786:G:GCGGC | NA | frameshift | c.1590_1602 | p.Phe535fs |
| CTCTGCATC | | | dupGGCCTCT | |
| | | | GCATCC | |
| 20:32433786:G:GCGGC | rs1439862866 | frameshift | c.1591_1609 | p.Glu537fs |
| CTCTGCATCCTTTCC | | | dupGCCTCTG | |
| | | | CATCCTTTCC | |
| | | | CG | |
| 20:32433787:C:CGGCCT | NA | frameshift | c.1591_1597 | p.Ala533fs |
| CT | | | dupGCCTCTG | |
| 20:32433789:G:GCCTCT | NA | frameshift | c.1592_1598 | p.Ser534fs |
| GC | | | dupCCTCTGC | |
| 20:32433790:C:CCTCTG | NA | frameshift | c.1605_1606 | p.Pro536fs |
| CATCCTTTTCTTTT | | | insTCTTTCT | |
| | | | CTGCATCCTT | |
| | | | T | |
| 20:32433791:CTCTGCAT | NA | frameshift | c.1596_1606 | p.Ala533fs |
| CCTT:C | | | delTGCATCC | |
| | | | TTTC | |
| 20:32433792:TCTGCATC | NA | frameshift | c.1596_1621 | p.Ser534fs |
| CTTTCCCGAAAAGAAGC | | | delTGCATCC | |
| CC:T | | | TTTCCCGAAA | |
| | | | AGAAGCCCC | |
| 20:32433793:C:CT | NA | frameshift | c.1596dupT | p.Ala533fs |
| 20:32433794:TGC:T | NA | frameshift | c.1597_1598 | p.Ala533fs |
| | | | delGC | |
| 20:32433800:C:CT | NA | frameshift | c.1605dupT | p.Pro536fs |
| 20:32433800:CTT:C | NA | frameshift | c.1604_1605 | p.Phe535fs |
| | | | delTT | |
| 20:32433803:T:TCCCGA | NA | frameshift | c.1610_1622 | p.Leu542fs |
| AAAGAAGC | | | dupAAAAGA | |
| | | | AGCCCCG | |
| 20:32433805:C:CCGAAA | NA | frameshift | c.1610_1634 | p.Gln546fs |
| AGAAGCCCCGGCTTGAA | | | dupAAAAGA | |
| GAT | | | AGCCCCGGC | |
| | | | TTGAAGATC | |
| | | | G | |
| 20:32433806:C:CGAAAA | NA | frameshift | c.1609_1615 | p.Lys539fs |
| GA | | | dupGAAAAG | |
| | | | A | |
| 20:32433806:CGAAAA:C | NA | frameshift | c.1612_1616 | p.Lys538fs |
| | | | delAAGAA | |
| 20:32433807:G:T | rs369425922 | stop_gained | c.1609G > T | p.Glu537* |
| 20:32433809:A:AAAGA | NA | frameshift | c.1612_1621 | p.Arg541fs |
| AGCCCC | | | dupAAGAAG | |
| | | | CCCC | |
| 20:32433811:A:AGAAG | NA | frameshift | c.1614_1617 | p.Pro540fs |
| | | | dupGAAG | |
| 20:32433812:G:GA | NA | frameshift | c.1616dupA | p.Pro540fs |
| 20:32433812:G:GAAGC | NA | frameshift | c.1615_1621 | p.Arg541fs |
| CCC | | | dupAAGCCC | |
| | | | C | |
| 20:32433812:GAA:G | rs1388881234 | frameshift | c.1615_1616 | p.Lys539fs |
| | | | delAA | |
| 20:32433813:A:T | NA | stop_gained | c.1615A > T | p.Lys539* |
| 20:32433815:G:GC | | frameshift | c.1621dupC | p.Arg541fs |
| 20:32433815:GCC:G | NA | frameshift | c.1620_1621 | p.Arg541fs |
| | | | delCC | |
| 20:32433816:C:CCCCG | NA | frameshift | c.1619_1622 | p.Leu542fs |
| | | | dupCCCG | |
| 20:32433816:CCCCGG:C | NA | frameshift | c.1620_1624 | p.Arg541fs |
| | | | delCCGGC | |
| 20:32433818:CCG:C | NA | frameshift | c.1621_1622 | p.Arg541fs |
| | | | delCG | |
| 20:32433818:CCGGCTT | NA | frameshift | c.1621_1628 | p.Leu542fs |
| GA:C | | | delCGGCTTG | |
| | | | A | |
| 20:32433819:C:CGGCTT | NA | frameshift | c.1622_1628 | p.Asp544fs |
| GA | | | dupGGCTTG | |
| | | | A | |
| 20:32433819:C:CGGCTT | NA | frameshift | c.1622_1631 | p.Asp544fs |
| GAAGA | | | dupGGCTTG | |
| | | | AAGA | |
| 20:32433823:T:TTGAAG | NA | frameshift | c.1627_1639 | p.Ser547fs |
| ATCGTCAG | | | dupGAAGAT | |
| | | | CGTCAGT | |
| 20:32433824:T:TGAAGA | rs780939938 | frameshift | c.1628_1634 | p.Gln546fs |
| TC | | | dupAAGATC | |
| | | | G | |
| 20:32433825:G:T | NA | stop_gained | c.1627G > T | p.Glu543* |
| 20:32433826:AAGATCG | NA | frameshift | c.1631_1638 | p.Asp544fs |
| TC:A | | | delATCGTCA | |
| | | | G | |
| 20:32433827:A:AGATC | NA | frameshift | c.1631_1634 | p.Gln546fs |
| | | | dupATCG | |
| 20:32433829:A:AT | NA | frameshift | c.1632dupT | p.Arg545fs |
| 20:32433829:ATCGTCA | NA | frameshift | c.1633_1643 | p.Arg545fs |
| GTCCT:A | | | delCGTCAGT | |
| | | | CCTT | |
| 20:32433830:T:TG | NA | frameshift | c.1632_1633 | p.Arg545fs |
| | | | insG | |
| 20:32433830:TCGTCA:T | NA | frameshift | c.1633_1637 | p.Arg545fs |
| | | | delCGTCA | |
| 20:32433831:C:CT | NA | frameshift | c.1633_1634 | p.Arg545fs |
| | | | insT | |
| 20:32433832:G:GT | NA | frameshift | c.1635dupT | p.Gln546fs |
| 20:32433834:C:T | rs1264792645 | stop_gained | c.1636OT | p.Gln546* |
| 20:32433837:T:TC | NA | frameshift | c.1641dupC | p.Phe548fs |
| 20:32433839:C:CT | rs745845768 | frameshift | c.1644dupT | p.Arg549fs |
| 20:32433841:T:TA | rs1211392515 | frameshift | c.1643_1644 | p.Phe548fs |
| | | | insA | |
| 20:32433844:G:GT | NA | frameshift | c.1647dupT | p.Asn550fs |
| 20:32433846:A:AACACA | NA | frameshift | c.1649_1670 | p.Glu558fs |
| ATTGAAAGTGTTCACAC | | | dupACACAA | |
| | | | TTGAAAGTG | |
| | | | TTCACAC | |
| 20:32433846:AAC:A | NA | frameshift | c.1652_1653 | p.Thr551fs |
| | | | delCA | |
| 20:32433855:G:T | NA | stop_gained | c.1657G > T | p.Glu553* |
| 20:32433879:C:T | NA | stop_gained | c.1681C > T | p.Gln561* |
| 20:32433881:G:GC | NA | frameshift | c.1686dupC | p.Thr563fs |
| 20:32433887:TAA:T | NA | frameshift | c.1691_1692 | p.Lys564fs |
| | | | delAA | |
| 20:32433900:A:T | NA | stop_gained | c.1702A > T | p.Lys568* |
| 20:32433913:TCCGGGT | NA | frameshift | c.1716_1719 | p.Arg573fs |
| A:T | | | +3delCCGGG | |
| | | | TA | |
| 20:32433918:G:A | NA | splice_donor | c.1719+1G > A | |
| 20:32433918:G:C | NA | splice_donor | c.1719+1G > C | |
| 20:32433919:T:A | NA | splice_donor | c.1719+2T > A | |
| 20:32433919:T:C | NA | splice_donor | c.1719+2T > C | |
| 20:32434429:CAGATTC | NA | splice_acceptor | c.1720- | p.Ile574_Leu576 |
| A:C | | | 1_1725delG | del |
| | | | ATTCAA | |
| 20:32434430:A:G | rs376029425 | splice_acceptor | c.1720-2A > G | |
| 20:32434430:A:T | NA | splice_acceptor | c.1720-2A > T | |
| 20:32434431:G:A | NA | splice_acceptor | c.1720-1G > A | |
| 20:32434431:G:C | NA | splice_acceptor | c.1720-1G > C | |
| 20:32434431:G:T | rs1254271466 | splice_acceptor | c.1720-1G > T | |
| 20:32434435:C:T | rs747847938 | stop_gained | c.1723C > T | p.Gln575* |
| 20:32434442:C:A | NA | stop_gained | c.1730C > A | p.Ser577* |
| 20:32434442:C:G | NA | stop_gained | c.1730C > G | p.Ser577* |
| 20:32434448:TCAAACC | NA | frameshift | c.1737_1750 | p.Lys580fs |
| ACCCTGGG:T | | | delCAAACCA | |
| | | | CCCTGGG | |
| 20:32434449:CAAACCA | NA | frameshift | c.1738_1751 | p.Lys580fs |
| CCCTGGGT:C | | | delAAACCAC | |
| | | | CCTGGGT | |
| 20:32434450:AAACCAC | NA | frameshift | c.1741_1757 | p.Pro581fs |
| CCTGGGTGGTT:A | | | delCCACCCT | |
| | | | GGGTGGTTA | |
| | | | A | |
| 20:32434454:CACCCT:C | NA | frameshift | c.1743_1747 | p.Pro582fs |
| | | | delACCCT | |
| 20:32434454:CACCCTG | NA | frameshift | c.1743_1753 | p.Pro582fs |
| GGTGG:C | | | delACCCTGG | |
| | | | GTGG | |
| 20:32434455:ACCCTGG | NA | frameshift | c.1744_1751 | p.Pro582fs |
| GT:A | | | delCCCTGGG | |
| | | | T | |
| 20:32434456:CCCTGGG | NA | frameshift | c.1745_1752 | p.Pro582fs |
| TG:C | | | delCCTGGGT | |
| | | | G | |
| 20:32434457:C:CCTGGG | NA | frameshift | c.1747_1762 | p.Gln588fs |
| TGGTTAAAGGT | | | dupTGGGTG | |
| | | | GTTAAAGGT | |
| | | | C | |
| 20:32434459:TGGGTGG | NA | frameshift | c.1750_1760 | p.Va1584fs |
| TTAAA:T | | | delGTGGTTA | |
| | | | AAGG | |
| 20:32434460:G:A | rs763361634 | stop_gained | c.1748G > A | p.Trp583* |
| 20:32434461:G:A | rs1174760074 | stop_gained | c.1749G > A | p.Trp583* |
| 20:32434463:T:TAACC | rs768359987 | frameshift | c.1751_1752 | p.Va1585fs |
| | | | insAACC | |
| 20:32434465:G:GTTAAA | NA | frameshift | c.1757_1772 | p.Tyr591fs |
| GGTCAGCCCAC | | | dupAAGGTC | |
| | | | AGCCCACTT | |
| | | | A | |
| 20:32434466:T:TTAAA | NA | frameshift | c.1755_1758 | p.Gly587fs |
| | | | dupTAAA | |
| 20:32434467:T:TA | NA | frameshift | c.1758dupA | p.Gly587fs |
| 20:32434468:A:T | NA | stop_gained | c.1756A > T | p.Lys586* |
| 20:32434472:G:GT | NA | frameshift | c.1761dupT | p.Gln588fs |
| 20:32434473:T:TC | NA | frameshift | c.1762dupC | p.Gln588fs |
| 20:32434474:C:CA | NA | frameshift | c.1763dupA | p.Pro589fs |
| 20:32434474:C:T | rs1486082302 | stop_gained | c.1762C > T | p.Gln588* |
| 20:32434478:CCA:C | NA | frameshift | c.1768_1769 | p.Thr590fs |
| | | | delAC | |
| 20:32434481:C:CT | | frameshift | c.1771dupT | p.Tyr591fs |
| 20:32434481:CTT:C | rs774161869 | frameshift | c.1770_1771 | p.Tyr591fs |
| | | | delTT | |
| 20:32434483:T:TA | rs1491418513, | frameshift | c.1772dupA | p.Tyr591fs |
| | rs762036456 | | | |
| 20:32434483:T:TAA | NA | frameshift | c.1772_1773 | p.Tyr591fs |
| | | | insAA | |
| 20:32434483:T:TAG | NA | frameshift | c.1772_1773 | p.Tyr591fs |
| | | | insGA | |
| 20:32434483:TAC:T | NA | frameshift | c.1772_1773 | p.Tyr591fs |
| | | | delAC | |
| 20:32434483:TACC:T | NA | stop_gained | c.1773_1775 | p.Tyr591_Gln592 |
| | | | delCCA | delinsTer |
| 20:32434483:TACCAG:T | NA | frameshift | c.1773_1777 | p.Gln592fs |
| | | | delCCAGA | |
| 20:32434483:TACCAGA | NA | frameshift | c.1772_1785 | p.Tyr591fs |
| TATGCCCC:T | | | delACCAGAT | |
| | | | ATGCCCC | |
| 20:32434484:ACCAGAT | NA | frameshift | c.1773_1780 | p.Tyr591fs |
| AT:A | | | delCCAGATA | |
| | | | T | |
| 20:32434485:C:A | rs371369583 | stop_gained | c.1773C > A | p.Tyr591* |
| 20:32434485:C:G | rs371369583 | stop_gained | c.1773C > G | p.Tyr591* |
| 20:32434486:C:T | rs951716574 | stop_gained | c.1774C > T | p.Gln592* |
| 20:32434488:GA:G | NA | frameshift | c.1777delA | p.Ile593fs |
| 20:32434488:GAT:G | NA | frameshift | c.1779_1780 | p.Ile593fs |
| | | | delAT | |
| 20:32434490:T:TA | rs1314081658 | frameshift | c.1779dupA | p.Cys594fs |
| 20:32434493:G:GC | NA | frameshift | c.1786dupC | p.Arg596fs |
| 20:32434494:C:A | rs755974145 | stop_gained | c.1782C > A | p.Cys594* |
| 20:32434503:C:CATCCC | NA | frameshift | c.1793_1805 | p.Glu602fs |
| CACCACGG | | | dupTCCCCAC | |
| | | | CACGGA | |
| 20:32434505:T:TCCCCA | NA | frameshift | c.1796_1800 | p.Thr601fs |
| | | | dupCCACC | |
| 20:32434509:C:CA | NA | frameshift | c.1798dupA | p.Thr600fs |
| 20:32434514:C:CGGCA | NA | frameshift | c.1804_1805 | p.Glu602fs |
| | | | insCAGG | |
| 20:32434516:G:T | rs907795578 | stop_gained | c.1804G > T | p.Glu602* |
| 20:32434520:C:CCAACC | NA | frameshift | c.1809_1810 | p.Ser604fs |
| CCGGCAGGAGGA | | | insAACCCCG | |
| | | | GCAGGAGGA | |
| | | | C | |
| 20:32434524:C:CG | NA | frameshift | c.1812_1813 | p.Cys605fs |
| | | | insG | |
| 20:32434530:G:GCAGG | NA | frameshift | c.1818_1819 | p.Gly607fs |
| AGGACTCCGT | | | insCAGGAGG | |
| | | | ACTCCGT | |
| 20:32434543:G:GC | NA | frameshift | c.1833dupC | p.Arg612fs |
| 20:32434543:GCC:G | | frameshift | c.1832_1833 | p.Ala611fs |
| | | | delCC | |
| 20:32434549:A:AC | NA | frameshift | c.1840dupC | p.Leu614fs |
| 20:32434550:CCCTCGCA | NA | frameshift | c.1841_1857 | p.Leu614fs |
| GACATTAAAG:C | | | delTCGCAGA | |
| | | | CATTAAAGC | |
| | | | C | |
| 20:32434553:TCG:T | NA | frameshift | c.1843_1844 | p.Ala615fs |
| | | | delGC | |
| 20:32434558:GAC:G | NA | frameshift | c.1848_1849 | p.Ile617fs |
| | | | delCA | |
| 20:32434560:C:CT | rs767955952 | frameshift | c.1848_1849 | p.Ile617fs |
| | | | insT | |
| 20:32434560:C:CTA | NA | frameshift | c.1848_1849 | p.Ile617fs |
| | | | insTA | |
| 20:32434560:C:CTAAT | NA | frameshift | c.1848_1849 | p.Ile617fs |
| | | | insTA AT | |
| 20:32434560:CAT:C | | frameshift | c.1849_1850 | p.Ile617fs |
| | | | delAT | |
| 20:32434561:A:AT | NA | frameshift | c.1851dupT | p.Lys618fs |
| 20:32434562:T:TA | rs773562079 | frameshift | c.1850_1851 | p.Lys618fs |
| | | | insA | |
| 20:32434563:T:TA | NA | frameshift | c.1854dupA | p.Ala619fs |
| 20:32434563:TAA:T | NA | frameshift | c.1853_1854 | p.Lys618fs |
| | | | delAA | |
| 20:32434567:G:GC | NA | frameshift | c.1858dupC | p.Arg620fs |
| 20:32434567:GCC:G | NA | frameshift | c.1857_1858 | p.Arg620fs |
| | | | delCC | |
| 20:32434567:GCCCGTG | NA | frameshift | c.1857_1891 | p.Arg620fs |
| CTCTGCAGGTCCGAGGG | | | delCCGTGCT | |
| GCGAGAGGTCAC:G | | | CTGCAGGTC | |
| | | | CGAGGGGCG | |
| | | | AGAGGTCAC | |
| | | | c | |
| 20:32434571:GTGCTC:G | NA | frameshift | c.1863_1867 | p.Leu622fs |
| | | | delTCTGC | |
| 20:32434574:CTCTGCA | NA | frameshift | c.1865_1872 | p.Leu622fs |
| GG:C | | | delTGCAGGT | |
| | | | C | |
| 20:32434579:C:CA | NA | frameshift | c.1868dupA | p.Va1624fs |
| 20:32434579:C:CAGGTC | NA | frameshift | c.1871_1877 | p.Ala627fs |
| CG | | | dupTCCGAG | |
| | | | G | |
| 20:32434579:C:T | rs111316898 | stop_gained | c.1867C > T | p.Gln623* |
| 20:32434580:AGGTCC:A | NA | frameshift | c.1870_1874 | p.Va1624fs |
| | | | delGTCCG | |
| 20:32434582:G:GT | NA | frameshift | c.1871dupT | p.Arg625fs |
| 20:32434583:T:TA | NA | frameshift | c.1871_1872 | p.Arg625fs |
| | | | insA | |
| 20:32434587:A:AG | NA | frameshift | c.1879dupG | p.Ala627fs |
| 20:32434592:C:CGAGA | NA | frameshift | c.1882_1885 | p.Gly629fs |
| | | | dupAGAG | |
| 20:32434592:CGA:C | NA | frameshift | c.1884_1885 | p.Gly629fs |
| | | | delAG | |
| 20:32434596:AGGTCAC | NA | frameshift | c.1887_1909 | p.Glu635fs |
| CACTGCCATAGAGAGGC: | | | delTCACCAC | |
| A | | | TGCCATAGA | |
| | | | GAGGCGG | |
| 20:32434599:TCACCACT | rs766433101 | frameshift | c.1900_1922 | p.Glu635fs |
| GCCATAGAGAGGCGGC: | | | delAGAGAG | |
| T | | | GCGGCCACC | |
| | | | ACTGCCAT | |
| 20:32434608:C:A | rs1356224550 | stop_gained | c.1896C > A | p.Cys632* |
| 20:32434610:A:AT | NA | frameshift | c.l899dupT | p.Arg634fs |
| 20:32434611:T:TA | NA | frameshift | c.l900dupA | p.Arg634fs |
| 20:32434611:TAG:T | rs753983192 | frameshift | c.1904_1905 | p.Glu635fs |
| | | | delAG | |
| 20:32434612:A:T | NA | stop_gained | c.1900A > T | p.Arg634* |
| 20:32434612:AGAGAGG | rs755053984 | frameshift | c.1902_1924 | p.Glu635fs |
| CGGCCACCACTGCCATC: | | | delAGAGGC | |
| A | | | GGCCACCAC | |
| | | | TGCCATCG | |
| 20:32434615:G:T | NA | stop_gained | c.1903G > T | p.Glu635* |
| 20:32434621:G:GC | NA | frameshift | c.1911dupC | p.Thr638fs |
| 20:32434622:CCA:C | NA | frameshift | c.1912_1913 | p.Thr638fs |
| | | | delAC | |
| 20:32434623:CACCACT | NA | frameshift | c.1912_1928 | p.Thr638fs |
| GCCATCGGAGG:C | | | delACCACTG | |
| | | | CCATCGGAG | |
| | | | G | |
| 20:32434624:ACCACTG | NA | frameshift | c.1913_1932 | p.Thr638fs |
| CCATCGGAGGGGGG:A | | | delCCACTGC | |
| | | | CATCGGAGG | |
| | | | GGGG | |
| 20:32434624:ACCACTG | NA | frameshift | c.1913_1935 | p.Thr638fs |
| CCATCGGAGGGGGGGG | | | delCCACTGC | |
| T:A | | | CATCGGAGG | |
| | | | GGGGGGT | |
| 20:32434626:C:CACTA | NA | frameshift | c.1917_1918 | p.Ala640fs |
| | | | insAACT | |
| 20:32434626:CA:C | NA | frameshift | c.1915delA | p.Thr639fs |
| 20:32434626:CACTGCC | NA | frameshift | c.1915_1928 | p.Thr639fs |
| ATCGGAGG:C | | | delACTGCCA | |
| | | | TCGGAGG | |
| 20:32434626:CACTGCC | NA | frameshift | c.1915_1931 | p.Thr639fs |
| ATCGGAGGGGG:C | | | delACTGCCA | |
| | | | TCGGAGGGG | |
| | | | G | |
| 20:32434627:ACTGCCAT | NA | frameshift | c.1916_1929 | p.Thr639fs |
| CGGAGGG:A | | | delCTGCCAT | |
| | | | CGGAGGG | |
| 20:32434627:ACTGCCAT | NA | frameshift | c.1916_1935 | p.Thr639fs |
| CGGAGGGGGGGGT:A | | | delCTGCCAT | |
| | | | CGGAGGGG | |
| | | | GGGGT | |
| 20:32434628:CTGCCA:C | NA | frameshift | c.1918_1922 | p.Ala640fs |
| | | | delGCCAT | |
| 20:32434628:CTGCCATC | NA | frameshift | c.1917_1927 | p.Ala640fs |
| GGAG:C | | | delTGCCATC | |
| | | | GGAG | |
| 20:32434628:CTGCCATC | NA | frameshift | c.1917_1930 | p.Ala640fs |
| GGAGGGG:C | | | delTGCCATC | |
| | | | GGAGGGG | |
| 20:32434629:TGCCATC | rs1262176022 | frameshift | c.1919_1929 | p.Ala640fs |
| GGAGG:T | | | delCCATCGG | |
| | | | AGGG | |
| 20:32434629:TGCCATC | NA | frameshift | c.1918_1937 | p.Ala640fs |
| GGAGGGGGGGGTGG:T | | | delGCCATCG | |
| | | | GAGGGGGG | |
| | | | GGTGG | |
| 20:32434632:CAT:C | NA | frameshift | c.1921_1922 | p.Ile641fs |
| | | | delAT | |
| 20:32434632:CATCGGA | NA | frameshift | c.1921_1928 | p.Ile641fs |
| GG:C | | | delATCGGAG | |
| | | | G | |
| 20:32434635:CGGAG:C | NA | frameshift | c.1926_1929 | p.Gly644fs |
| | | | delAGGG | |
| 20:32434635:CGGAGG: | NA | frameshift | c.1926_1930 | p.Gly644fs |
| C | | | delAGGGG | |
| 20:32434636:G:T | rs892732207 | stop_gained | c.1924G > T | p.Gly642* |
| 20:32434637:G:GA | rs752984377 | frameshift | c.1926dupA | p.Gly643fs |
| 20:32434637:G:GT | NA | frameshift | c.1925_1926 | p.Gly643fs |
| | | | insT | |
| 20:32434638:A:AG | rs1085307856, | frameshift | c.1934dupG | p.Gly646fs |
| | rs756958159 | | | |
| 20:32434638:A:AGAGG | NA | frameshift | c.1927_1928 | p.Gly643fs |
| | | | insAGGG | |
| 20:32434638:AGGGGG: | NA | frameshift | c.1930_1934 | p.Gly644fs |
| A | | | delGGGGG | |
| 20:32434639:G:GA | NA | frameshift | c.1927_1928 | p.Gly643fs |
| | | | insA | |
| 20:32434639:G:GC | NA | frameshift | c.1927_1928 | p.Gly643fs |
| | | | insC | |
| 20:32434639:GGGGGG | NA | frameshift | c.1930_1937 | p.Gly644fs |
| GGT:G | | | delGGGGGT | |
| | | | GG | |
| 20:32434646:G:GT | NA | frameshift | c.1935dupT | p.Gly646fs |
| 20:32434647:TGGCCCG | NA | frameshift | c.1940_1962 | p.Pro647fs |
| GGTGGAGGTGGCGGCG | | | delCGGGTGG | |
| G:T | | | AGGTGGCGG | |
| | | | CGGGGCC | |
| 20:32434649:G:GC | NA | frameshift | c.1940dupC | p.Gly649fs |
| 20:32434649:GCC:G | NA | frameshift | c.1939_1940 | p.Pro647fs |
| | | | delCC | |
| 20:32434649:GCCCGGG | NA | frameshift | c.1940_1965 | p.Pro647fs |
| TGGAGGTGGCGGCGGG | | | delCGGGTGG | |
| GCCA:G | | | AGGTGGCGG | |
| | | | CGGGGCCAC | |
| | | | C | |
| 20:32434652:CGG:C | NA | frameshift | c.1942_1943 | p.Gly648fs |
| | | | delGG | |
| 20:32434652:CGGGTG:C | NA | frameshift | c.1942_1946 | p.Gly648fs |
| | | | delGGTGG | |
| 20:32434655:G:GT | NA | frameshift | c.1944dupT | p.Gly649fs |
| 20:32434657:G:T | NA | stop_gained | c.1945G > T | p.Gly649* |
| 20:32434657:GGA:G | NA | frameshift | c.1947_1948 | p.Gly650fs |
| | | | delAG | |
| 20:32434662:T:TG | NA | frameshift | c.1952dupG | p.Gly652fs |
| 20:32434662:TGGCGGC | NA | frameshift | c.1953_1972 | p.Gly652fs |
| GGGGCCACCGATGA:T | | | delCGGCGG | |
| | | | GGCCACCGA | |
| | | | TGAGG | |
| 20:32434668:CGGGGCC | NA | frameshift | c.1960_1973 | p.Ala654fs |
| ACCGATGA:C | | | delGCCACCG | |
| | | | ATGAGGG | |
| 20:32434670:G:GT | NA | frameshift | c.1958_1959 | p.Ala654fs |
| | | | insT | |
| 20:32434672:GCCACCG | NA | frameshift | c.1961_1968 | p.Ala654fs |
| AT:G | | | delCCACCGA | |
| | | | T | |
| 20:32434675:A:AC | NA | frameshift | c.1965dupC | p.Asp656fs |
| 20:32434676:CCGATG:C | NA | frameshift | c.1965_1969 | p.Asp656fs |
| | | | delCGATG | |
| 20:32434680:T:TG | NA | frameshift | c.1969dupG | p.Glu657fs |
| 20:32434680:T:TGAGG | NA | frameshift | c.1973_1976 | p.Gly660fs |
| | | | dupGAGG | |
| 20:32434681:G:T | NA | stop_gained | c.1969G > T | p.Glu657* |
| 20:32434684:G:T | NA | stop_gained | c.1972G > T | p.Gly658* |
| 20:32434692:C:CA | NA | frameshift | c.1981dupA | p.Arg661fs |
| 20:32434693:A:T | NA | stop_gained | c.1981A > T | p.Arg661* |
| 20:32434702:AGC:A | NA | frameshift | c.1991_1992 | p.Ser664fs |
| | | | delGC | |
| 20:32434702:AGCAGTG | NA | frameshift | c.1992_2002 | p.Ser664fs |
| GTGAT:A | | | delCAGTGGT | |
| | | | GATG | |
| 20:32434712:A:ATGGTG | NA | frameshift | c.2003_2015 | p.Cys672fs |
| GTGAGGCC | | | dupGTGGTG | |
| | | | AGGCCTG | |
| 20:32434718:G:GTGAG | NA | frameshift | c.2009_2015 | p.Cys672fs |
| GCC | | | dupAGGCCT | |
| | | | G | |
| 20:32434721:AGG:A | NA | frameshift | c.2010_2011 | p.Glu670fs |
| | | | delGG | |
| 20:32434723:G:GC | NA | frameshift | c.2013dupC | p.Cys672fs |
| 20:32434728:T:A | NA | stop_gained | c.2016T > A | p.Cys672* |
| 20:32434728:T:TGGCCT | NA | frameshift | c.2020_2021 | p.His674fs |
| CCCCTGGGCTCAGGGTG | | | insTCCCCTG | |
| GCCACA | | | GGCTCAGGG | |
| | | | TGGCCACAG | |
| | | | GCC | |
| 20:32434731:CCACCCTG | NA | frameshift | c.2020_2027 | p.His674fs |
| A:C | | | delCACCCTG | |
| | | | A | |
| 20:32434732:C:CT | NA | frameshift | c.2020_2021 | p.His674fs |
| | | | insT | |
| 20:32434734:CCCTGAG | NA | frameshift | c.2025_2041 | p.Pro677fs |
| CCCAGGGGAGG:C | | | delTGAGCCC | |
| | | | AGGGGAGG | |
| | | | CC | |
| 20:32434737:TGAGCCC | NA | frameshift | c.2027_2034 | p.Glu676fs |
| AG:T | | | delAGCCCAG | |
| | | | G | |
| 20:32434738:G:T | NA | stop_gained | c.2026G > T | p.Glu676* |
| 20:32434744:A:AG | NA | frameshift | c.2036dupG | p.Gly680fs |
| 20:32434744:AG:A | NA | frameshift | c.2036delG | p.Gly679fs |
| 20:32434747:G:T | NA | stop_gained | c.2035G > T | p.Gly679* |
| 20:32434757:G:GT | NA | frameshift | c.2045_2046 | p.Thr683fs |
| | | | insT | |
| 20:32434759:A:AT | NA | frameshift | c.2047_2048 | p.Thr683fs |
| | | | insT | |
| 20:32434763:C:CT | NA | frameshift | c.2052dupT | p.Gly685fs |
| 20:32434765:G:T | NA | stop_gained | c.2053G > T | p.Gly685* |
| 20:32434768:A:T | NA | stop_gained | c.2056A > T | p.Lys686* |
| 20:32434769:AGT:A | rs780146542 | frameshift | c.2060_2061 | p.Cys687fs |
| | | | delGT | |
| 20:32434772:GTACGTC | NA | frameshift | c.2064_2074 | p.Ser689fs |
| AGATC:G | | | delGTCAGAT | |
| | | | CTAC | |
| 20:32434773:T:A | rs1160049111 | stop_gained | c.2061T > A | p.Cys687* |
| 20:32434778:C:CA | NA | frameshift | c.2067dupA | p.Asp690fs |
| 20:32434778:C:G | rs1387681483 | stop_gained | c.2066C > G | p.Ser689* |
| 20:32434786:C:T | rs1478929932 | stop_gained | c.2074C > T | p.Gln692* |
| 20:32434789:C:T | rs373221034 | stop_gained | c.2077C > T | p.Arg693* |
| 20:32434789:CG:C | NA | frameshift | c.2078delG | p.Arg693fs |
| 20:32434790:GAACAC:G | NA | frameshift | c.2082_2086 | p.Gln695fs |
| | | | delACAAC | |
| 20:32434792:AC:A | NA | frameshift | c.2081delC | p.Thr694fs |
| 20:32434795:C:CA | NA | frameshift | c.2085dupA | p.Leu696fs |
| 20:32434795:C:CAACT | NA | frameshift | c.2085_2088 | p.Leu697fs |
| | | | dupACTA | |
| 20:32434795:C:CT | NA | frameshift | c.2083_2084 | p.Gln695fs |
| | | | insT | |
| 20:32434795:C:T | NA | stop_gained | c.2083C > T | p.Gln695* |
| 20:32434795:CA:C | NA | frameshift | c.2085delA | p.Gln695fs |
| 20:32434797:A:AG | NA | frameshift | c.2085_2086 | p.Leu696fs |
| | | | insG | |
| 20:32434797:AC:A | NA | frameshift | c.2086delC | p.Leu696fs |
| 20:32434798:C:CA | NA | frameshift | c.2086_2087 | p.Leu696fs |
| | | | insA | |
| 20:32434799:T:TA | rs1284591534 | frameshift | c.2088dupA | p.Leu697fs |
| 20:32434800:ACTGCCG | NA | frameshift | c.2091_2097 | p.Pro698fs |
| C:A | | | delGCCGCCT | |
| 20:32434804:CCGCCTTA | NA | frameshift | c.2094_2104 | p.Pro699fs |
| TCCT:C | | | delGCCTTAT | |
| | | | CCTC | |
| 20:32434809:TTATCCTC | NA | frameshift | c.2100_2106 | p.Tyr700fs |
| T | | | delTCCTCTA | |
| 20:32434810:T:TA | NA | frameshift | c.2099dupA | p.Tyr700fs |
| 20:32434810:T:TATCCT | NA | frameshift | c.2099_2127 | p.Gly710fs |
| CTAAATGGGGAGCATAC | | | dupATCCTCT | |
| CCAGGCC | | | AAATGGGGA | |
| | | | GCATACCCA | |
| | | | GGCC | |
| 20:32434813:C:CCT | NA | frameshift | c.2104_2105 | p.Asn703fs |
| | | | dupCT | |
| 20:32434821:T:TG | NA | frameshift | c.2113dupG | p.Glu705fs |
| 20:32434821:TG:T | rs778670589 | frameshift | c.2113delG | p.Glu705fs |
| 20:32434825:G:T | NA | stop_gained | c.2113G > T | p.Glu705* |
| 20:32434825:GA:G | NA | frameshift | c.2114delA | p.Glu705fs |
| 20:32434827:GCATA:G | NA | frameshift | c.2117_2120 | p.His706fs |
| | | | delATAC | |
| 20:32434830:T:TA | NA | frameshift | c.2119dupA | p.Thr707fs |
| 20:32434831:AC:A | NA | frameshift | c.2122delC | p.Gln708fs |
| 20:32434831:ACC:A | NA | frameshift | c.2121_2122 | p.Gln708fs |
| | | | delCC | |
| 20:32434832:CCCAGGC | NA | frameshift | c.2124_2137 | p.Gln708fs |
| CGGAACTG:C | | | delGGCCGGA | |
| | | | ACTGCCA | |
| 20:32434834:C:T | rs755789372 | stop_gained | c.2122C > T | p.Gln708* |
| 20:32434839:CG:C | rs772303842 | frameshift | c.2129delG | p.Gly710fs |
| 20:32434840:G:T | NA | stop_gained | c.2128G > T | p.Gly710* |
| 20:32434840:GGAACTG | NA | frameshift | c.2130_2139 | p.Thr711fs |
| CCAT:G | | | delAACTGCC | |
| | | | ATG | |
| 20:32434841:GA:G | NA | frameshift | c.2131delA | p.Thr711fs |
| 20:32434842:AAC:A | NA | frameshift | c.2131_2132 | p.Thr711fs |
| | | | delAC | |
| 20:32434843:AC:A | NA | frameshift | c.2132delC | p.Thr711fs |
| 20:32434843:ACTGCCAT | NA | frameshift | c.2132_2139 | p.Thr711fs |
| G:A | | | delCTGCCAT | |
| | | | G | |
| 20:32434845:TG:T | NA | frameshift | c.2134delG | p.Ala712fs |
| 20:32434845:TGCCATGT | NA | frameshift | c.2135_2144 | p.Ala712fs |
| CCA:T | | | delCCATGTC | |
| | | | CAG | |
| 20:32434846:GC:G | NA | frameshift | c.2136delC | p.Met713fs |
| 20:32434847:CCA:C | NA | frameshift | c.2136_2137 | p.Met713fs |
| | | | delCA | |
| 20:32434851:G:GTC | NA | frameshift | c.2140_2141 | p.Arg715fs |
| | | | dupTC | |
| 20:32434852:TC:T | NA | frameshift | c.2142delC | p.Arg715fs |
| 20:32434852:TCCA:T | NA | stop_gained | c.2141_2143 | p.Ser714_Arg715 |
| | | | delCCA | delinsTer |
| 20:32434856:GA:G | NA | frameshift | c.2145delA | p.Ala716fs |
| 20:32434857:AG:A | NA | frameshift | c.2146delG | p.Ala716fs |
| 20:32434858:GC:G | NA | frameshift | c.2147delC | p.Ala716fs |
| 20:32434859:CT:C | NA | frameshift | c.2148delT | p.Arg717fs |
| 20:32434860:T:TAG | NA | frameshift | c.2149_2150 | p.Arg718fs |
| | | | dupAG | |
| 20:32434862:GGA:G | NA | frameshift | c.2156_2157 | p.Glu719fs |
| | | | delAG | |
| 20:32434862:GGAGA:G | NA | frameshift | c.2154_2157 | p.Glu719fs |
| | | | delAGAG | |
| 20:32434863:GA:G | NA | frameshift | c.2152delA | p.Arg718fs |
| 20:32434864:A:T | NA | stop_gained | c.2152A > T | p.Arg718* |
| 20:32434864:AG:A | NA | frameshift | c.2153delG | p.Arg718fs |
| 20:32434867:G:T | NA | stop_gained | c.2155G > T | p.Glu719* |
| 20:32434867:GA:G | NA | frameshift | c.2156delA | p.Glu719fs |
| 20:32434868:A:AG | NA | frameshift | c.2158dupG | p.Asp720fs |
| 20:32434868:AG:A | NA | frameshift | c.2158delG | p.Asp720fs |
| 20:32434874:TG:T | NA | frameshift | c.2163delG | p.Pro722fs |
| 20:32434875:GC:G | NA | frameshift | c.2165delC | p.Pro722fs |
| 20:32434877:CT:C | NA | frameshift | c.2167delT | p.Ser723fs |
| 20:32434878:T:TTCTCA | NA | frameshift | c.2170_2171 | p.Leu724fs |
| GAGAAGGCAGGTCC | | | insAGAGAAG | |
| | | | GCAGGTCCT | |
| | | | CTC | |
| 20:32434878:T:TTTCTCA | NA | frameshift | c.2167_2168 | p.Ser723fs |
| GAGAAGGCAGGTCC | | | insTCTCAGA | |
| | | | GAAGGCAGG | |
| | | | TCCT | |
| 20:32434885:A:T | rs757533853 | stop_gained | c.2173A > T | p.Arg725* |
| 20:32434886:GA:G | NA | frameshift | c.2177delA | p.Lys726fs |
| 20:32434888:A:T | rs1415948169 | stop_gained | c.2176A > T | p.Lys726* |
| 20:32434889:AG:A | NA | frameshift | c.2179delG | p.Glu727fs |
| 20:32434891:G:T | rs1264581343 | stop_gained | c.2179G > T | p.Glu727* |
| 20:32434891:GA:G | NA | frameshift | c.2180delA | p.Glu727fs |
| 20:32434894:G:T | NA | stop_gained | c.2182G > T | p.Glu728* |
| 20:32434894:GA:G | NA | frameshift | c.2185delA | p.Ser729fs |
| 20:32434898:GC:G | NA | frameshift | c.2187delC | p.Cys730fs |
| 20:32434901:GCCTACTA: | NA | frameshift | c.2191_2197 | p.Leu731fs |
| G | | | delCTACTAC | |
| 20:32434902:C:A | rs1297030768 | stop_gained | c.2190C > A | p.Cys730* |
| 20:32434903:C:CTA | NA | frameshift | c.2192_2193 | p.Leu732fs |
| | | | dupTA | |
| 20:32434904:TACTACA | NA | frameshift | c.2195_2219 | p.Leu732fs |
| GAGGGCTACAGTTGGAC | | | delTACAGAG | |
| TC:T | | | GGCTACAGT | |
| | | | TGGACTCAC | |
| 20:32434906:C:CTA | NA | frameshift | c.2195_2196 | p.Gln733fs |
| | | | dupTA | |
| 20:32434908:AC:A | rs777135381 | frameshift | c.2197delC | p.Gln733fs |
| 20:32434909:C:T | rs387907078 | stop_gained | c.2197C > T | p.Gln733* |
| 20:32434912:A:AGG | NA | frameshift | c.2202_2203 | p.Ala735fs |
| | | | dupGG | |
| 20:32434912:AG:A | rs1476818548 | frameshift | c.2203delG | p.Ala735fs |
| 20:32434920:AG:A | NA | frameshift | c.2209delG | p.Va1737fs |
| 20:32434923:TG:T | NA | frameshift | c.2213delG | p.Gly738fs |
| 20:32434924:G:T | rs1165142818 | stop_gained | c.2212G > T | p.Gly738* |
| 20:32434925:GACTC:G | NA | frameshift | c.2216_2219 | p.Leu739fs |
| | | | delTCAC | |
| 20:32434927:CT:C | NA | frameshift | c.2216delT | p.Leu739fs |
| 20:32434928:T:TCA | NA | frameshift | c.2219_2220 | p.Asp741fs |
| | | | dupCA | |
| 20:32434934:A:ATG | NA | frameshift | c.2223_2224 | p.Gly742fs |
| | | | dupTG | |
| 20:32434938:GC:G | NA | frameshift | c.2227delC | p.Leu743fs |
| 20:32434940:TA:T | NA | frameshift | c.2229delA | p.Gly744fs |
| 20:32434941:A:AG | NA | frameshift | c.2231dupG | p.Asp745fs |
| 20:32434943:GA:G | NA | frameshift | c.2232delA | p.Asp745fs |
| 20:32434946:AT:A | NA | frameshift | c.2235delT | p.Asp745fs |
| 20:32434948:GC:G | NA | frameshift | c.2238delC | p.Ser747fs |
| 20:32434951:TC:T | NA | frameshift | c.2242delC | p.Gln748fs |
| 20:32434954:C:T | rs1202551247 | stop_gained | c.2242C > T | p.Gln748* |
| 20:32434954:CA:C | NA | frameshift | c.2244delA | p.Gln748fs |
| 20:32434957:CT:C | NA | frameshift | c.2246delT | p.Leu749fs |
| 20:32434958:TC:T | rs1481246277 | frameshift | c.2250delC | p.Va1751fs |
| 20:32434962:C:CA | NA | frameshift | c.2250_2251 | p.Va1751fs |
| | | | insA | |
| 20:32434963:GT:G | NA | frameshift | c.2253delT | p.Ala752fs |
| 20:32434963:GTTGCTCC | NA | frameshift | c.2253_2262 | p.Ala752fs |
| CAC:G | | | delTGCTCCC | |
| | | | ACT | |
| 20:32434965:T:TA | NA | frameshift | c.2253_2254 | p.Ala752fs |
| | | | insA | |
| 20:32434965:T:TG | rs1214641052 | frameshift | c.2254dupG | p.Ala752fs |
| 20:32434965:TG:T | NA | frameshift | c.2254delG | p.Ala752fs |
| 20:32434968:TC:T | NA | frameshift | c.2259delC | p.Thr754fs |
| 20:32434968:TCCCACTG | NA | frameshift | c.2259_2278 | p.Thr754fs |
| GGGACCAGCCATG:T | | | delCACTGGG | |
| | | | GACCAGCCA | |
| | | | TGCC | |
| 20:32434970:C:CCCAGT | NA | frameshift | c.2259_2260 | p.Thr754fs |
| GGGAG | | | insCAGTGGG | |
| | | | AGC | |
| 20:32434970:CCACTG:C | NA | frameshift | c.2259_2263 | p.Thr754fs |
| | | | delCACTG | |
| 20:32434970:CCACTGG | NA | frameshift | c.2259_2274 | p.Thr754fs |
| GGACCAGCCA:C | | | delCACTGGG | |
| | | | GACCAGCCA | |
| 20:32434971:C:CCAGTG | NA | frameshift | c.2259_2260 | p.Thr754fs |
| GGAGCA | | | insCAGTGGG | |
| | | | AGCA | |
| 20:32434971:CA:C | NA | frameshift | c.2260delA | p.Thr754fs |
| 20:32434972:AC:A | NA | frameshift | c.2261delC | p.Thr754fs |
| 20:32434973:CT:C | NA | frameshift | c.2262delT | p.Asp756fs |
| 20:32434974:TG:T | NA | frameshift | c.2266delG | p.Asp756fs |
| 20:32434979:AC:A | rs1449831583 | frameshift | c.2269delC | p.Gln757fs |
| 20:32434981:C:T | rs779078826 | stop_gained | c.2269C > T | p.Gln757* |
| 20:32434985:CA:C | NA | frameshift | c.2274delA | p.Cys759fs |
| 20:32434986:A:AT | | frameshift | c.2275dupT | p.Cys759fs |
| 20:32434986:A:ATG | NA | frameshift | c.2275_2276 | p.Gln760fs |
| | | | dupTG | |
| 20:32434988:GCCAGGC | NA | frameshift | c.2278_2290 | p.Gln760fs |
| CTTGCCC:G | | | delCAGGCCT | |
| | | | TGCCCC | |
| 20:32434989:C:A | NA | stop_gained | c.2277C > A | p.Cys759* |
| 20:32434990:C:T | rs1167715259 | stop_gained | c.2278C > T | p.Gln760* |
| 20:32434990:CA:C | NA | frameshift | c.2279delA | p.Gln760fs |
| 20:32434993:GC:G | NA | frameshift | c.2283delC | p.Leu762fs |
| 20:32434995:C:CT | NA | frameshift | c.2285dupT | p.Leu762fs |
| 20:32434997:T:A | NA | stop_gained | c.2285T > A | p.Leu762* |
| 20:32434998:GC:G | NA | frameshift | c.2290delC | p.Leu764fs |
| 20:32434998:GCC:G | NA | frameshift | c.2289_2290 | p.Leu764fs |
| | | | delCC | |
| 20:32435003:T:TA | NA | frameshift | c.2292dupA | p.Leu765fs |
| 20:32435004:AC:A | NA | frameshift | c.2293delC | p.Leu765fs |
| 20:32435004:ACTGTCCT | NA | frameshift | c.2294_2301 | p.Leu765fs |
| C:A | | | delTGTCCTC | |
| | | | C | |
| 20:32435004:ACTGTCCT | NA | frameshift | c.2296_2317 | p.Ser766fs |
| CCCAAACCTCAGTAG:A | | | delTCCTCCC | |
| | | | AAACCTCAG | |
| | | | TAGCTG | |
| 20:32435005:C:CT | NA | frameshift | c.2294dupT | p.Ser766fs |
| 20:32435005:CT:C | NA | frameshift | c.2294delT | p.Leu765fs |
| 20:32435008:TC:T | rs1315414366 | frameshift | c.2298delC | p.Ser767fs |
| 20:32435011:TC:T | NA | frameshift | c.2302delC | p.Gln768fs |
| 20:32435014:C:T | rs770762273 | stop_gained | c.2302C > T | p.Gln768* |
| 20:32435014:CA:C | NA | frameshift | c.2305delA | p.Thr769fs |
| 20:32435021:C:G | NA | stop_gained | c.2309C > G | p.Ser770* |
| 20:32435023:GT:G | NA | frameshift | c.2312delT | p.Va1771fs |
| 20:32435025:A:AGCTGA | NA | frameshift | c.2316_2344 | p.His782fs |
| GAGATTAGTGGAGCAGC | | | dupTGAGAG | |
| CTCAGTT | | | ATTAGTGGA | |
| | | | GCAGCCTCA | |
| | | | GTTGC | |
| 20:32435028:TGA:T | NA | frameshift | c.2321_2322 | p.Arg774fs |
| | | | delGA | |
| 20:32435029:G:GA | NA | frameshift | c.2318dupA | p.Arg774fs |
| 20:32435029:G:T | rs759218892 | stop_gained | c.2317G > T | p.Glu773* |
| 20:32435032:A:T | NA | stop_gained | c.2320A > T | p.Arg774* |
| 20:32435034:AT:A | rs765327792 | frameshift | c.2324delT | p.Leu775fs |
| 20:32435036:T:A | rs752263134 | stop_gained | c.2324T > A | p.Leu775* |
| 20:32435036:T:G | rs752263134 | stop_gained | c.2324T > G | p.Leu775* |
| 20:32435041:G:T | NA | stop_gained | c.2329G > T | p.Glu777* |
| 20:32435044:C:T | NA | stop_gained | c.2332C > T | p.Gln778* |
| 20:32435050:C:T | rs751021760 | stop_gained | c.2338C > T | p.Gln780* |
| 20:32435054:T:TG | NA | frameshift | c.2343dupG | p.His782fs |
| 20:32435065:GT:G | NA | frameshift | c.2355delT | p.Arg786fs |
| 20:32435066:T:TTA | NA | frameshift | c.2355_2356 | p.Arg786fs |
| | | | dupTA | |
| 20:32435074:G:GA | NA | frameshift | c.2364dupA | p.Cys789fs |
| 20:32435074:G:T | rs1402270258 | stop_gained | c.2362G > T | p.Glu788* |
| 20:32435074:GA:G | NA | frameshift | c.2364delA | p.Glu788fs |
| 20:32435075:A:AT | NA | frameshift | c.2363_2364 | p.Glu788fs |
| | | | insT | |
| 20:32435076:ATG:A | NA | frameshift | c.2367_2368 | p.Cys789fs |
| | | | delTG | |
| 20:32435078:G:GT | NA | frameshift | c.2367dupT | p.Glu790fs |
| 20:32435078:GT:G | NA | frameshift | c.2367delT | p.Cys789fs |
| 20:32435079:T:A | NA | stop_gained | c.2367T > A | p.Cys789* |
| 20:32435080:G:T | NA | stop_gained | c.2368G > T | p.Glu790* |
| 20:32435081:AG:A | NA | frameshift | c.2370delG | p.Glu790fs |
| 20:32435089:AC:A | NA | frameshift | c.2379delC | p.Thr794fs |
| 20:32435093:CT:C | NA | frameshift | c.2383delT | p.Ser795fs |
| 20:32435095:T:TA | NA | frameshift | c.2383_2384 | p.Ser795fs |
| | | | insA | |
| 20:32435095:TC:T | rs752828734 | frameshift | c.2385delC | p.Trp796fs |
| 20:32435097:CT:C | NA | frameshift | c.2386delT | p.Trp796fs |
| 20:32435099:G:A | rs770674396 | stop_gained | c.2387G > A | p.Trp796* |
| 20:32435100:G:A | NA | stop_gained | c.2388G > A | p.Trp796* |
| 20:32435100:GGA:G | NA | frameshift | c.2389_2390 | p.Glu797fs |
| | | | delGA | |
| 20:32435101:G:T | NA | stop_gained | c.2389G > T | p.Glu797* |
| 20:32435104:AG:A | NA | frameshift | c.2393delG | p.Ser798fs |
| 20:32435106:T:TGATGA | NA | frameshift | c.2406_2407 | p.Gln803fs |
| TGAGGAGG | | | insGGATGAT | |
| | | | GAGGAG | |
| 20:32435108:AT:A | NA | frameshift | c.2397delT | p.Asp799fs |
| 20:32435109:TG:T | NA | frameshift | c.2398delG | p.Asp800fs |
| 20:32435113:G:T | NA | stop_gained | c.2401G > T | p.Glu801* |
| 20:32435113:GA:G | NA | frameshift | c.2402delA | p.Glu801fs |
| 20:32435116:G:T | NA | stop_gained | c.2404G > T | p.Glu802* |
| 20:32435118:G:GCC | NA | frameshift | c.2407_2408 | p.Gln803fs |
| | | | insCC | |
| 20:32435118:GC:G | NA | frameshift | c.2407delC | p.Gln803fs |
| 20:32435119:C:T | rs775071544 | stop_gained | c.2407C > T | p.Gln803* |
| 20:32435121:AG:A | NA | frameshift | c.2411delG | p.Gly804fs |
| 20:32435124:A:AC | NA | frameshift | c.2415dupC | p.Thr806fs |
| 20:32435131:GT:G | | frameshift | c.2421delT | p.Pro808fs |
| 20:32435133:T:TC | NA | frameshift | c.2423dupC | p.Ala809fs |
| 20:32435133:TC:T | rs763280972 | frameshift | c.2423delC | p.Pro808fs |
| 20:32435133:TCC:T | NA | frameshift | c.2422_2423 | p.Pro808fs |
| | | | delCC | |
| 20:32435133:TCCTGC:T | NA | frameshift | c.2422_2426 | p.Pro808fs |
| | | | delCCTGC | |
| 20:32435140:GA:G | NA | frameshift | c.2429delA | p.Asp810fs |
| 20:32435141:ACAATGG | NA | frameshift | c.2431_2437 | p.Asn811fs |
| T:A | | | delAATGGTC | |
| 20:32435142:CAA:C | NA | frameshift | c.2431_2432 | p.Asn811fs |
| | | | delAA | |
| 20:32435145:T:TG | NA | frameshift | c.2435dupG | p.Pro813fs |
| 20:32435148:TC:T | NA | frameshift | c.2439delC | p.Ile814fs |
| 20:32435154:TC:T | NA | frameshift | c.2444delC | p.Pro815fs |
| 20:32435156:CG:C | NA | frameshift | c.2445delG | p.Ser816fs |
| 20:32435159:CTCTA:C | NA | frameshift | c.2448_2451 | p.Leu817fs |
| | | | delTCTA | |
| 20:32435159:CTCTAG:C | NA | frameshift | c.2449_2453 | p.Leu817fs |
| | | | delCTAGT | |
| 20:32435161:C:CT | NA | frameshift | c.2450dupT | p.Val818fs |
| 20:32435161:CT:C | NA | frameshift | c.2450delT | p.Leu817fs |
| 20:32435164:GT:G | NA | frameshift | c.2453delT | p.Val818fs |
| 20:32435165:TG:T | NA | frameshift | c.2456delG | p.Gly819fs |
| 20:32435170:G:GA | NA | frameshift | c.2459dupA | p.Asp820fs |
| 20:32435171:AT:A | NA | frameshift | c.2460delT | p.Asp820fs |
| 20:32435174:A:AT | NA | frameshift | c.2463dupT | p.Thr822fs |
| 20:32435174:ATACATTA | NA | frameshift | c.2463_2484 | p.Thr822fs |
| GAGAAAGGAACTGGC:A | | | delTACATTA | |
| | | | GAGAAAGGA | |
| | | | ACTGGC | |
| 20:32435175:T:TA | rs1221361018 | frameshift | c.2464dupA | p.Thr822fs |
| 20:32435176:A:AC | NA | frameshift | c.2465dupC | p.Leu823fs |
| 20:32435178:AT:A | rs767215739 | frameshift | c.2468delT | p.Leu823fs |
| 20:32435179:T:TA | NA | frameshift | c.2467_2468 | p.Leu823fs |
| | | | insA | |
| 20:32435180:T:A | NA | stop_gained | c.2468T > A | p.Leu823* |
| 20:32435180:T:G | NA | stop_gained | c.2468T > G | p.Leu823* |
| 20:32435180:T:TA | NA | frameshift | c.2469dupA | p.Glu824fs |
| 20:32435184:GA:G | NA | frameshift | c.2475delA | p.Gly826fs |
| 20:32435187:A:AG | NA | frameshift | c.2477dupG | p.Thr827fs |
| 20:32435187:AG:A | rs1064796100 | frameshift | c.2477delG | p.Gly826fs |
| 20:32435188:G:T | rs774018728 | stop_gained | c.2476G > T | p.Gly826* |
| 20:32435189:G:GAACT | NA | frameshift | c.2478_2481 | p.Gly828fs |
| | | | dupAACT | |
| 20:32435192:CT:C | NA | frameshift | c.2481delT | p.Gly828fs |
| 20:32435193:T:TG | NA | frameshift | c.2483dupG | p.Gln829fs |
| 20:32435197:C:T | rs1013267324 | stop_gained | c.2485C > T | p.Gln829* |
| 20:32435205:TGACA:T | NA | frameshift | c.2495_2498 | p.Asp832fs |
| | | | delACAG | |
| 20:32435208:CAGTCATC | NA | frameshift | c.2498_2523 | p.Ser833fs |
| CCACTATGAAGGATCCT | | | delGTCATCC | |
| GT:C | | | CACTATGAA | |
| | | | GGATCCTGT | |
| | | | A | |
| 20:32435210:GT:G | NA | frameshift | c.2499delT | p.His834fs |
| 20:32435212:C:CTG | NA | frameshift | c.2500_2501 | p.His834fs |
| | | | insTG | |
| 20:32435219:CT:C | NA | frameshift | c.2508delT | p.Met837fs |
| 20:32435221:AT:A | NA | frameshift | c.2510delT | p.Met837fs |
| 20:32435222:TG:T | NA | frameshift | c.2511delG | p.Met837fs |
| 20:32435225:AG:A | NA | frameshift | c.2515delG | p.Asp839fs |
| 20:32435233:GTA:G | NA | frameshift | c.2522_2523 | p.Val841fs |
| | | | delTA | |
| 20:32435234:TA:T | rs773442382 | frameshift | c.2525delA | p.Asn842fs |
| 20:32435238:TG:T | NA | frameshift | c.2527delG | p.Val843fs |
| 20:32435238:TGTGACC | NA | frameshift | c.2527_2534 | p.Val843fs |
| CC:T | | | delGTGACCC | |
| | | | C | |
| 20:32435242:A:AC | rs750170870 | frameshift | c.2535dupC | p.Ser846fs |
| 20:32435242:AC:A | NA | frameshift | c.2535delC | p.Ser846fs |
| 20:32435248:AGTTCCAC | NA | frameshift | c.2537_2555 | p.Ser846fs |
| ACCTGAATCCTC:A | | | delGTTCCAC | |
| | | | ACCTGAATC | |
| | | | CTC | |
| 20:32435251:TC:T | NA | frameshift | c.2541delC | p.Thr848fs |
| 20:32435252:CCA:C | NA | frameshift | c.2544_2545 | p.Pro849fs |
| | | | delAC | |
| 20:32435256:AC:A | NA | frameshift | c.2546delC | p.Pro849fs |
| 20:32435260:G:T | rs914002110 | stop_gained | c.2548G > T | p.Glu850* |
| 20:32435263:TC:T | NA | frameshift | c.2553delC | p.Ser852fs |
| 20:32435265:C:CT | NA | frameshift | c.2554dupT | p.Ser852fs |
| 20:32435267:C:G | NA | stop_gained | c.2555OG | p.Ser852* |
| 20:32435268:A:ACCGAC | NA | frameshift | c.2559_2617 | p.Pro873fs |
| TGATTGCCTGCAGAACA | | | dupGACTGA | |
| GAGCATTTGATGACGAA | | | TTGCCTGCA | |
| TTAGGGCTTGGTGGCTC | | | GAACAGAGC | |
| ATG | | | ATTTGATGA | |
| | | | CGAATTAGG | |
| | | | GCTTGGTGG | |
| | | | CTCATGCC | |
| 20:32435270:C:CTG | NA | frameshift | c.2558_2559 | p.Thr854fs |
| | | | insTG | |
| 20:32435273:CTGAT:C | rs755989932 | frameshift | c.2564_2567 | p.Asp855fs |
| | | | delATTG | |
| 20:32435274:T:TG | NA | frameshift | c.2563dupG | p.Asp855fs |
| 20:32435274:T:TGATTG | NA | frameshift | c.2565_2575 | p.Asn859fs |
| CCTGCA | | | dupTTGCCTG | |
| | | | CAGA | |
| 20:32435275:GATTGCCT | NA | frameshift | c.2565_2578 | p.Asp855fs |
| GCAGAAC:G | | | delTTGCCTG | |
| | | | CAGAACA | |
| 20:32435277:TTGCC:T | NA | frameshift | c.2569_2572 | p.Leu857fs |
| | | | delCTGC | |
| 20:32435283:GCAGAA: | NA | frameshift | c.2576_2580 | p.Asn859fs |
| G | | | delACAGA | |
| 20:32435284:C:T | NA | stop_gained | c.2572C > T | p.Gln858* |
| 20:32435286:GA:G | NA | frameshift | c.2576delA | p.Asn859fs |
| 20:32435288:AC:A | NA | frameshift | c.2577delC | p.Asn859fs |
| 20:32435289:CA:C | NA | frameshift | c.2578delA | p.Arg860fs |
| 20:32435289:CAG:C | rs1384553029 | frameshift | c.2580_2581 | p.Arg860fs |
| | | | delAG | |
| 20:32435290:A:T | NA | stop_gained | c.2578A > T | p.Arg860* |
| 20:32435292:AG:A | NA | frameshift | c.2581delG | p.Ala861fs |
| 20:32435293:GC:G | NA | frameshift | c.2582delC | p.Ala861fs |
| 20:32435294:C:CA | NA | frameshift | c.2583dupA | p.Phe862fs |
| 20:32435299:GA:G | NA | frameshift | c.2588delA | p.Asp863fs |
| 20:32435299:GATGACG | NA | frameshift | c.2588_2595 | p.Asp863fs |
| AA:G | | | delATGACGA | |
| | | | A | |
| 20:32435305:G:T | rs147895689 | stop_gained | c.2593G > T | p.Glu865* |
| 20:32435309:T:G | NA | stop_gained | c.2597T > G | p.Leu866* |
| 20:32435310:AG:A | NA | frameshift | c.2601delG | p.Gly869fs |
| 20:32435316:TG:T | NA | frameshift | c.2606delG | p.Gly869fs |
| 20:32435320:G:GGCTCA | NA | frameshift | c.2610_2655 | p.Lys886fs |
| TGCCCTCCTATGAGGGA | | | dupCTCATGC | |
| AAGTGATACTAGACAAG | | | CCTCCTATGA | |
| AAAACTT | | | GGGAAAGTG | |
| | | | ATACTAGAC | |
| | | | AAGAAAACT | |
| | | | TG | |
| 20:32435322:C:CCA | NA | frameshift | c.2610_2611 | p.Ser871fs |
| | | | insCA | |
| 20:32435324:CA:C | rs780091312 | frameshift | c.2613delA | p.Cys872fs |
| 20:32435327:G:GC | NA | frameshift | c.2618dupC | p.Pro874fs |
| 20:32435328:C:A | NA | stop_gained | c.2616C > A | p.Cys872* |
| 20:32435328:CCCTCCTA | NA | frameshift | c.2617_2626 | p.Pro873fs |
| TGA:C | | | delCCTCCTAT | |
| | | | GA | |
| 20:32435338:AG:A | NA | frameshift | c.2629delG | p.Glu877fs |
| 20:32435341:G:GA | NA | frameshift | c.2632dupA | p.Ser878fs |
| 20:32435341:GA:G | NA | frameshift | c.2632delA | p.Ser878fs |
| 20:32435344:AGT:A | NA | frameshift | c.2634_2635 | p.Ser878fs |
| | | | delTG | |
| 20:32435349:T:TA | NA | frameshift | c.2638dupA | p.Thr880fs |
| 20:32435350:AC:A | NA | frameshift | c.2639delC | p.Thr880fs |
| 20:32435356:C:T | rs770209084 | stop_gained | c.2644C > T | p.Gln882* |
| 20:32435363:AC:A | NA | frameshift | c.2652delC | p.Leu885fs |
| 20:32435366:T:A | NA | stop_gained | c.2654T > A | p.Leu885* |
| 20:32435368:A:T | NA | stop_gained | c.2656A > T | p.Lys886* |
| 20:32435374:A:T | NA | stop_gained | c.2662A > T | p.Lys888* |
| 20:32435375:A:AG | NA | frameshift | c.2665dupG | p.Ala889fs |
| 20:32435380:C:CT | rs1432318140 | frameshift | c.2669dupT | p.Val891fs |
| 20:32435385:TTC:T | NA | frameshift | c.2675_2676 | p.Ser892fs |
| | | | delCT | |
| 20:32435387:C:CT | NA | frameshift | c.2676dupT | p.Asn893fs |
| 20:32435390:AC:A | NA | frameshift | c.2679delC | p.Asn893fs |
| 20:32435393:GT:G | rs778535367 | frameshift | c.2683delT | p.Ser895fs |
| 20:32435395:T:TCTTTG | NA | frameshift | c.2685_2689 | p.His897fs |
| | | | dupTTTGC | |
| 20:32435395:TC:T | NA | frameshift | c.2684delC | p.Ser895fs |
| 20:32435396:C:CT | NA | frameshift | c.2687dupT | p.Leu896fs |
| 20:32435405:G:A | NA | stop_gained | c.2693G > A | p.Trp898* |
| 20:32435406:G:A | rs760592730 | stop_gained | c.2694G > A | p.Trp898* |
| 20:32435407:A:ATACCC | NA | frameshift | c.2696_2700 | p.Ile901fs |
| | | | dupTACCC | |
| 20:32435407:AT:A | NA | frameshift | c.2696delT | p.Ile899fs |
| 20:32435408:T:TA | NA | frameshift | c.2697dupA | p.Pro900fs |
| 20:32435409:A:AC | NA | frameshift | c.2700dupC | p.Ile901fs |
| 20:32435409:AC:A | NA | frameshift | c.2700delC | p.Ile901fs |
| 20:32435414:TC:T | NA | frameshift | c.2705delC | p.Pro902fs |
| 20:32435418:A:ATCAT | NA | frameshift | c.2708_2709 | p.Asn904fs |
| | | | insATTC | |
| 20:32435419:TCGAATG | NA | frameshift | c.2708_2714 | p.Ser903fs |
| A:T | | | delCGAATGA | |
| 20:32435421:GA:G | NA | frameshift | c.2711delA | p.Asn904fs |
| 20:32435428:GA:G | NA | frameshift | c.2717delA | p.Glu906fs |
| 20:32435436:G:GA | NA | frameshift | c.2727dupA | p.Gln910fs |
| 20:32435436:GAA:G | NA | frameshift | c.2726_2727 | p.Lys909fs |
| | | | delAA | |
| 20:32435440:C:T | NA | stop_gained | c.2728C > T | p.Gln910* |
| 20:32435452:G:T | NA | stop_gained | c.2740G > T | p.Glu914* |
| 20:32435458:A:AT | NA | frameshift | c.2746_2747 | p.Arg916fs |
| | | | insT | |
| 20:32435460:AG:A | NA | frameshift | c.2749delG | p.Glu917fs |
| 20:32435462:A:AAC | NA | frameshift | c.2754_2755 | p.Ile919fs |
| | | | dupCA | |
| 20:32435462:AAC:A | NA | frameshift | c.2754_2755 | p.Ile919fs |
| | | | delCA | |
| 20:32435464:CACAT:C | NA | frameshift | c.2755_2758 | p.Ile919fs |
| | | | delATAC | |
| 20:32435468:T:TA | rs771822198 | frameshift | c.2757dupA | p.Pro920fs |
| 20:32435474:C:CT | NA | frameshift | c.2763dupT | p.Val922fs |
| 20:32435478:TGAGCC:T | NA | frameshift | c.2767_2771 | p.Glu923fs |
| | | | delGAGCC | |
| 20:32435481:G:GC | NA | frameshift | c.2773dupC | p.Gln925fs |
| 20:32435481:GC:G | NA | frameshift | c.2773delC | p.Gln925fs |
| 20:32435483:C:CCCAG | NA | frameshift | c.2772_2775 | p.Val926fs |
| | | | dupCCAG | |
| 20:32435485:C:T | rs387907077 | stop_gained | c.2773C > T | p.Gln925* |
| 20:32435487:GGTTGGA | NA | frameshift | c.2777_2789 | p.Val926fs |
| GAGGAGT:G | | | delTTGGAGA | |
| | | | GGAGTG | |
| 20:32435490:TG:T | NA | frameshift | c.2780delG | p.Gly927fs |
| 20:32435493:A:AG | NA | frameshift | c.2782dupG | p.Glu928fs |
| 20:32435494:G:T | NA | stop_gained | c.2782G > T | p.Glu928* |
| 20:32435499:GTGGGAG | NA | frameshift | c.2790_2815 | p.Trp930fs |
| AAAGCTGCTCCCACCCCT | | | delGGAGAA | |
| CCG | | | AGCTGCTCC | |
| | | | CACCCCTCCT | |
| | | | G | |
| 20:32435501:G:A | NA | stop_gained | c.2789G > A | p.Trp930* |
| 20:32435503:G:T | NA | stop_gained | c.2791G > T | p.Glu931* |
| 20:32435505:GA:G | NA | frameshift | c.2796delA | p.Ala933fs |
| 20:32435505:GAAAGCT | NA | frameshift | c.2794_2803 | p.Lys932fs |
| GCTC:G | | | delAAAGCTG | |
| | | | CTC | |
| 20:32435510:C:CT | NA | frameshift | c.2799dupT | p.Ala934fs |
| 20:32435514:T:TCCCAC | NA | frameshift | c.2806_2810 | p.Ala939fs |
| | | | dupACCCC | |
| 20:32435518:A:AAG | NA | frameshift | c.2806_2807 | p.Thr936fs |
| | | | insAG | |
| 20:32435521:C:CT | NA | frameshift | c.2809_2810 | p.Pro937fs |
| | | | insT | |
| 20:32435521:CCTCCTGC | NA | frameshift | c.2810_2817 | p.Pro937fs |
| A:C | | | delCTCCTGC | |
| | | | A | |
| 20:32435522:C:CT | NA | frameshift | c.2811dupT | p.Pro938fs |
| 20:32435523:TC:T | NA | frameshift | c.2813delC | p.Pro938fs |
| 20:32435525:CTGCA:C | NA | frameshift | c.2815_2818 | p.Ala939fs |
| | | | delGCAT | |
| 20:32435528:CA:C | NA | frameshift | c.2817delA | p.Leu940fs |
| 20:32435529:AT:A | NA | frameshift | c.2819delT | p.Leu940fs |
| 20:32435529:ATT:A | NA | frameshift | c.2818_2819 | p.Leu940fs |
| | | | delTT | |
| 20:32435540:AT:A | NA | frameshift | c.2831delT | p.Leu944fs |
| 20:32435545:ACAGCTG | NA | frameshift | c.2834_2841 | p.Thr945fs |
| AG:A | | | delCAGCTGA | |
| | | | G | |
| 20:32435551:G:GAGGA | NA | frameshift | c.2840_2843 | p.Leu950fs |
| | | | dupAGGA | |
| 20:32435551:G:T | NA | stop_gained | c.2839G > T | p.Glu947* |
| 20:32435558:G:GTCTA | NA | frameshift | c.2847_2850 | p.Asp951fs |
| | | | dupTCTA | |
| 20:32435561:T:TA | NA | frameshift | c.2850dupA | p.Asp951fs |
| 20:32435569:C:CT | NA | frameshift | c.2859dupT | p.Asp954fs |
| 20:32435577:CCTTA:C | rs747315609 | frameshift | c.2869_2872 | p.Thr957fs |
| | | | delACTT | |
| 20:32435580:T:TA | NA | frameshift | c.2869dupA | p.Thr957fs |
| 20:32435581:AC:A | NA | frameshift | c.2870delC | p.Thr957fs |
| 20:32435582:C:CT | NA | frameshift | c.2872dupT | p.Ser958fs |
| 20:32435585:C:A | NA | stop_gained | c.2873C > A | p.Ser958* |
| 20:32435585:C:G | NA | stop_gained | c.2873C > G | p.Ser958* |
| 20:32435587:CT:C | NA | frameshift | c.2876delT | p.Leu959fs |
| 20:32435591:G:A | rs1438791925 | stop_gained | c.2879G > A | p.Trp960* |
| 20:32435592:G:A | rs756688220 | stop_gained | c.2880G > A | p.Trp960* |
| 20:32435595:TGTGCCAT | NA | frameshift | c.2885_2897 | p.Val962fs |
| CTCGAG:T | | | delTGCCATC | |
| | | | TCGAGG | |
| 20:32435596:GT:G | NA | frameshift | c.2885delT | p.Val962fs |
| 20:32435605:C:T | rs397515401 | stop_gained | c.2893C > T | p.Arg965* |
| 20:32435608:G:T | NA | stop_gained | c.2896G > T | p.Gly966* |
| 20:32435615:GT:G | NA | frameshift | c.2904delT | p.Ser968fs |
| 20:32435617:GA:G | NA | frameshift | c.2906delA | p.Asp969fs |
| 20:32435630:G:GT | NA | frameshift | c.2920dupT | p.Tyr974fs |
| 20:32435630:G:GTT | NA | frameshift | c.2919_2920 | p.Tyr974fs |
| | | | dupTT | |
| 20:32435632:T:TA | NA | frameshift | c.2921dupA | p.Tyr974fs |
| 20:32435633:AC:A | NA | frameshift | c.2922delC | p.Cys975fs |
| 20:32435634:C:A | rs886039722 | stop_gained | c.2922C > A | p.Tyr974* |
| 20:32435634:CT:C | NA | frameshift | c.2923delT | p.Cys975fs |
| 20:32435638:C:T | rs776868653 | stop_gained | c.2926C > T | p.Gln976* |
| 20:32435641:C:T | rs1366953593 | stop_gained | c.2929C > T | p.Gln977* |
| 20:32435649:C:CA | NA | frameshift | c.2938dupA | p.Ile980fs |
| 20:32435653:GA:G | NA | frameshift | c.2945delA | p.Lys982fs |
| 20:32435658:GCTGAAA | NA | frameshift | c.2949_2982 | p.Lys984fs |
| ATCAACGGAGACTCTGA | | | delGAAAATC | |
| AGCACTGAGTC:G | | | AACGGAGAC | |
| | | | TCTGAAGCA | |
| | | | CTGAGTCCT | |
| 20:32435666:TC:T | NA | frameshift | c.2955delC | p.Asn986fs |
| 20:32435667:CA:C | NA | frameshift | c.2957delA | p.Asn986fs |
| 20:32435669:AC:A | NA | frameshift | c.2958delC | p.Asn986fs |
| 20:32435670:C:CG | NA | frameshift | c.2960dupG | p.Asp988fs |
| 20:32435670:CG:C | NA | frameshift | c.2960delG | p.Gly987fs |
| 20:32435673:AGACTCT | NA | frameshift | c.2963_2987 | p.Asp988fs |
| GAAGCACTGAGTCCTCA | | | delACTCTGA | |
| CG:A | | | AGCACTGAG | |
| | | | TCCTCACGG | |
| 20:32435675:ACT:A | rs1064796772 | frameshift | c.2966_2967 | p.Ser989fs |
| | | | delCT | |
| 20:32435680:G:T | NA | stop_gained | c.2968G > T | p.Glu990* |
| 20:32435682:AGCACT:A | NA | frameshift | c.2972_2976 | p.Ala991fs |
| | | | delCACTG | |
| 20:32435683:GC:G | NA | frameshift | c.2972delC | p.Ala991fs |
| 20:32435684:CA:C | NA | frameshift | c.2973delA | p.Leu992fs |
| 20:32435689:A:AG | NA | frameshift | c.2978dupG | p.Ser993fs |
| 20:32435695:C:CA | NA | frameshift | c.2984dupA | p.His995fs |
| 20:32435696:ACGGT:A | NA | frameshift | c.2985_2988 | p.His995fs |
| | | | delCGGT | |
| 20:32435699:G:GT | NA | frameshift | c.2988dupT | p.Glu997fs |
| 20:32435701:G:T | NA | stop_gained | c.2989G > T | p.Glu997* |
| 20:32435710:GA:G | NA | frameshift | c.2999delA | p.Asp1000fs |
| 20:32435712:T:TA | NA | frameshift | c.3001dupA | p.Thr1001fs |
| 20:32435716:GC:G | NA | frameshift | c.3006delC | p.Ser1003fs |
| 20:32435717:CCT:C | NA | frameshift | c.3008_3009 | p.Ser1003fs |
| | | | delCT | |
| 20:32435724:C:CT | NA | frameshift | c.3015dupT | p.Glu1006fs |
| 20:32435724:CT:C | rs775709283 | frameshift | c.3015delT | p.Phe1005fs |
| 20:32435728:GA:G | NA | frameshift | c.3018delA | p.Gly1007fs |
| 20:32435731:G:GCA | NA | frameshift | c.3019_3020 | p.Gly1007fs |
| | | | insCA | |
| 20:32435731:GGT:G | NA | frameshift | c.3020_3021 | p.Gly1007fs |
| | | | delGT | |
| 20:32435739:C:CA | NA | frameshift | c.3028dupA | p.Thr1010fs |
| 20:32435742:G:GCTGTC | NA | frameshift | c.3030_3031 | p.Glu1011fs |
| CTCCGT | | | insCTGTCCTC | |
| | | | CGT | |
| 20:32435745:G:GGT | NA | frameshift | c.3034_3035 | p.Asp1012fs |
| | | | insTG | |
| 20:32435756:AG:A | NA | frameshift | c.3046delG | p.Ala1016fs |
| 20:32435761:G:GGT | NA | frameshift | c.3049_3050 | p.Asp1017fs |
| | | | insGT | |
| 20:32435763:C:CA | NA | frameshift | c.3052dupA | p.Thr1018fs |
| 20:32435764:A:AT | NA | frameshift | c.3052_3053 | p.Thr1018fs |
| | | | insT | |
| 20:32435765:CTA:C | NA | frameshift | c.3054_3055 | p.Glu1020fs |
| | | | delTA | |
| 20:32435767:A:T | NA | stop_gained | c.3055A > T | p.Arg1019* |
| 20:32435774:CT:C | NA | frameshift | c.3063delT | p.Ala1022fs |
| 20:32435780:TGACAAA | NA | frameshift | c.3074_3092 | p.Lys1025fs |
| GGGATCTTCGGTG:T | | | delAGGGATC | |
| | | | TTCGGTGGA | |
| | | | CAA | |
| 20:32435783:CA:C | NA | frameshift | c.3074delA | p.Lys1025fs |
| 20:32435790:AT:A | NA | frameshift | c.3079delT | p.Ser1027fs |
| 20:32435793:TTC:T | NA | frameshift | c.3082_3083 | p.Ser1028fs |
| | | | delTC | |
| 20:32435795:C:A | rs200702600 | stop_gained | c.3083C > A | p.Ser1028* |
| 20:32435796:G:GGTGG | NA | frameshift | c.3085_3125 | p.Leul043fs |
| ACAAGGATGAGAAACCC | | | dupGTGGAC | |
| AATTGGAACCAATCTGC | | | AAGGATGAG | |
| CCC | | | AAACCCAAT | |
| | | | TGGAACCAA | |
| | | | TCTGCCCC | |
| 20:32435808:TG:T | NA | frameshift | c.3097delG | p.Glu1033fs |
| 20:32435809:G:T | NA | stop_gained | c.3097G > T | p.Glu1033* |
| 20:32435810:AG:A | NA | frameshift | c.3099delG | p.Lys1034fs |
| 20:32435811:GA:G | NA | frameshift | c.3102delA | p.Lys1034fs |
| 20:32435812:A:AAACCC | NA | frameshift | c.3103_3140 | p.Gly1048fs |
| AATTGGAACCAATCTGC | | | dupCCCAATT | |
| CCCACTGTCCAAGGTG | | | GGAACCAAT | |
| | | | CTGCCCCACT | |
| | | | GTCCAAGGT | |
| | | | GAA | |
| 20:32435812:A:T | NA | stop_gained | c.3100A > T | p.Lys1034* |
| 20:32435819:AT:A | NA | frameshift | c.3109delT | p.Trp1037fs |
| 20:32435822:G:A | NA | stop_gained | c.3110G > A | p.Trp1037* |
| 20:32435823:G:A | NA | stop_gained | c.3111G > A | p.Trp1037* |
| 20:32435825:ACCAATCT | NA | frameshift | c.3117_3126 | p.Gln1039fs |
| GCC:A | | | delATCTGCC | |
| | | | CCA | |
| 20:32435827:C:T | rs1221031683 | stop_gained | c.3115C > T | p.Gln1039* |
| 20:32435833:GC:G | NA | frameshift | c.3125delC | p.Pro1042fs |
| 20:32435842:T:TC | NA | frameshift | c.3132dupC | p.Lys1045fs |
| 20:32435842:TC:T | NA | frameshift | c.3132delC | p.Lys1045fs |
| 20:32435844:CA:C | NA | frameshift | c.3134delA | p.Lys1045fs |
| 20:32435846:AG:A | NA | frameshift | c.3136delG | p.Val1046fs |
| 20:32435858:A:ACATG | NA | frameshift | c.3148_3151 | p.Arg1051fs |
| | | | dupATGC | |
| 20:32435867:T:TC | NA | frameshift | c.3155_3156 | p.Val1053fs |
| | | | insC | |
| 20:32435899:C:CA | NA | frameshift | c.3188dupA | p.Ser1064fs |
| 20:32435899:C:T | rs1311033207 | stop_gained | c.3187C > T | p.Gln1063* |
| 20:32435902:AGCTGG:A | NA | frameshift | c.3192_3196 | p.Trp1065fs |
| | | | delCTGGG | |
| 20:32435907:G:A | NA | stop_gained | c.3195G > A | p.Trp1065* |
| 20:32435910:GTC:G | NA | frameshift | c.3201_3202 | p.Arg1068fs |
| | | | delTC | |
| 20:32435914:C:T | rs764651405 | stop_gained | c.3202C > T | p.Arg1068* |
| 20:32436044:T:A | NA | stop_gained | c.3332T > A | p.Leu1111* |
| 20:32436053:GTAGCTT | NA | frameshift | c.3345_3355 | p.Ser1115fs |
| GCCCC:G | | | delCTTGCCC | |
| | | | CTAG | |
| 20:32436078:TCC:T | NA | frameshift | c.3367_3368 | p.Pro1123fs |
| | | | delCC | |
| 20:32436115:C:T | NA | stop_gained | c.3403C > T | p.Gln1135* |
| 20:32436130:A:T | NA | stop_gained | c.3418A > T | p.Lys1140* |
| 20:32436136:C:T | NA | stop_gained | c.3424C > T | p.Gln1142* |
| 20:32436137:A:AGAGCC | NA | frameshift | c.3433_3434 | p.Gly1145fs |
| ATGCGTAGC | | | insCGTAGCG | |
| | | | AGCCATG | |
| 20:32436188:GTGGCA: | NA | frameshift | c.3480_3484 | p.Met1161fs |
| G | | | delCATGG | |
| 20:32436208:AG:A | NA | frameshift | c.3497delG | p.Ser1166fs |
| 20:32436213:CA:C | NA | frameshift | c.3502delA | p.Ser1168fs |
| 20:32436215:GT:G | NA | frameshift | c.3505delT | p.Ser1169fs |
| 20:32436223:AG:A | NA | frameshift | c.3514delG | p.Ala1172fs |
| 20:32436258:T:A | NA | stop_gained | c.3546T > A | p.Cys1182* |
| 20:32436286:G:T | NA | stop_gained | c.3574G > T | p.Glu1192* |
| 20:32436295:C:T | NA | stop_gained | c.3583C > T | p.Gln1195* |
| 20:32436300:TC:T | NA | frameshift | c.3590delC | p.Pro1197fs |
| 20:32436303:T:TGGAGC | NA | frameshift | c.3592_3602 | p.Lys1202fs |
| ACCCCA | | | dupGGAGCA | |
| | | | CCCCA | |
| 20:32436305:GA:G | NA | frameshift | c.3594delA | p.Ala1199fs |
| 20:32436309:AC:A | NA | frameshift | c.3601delC | p.Gln1201fs |
| 20:32436344:A:ACTCC | NA | frameshift | c.3637_3640 | p.His1214fs |
| | | | dupCTCC | |
| 20:32436385:A:T | NA | stop_gained | c.3673A > T | p.Lys1225* |
| 20:32436397:ATGGATT | NA | frameshift | c.3686_3714 | p.Met1229fs |
| CCAAAGAGCAGTTTCTCTT | | | delTGGATTC | |
| CCTTT:A | | | CAAAGAGCA | |
| | | | GTTCTCTTCC | |
| 20:32436407:AAGAGC: | NA | frameshift | c.3698_3702 | p.Glu1233fs |
| A | | | delAGCAG | |
| 20:32436411:GCAGTTCT | NA | frameshift | c.3701_3711 | p.Gln1234fs |
| CTTC:G | | | delAGTTCTC | |
| | | | TTCC | |
| 20:32436412:C:T | NA | stop_gained | c.3700C > T | p.Gln1234* |
| 20:32436432:T:A | NA | stop_gained | c.3720T > A | p.Cys1240* |
| 20:32436439:C:T | NA | stop_gained | c.3727C > T | p.Gln1243* |
| 20:32436463:C:T | NA | stop_gained | c.3751C > T | p.Gln1251* |
| 20:32436497:GC:G | NA | frameshift | c.3788delC | p.Pro1263fs |
| 20:32436501:AG:A | NA | frameshift | c.3791delG | p.Gly1264fs |
| 20:32436508:CT:C | NA | frameshift | c.3798delT | p.Thr1267fs |
| 20:32436510:T:TA | rs1341638621 | frameshift | c.3799dupA | p.Thr1267fs |
| 20:32436523:A:AACGA | NA | frameshift | c.3811_3812 | p.Thr1271fs |
| GGTGTTCTCGAGGTAGT | | | insACGAGGT | |
| AAGAT | | | GTTCTCGAG | |
| | | | GTAGTAAGA | |
| | | | T | |
| 20:32436523:AC:A | NA | frameshift | c.3812delC | p.Thr1271fs |
| 20:32436527:CT:C | NA | frameshift | c.3816delT | p.Arg1273fs |
| 20:32436530:GT:G | NA | frameshift | c.3821delT | p.Phe1274fs |
| 20:32436536:C:G | NA | stop_gained | c.3824C > G | p.Ser1275* |
| 20:32436568:C:T | NA | stop_gained | c.3856C > T | p.Gln1286* |
| 20:32436573:AGGTC:A | NA | frameshift | c.3864_3867 | p.Arg1289fs |
| | | | delTCGG | |
| 20:32436580:GCC:G | NA | frameshift | c.3870_3871 | p.Leu1291fs |
| | | | delCC | |
| 20:32436582:CCT:C | NA | frameshift | c.3871_3872 | p.Leu1291fs |
| | | | delCT | |
| 20:32436584:TG:T | NA | frameshift | c.3875delG | p.Gly1292fs |
| 20:32436592:C:T | NA | stop_gained | c.3880C > T | p.Gln1294* |
| 20:32436610:C:T | NA | stop_gained | c.3898C > T | p.Gln1300* |
| 20:32436649:ACC:A | NA | frameshift | c.3939_3940 | p.Leu1314fs |
| | | | delCC | |
| 20:32436652:C:CT | NA | frameshift | c.3942dupT | p.Gln1315fs |
| 20:32436655:C:T | NA | stop_gained | c.3943C > T | p.Gln1315* |
| 20:32436656:AGCGCC:A | NA | frameshift | c.3945_3949 | p.Gln1315fs |
| | | | delGCGCC | |
| 20:32436674:A:AC | NA | frameshift | c.3965dupC | p.Met1323fs |
| 20:32436680:T:TA | NA | frameshift | c.3968_3969 | p.Met1323fs |
| | | | insA | |
| 20:32436681:G:GC | NA | frameshift | c.3971dupC | p.Leu1325fs |
| 20:32436727:G:T | NA | stop_gained | c.4015G > T | p.Gly1339* |
| 20:32436760:C:T | rs763386297 | stop_gained | c.4048C > T | p.Gln1350* |
| 20:32436764:C:CATGGA | NA | frameshift | c.4052_4053 | p.Pro1352fs |
| GT | | | insATGGAGT | |
| 20:32436772:G:T | NA | stop_gained | c.4060G > T | p.Glu1354* |
| 20:32436777:CTG:C | NA | frameshift | c.4066_4067 | p.Trp1356fs |
| | | | delTG | |
| 20:32436780:G:A | NA | stop_gained | c.4068G > A | p.Trp1356* |
| 20:32436795:T:TG | NA | frameshift | c.4084dupG | p.Ala1362fs |
| 20:32436820:G:T | NA | stop_gained | c.4108G > T | p.Glu1370* |
| 20:32436823:A:T | NA | stop_gained | c.4111A > T | p.Lys1371* |
| 20:32436833:T:TG | NA | frameshift | c.4127dupG | p.Pro1377fs |
| 20:32436833:TG:T | NA | frameshift | c.4127delG | p.Gly1376fs |
| 20:32436848:A:AG | NA | frameshift | c.4138dupG | p.Ala1380fs |
| 20:32436851:CAA:C | NA | frameshift | c.4141_4142 | p.Asn1381fs |
| | | | delAA | |
| 20:32436858:CGA:C | NA | frameshift | c.4149_4150 | p.Asn1384fs |
| | | | delGA | |
| 20:32436862:AACAGG: | NA | frameshift | c.4152_4156 | p.Asn1384fs |
| A | | | delCAGGA | |
| 20:32436884:GTCCCC:G | NA | frameshift | c.4174_4178 | p.Pro1392fs |
| | | | delCCCCT | |
| 20:32436885:TC:T | NA | frameshift | c.4177delC | p.Leu1393fs |
| 20:32436944:G:A | NA | stop_gained | c.4232G > A | p.Trp1411* |
| 20:32436944:G:GGA | NA | frameshift | c.4233_4234 | p.Lys1412fs |
| | | | dupGA | |
| 20:32436945:G:A | rs1l89588116 | stop_gained | c.4233G > A | p.Trp1411* |
| 20:32436948:ATT:A | NA | frameshift | c.4237_4238 | p.Leu1413fs |
| | | | delTT | |
| 20:32436949:T:TGGGG | NA | frameshift | c.4237_4238 | p.Leu1413fs |
| | | | insGGGG | |
| 20:32436950:T:G | NA | stop_gained | c.4238T > G | p.Leu1413* |
| 20:32436950:T:TA | NA | frameshift | c.4239dupA | p.Pro1414fs |
| 20:32436955:C:T | NA | stop_gained | c.4243C > T | p.Arg1415* |
| 20:32436977:G:GAGGC | NA | frameshift | c.4265_4266 | p.Ser1422fs |
| TCAC | | | insAGGCTCA | |
| | | | C | |
| 20:32436985:CTG:C | rs1463049576 | frameshift | c.4274_4275 | p.Leu1425fs |
| | | | delTG | |
| 20:32437024:C:T | NA | stop_gained | c.4312C > T | p.Gln1438* |
| 20:32437035:T:G | NA | stop_gained | c.4323T > G | p.Tyr1441* |
| 20:32437101:T:TC | NA | frameshift | c.4392dupC | p.Lys1465fs |
| 20:32437129:C:T | NA | stop_gained | c.4417C > T | p.Gln1473* |
| 20:32437240:C:CT | NA | frameshift | c.4529dupT | p.Lys1511fs |
| 20:32437256:TGTGCCA | NA | frameshift | c.4546_4552 | p.Cys1516fs |
| A:T | | | delTGCCAAG | |
| 20:32437257:GT:G | NA | frameshift | c.4546delT | p.Cys1516fs |
| 20:32437260:C:A | NA | stop_gained | c.4548OA | p.Cys1516* |
| 20:32437297:G:T | NA | stop_gained | c.4585G > T | p.Gly1529* |
| 20:32437323:C:A | NA | stop_gained | c.4611OA | p.Cys1537* |
| 20:32437329:GGT:G | NA | frameshift | c.4619_4620 | p.Val1540fs |
| | | | delTG | |

In some embodiments, the subject's burden of having any one or more somatic mutations in DNMT3A and/or ASXL1 can represent a weighted sum of a plurality of any of the DNMT3A and/or ASXL1 somatic mutations. In some embodiments, the burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 mutations present in or around (up to 10 Mb) the DNMT3A gene and/or the ASXL1 gene where the genetic burden is the number of mutations multiplied by the association estimate with lung cancer for each mutation (e.g., a weighted burden score). This can include any somatic mutations in proximity to the DNMT3A gene and/or the ASXL1 gene (up to 10 Mb around the gene) that show a non-zero association with lung cancer. In some embodiments, when the subject has a burden above a desired threshold score, the subject has an increased risk of developing lung cancer. In some embodiments, when the subject has a burden below a desired threshold score, the subject does not have an increased risk of developing lung cancer.

In some embodiments, the burden may be divided into quintiles, e.g., top quintile, intermediate quintile, and bottom quintile, wherein the bottom quintile of burden corresponds to the lowest risk group and the top quintile of burden corresponds to the highest risk group. In some embodiments, a subject having a greater burden comprises the highest weighted burdens, including, but not limited to the top 10%, top 20%, top 30%, top 40%, or top 50% of burdens from a subject population. In some embodiments, the somatic mutations comprise the somatic mutations having association with lung cancer in the top 10%, top 20%, top 30%, top 40%, or top 50% of p-value range for the association. In some embodiments, each of the identified somatic mutations comprise the somatic mutations having association with lung cancer with p-value of about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, or 10⁻¹⁵. In some embodiments, the identified somatic mutations comprise the somatic mutations having association with lung cancer with p-value of less than 5×10⁻⁸. In some embodiments, the identified somatic mutations comprise somatic mutations having association with lung cancer in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the OR may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having burdens in the top decile, quintile, or tertile in a reference population. The threshold of the burden is determined on the basis of the nature of the intended practical application and the risk difference that would be considered meaningful for that practical application.

In some embodiments, when a subject is identified as having an increased risk of developing lung cancer, the subject can undergo any of the procedures described herein related to lung cancer. In some embodiments, the gene burden can be replaced with a survival analysis whereby carriers of the somatic mutation(s) are examiner to determine whether they are more likely of less likely to develop lung cancer over time.

The gene burden analyses described herein can also be used as masks for screening a subject for the risk for developing any of the indications.

The present disclosure also provides methods of detecting the presence or absence of an LY75 variant nucleic acid molecule, a CD164 variant nucleic acid molecule, and/or a PARP1 variant nucleic acid molecule (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms. The sequences provided herein for the LY75, CD164, and PARP1 variant genomic nucleic acid molecules, LY75, CD164, and PARP1 variant mRNA molecules, and LY75, CD164, and PARP1 variant cDNA molecules are only exemplary sequences. Other sequences for the LY75, CD164, and PARP1 variant genomic nucleic acid molecules, variant mRNA molecules, and variant cDNA molecules are also possible.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any an LY75 variant nucleic acid molecule, a CD164 variant nucleic acid molecule, and/or a PARP1 variant nucleic acid molecule, preliminary processing designed to isolate or enrich the biological sample for the genomic DNA can be employed. A variety of techniques may be used for this purpose. When detecting the level of any LY75, CD164, and/or PARP1 variant mRNA molecule, different techniques can be used enrich the biological sample with mRNA molecules. Various methods to detect the presence or level of an mRNA molecule or the presence of a particular variant genomic DNA locus can be used.

In some embodiments, detecting an LY75 predicted loss-of-function polypeptide in a subject comprises performing a sequence analysis on a biological sample obtained from the subject to determine whether an LY75 genomic nucleic acid molecule in the biological sample, and/or an LY75 mRNA molecule in the biological sample, and/or an LY75 cDNA molecule produced from an mRNA molecule in the biological sample, comprises one or more variations that cause a loss-of-function (partial or complete) or are predicted to cause a loss-of-function (partial or complete).

In some embodiments, detecting a CD164 variant nucleic acid molecule in a subject comprises performing a sequence analysis on a biological sample obtained from the subject to determine whether a CD164 genomic nucleic acid molecule in the biological sample, and/or a CD164 mRNA molecule in the biological sample, and/or a CD164 cDNA molecule produced from an mRNA molecule in the biological sample, comprises one or more variations that cause a loss-of-function (partial or complete) or are predicted to cause a loss-of-function (partial or complete).

In some embodiments, detecting a PARP1 variant nucleic acid molecule in a subject comprises performing a sequence analysis on a biological sample obtained from the subject to determine whether a PARP1 genomic nucleic acid molecule in the biological sample, and/or a PARP1 mRNA molecule in the biological sample, and/or a PARP1 cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of an LY75 variant nucleic acid molecule (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the methods of detecting the presence or absence of a CD164 variant nucleic acid molecules (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the methods of detecting the presence or absence of a PARP1 variant nucleic acid molecule (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an LY75 genomic nucleic acid molecule or mRNA molecule, a CD164 genomic nucleic acid molecule or mRNA molecule, and/or a PARP1 genomic nucleic acid molecule or mRNA molecule, and if mRNA, optionally reverse transcribing the mRNA into cDNA. Such assays can comprise, for example determining the identity of these positions of the particular LY75 nucleic acid molecule, particular CD164 nucleic acid molecule, and/or particular PARP1 nucleic acid molecule. In some embodiments, the method is an in vitro method.

In some embodiments, the determining step, detecting step, or sequence analysis comprises sequencing at least a portion of the nucleotide sequence of the LY75 genomic nucleic acid molecule, the LY75 mRNA molecule, or the LY75 cDNA molecule in the biological sample, wherein the sequenced portion comprises one or more variations that cause a loss-of-function (partial or complete) or are predicted to cause a loss-of-function (partial or complete).

In some embodiments, the determining step, detecting step, or sequence analysis comprises sequencing at least a portion of the nucleotide sequence of the CD164 genomic nucleic acid molecule, the CD164 mRNA molecule, or the CD164 cDNA molecule in the biological sample, wherein the sequenced portion comprises one or more variations that cause a loss-of-function (partial or complete) or are predicted to cause a loss-of-function (partial or complete).

In some embodiments, the determining step, detecting step, or sequence analysis comprises sequencing at least a portion of the nucleotide sequence of the PARP1 genomic nucleic acid molecule, the PARP1 mRNA molecule, or the PARP1 cDNA molecule in the biological sample, wherein the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only an LY75 genomic nucleic acid molecule is analyzed. In some embodiments, only an LY75 mRNA is analyzed. In some embodiments, only an LY75 cDNA obtained from LY75 mRNA is analyzed.

In some embodiments, the assay comprises sequencing the entire nucleic acid molecule. In some embodiments, only a CD164 genomic nucleic acid molecule is analyzed. In some embodiments, only a CD164 mRNA is analyzed. In some embodiments, only a CD164 cDNA obtained from CD164 mRNA is analyzed.

In some embodiments, the assay comprises sequencing the entire nucleic acid molecule. In some embodiments, only a PARP1 genomic nucleic acid molecule is analyzed. In some embodiments, only a PARP1 mRNA is analyzed. In some embodiments, only a PARP1 cDNA obtained from PARP1 mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an LY75 variant genomic sequence, variant mRNA sequence, or variant cDNA sequence and not the corresponding LY75 reference sequence under stringent conditions, and determining whether hybridization has occurred.

In some embodiments, the assay comprises contacting the biological sample with a primer or probe, such as an alteration-specific primer or alteration-specific probe, that specifically hybridizes to a CD164 variant genomic sequence, variant mRNA sequence, or variant cDNA sequence and not the corresponding CD164 reference sequence under stringent conditions, and determining whether hybridization has occurred.

In some embodiments, the assay comprises contacting the biological sample with a primer or probe, such as an alteration-specific primer or alteration-specific probe, that specifically hybridizes to a PARP1 variant genomic sequence, variant mRNA sequence, or variant cDNA sequence and not the corresponding PARP1 reference sequence under stringent conditions, and determining whether hybridization has occurred.

In some embodiments, the determining step, detecting step, or sequence analysis comprises: a) amplifying at least a portion of the nucleic acid molecule that encodes the LY75 polypeptide; b) labeling the amplified nucleic acid molecule with a detectable label; c) contacting the labeled nucleic acid molecule with a support comprising an alteration-specific probe; and d) detecting the detectable label.

In some embodiments, the determining step, detecting step, or sequence analysis comprises: a) amplifying at least a portion of the nucleic acid molecule that encodes the CD164 polypeptide; b) labeling the amplified nucleic acid molecule with a detectable label; c) contacting the labeled nucleic acid molecule with a support comprising an alteration-specific probe; and d) detecting the detectable label.

In some embodiments, the determining step, detecting step, or sequence analysis comprises: a) amplifying at least a portion of the nucleic acid molecule that encodes the PARP1 polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an LY75 variant genomic nucleic acid molecule, variant mRNA molecule, or variant cDNA molecule; a CD164 variant genomic nucleic acid molecule, variant mRNA molecule, or variant cDNA molecule; and/or a PARP1 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to LY75 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, such isolated nucleic acid molecules hybridize to CD164 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, such isolated nucleic acid molecules hybridize to PARP1 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to LY75 variant genomic nucleic acid molecules, LY75 variant mRNA molecules, and/or LY75 variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to CD164 variant genomic nucleic acid molecules, CD164 variant mRNA molecules, and/or CD164 variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to PARP1 variant genomic nucleic acid molecules, PARP1 variant mRNA molecules, and/or PARP1 variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the LY75 variant genomic nucleic acid molecules, LY75 variant mRNA molecules, and/or LY75 variant cDNA molecules disclosed herein. The primers described herein can be used to amplify LY75 variant genomic nucleic acid molecules, LY75 variant mRNA molecules, or LY75 variant cDNA molecules, or a fragment thereof.

The probes and primers described herein can also be used to detect a nucleotide variation within any of the CD164 variant genomic nucleic acid molecules, CD164 variant mRNA molecules, and/or CD164 variant cDNA molecules disclosed herein. The primers described herein can be used to amplify CD164 variant genomic nucleic acid molecules, CD164 variant mRNA molecules, or CD164 variant cDNA molecules, or a fragment thereof.

The probes and primers described herein can also be used to detect a nucleotide variation within any of the PARP1 variant genomic nucleic acid molecules, PARP1 variant mRNA molecules, and/or PARP1 variant cDNA molecules disclosed herein. The primers described herein can be used to amplify CD164 variant genomic nucleic acid molecules, PARP1 variant mRNA molecules, or PARP1 variant cDNA molecules, or a fragment thereof.

In the context of the disclosure “specifically hybridizes” means that the probe or primer (such as, for example, the alteration-specific probe or alteration-specific primer) does not hybridize to a nucleic acid sequence encoding an LY75 reference genomic nucleic acid molecule, a CD164 reference genomic nucleic acid molecule, a PARP1 reference genomic nucleic acid molecule, an LY75 reference mRNA molecule, or a CD164 reference mRNA molecule, a PARP1 reference mRNA molecule, an LY75 reference cDNA molecule, a CD164 reference cDNA molecule, and/or PARP reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The nucleotide sequence of an LY75 reference genomic nucleic acid molecule is set forth in SEQ ID NO:1 (ENSG00000054219.11 encompassing chr2:159,803,355-159,904,756 in the GRCh38/hg38 human genome assembly). The nucleotide sequence of an LY75 variant genomic nucleic acid molecule is set forth in SEQ ID NO:2 (r578446341; C70,612T; codon 70,611-70,613 CCG to CTG; 101,402 bp). In some embodiments, an LY75 variant genomic nucleic acid molecule is rs147820690 comprising a C>T variation at position chr2:159878663 (GRCh38.p13; NC_000002.12: g.159878663C>T).

The nucleotide sequence of an LY75 reference mRNA molecule is set forth in SEQ ID NO:3 (NM_002349.4; Isoform 1; 6,932 nt; LY75 Segment). The nucleotide sequence of another LY75 reference mRNA molecule is set forth in SEQ ID NO:4 (ENST00000504764.5; Isoform 2; 5,650 nt; LY75-CD302). The nucleotide sequence of another LY75 reference mRNA molecule is set forth in SEQ ID NO:5 (ENST00000505052.1; Isoform 3; 5,482 nt; LY75-CD302). The nucleotide sequence of another LY75 reference mRNA molecule is set forth in SEQ ID NO:6 (NM_001198759.1; Isoform 4; 8,919 nt; LY75-CD302). The nucleotide sequence of another LY75 reference mRNA molecule is set forth in SEQ ID NO:7 (NM_001198760.1; Isoform 5; 8,751 nt; LY75-CD302). The nucleotide sequence of another LY75 reference mRNA molecule is set forth in SEQ ID NO:8 (AY184222.1; Isoform 6; 5,622 nt; LY75-CD302). The nucleotide sequence of another LY75 reference mRNA molecule is set forth in SEQ ID NO:9 (AY314006.1; Isoform 7; 5,454 nt; LY75-CD302). The nucleotide sequence of another LY75 reference mRNA molecule is set forth in SEQ ID NO:10 (A13208915.1; Isoform 8; 5,713 nt; LY75-Segment). The nucleotide sequence of another LY75 reference mRNA molecule is set forth in SEQ ID NO:11 (AF011333.1; Isoform 9; 6,928 nt; LY75-Segment). The nucleotide sequence of another LY75 reference mRNA molecule is set forth in SEQ ID NO:12 (AF064827.1; Isoform 10; 5,169 nt; LY75-Segment). The nucleotide sequence of another LY75 reference mRNA molecule is set forth in SEQ ID NO:13 (ENST00000263636.4; Isoform 11; 6,886 nt; LY75-Segment).

The nucleotide sequence of an LY75 variant mRNA molecule is set forth in SEQ ID NO:14 (NM_002349.4; Isoform 1; r578446341; C3,814T; Codon 3,813-3,815 CCG to CUG; 6,932 nt; LY75 Segment). The nucleotide sequence of another LY75 variant mRNA molecule is set forth in SEQ ID NO:15 (ENST00000504764.5; Isoform 2; r578446341; C3,768T; Codon 3,767-3,769 CCG to CUG; 5,650 nt; LY75-CD302). The nucleotide sequence of another LY75 variant mRNA molecule is set forth in SEQ ID NO:16 (ENST00000505052.1; Isoform 3; r578446341; C3,768T; Codon 3,767-3,769 CCG to CUG; 5,482 nt; LY75-CD302). The nucleotide sequence of another LY75 variant mRNA molecule is set forth in SEQ ID NO:17 (NM_001198759.1; Isoform 4; r578446341; C3,814T; Codon 3,813-3,815 CCG to CUG; 8,919 nt; LY75-CD302). The nucleotide sequence of another LY75 variant mRNA molecule is set forth in SEQ ID NO:18 (NM_001198760.1; Isoform 5; r578446341; C3,814T; Codon 3,813-3,815 CCG to CUG; 8,751 nt; LY75-CD302). The nucleotide sequence of another LY75 variant mRNA molecule is set forth in SEQ ID N0:19 (AY184222.1; Isoform 6; r578446341; C3,740T; Codon 3,739-3,741 CCG to CUG; 5,622 nt; LY75-CD302). The nucleotide sequence of another LY75 variant mRNA molecule is set forth in SEQ ID NO:20 (AY314006.1; Isoform 7; r578446341; C3,740T; Codon 3,739-3,741 CCG to CUG; 5,454 nt; LY75-CD302). The nucleotide sequence of another LY75 variant mRNA molecule is set forth in SEQ ID NO:21 (AB208915.1; Isoform 8; C2,594T; Codon 2,593-2,595 CCG to CUG; 5,713 nt; LY75-Segment). The nucleotide sequence of another LY75 variant mRNA molecule is set forth in SEQ ID NO:22 (AF011333.1; Isoform 9; C3,793T; Codon 3,792-3,794 CCG to CUG; 6,928 nt; LY75-Segment). The nucleotide sequence of another LY75 variant mRNA molecule is set forth in SEQ ID NO:23 (AF064827.1; Isoform 10; C3,740T; Codon 3,739-3,741 CCG to CUG; 5,169 nt; LY75-Segment). The nucleotide sequence of another LY75 variant mRNA molecule is set forth in SEQ ID NO:24 (ENST00000263636.4; Isoform 11; C3,768T; Codon 3,767-3,769 CCG to CUG; 6,886 nt; LY75-Segment). In some embodiments, an LY75 variant mRNA molecule is any of the mRNA molecule isoforms described above produced from the LY75 variant genomic nucleic acid molecule rs147820690 comprising a C>T variation at position chr2:159878663 (GRCh38.p13; NC_000002.12: g.159878663C>T).

The nucleotide sequence of an LY75 reference cDNA molecule is set forth in SEQ ID NO:25. The nucleotide sequence of another LY75 reference cDNA molecule is set forth in SEQ ID NO:26. The nucleotide sequence of another LY75 reference cDNA molecule is set forth in SEQ ID NO:27. The nucleotide sequence of another LY75 reference cDNA molecule is set forth in SEQ ID NO:28. The nucleotide sequence of another LY75 reference cDNA molecule is set forth in SEQ ID NO:29. The nucleotide sequence of another LY75 reference cDNA molecule is set forth in SEQ ID NO:30. The nucleotide sequence of another LY75 reference cDNA molecule is set forth in SEQ ID NO:31. The nucleotide sequence of another LY75 reference cDNA molecule is set forth in SEQ ID NO:32. The nucleotide sequence of another LY75 reference cDNA molecule is set forth in SEQ ID NO:33. The nucleotide sequence of another LY75 reference cDNA molecule is set forth in SEQ ID NO:34. The nucleotide sequence of another LY75 reference cDNA molecule is set forth in SEQ ID NO:35.

The nucleotide sequence of an LY75 variant cDNA molecule is set forth in SEQ ID NO:36. The nucleotide sequence of another LY75 variant cDNA molecule is set forth in SEQ ID NO:37. The nucleotide sequence of another LY75 variant cDNA molecule is set forth in SEQ ID NO:38. The nucleotide sequence of another LY75 variant cDNA molecule is set forth in SEQ ID NO:39. The nucleotide sequence of another LY75 variant cDNA molecule is set forth in SEQ ID NO:40. The nucleotide sequence of another LY75 variant cDNA molecule is set forth in SEQ ID NO:41. The nucleotide sequence of another LY75 variant cDNA molecule is set forth in SEQ ID NO:42. The nucleotide sequence of another LY75 variant cDNA molecule is set forth in SEQ ID NO:43. The nucleotide sequence of another LY75 variant cDNA molecule is set forth in SEQ ID NO:44. The nucleotide sequence of another LY75 variant cDNA molecule is set forth in SEQ ID NO:45. The nucleotide sequence of another LY75 variant cDNA molecule is set forth in SEQ ID NO:46. In some embodiments, an LY75 variant cDNA molecule is any cDNA molecule produced from any mRNA isoform molecule produced from the LY75 variant genomic nucleic acid molecule rs147820690 comprising a C>T variation at position chr2:159878663 (GRCh38.p13; NC_000002.12: g.159878663C>T).

The amino acid sequence of an LY75 reference polypeptide is set forth in SEQ ID NO:47 (Isoform 1; AAC17636.1) and is 1,722 amino acids in length. The amino acid sequence of another LY75 reference polypeptide is set forth in SEQ ID NO:48 (Isoform 2; NP_001185688.1) and is 1,873 amino acids in length. The amino acid sequence of another LY75 reference polypeptide is set forth in SEQ ID NO:49 (Isoform 3; NP_001185689.1) and is 1,817 amino acids in length. The amino acid sequence of another LY75 reference polypeptide is set forth in SEQ ID NO:50 (Isoform 4; BAD92152.1) and is 1,340 amino acids in length.

The amino acid sequence of an LY75 variant polypeptide is set forth in SEQ ID NO:51 (Isoform 1; AAC17636.1; Pro1,247Leu) and is 1,722 amino acids in length. The amino acid sequence of another LY75 variant polypeptide is set forth in SEQ ID NO:52 (Isoform 2; NP_001185688.1; Pro1,247Leu) and is 1,873 amino acids in length. The amino acid sequence of another LY75 variant polypeptide is set forth in SEQ ID NO:53 (Isoform 3; NP_001185689.1; Pro1,247Leu) and is 1,817 amino acids in length. The amino acid sequence of another LY75 variant polypeptide is set forth in SEQ ID NO:54 (Isoform 4; BAD92152.1; Pro865Leu) and is 1,340 amino acids in length. In some embodiments, an LY75 variant polypeptide is G525E produced from the LY75 variant genomic nucleic acid molecule rs147820690 comprising a C>T variation at position chr2:159878663 (GRCh38.p13; NC_000002.12: g.159878663C>T).

The nucleotide sequence of a CD164 reference genomic nucleic acid molecule is set forth in SEQ ID NO:55 (ENSG00000135535.1 encompassing chr6:109,366,514-109,381,739 in the GRCh38/hg38 human genome assembly). The nucleotide sequence of a CD164 variant genomic nucleic acid molecule is set forth in SEQ ID NO:56 (r53799840; T297A; 15,226 bp).

The nucleotide sequence of a CD164 reference mRNA molecule is set forth in SEQ ID NO:57 (NM_001346500; Isoform 1; 2,992 nt). The nucleotide sequence of another CD164 reference mRNA molecule is set forth in SEQ ID NO:58 (ENST00000413644.6; Isoform 2; 2,414 nt). The nucleotide sequence of another CD164 reference mRNA molecule is set forth in SEQ ID NO:59 (NM_006016.6; Isoform; 3 3,020 nt). The nucleotide sequence of another CD164 reference mRNA molecule is set forth in SEQ ID NO:60 (ENST00000275080.11; Isoform 4; 2,954 nt). The nucleotide sequence of another CD164 reference mRNA molecule is set forth in SEQ ID NO:61 (ENST00000324953.9; Isoform 5; 2,936 nt). The nucleotide sequence of another CD164 reference mRNA molecule is set forth in SEQ ID NO:62 (ENST00000512821.5; Isoform 6; 964 nt). The nucleotide sequence of another CD164 reference mRNA molecule is set forth in SEQ ID NO:63 (NM_001142403.3; Isoform 7; 2,424 nt). The nucleotide sequence of another CD164 reference mRNA molecule is set forth in SEQ ID NO:64 (NM_001142402.3; Isoform 8; 2,963 nt). The nucleotide sequence of another CD164 reference mRNA molecule is set forth in SEQ ID NO:65 (NM_001142401.3; Isoform 9; 2,981 nt). The nucleotide sequence of another CD164 reference mRNA molecule is set forth in SEQ ID NO:66 (D14043.1; Isoform 10; 2,427 nt). The nucleotide sequence of another CD164 reference mRNA molecule is set forth in SEQ ID NO:67 (AF299341.1; Isoform 11; 2,929 nt). The nucleotide sequence of another CD164 reference mRNA molecule is set forth in SEQ ID NO:68 (AF299342.1; Isoform 12; 2,950 nt). The nucleotide sequence of another CD164 reference mRNA molecule is set forth in SEQ ID NO:69 (AF299343.1; Isoform 13; 2,968 nt). The nucleotide sequence of another CD164 reference mRNA molecule is set forth in SEQ ID NO:70 (BC011522.3; Isoform 14; 3,010 nt). The nucleotide sequence of another CD164 reference mRNA molecule is set forth in SEQ ID NO:71 (AK301692.1; Isoform 15; 1,294 nt). The nucleotide sequence of another CD164 reference mRNA molecule is set forth in SEQ ID NO:72 (AK303525.1; Isoform 16; 1,386 nt). The nucleotide sequence of another CD164 reference mRNA molecule is set forth in SEQ ID NO:73 (AK315908.1; Isoform 17; 1,386 nt). The nucleotide sequence of another CD164 reference mRNA molecule is set forth in SEQ ID NO:74 (AF106518.1; Isoform; 18 537 nt). The nucleotide sequence of another CD164 reference mRNA molecule is set forth in SEQ ID NO:75 (AF263279.1; Isoform 19; 594 nt). The nucleotide sequence of another CD164 reference mRNA molecule is set forth in SEQ ID NO:76 (FJ200494.1; Isoform 20; 590 nt). The nucleotide sequence of another CD164 reference mRNA molecule is set forth in SEQ ID NO:77 (AK312357.1; Isoform 21; 683 nt). The nucleotide sequence of another CD164 reference mRNA molecule is set forth in SEQ ID NO:78 (ENST00000368961.6; Isoform 22; 3,106 nt). The nucleotide sequence of another CD164 reference mRNA molecule is set forth in SEQ ID NO:79 (ENST00000310786.5; Isoform 23; 2,993 nt). The nucleotide sequence of another CD164 reference mRNA molecule is set forth in SEQ ID NO:80 (ENST00000504373.1; Isoform 24; 1,402 nt).

The nucleotide sequence of a CD164 reference cDNA molecule is set forth in SEQ ID NO:81. The nucleotide sequence of another CD164 reference cDNA molecule is set forth in SEQ ID NO:82. The nucleotide sequence of another CD164 reference cDNA molecule is set forth in SEQ ID NO:83 The nucleotide sequence of another CD164 reference cDNA molecule is set forth in SEQ ID NO:84. The nucleotide sequence of another CD164 reference cDNA molecule is set forth in SEQ ID NO:85. The nucleotide sequence of another CD164 reference cDNA molecule is set forth in SEQ ID NO:86. The nucleotide sequence of another CD164 reference cDNA molecule is set forth in SEQ ID NO:87. The nucleotide sequence of another CD164 reference cDNA molecule is set forth in SEQ ID NO:88. The nucleotide sequence of another CD164 reference cDNA molecule is set forth in SEQ ID NO:89. The nucleotide sequence of another CD164 reference cDNA molecule is set forth in SEQ ID NO:90. The nucleotide sequence of another CD164 reference cDNA molecule is set forth in SEQ ID NO:91. The nucleotide sequence of another CD164 reference cDNA molecule is set forth in SEQ ID NO:92. The nucleotide sequence of another CD164 reference cDNA molecule is set forth in SEQ ID NO:93. The nucleotide sequence of another CD164 reference cDNA molecule is set forth in SEQ ID NO:94. The nucleotide sequence of another CD164 reference cDNA molecule is set forth in SEQ ID NO:95. The nucleotide sequence of another CD164 reference cDNA molecule is set forth in SEQ ID NO:96. The nucleotide sequence of another CD164 reference cDNA molecule is set forth in SEQ ID NO:97. The nucleotide sequence of another CD164 reference cDNA molecule is set forth in SEQ ID NO:98. The nucleotide sequence of another CD164 reference cDNA molecule is set forth in SEQ ID NO:99. The nucleotide sequence of another CD164 reference cDNA molecule is set forth in SEQ ID NO:100. The nucleotide sequence of another CD164 reference cDNA molecule is set forth in SEQ ID NO:101. The nucleotide sequence of another CD164 reference cDNA molecule is set forth in SEQ ID NO:102. The nucleotide sequence of another CD164 reference cDNA molecule is set forth in SEQ ID NO:103. The nucleotide sequence of another CD164 reference cDNA molecule is set forth in SEQ ID NO:104.

The amino acid sequence of a CD164 reference polypeptide is set forth in SEQ ID NO:105 (Isoform 1), and is 163 amino acids in length. The amino acid sequence of another CD164 reference polypeptide is set forth in SEQ ID NO:106 (NP_001135875.1; Isoform 2), and is 189 amino acids in length. The amino acid sequence of another CD164 reference polypeptide is set forth in SEQ ID NO:107 (AAG53906.1; Isoform 3), and is 197 amino acids in length. The amino acid sequence of another CD164 reference polypeptide is set forth in SEQ ID NO:108 (NP_001135873.1; Isoform 4), and is 184 amino acids in length. The amino acid sequence of another CD164 reference polypeptide is set forth in SEQ ID NO:109 (NP_001135874.1; Isoform 5), and is 178 amino acids in length. The amino acid sequence of another CD164 reference polypeptide is set forth in SEQ ID NO:110 (Isoform 6), and is 157 amino acids in length. The amino acid sequence of another CD164 reference polypeptide is set forth in SEQ ID NO:111 (BAG63164.1; Isoform 7), and is 156 amino acids in length. The amino acid sequence of another CD164 reference polypeptide is set forth in SEQ ID NO:112 (AC054891.1; Isoform 8), and is 147 amino acids in length.

The nucleotide sequence of a PARP1 reference genomic nucleic acid molecule is set forth in SEQ ID NO:113 (ENSG00000143799.14 encompassing chr1:226,360,691-226,408,093 in the GRCh38/hg38 human genome assembly).

The nucleotide sequence of a PARP1 reference mRNA molecule is set forth in SEQ ID NO:114 (ENST00000366794.10; Isoform 1; 3,978 nt). The nucleotide sequence of another PARP1 reference mRNA molecule is set forth in SEQ ID NO:115 (ENST00000677203.1; Isoform 2; 3,850 nt). The nucleotide sequence of another PARP1 reference mRNA molecule is set forth in SEQ ID NO:116 (J03473.1; Isoform 3; 3,795 nt). The nucleotide sequence of another PARP1 reference mRNA molecule is set forth in SEQ ID NO:117 (BC037545; Isoform 4; 3,677 nt). The nucleotide sequence of another PARP1 reference mRNA molecule is set forth in SEQ ID NO:118 (M18112.1; Isoform 5; 3,640 nt). The nucleotide sequence of another PARP1 reference mRNA molecule is set forth in SEQ ID NO:119 (M32721.1; Isoform 6; 3,660 nt). The nucleotide sequence of another PARP1 reference mRNA molecule is set forth in SEQ ID NO:120 (AK303340.1; Isoform 7; 3,371 nt). The nucleotide sequence of another PARP1 reference mRNA molecule is set forth in SEQ ID NO:121 (M17081.1; Isoform 8; 1,771 nt). The nucleotide sequence of another PARP1 reference mRNA molecule is set forth in SEQ ID NO:122 (AK312339.1; Isoform 9; 3,132 nt). The nucleotide sequence of another PARP1 reference mRNA molecule is set forth in SEQ ID NO:123 (BC018620.1; Isoform 10; 827 nt). The nucleotide sequence of another PARP1 reference mRNA molecule is set forth in SEQ ID NO:124 (BC014206; Isoform 11; 902 nt). The nucleotide sequence of another PARP1 reference mRNA molecule is set forth in SEQ ID NO:125 (ENST00000366792.3; Isoform 12; 553 nt). The nucleotide sequence of another PARP1 reference mRNA molecule is set forth in SEQ ID NO:126 (ENST00000629232.1; Isoform 13; 477 nt). The nucleotide sequence of another PARP1 reference mRNA molecule is set forth in SEQ ID NO:127 (ENST00000366790.3; Isoform 14; 570 nt). The nucleotide sequence of another PARP1 reference mRNA molecule is set forth in SEQ ID NO:128 (ENST00000366794.6; Isoform 15; 3,958 nt).

The nucleotide sequence of a PARP1 reference cDNA molecule is set forth in SEQ ID NO:129. The nucleotide sequence of another PARP1 reference cDNA molecule is set forth in SEQ ID NO:130. The nucleotide sequence of another PARP1 reference cDNA molecule is set forth in SEQ ID NO:131 The nucleotide sequence of another PARP1 reference cDNA molecule is set forth in SEQ ID NO:132. The nucleotide sequence of another PARP1 reference cDNA molecule is set forth in SEQ ID NO:133. The nucleotide sequence of another PARP1 reference cDNA molecule is set forth in SEQ ID NO:134. The nucleotide sequence of another PARP1 reference cDNA molecule is set forth in SEQ ID NO:135. The nucleotide sequence of another PARP1 reference cDNA molecule is set forth in SEQ ID NO:136. The nucleotide sequence of another PARP1 reference cDNA molecule is set forth in SEQ ID NO:137. The nucleotide sequence of another PARP1 reference cDNA molecule is set forth in SEQ ID NO:138. The nucleotide sequence of another PARP1 reference cDNA molecule is set forth in SEQ ID NO:139. The nucleotide sequence of another PARP1 reference cDNA molecule is set forth in SEQ ID NO:140. The nucleotide sequence of another PARP1 reference cDNA molecule is set forth in SEQ ID NO:141. The nucleotide sequence of another PARP1 reference cDNA molecule is set forth in SEQ ID NO:142. The nucleotide sequence of another PARP1 reference cDNA molecule is set forth in SEQ ID NO:143.

The amino acid sequence of a PARP1 reference polypeptide is set forth in SEQ ID NO:144 (AAB59447.1; Isoform 1), and is 1,014 amino acids in length. The amino acid sequence of another PARP1 reference polypeptide is set forth in SEQ ID NO:145 (Isoform 2), and is 971 amino acids in length. The amino acid sequence of another PARP1 reference polypeptide is set forth in SEQ ID NO:146 (BAG64403.1; Isoform 3), and is 993 amino acids in length. The amino acid sequence of another PARP1 reference polypeptide is set forth in SEQ ID NO:147 (AAA51599.1; Isoform 4), and is 574 amino acids in length. The amino acid sequence of another PARP1 reference polypeptide is set forth in SEQ ID NO:148 (AAH18620.1; Isoform 5), and is 232 amino acids in length. The amino acid sequence of another PARP1 reference polypeptide is set forth in SEQ ID NO:149 (AAH14206.1; Isoform 6), and is 250 amino acids in length. The amino acid sequence of another PARP1 reference polypeptide is set forth in SEQ ID NO:150 (Isoform 7), and is 108 amino acids in length. The amino acid sequence of another PARP1 reference polypeptide is set forth in SEQ ID NO:151 (Isoform 8), and is 155 amino acids in length.

The nucleotide sequence of a DNMT3A reference genomic nucleic acid molecule is set forth in SEQ ID NO:212 (ENSG00000119772.17 encompassing chr2:25,227,855-25,342,590 in the GRCh38/hg38 human genome assembly).

The nucleotide sequence of a DNMT3A reference mRNA molecule is set forth in SEQ ID NO:213 (ENST00000264709.7; Isoform 1; 9,501 nt). The nucleotide sequence of another DNMT3A reference mRNA molecule is set forth in SEQ ID NO:214 (ENST00000321117.10; Isoform 2; 9,421 nt). The nucleotide sequence of another DNMT3A reference mRNA molecule is set forth in SEQ ID NO:215 (ENST00000406659.3; Isoform 3; 1,775 nt). The nucleotide sequence of another DNMT3A reference mRNA molecule is set forth in SEQ ID NO:216 (ENST00000380746.8; Isoform 4; 3,589 nt). The nucleotide sequence of another DNMT3A reference mRNA molecule is set forth in SEQ ID NO:217 (ENST00000402667.1; Isoform 5; 2,300 nt). The nucleotide sequence of another DNMT3A reference mRNA molecule is set forth in SEQ ID NO:218 (NM_175629.2; Isoform 6; 4,395 nt). The nucleotide sequence of another DNMT3A reference mRNA molecule is set forth in SEQ ID NO:219 (NM_001320892.2; Isoform 7; 1,714 nt). The nucleotide sequence of another DNMT3A reference mRNA molecule is set forth in SEQ ID NO:220 (NM_175630.1; Isoform 8; 1,808 nt). The nucleotide sequence of another DNMT3A reference mRNA molecule is set forth in SEQ ID NO:221 (NM_001320893.1; Isoform 9; 3,638 nt). The nucleotide sequence of another DNMT3A reference mRNA molecule is set forth in SEQ ID NO:222 (NM_153759.3; Isoform 10; 3,608 nt). The nucleotide sequence of another DNMT3A reference mRNA molecule is set forth in SEQ ID NO:223 (NM_001375819.1; Isoform 11; 3,473 nt). The nucleotide sequence of another DNMT3A reference mRNA molecule is set forth in SEQ ID NO:224 (BC043617.1; Isoform 12; 4,294 nt). The nucleotide sequence of another DNMT3A reference mRNA molecule is set forth in SEQ ID NO:225 (AF331856.1; Isoform 13; 4,258 nt). The nucleotide sequence of another DNMT3A reference mRNA molecule is set forth in SEQ ID NO:226 (A13208833.1; Isoform 14; 4,476 nt). The nucleotide sequence of another DNMT3A reference mRNA molecule is set forth in SEQ ID NO:227 (BC018214.1; Isoform 15; 1,758 nt). The nucleotide sequence of another DNMT3A reference mRNA molecule is set forth in SEQ ID NO:228 (AF480163.1; Isoform 16; 2,371 nt). The nucleotide sequence of another DNMT3A reference mRNA molecule is set forth in SEQ ID NO:229 (BC023612.2; Isoform 17; 1,113 nt). The nucleotide sequence of another DNMT3A reference mRNA molecule is set forth in SEQ ID NO:230 (AF067972.2; Isoform 18; 3,005 nt). The nucleotide sequence of another DNMT3A reference mRNA molecule is set forth in SEQ ID NO:231 (BC051864.1; Isoform 19; 943 nt). The nucleotide sequence of another DNMT3A reference mRNA molecule is set forth in SEQ ID NO:232 (ENST00000321117.9; Isoform 20; 4,279 nt). The nucleotide sequence of another DNMT3A reference mRNA molecule is set forth in SEQ ID NO:233 (ENST00000380756.4; Isoform 21; 4,477 nt). The nucleotide sequence of another DNMT3A reference mRNA molecule is set forth in SEQ ID NO:234 (ENST00000683760.1; Isoform 22; 3,585 nt).

The nucleotide sequence of a DNMT3A reference cDNA molecule is set forth in SEQ ID NO:235. The nucleotide sequence of another DNMT3A reference cDNA molecule is set forth in SEQ ID NO:236. The nucleotide sequence of another DNMT3A reference cDNA molecule is set forth in SEQ ID NO:237. The nucleotide sequence of another DNMT3A reference cDNA molecule is set forth in SEQ ID NO:238. The nucleotide sequence of another DNMT3A reference cDNA molecule is set forth in SEQ ID NO:239. The nucleotide sequence of another DNMT3A reference cDNA molecule is set forth in SEQ ID NO:240. The nucleotide sequence of another DNMT3A reference cDNA molecule is set forth in SEQ ID NO:241. The nucleotide sequence of another DNMT3A reference cDNA molecule is set forth in SEQ ID NO:242. The nucleotide sequence of another DNMT3A reference cDNA molecule is set forth in SEQ ID NO:243. The nucleotide sequence of another DNMT3A reference cDNA molecule is set forth in SEQ ID NO:244. The nucleotide sequence of another DNMT3A reference cDNA molecule is set forth in SEQ ID NO:245. The nucleotide sequence of another DNMT3A reference cDNA molecule is set forth in SEQ ID NO:246. The nucleotide sequence of another DNMT3A reference cDNA molecule is set forth in SEQ ID NO:247. The nucleotide sequence of another DNMT3A reference cDNA molecule is set forth in SEQ ID NO:248. The nucleotide sequence of another DNMT3A reference cDNA molecule is set forth in SEQ ID NO:249. The nucleotide sequence of another DNMT3A reference cDNA molecule is set forth in SEQ ID NO:250. The nucleotide sequence of another DNMT3A reference cDNA molecule is set forth in SEQ ID NO:251. The nucleotide sequence of another DNMT3A reference cDNA molecule is set forth in SEQ ID NO:252. The nucleotide sequence of another DNMT3A reference cDNA molecule is set forth in SEQ ID NO:253. The nucleotide sequence of another DNMT3A reference cDNA molecule is set forth in SEQ ID NO:254. The nucleotide sequence of another DNMT3A reference cDNA molecule is set forth in SEQ ID NO:255. The nucleotide sequence of another DNMT3A reference cDNA molecule is set forth in SEQ ID NO:256.

The amino acid sequence of a DNMT3A reference polypeptide is set forth in SEQ ID NO:257 (NP_783328.1; Isoform 1), and is 912 amino acids in length. The amino acid sequence of another DNMT3A reference polypeptide is set forth in SEQ ID NO:258 (NP_001307821.1; Isoform 2), and is 166 amino acids in length. The amino acid sequence of another DNMT3A reference polypeptide is set forth in SEQ ID NO:259 (NP_715640.2; Isoform 3), and is 723 amino acids in length. The amino acid sequence of another DNMT3A reference polypeptide is set forth in SEQ ID NO:260 (NP_001362748.1; Isoform 4), and is 689 amino acids in length. The amino acid sequence of another DNMT3A reference polypeptide is set forth in SEQ ID NO:261 (NP_001307822.1; Isoform 5), and is 760 amino acids in length. The amino acid sequence of another DNMT3A reference polypeptide is set forth in SEQ ID NO:262 (AAL57039.1; Isoform 6), and is 909 amino acids in length. The amino acid sequence of another DNMT3A reference polypeptide is set forth in SEQ ID NO:263 (BAD92070.1; Isoform 7), and is 811 amino acids in length. The amino acid sequence of another DNMT3A reference polypeptide is set forth in SEQ ID NO:264 (AAH18214.1; Isoform 8), and is 285 amino acids in length. The amino acid sequence of another DNMT3A reference polypeptide is set forth in SEQ ID NO:265 (AAH23612.1; Isoform 9), and is 351 amino acids in length. The amino acid sequence of another DNMT3A reference polypeptide is set forth in SEQ ID NO:266 (AAH23612.1; Isoform 10), and is 781 amino acids in length.

The nucleotide sequence of an ASXL1 reference genomic nucleic acid molecule is set forth in SEQ ID NO:267 (ENSG00000171456.20 encompassing chr20:32,358,330-32,439,260 in the GRCh38/hg38 human genome assembly).

The nucleotide sequence of an ASXL1 reference mRNA molecule is set forth in SEQ ID NO:268 (ENST00000651418.1; Isoform 1; 3,146 nt). The nucleotide sequence of another ASXL1 reference mRNA molecule is set forth in SEQ ID NO:269 (ENST00000375687.10; Isoform 2; 7,052 nt). The nucleotide sequence of another ASXL1 reference mRNA molecule is set forth in SEQ ID NO:270 (ENST00000542461.5; Isoform 3; 1,068 nt). The nucleotide sequence of another ASXL1 reference mRNA molecule is set forth in SEQ ID NO:271 (ENST00000613218.4; Isoform 4; 7,038 nt). The nucleotide sequence of another ASXL1 reference mRNA molecule is set forth in SEQ ID NO:272 (ENST00000646367.1; Isoform 5; 1,065 nt). The nucleotide sequence of another ASXL1 reference mRNA molecule is set forth in SEQ ID NO:273 (ENST00000620121.4; Isoform 6; 5,374 nt). The nucleotide sequence of another ASXL1 reference mRNA molecule is set forth in SEQ ID NO:274 (ENST00000646985.1; Isoform 7; 6,666 nt). The nucleotide sequence of another ASXL1 reference mRNA molecule is set forth in SEQ ID NO:275 (ENST00000497249.6; Isoform 8; 495 nt). The nucleotide sequence of another ASXL1 reference mRNA molecule is set forth in SEQ ID NO:276 (ENST00000375689.5; Isoform 9; 812 nt). The nucleotide sequence of another ASXL1 reference mRNA molecule is set forth in SEQ ID NO:277 (ENST00000306058.9; Isoform 10; 6,591 nt). The nucleotide sequence of another ASXL1 reference mRNA molecule is set forth in SEQ ID NO:278 (NM_001164603.1; Isoform 11; 1,084 nt). The nucleotide sequence of another ASXL1 reference mRNA molecule is set forth in SEQ ID NO:279 (BC100280.1; Isoform 12; 1,078 nt). The nucleotide sequence of another ASXL1 reference mRNA molecule is set forth in SEQ ID NO:280 (BC064984.1; Isoform 13; 1,009 nt). The nucleotide sequence of another ASXL1 reference mRNA molecule is set forth in SEQ ID NO:281 (AJ438952.2; Isoform 14; 6,864 nt). The nucleotide sequence of another ASXL1 reference mRNA molecule is set forth in SEQ ID NO:282 (AK122923.1; Isoform 15; 4,685 nt). The nucleotide sequence of another ASXL1 reference mRNA molecule is set forth in SEQ ID NO:283 (AB023195.2; Isoform 16; 6,088 nt). The nucleotide sequence of another ASXL1 reference mRNA molecule is set forth in SEQ ID NO:284 (AL117518.1; Isoform 17; 4,055 nt). The nucleotide sequence of another ASXL1 reference mRNA molecule is set forth in SEQ ID NO:285 (ENST00000375687.5; Isoform 18; 7,031 nt). The nucleotide sequence of another ASXL1 reference mRNA molecule is set forth in SEQ ID NO:286 (ENST00000497249.2; Isoform 19; 296 nt). The nucleotide sequence of another ASXL1 reference mRNA molecule is set forth in SEQ ID NO:287 (ENST00000555343.2; Isoform 20; 1,034 nt).

The nucleotide sequence of an ASXL1 reference cDNA molecule is set forth in SEQ ID NO:288. The nucleotide sequence of another ASXL1 reference cDNA molecule is set forth in SEQ ID NO:289. The nucleotide sequence of another ASXL1 reference cDNA molecule is set forth in SEQ ID NO:290. The nucleotide sequence of another ASXL1 reference cDNA molecule is set forth in SEQ ID NO:291. The nucleotide sequence of another ASXL1 reference cDNA molecule is set forth in SEQ ID NO:292. The nucleotide sequence of another ASXL1 reference cDNA molecule is set forth in SEQ ID NO:293. The nucleotide sequence of another ASXL1 reference cDNA molecule is set forth in SEQ ID NO:294. The nucleotide sequence of another ASXL1 reference cDNA molecule is set forth in SEQ ID NO:295. The nucleotide sequence of another ASXL1 reference cDNA molecule is set forth in SEQ ID NO:296. The nucleotide sequence of another ASXL1 reference cDNA molecule is set forth in SEQ ID NO:297. The nucleotide sequence of another ASXL1 reference cDNA molecule is set forth in SEQ ID NO:298. The nucleotide sequence of another ASXL1 reference cDNA molecule is set forth in SEQ ID NO:299. The nucleotide sequence of another ASXL1 reference cDNA molecule is set forth in SEQ ID NO:300. The nucleotide sequence of another ASXL1 reference cDNA molecule is set forth in SEQ ID NO:301. The nucleotide sequence of another ASXL1 reference cDNA molecule is set forth in SEQ ID NO:302. The nucleotide sequence of another ASXL1 reference cDNA molecule is set forth in SEQ ID NO:303. The nucleotide sequence of another ASXL1 reference cDNA molecule is set forth in SEQ ID NO:304. The nucleotide sequence of another ASXL1 reference cDNA molecule is set forth in SEQ ID NO:305. The nucleotide sequence of another ASXL1 reference cDNA molecule is set forth in SEQ ID NO:306. The nucleotide sequence of another ASXL1 reference cDNA molecule is set forth in SEQ ID NO:307.

The amino acid sequence of an ASXL1 reference polypeptide is set forth in SEQ ID NO:308 (Isoform 1), and is 625 amino acids in length. The amino acid sequence of another ASXL1 reference polypeptide is set forth in SEQ ID NO:309 (CAD27708.1; Isoform 2), and is 1,541 amino acids in length. The amino acid sequence of another ASXL1 reference polypeptide is set forth in SEQ ID NO:310 (NP_001158075.1; Isoform 3), and is 85 amino acids in length. The amino acid sequence of another ASXL1 reference polypeptide is set forth in SEQ ID NO:311 (Isoform 4), and is 1,480 amino acids in length. The amino acid sequence of another ASXL1 reference polypeptide is set forth in SEQ ID NO:312 (Isoform 5), and is 75 amino acids in length. The amino acid sequence of another ASXL1 reference polypeptide is set forth in SEQ ID NO:313 (Isoform 6), and is 81 amino acids in length. The amino acid sequence of another ASXL1 reference polypeptide is set forth in SEQ ID NO:314 (Isoform 7), and is 1,536 amino acids in length. The amino acid sequence of another ASXL1 reference polypeptide is set forth in SEQ ID NO:315 (AAH64984.1; Isoform 8), and is 84 amino acids in length. The amino acid sequence of another ASXL1 reference polypeptide is set forth in SEQ ID NO:316 (BAG53800.1; Isoform 9), and is 1,462 amino acids in length. The amino acid sequence of another ASXL1 reference polypeptide is set forth in SEQ ID NO:317 (BAA76822.2; Isoform 10), and is 1,368 amino acids in length. The amino acid sequence of another ASXL1 reference polypeptide is set forth in SEQ ID NO:318 (Isoform 11), and is 1,341 amino acids in length. The amino acid sequence of another ASXL1 reference polypeptide is set forth in SEQ ID NO:319 (Isoform 12), and is 60 amino acids in length. The amino acid sequence of another ASXL1 reference polypeptide is set forth in SEQ ID NO:320 (Isoform 13), and is 57 amino acids in length.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×his or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NO:55, or SEQ ID NO:113). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides methods of stratifying a suitable lung cancer patient for treatment with a PARP1 inhibitor. The methods comprise determining whether the patient carries a DNMT3A R882H somatic mutation or TET2 somatic mutation deficiency. The patient with the DNMT3A R882H somatic mutation or TET2 somatic mutation deficiency can be excluded from a PARP1 inhibitor treatment regimen.

The present disclosure also provides therapeutic agents that prevent or reduce CHIP for use in the prevention and/or reduction of CHIP in a subject having: an LY75 variant genomic nucleic acid molecule, a CD164 variant genomic nucleic acid molecule, and/or a PARP1 variant genomic nucleic acid molecule; an LY75 variant mRNA molecule, a CD164 variant mRNA molecule, and/or a PARP1 variant mRNA molecule; or an LY75 variant cDNA molecule, a CD164 variant cDNA molecule, and/or a PARP1 variant cDNA molecule. Any of the therapeutic agents that prevent or reduce CHIP described herein can be used in these methods.

The present disclosure also provides uses of therapeutic agents that prevent or reduce CHIP for use in the preparation of a medicament for prevention or reduction of CHIP in a subject having: an LY75 variant genomic nucleic acid molecule, a CD164 variant genomic nucleic acid molecule, and/or a PARP1 variant genomic nucleic acid molecule; an LY75 variant mRNA molecule, a CD164 variant mRNA molecule, and/or a PARP1 variant mRNA molecule; or an LY75 variant cDNA molecule, a CD164 variant cDNA molecule, and/or a PARP1 variant cDNA molecule. Any of the therapeutic agents that prevent or reduce CHIP described herein can be used in these methods.

The present disclosure also provides an LY75 inhibitor for use in the prevention or reduction of CHIP in a subject having: an LY75 variant genomic nucleic acid, an LY75 variant mRNA molecule, or an LY75 variant cDNA molecule. Any of the LY75 inhibitors described herein can be used in these methods.

The present disclosure also provides a CD164 inhibitor for use in the prevention or reduction of CHIP CHIP in a subject having: a CD164 variant genomic nucleic acid molecule, a CD164 variant mRNA molecule, or a CD164 variant cDNA molecule. Any of the CD164 inhibitors described herein can be used in these methods.

The present disclosure also provides a PARP1 inhibitor for use in the prevention or reduction of CHIP in a subject having: a PARP1 variant genomic nucleic acid molecule, a PARP1 variant mRNA molecule, or a PARP1 variant cDNA molecule. Any of the PARP1 inhibitors described herein can be used in these methods.

The present disclosure also provides an LY75 inhibitor for use in the preparation of a medicament for the prevention or reduction of CHIP in a subject having: an LY75 variant genomic nucleic acid molecule, an LY75 variant mRNA molecule, or an LY75 variant cDNA molecule. Any of the LY75 inhibitors described herein can be used in these methods.

The present disclosure also provides a CD164 inhibitor for use in the preparation of a medicament for the prevention or reduction of CHIP in a subject having: a CD164 variant genomic nucleic acid molecule, a CD164 variant mRNA molecule, or a CD164 variant cDNA molecule. Any of the CD164 inhibitors described herein can be used in these methods.

The present disclosure also provides a PARP1 inhibitor for use in preparation of a medicament for the prevention or reduction of CHIP in a subject having: a PARP1 variant genomic nucleic acid molecule, a PARP1 variant mRNA molecule, or a PARP1 variant cDNA molecule. Any of the PARP1 inhibitors described herein can be used in these methods.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Common Variants in LY75/CD302/LY75-CD302 Locus Associate with CHIP

Several common variants in LY75/CD302/LY75-CD302 locus associate with CHIP, including LY75 missense rs78446341 (see, Table 4).

TABLE 4
LY75/CD302/LY75-CD302 locus associations with CHIP

| Variant | rsID | P-value | Effect | MAF | CADD | varEffect | Lof |
|---|---|---|---|---|---|---|---|
| 2:159868900:C:A | rs56186131 | 2.83E−12 | 1.058 | 0.4569 | 0.371 | intronic | No |
| 2:159861821:C:G | rs72955755 | 3.13E−12 | 1.058 | 0.4562 | 0.676 | intronic | No |
| 2:159870033:T:G | rs17231342 | 3.94E−12 | 1.058 | 0.4560 | 4.066 | intronic | No |
| 2:159834145:G:A | rs78446341 | 2.48E−08 | 0.8541 | 0.0227 | 24.5 | missense | No |

| | | | | | | | nGWASctlg | |
|---|---|---|---|---|---|---|---|---|
| Variant | rsID | eQTL | min_eQTLPval | ls Path | lnHGMD | lnGWASctlg | Traits | minGWASctlgPval |
| 2:159868900:C:A | rs56186131 | FALSE | NA | FALSE | FALSE | FALSE | 0 | NA |
| 2:159861821:C:G | rs72955755 | TRUE | 2.51E−14 | FALSE | FALSE | FALSE | 0 | NA |
| 2:159870033:T:G | rs17231342 | TRUE | 9.17E−14 | FALSE | FALSE | FALSE | 0 | NA |
| 2:159834145:G:A | rs78446341 | FALSE | NA | FALSE | TRUE | TRUE | 1 | 4.00E−29 |

Common LY75-CD302 locus variants that are in eQTLs for nearby genes are shown in Table 5.

TABLE 5
Common LY75-CD302 locus variants are in eQTLs for nearby genes

| uid | geneName | Pval | Dataset |
|---|---|---|---|
| 2:159861821:C:G | AC009961.1 | 2.51e−14 | RegnGHS_Liver |
| 2:159870033:T:G | MARCH7 | 3.403e−13 | Westra_Whole_ Blood |
| 2:159870033:T:G | MARCH7 | 9.1657e−14 | Vosa_Whole_ Blood |

Common LY75-CD302 locus variants are in eQTLs for nearby genes

| | | Effect_ | | |
|---|---|---|---|---|
| uid | Cis_Trans | Allele | Ref_Allele | Effects |
| 2:159861821:C:G | CIS | G | C | 0.214 |
| 2:159870033:T:G | CIS | G | T | −7.2774 |
| 2:159870033:T:G | CIS | G | T | −7.4524 |

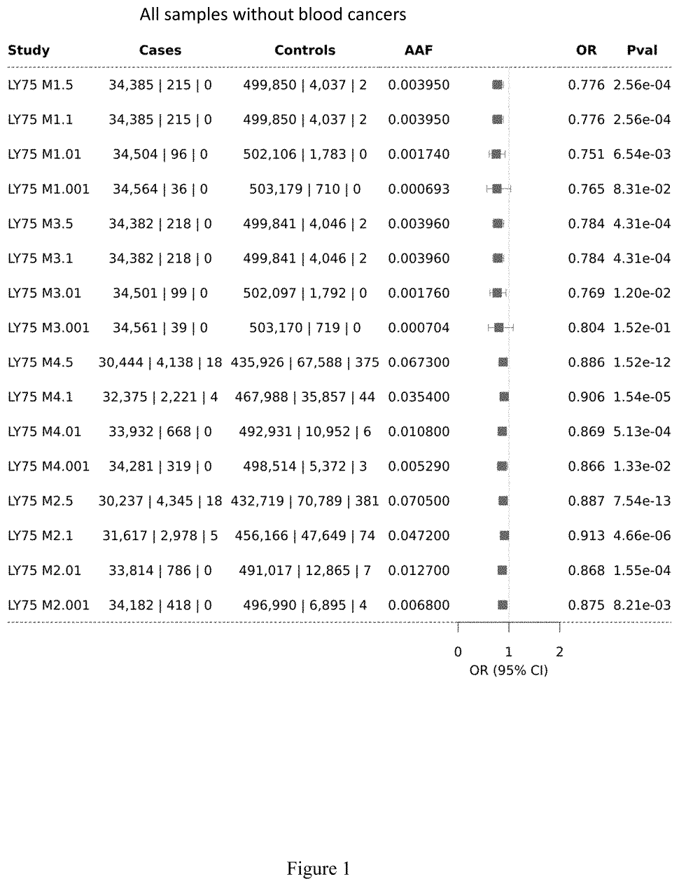

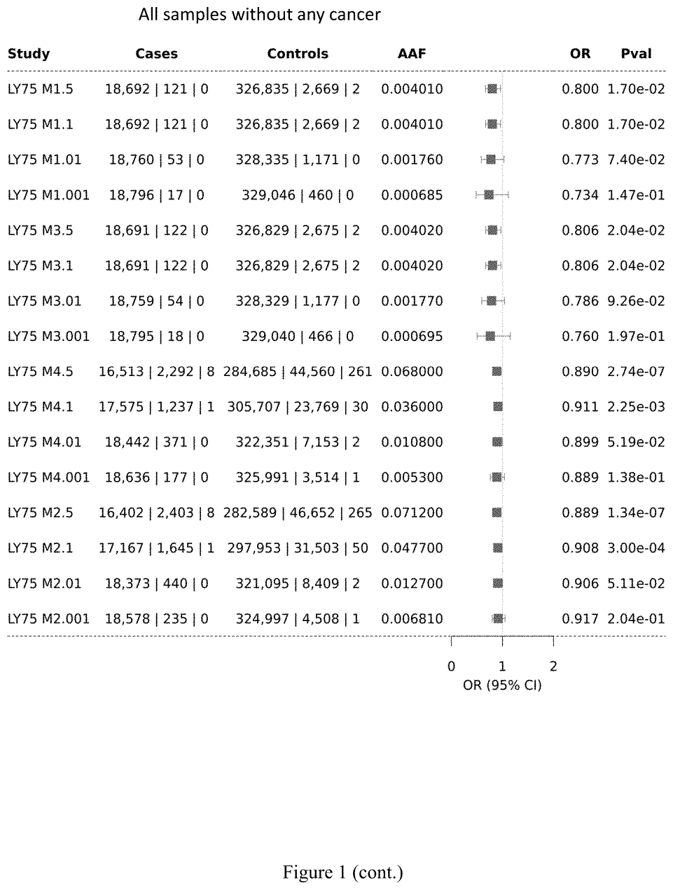

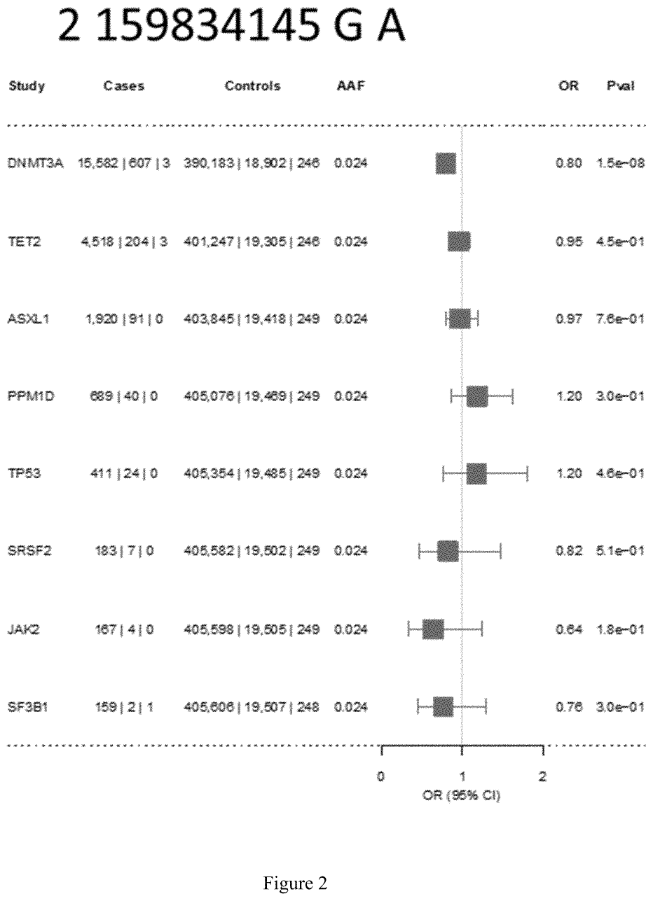

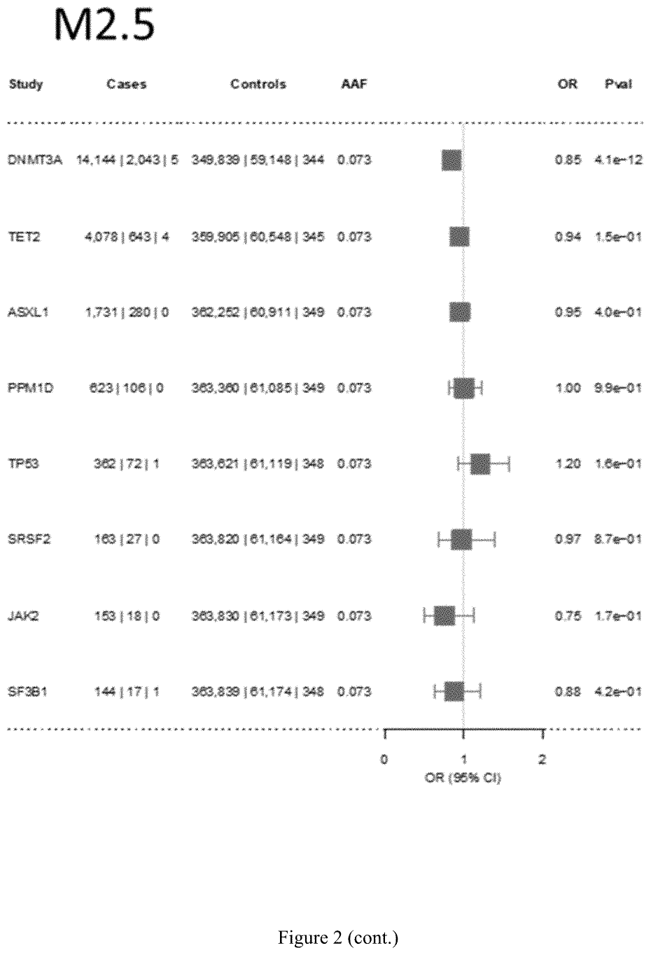

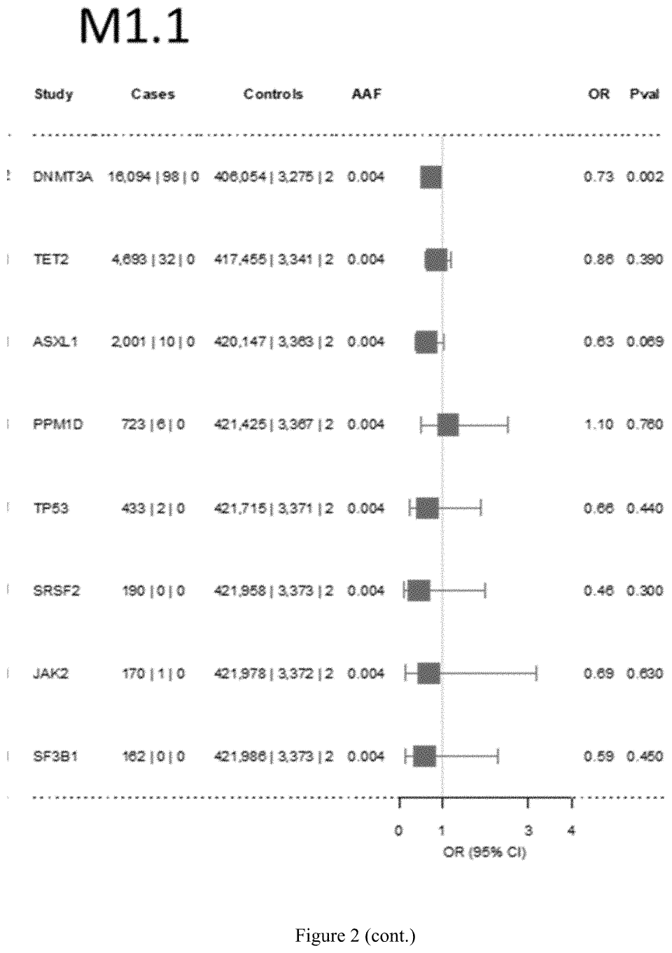

Finemapping the LY75-CD302 locus identified rs78446341 as highly likely (^{˜}80%) to be driving the association signal at this locus (data not shown). In addition, all LY75 burden masks suggest a reduced CHIP risk (see, FIG. 1). Moreover, the effect of the ly75 locus varies by CHIP gene mutation carrier (see, FIG. 2).

LY75 missense p.Pro1247Leu significantly associated with platelets, neutrophils, and lymphocytes in UKB 500K (see, Table 6; Variant=2:159834145:G:A; HGVS=p.Pro1247Leu).

TABLE 6
LY75 missense p.Pro1247Leu significantly associates with platelets,
neutrophils, and lymphocytes in UKB 500K

| | Effect | | Cases | Controls | |
|---|---|---|---|---|---|
| Phenotype | (95% Cl) | P-value | RR\|RA\|AA | RR\|RA\|AA | AAF |
| Platelet crit | ↑ 0.115 | 8.3E−76 | NA | 399599\|19216\| | 0.024 |
| | (0.103, 0.128) | | | 245 | |
| Neutrophils | ↑ 0.047 | 2.7E−12 | NA | 398255\|19282\| | 0.024 |
| | (0.034, 0.061) | | | 242 | |
| Lymphocytes | ↓ −0.046 | 4.5E−12 | NA | 398260\|19282\| | 0.024 |
| | (−0.060, −0.033) | | | 242 | |

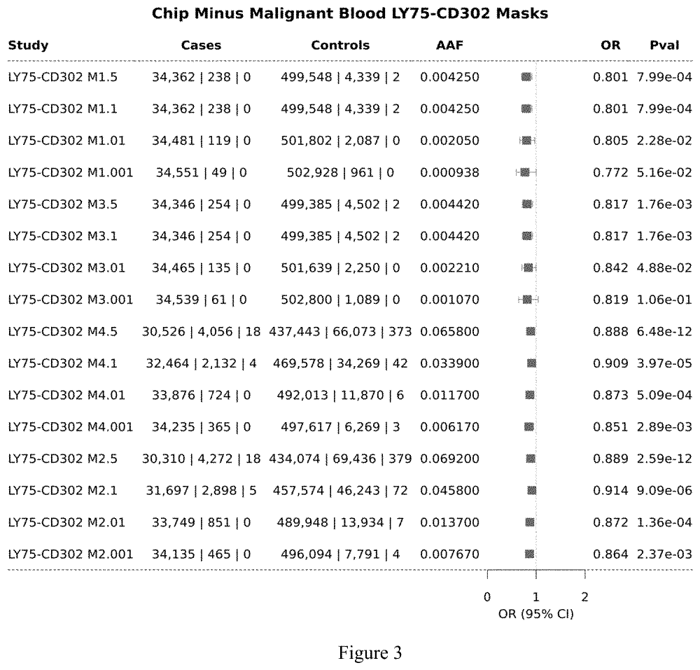

Additional rare pLoFs and rare missense variants in LY75 also associated with reduced odds of CHIP (see, Table 7, Phenotype=CHIP; and FIG. 3).

TABLE 7
Additional rare pLoFs and rare missense variants
in LY75 also associate with reduced odds of CHIP

| | Variants in | | | Cases | Controls | |
|---|---|---|---|---|---|---|
| Mask | Mask | Effect (95% CI) | P-value | RR \| RA \| AA | RR \| RA \| AA | AAF |
| M1.1 | pLoFs, <1% | 0.78 | 2.6E−4 | 34385 \| 215 \| 0 | 499850 \| 4037 \| 2 | 0.004 |
| | AAF | (0.68, 0.89) | | | | |
| M1.01 | pLoFs, <0.1% | 0.75 | 6.5E−3 | 34504 \| 96 \| 0 | 502106 \| 1783 \| 0 | 0.002 |
| | AAF | (0.61, 0.92) | | | | |
| M3.1 | pLoFs & | 0.78 | 4.3E−4 | 34283 \| 218 \| 0 | 499841 \| 4046 \| 2 | 0.004 |
| | missense, <1% | (0.68, 0.90) | | | | |
| | AAF | | | | | |
| M3.01 | pLoFs & | 0.77 | 0.012 | 34501 \| 99 \| 0 | 502096 \| 1792 \| 0 | 0.002 |
| | missense, <0.1% | (0.63, 0.94) | | | | |
| | AAF | | | | | |

### Example 2: Common Variants in CD164 Locus Associate with CHIP

Multiple common variant signals in CD164 locus associate with CHIP, although fine-mapping does not point to a causal gene (data not shown). In addition, eQTL data supports variant effects on CD164 expression (see, Table 8).

TABLE 8
eQTL data variant effects on CD164 expression

| Uid | geneName | Pval | Dataset |
|---|---|---|---|
| 6:109287294:C:T | CD164 | 5.56e−14 | Fairfax_Monocytes_LPS24 |
| 6:109326927:T:G | CD164 | 5.58008e−19 | GTeXv*_Whole_Blood |
| 6:109381443:A:T | CD164 | 4.037e−29 | Chen_Neutrophils |

eQTL data variant effects on CD164 expression

| uid | Cis_Trans | Effect_Allele | Ref_Allele | Effects |
|---|---|---|---|---|
| 6:109287294:C:T | CIS | NA | NA | NA |
| 6:109326927:T:G | CIS | G | T | 0.123912 |
| 6:109381443:A:T | CIS | T | A | −1.117 |

### Example 3: Common Variants in PARP1 Locus Associate with CHIP

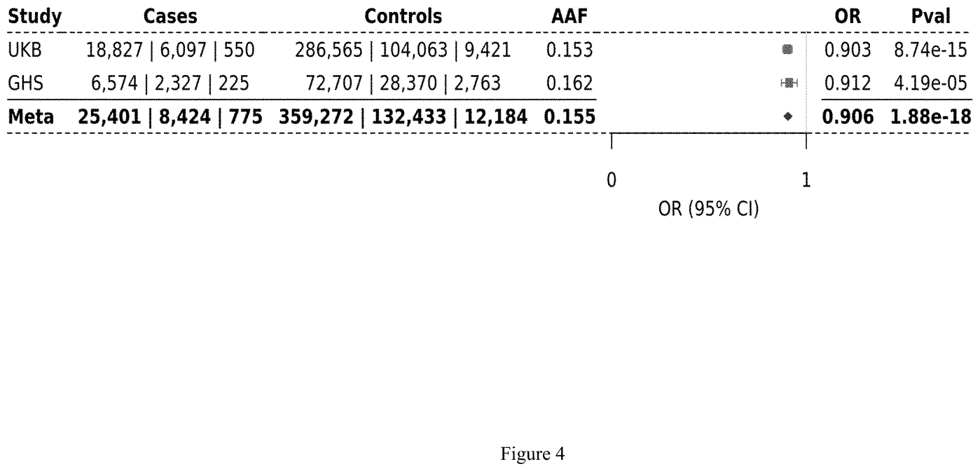

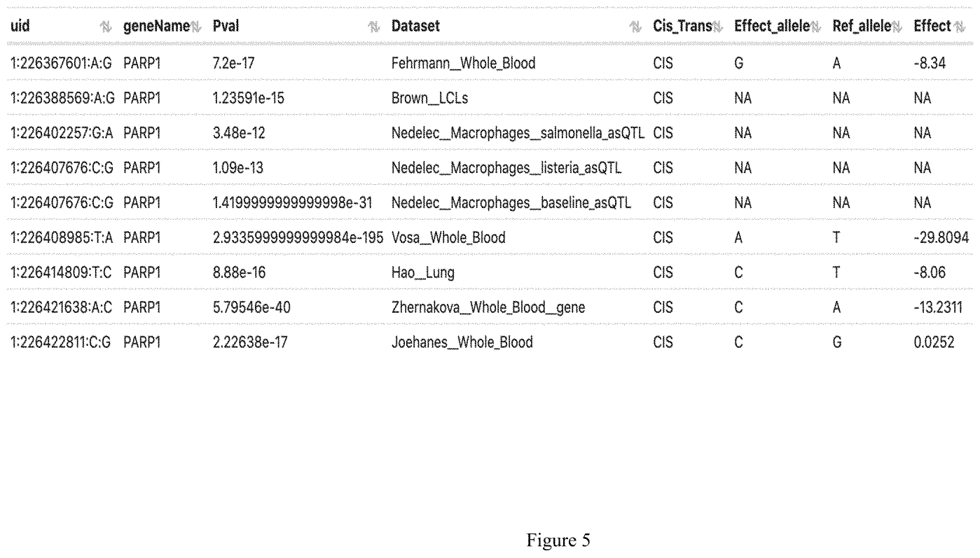

A PARP1 1:226367601:A:C missense variant was in perfect LD with index SNP and showed a protective association (see, FIG. 4). Other CHIP-associated variants were significant eQTLs for PARP1 (see, FIG. 5).

### Example 4: Somatic Mutations in DNMT3A and/or ASXL1 Associate with Increased Risk of Developing Lung Cancer

Exome sequencing data from the UKB Exome Sequencing Consortium was used to identify CHIP somatic mutation carriers across 454,787 UKB participants. This was complemented by generating an additional CHIP callset across 133,370 individuals from the DiscoverEHR Cohort. These represent the largest CHIP callsets to date, and were used to conduct genetic association analyses of CHIP across 29,669 CHIP mutation carriers from UKB, and to perform replication in 14,766 CHIP mutation carriers from the DiscoverEHR cohort. 27 loci associated with CHIP were identified in UKB at genome-wide significance, which was replicated in DiscoverEHR. Additionally, phenotypic associations for both CHIP somatic mutation carriers and germline CHIP risk loci were investigated across 35,000 traits from the UK Biobank.

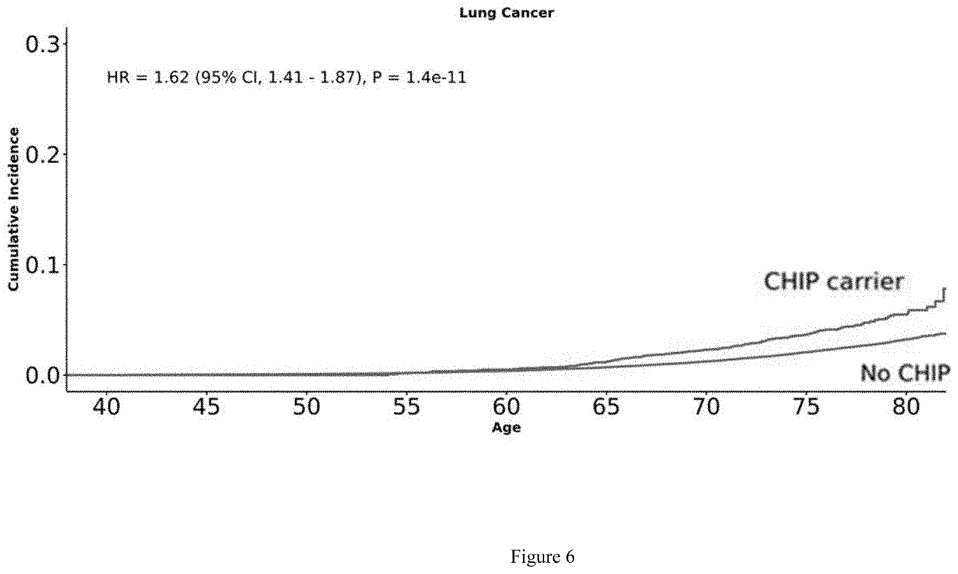

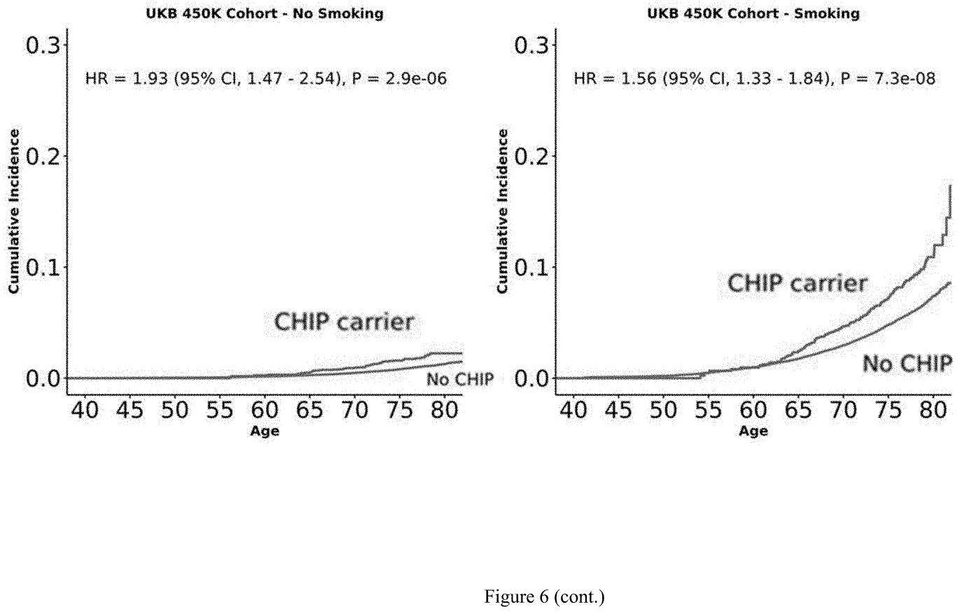

The analysis described herein only includes CHIP carriers with the largest expansion of blood cells with the CHIP mutations (estimated to have ^{˜}20% of their blood cells with the CHIP mutations. It was determined whether CHIP carriers were at an elevated risk of developing solid tumors (see FIG. 6), and it was found that high variant allele fraction (VAF) carriers are at significantly elevated risk of developing lung cancer (HR=1.62 (1.41-1.87), p=1.36*10-11), and modestly increased risk of developing prostate (HR=1.17 (1.04-1.31), p=7.1*10−3) and breast cancers (HR=1.15 (1.002-1.322), p=0.047). No increased risk for the development of colon cancer was found (HR=0.96 (0.79-1.16), p=0.66). Models estimating event risk on the basis of CHIP mutational subtype (e.g., carriers must have DNMT3A mutations) suggest that these associations with prostate and breast cancer are driven primarily by DNMT3A mutations.

Given the strong associations between CHIP and blood cancer and lung cancer, and the associations between smoking and both CHIP and lung cancer, additional analyses stratified by smoking status were performed to test whether these associations were driven by smoking and merely marked by CHIP mutations. High VAF CHIP carriers are at an elevated risk of developing blood cancers in smokers (2.37 (1.86-3.03), p=5.24*10-12) and non-smokers (2.05 (1.68-2.49), P=7.47*10-13), and this pattern was similar among all CHIP carriers and across associations with CHIP mutation subtype ASXL1-CHIP. Lung cancer risk among all CHIP carriers is the same among smokers (HR=1.45 (1.28-1.63), p=1.10*10-9) and non-smokers (HR=1.45 (1.18-1.80), P=5.46*10-4), and is actually higher in non-smokers among CHIP carriers with VAF 10% (HRsmokers=1.56 (1.33-1.84), p=7.32*10-8 compared to HRnon-smokers=1.93 (1.47-2.54), P=2.86*10-6). When sub-setting to DNMT3A-CHIP and ASXL1-CHIP, the patterns were similar. Overall, the models suggest that CHIP mutation carriers are at an elevated risk of blood cancer and lung cancer independent of smoking, but that CHIP is likely also marking additional blood cancer risk that results from smoking.

In summary, in longitudinal analyses, it was found that individuals who carry CHIP somatic mutations in either DNMT3A or ASXL1 have an increased risk of lung cancer in both smokers and non-smokers, which indicates that CHIP is an independent risk factor for malignant neoplasms outside of the hemopoietic lineage.

### Example 5: Effects of PARPi on Cells Harboring CHIP Gene Mutation

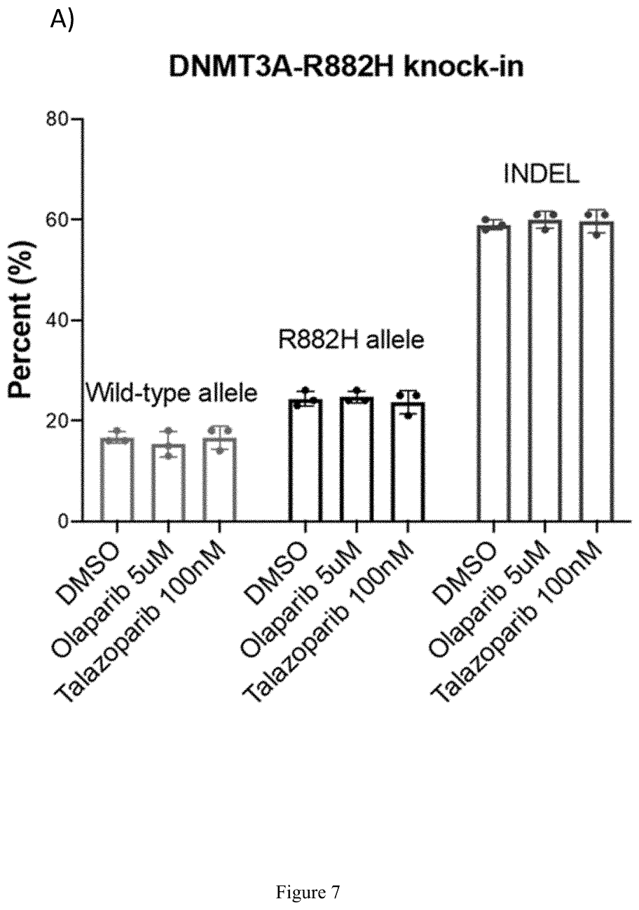

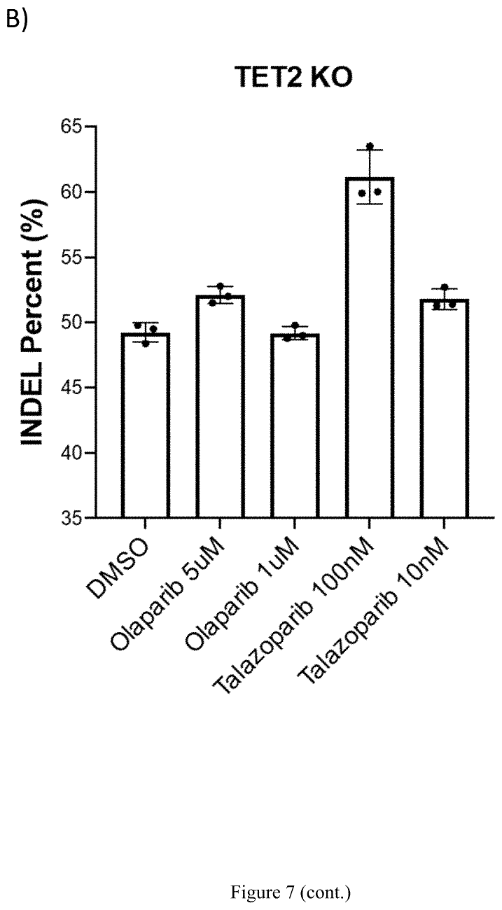

To determine the effect, if any, that PARP1 inhibitors have on cells in vitro that carry CHIP gene mutations, variants/mutations were engineered into cell lines using CRISPR-Cas9 and specifically optimized sgRNA target sequence as well donor sequence for the DNMT3A Knockin. Functional modeling of identified CHIP-GWAS locus was performed. Selected PARP1 locus from CHIP-GWAS analysis showed significant association of DNMT3A to protective PARP1 germline variant (data not shown). FIG. 7, Panels A and B show systematic tracking of HDR and INDEL percentages in PARPi treated cells. Cells were treated with PARPi for 8 days and then subjected to Sanger sequencing across each locus to determine population dynamics. FIG. 7, Panel A shows RPE-1 hTERT cells harboring CRISPR mediated knock-in of DNMT3A-R882H allele. This shows that there is no effect of PARP1 inhibitor treatment on cells carrying the DNMT3A variant allele and it remains unchanged in the population as compared to DMSO (control/vehicle) alone. Thus, PARP1 inhibitors had no effect on cells carrying the DNMT3A-R882H variant. Referring to FIG. 7, Panel A, the percentage represents the percent of sequences that derive from that represented allele in the legend, with all three legend items adding up to roughly 100% for each of the samples. Thus, the wild-type allele (blue group) for the DMSO group has an average of ^{˜}16% (16% of the sequences in the population of cells are wild-type). The R882H allele (black group) in DMSO has ^{˜}24% (24% of the sequences derived from the population of cells have the R882H mutation). The INDEL (red group) in DMSO has ^{˜}60% (60% of sequences in the population carry INDEL (nucleotide insertion/deletion) alleles). The total for wild-type, R882H, and INDEL for the DMSO group is ^{˜}100% of the sequences analyzed.

FIG. 7, Panel B shows CRISPR-mediated TET2-deficieny in HEK293T cells. Cells carrying potential loss of function alleles (INDELs/KO alleles) are resistant to PARP1 inhibitor treatment—in olaparib and talazoparib conditions, the percentage of INDELs (mutant alleles) increased compared to DMSO (control/vehicle). This means that cells carrying TET2 KO/INDEL alleles survive better than wild-type alleles in response to treatment with PARP1 inhibitors. Thus, in this case, it may be advisable to avoid PARP1 inhibitor treatment in order to not increase clonal selection for those TET2 mutant/variant alleles that cause deficiency.

### Example 6: Jointly Fine-Mapping Common and Rare Variants at the LY75 Locus

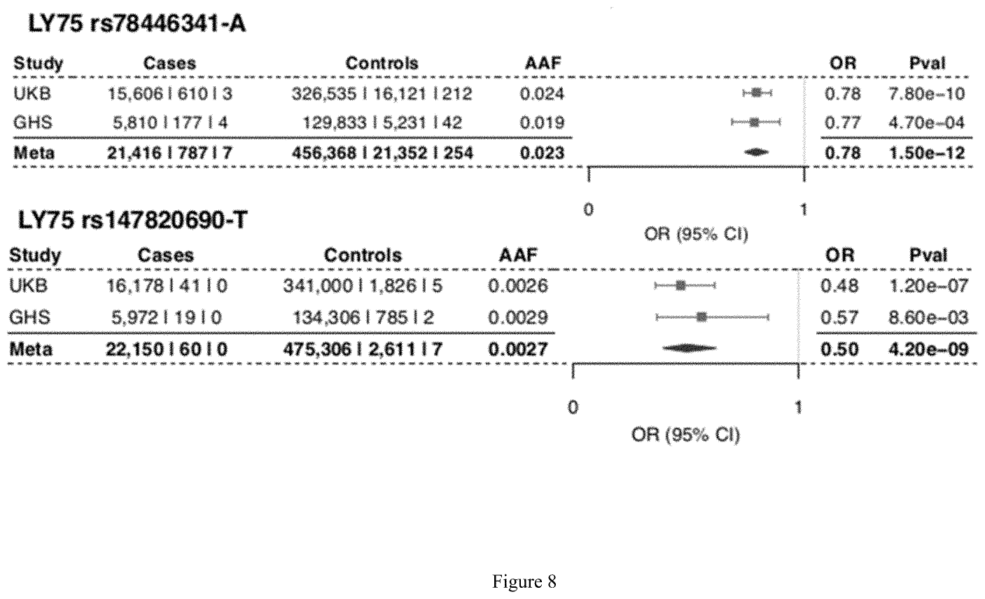

DNMT3A, which was the most commonly mutated gene in the overall CHIP phenotype, had the largest number of significantly associated loci (n=23), most of which overlapped with the overall CHIP association signals. At a locus on chromosome 2, rs78446341 (P1247L in LY75) was associated with reduced risk of DNMT3A-CHIP (OR=0.78 (0.72-0.84), P=3.70×10⁻⁹, and was prioritized by fine-mapping. LY75 features lymphocyte specific expression, and is thought to be involved in antigen presentation and lymphocyte proliferation. A second rare (AAF=0.002) missense variant (r5147820690-T, G525E) was also identified that associated with reduced risk of DNMT3A-CHIP at close to genome-wide significance (OR=0.48 (0.36-0.63), P=1.15×10⁻⁷). This variant was predicted as likely to be damaging (CADD=23.6) and remains associated (OR=0.63 (0.51-0.77), P=4.80×10⁻⁶) when conditioning on common variant signal in this locus (i.e., this rare variant signal is independent of the common variant signal in this locus). This variant was also prioritized by fine-mapping. Finally, these signals in PARP1 and LY75 replicated in GHS (see, FIG. 8).

The more common LY75 missense variant (r578446341-A, P1247L) is located in the extracellular domain of lymphocytic antigen 75, which is also known as DEC-205/CD205, and plays a role in antigenic capture, processing, and presentation. The rarer LY75 missense variant (r5147820690-T, G525E) is located in a C-type lectin domain and reported to interact directly with this receptor's ligand. The protective associations with this variant that weRE identified HEREIN appear to be most pronounced for DNMT3A-CHIP and mLOY and highlight LY75 as a therapeutic target for the antagonization of CH in general.

To further evaluate whether the rare variant association at the LY75 locus (r5147820690-T) was independent of other common and rare variant signals, joint fine-mapping (with FINEMAP) was performed on common and rare variants at this locus while including rarer variants then used in our genome-wide fine-mapping. In contrast to the genome-wide fine-mapping described above, this fine-mapping sensitivity analysis was done only in the UKB, was focused on the LY75 locus, and included all variants in the dataset. That is, the fine-mapping analysis was run as described above, but with a MAF >0.0000000001. While FINEMAP suggests 3 credible sets are most parsimonious at this locus (posterior probability=0.8), which is consistent with the results we report when preforming genome-wide fine-mapping, the fourth credible set (posterior probability=0.11) identifies rs147820690-T as the top signal (PIP=0.133) among 9,417 variants in the 95% credible set. This fine-mapping approach also prioritizes rs78446341-A (CPIP=0.92, CS=2). Furthermore, the median pairwise LD between SNPs in this fourth credible set is very low (6.7×10⁻⁴, compared with 0.995, 0.962, and 0.831 for the first three credible sets, respectively). Therefore, these fine-mapping results provide additional support for both LY75 missense variants, as well as the fact that the rs147820690-T rare variant signal is not driven by the tagging of other rare variants.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of preventing or reducing the development of clonal hematopoiesis of indeterminate potential (CHIP) in a subject, the method comprising administering a Lymphocyte Antigen 75 (LY75) inhibitor, a Cluster of Differentiation 164 (CD164) inhibitor, or a Poly(ADP-Ribose) Polymerase 1 (PARP1) inhibitor, or any combination thereof, to the subject.

2. The method according to claim 1, wherein the subject is at risk of developing a hematologic cancer, a myeloid neoplasia, a lymphoid neoplasia, an atherosclerotic cardiovascular disease, a coronary heart disease, a myocardial infarction, or severe calcified aortic valve stenosis.

3. The method according to claim 1, wherein: i) the LY75 inhibitor comprises an inhibitory nucleic acid molecule that hybridizes to an LY75 nucleic acid molecule; ii) the CD164 inhibitor comprises an inhibitory nucleic acid molecule that hybridizes to a CD164 nucleic acid molecule; and/or iii) the PARP1 inhibitor comprises an inhibitory nucleic acid molecule that hybridizes to a PARP1 nucleic acid molecule.

4. The method according to claim 3, wherein the inhibitory nucleic acid molecule comprises an antisense nucleic acid molecule, a small interfering RNA (siRNA), or a short hairpin RNA (shRNA).

5-26. (canceled)

27. The method according to claim 1, further comprising detecting the presence or absence of an LY75 variant nucleic acid molecule, a CD164 variant nucleic acid molecule, or a PARP1 variant nucleic acid molecule, in a biological sample from the subject.

28. The method according to claim 27, further comprising administering a therapeutic agent that prevents or reduces development of CHIP in a standard dosage amount to a subject wherein one or more of the LY75 variant nucleic acid molecule, CD164 variant nucleic acid molecule, or PARP1 variant nucleic acid molecule is absent from the biological sample.

29. The method according to claim 27, further comprising administering a therapeutic agent that prevents or reduces development of CHIP in a dosage amount that is the same as or less than a standard dosage amount to a subject that is heterozygous for one or more of the LY75 variant nucleic acid molecule, CD164 variant nucleic acid molecule, or PARP1 variant nucleic acid molecule.

30. The method according to claim 27, wherein the LY75 variant nucleic acid molecule, CD164 variant nucleic acid molecule, or PARP1 variant nucleic acid molecule is a missense variant, splice-site variant, a stop-gain variant, a start-loss variant, a stop-loss variant, a frameshift variant, or an in-frame indel variant, or a variant that encodes a truncated predicted loss-of-function polypeptide.

31. The method according to claim 30, wherein the LY75 variant nucleic acid molecule encodes a truncated LY75 predicted loss-of-function polypeptide, the CD164 variant nucleic acid molecule encodes a truncated CD164 predicted loss-of-function polypeptide, or the PARP1 variant nucleic acid molecule encodes a truncated PARP1 predicted loss-of-function polypeptide.

32. A method of treating a subject with a therapeutic agent that prevents or reduces development of CHIP, wherein the subject has CHIP or is at risk of developing CHIP, the method comprising the steps of:
determining whether the subject has an LY75 variant nucleic acid molecule, a CD164 variant nucleic acid molecule, and/or a PARP1 variant nucleic acid molecule by:
obtaining or having obtained a biological sample from the subject; and
performing or having performed a sequence analysis on the biological sample to determine if the subject has a genotype comprising the LY75 variant nucleic acid molecule, the CD164 variant nucleic acid molecule, and/or the PARP1 variant nucleic acid molecule; and
administering or continuing to administer the therapeutic agent that prevents or reduces development of CHIP in a standard dosage amount to a subject that is LY75 reference, CD164 reference, and/or PARP1 reference; and/or administering an LY75 inhibitor, a CD164 inhibitor, or a PARP1 inhibitor, or any combination thereof, to the subject;
administering or continuing to administer the therapeutic agent that prevents or reduces development of CHIP in an amount that is the same as or less than a standard dosage amount to a subject that is heterozygous for the LY75 variant nucleic acid molecule, the CD164 variant nucleic acid molecule, and/or the PARP1 variant nucleic acid molecule; and/or administering an LY75 inhibitor, a CD164 inhibitor, or a PARP1 inhibitor, or any combination thereof, to the subject; or
administering or continuing to administer the therapeutic agent that prevents or reduces development of CHIP in an amount that is the same as or less than a standard dosage amount to a subject that is homozygous for the LY75 variant nucleic acid molecule, the CD164 variant nucleic acid molecule, and/or the PARP1 variant nucleic acid molecule;
wherein the presence of a genotype having the LY75 variant nucleic acid molecule, the CD164 variant nucleic acid molecule, and/or the PARP1 variant nucleic acid molecule indicates the subject has a decreased risk of developing CHIP.

33. The method according to claim 32, wherein the subject is at risk of developing a hematologic cancer, a myeloid neoplasia, a lymphoid neoplasia, an atherosclerotic cardiovascular disease, a coronary heart disease, a myocardial infarction, or severe calcified aortic valve stenosis.

34. The method according to claim 32, wherein: i) when the subject is LY75 reference, the subject is administered or continued to be administered the therapeutic agent that prevents or reduces development of CHIP in a standard dosage amount, and is administered the LY75 inhibitor; ii) when the subject is CD164 reference, the subject is administered or continued to be administered the therapeutic agent that prevents or reduces development of CHIP in a standard dosage amount, and is administered the CD164 inhibitor; and/or iii) when the subject is PARP1 reference, the subject is administered or continued to be administered the therapeutic agent that prevents or reduces development of CHIP in a standard dosage amount, and is administered the PARP1 inhibitor.

35-36. (canceled)

37. The method according to claim 32, wherein: i) when the subject is heterozygous for the LY75 variant nucleic acid molecule, and the subject is administered or continued to be administered the therapeutic agent that prevents or reduces development of CHIP in an amount that is the same as or less than a standard dosage amount, and is administered the LY75 inhibitor; ii) when the subject is heterozygous for the CD164 variant nucleic acid molecule, the subject is administered or continued to be administered the therapeutic agent that prevents or reduces development of CHIP in an amount that is the same as or less than a standard dosage amount, and is administered the CD164 inhibitor; and iii) when the subject is heterozygous for the PARP1 variant nucleic acid molecule, the subject is administered or continued to be administered the therapeutic agent that prevents or reduces development of CHIP in an amount that is the same as or less than a standard dosage amount, and is administered the PARP1 inhibitor.

38-39. (canceled)

40. The method according to claim 32, wherein the LY75 variant nucleic acid molecule, CD164 variant nucleic acid molecule, or PARP1 variant nucleic acid molecule is a missense variant, splice-site variant, a stop-gain variant, a start-loss variant, a stop-loss variant, a frameshift variant, or an in-frame indel variant, or a variant that encodes a truncated predicted loss-of-function polypeptide.

41. The method according to claim 40, wherein the LY75 variant nucleic acid molecule encodes a truncated LY75 predicted loss-of-function polypeptide, the CD164 variant nucleic acid molecule encodes a truncated CD164 predicted loss-of-function polypeptide, or the PARP1 variant nucleic acid molecule encodes a truncated PARP1 predicted loss-of-function polypeptide.

42. The method according to claim 32, wherein: i) the LY75 inhibitor comprises an inhibitory nucleic acid molecule that hybridizes to an LY75 nucleic acid molecule; ii) the CD164 inhibitor comprises an inhibitory nucleic acid molecule that hybridizes to a CD164 nucleic acid molecule; and/or iii) the PARP1 inhibitor comprises an inhibitory nucleic acid molecule that hybridizes to a PARP1 nucleic acid molecule.

43. The method according to claim 42, wherein the inhibitory nucleic acid molecule comprises an antisense nucleic acid molecule, a small interfering RNA (siRNA), or a short hairpin RNA (shRNA).

44-138. (canceled)

139. A method of stratifying a suitable lung cancer patient for treatment with a PARP1 inhibitor, the method comprising determining whether the patient carries a DNMT3A R882H somatic mutation or TET2 somatic mutation deficiency, wherein the patient with the DNMT3A R882H somatic mutation or TET2 somatic mutation deficiency is excluded from a PARP1 inhibitor treatment regimen.
